(19) United States
(12) Patent Application Publication
(10) Pub. No.: US 2016/0288090 A1
(43) Pub. Date: Oct. 6, 2016

(54) MOLECULARLY IMPRINTED POLYMERS

(71) Applicants: Commonwealth Scientific and Industrial Research Organisation, Campbell (AU); Monash University, Clayton (AU)

(72) Inventors: Milton T. W. Hearn, Balwyn (AU); Steven Langford, Narre Warren North (AU); Kellie Louise Tuck, Murrumbeena (AU); Simon Harris, Bulleen (AU); Reinhard Ingemar Boysen, Wheelers Hill (AU); Victoria Tamara Perchyonok, Glen Huntly (AU); Basil Danylec, Box Hill North (AU); Lachlan Schwarz, Rowville (AU); Jamil Chowdhury, Skye (AU)

(73) Assignees: Commonwealth Scientific and Industrial Research Organisation, Campbell (AU); Monash University, Clayton (AU)

(21) Appl. No.: 15/017,737

(22) Filed: Feb. 8, 2016

Related U.S. Application Data
(62) Division of application No. 13/146,970, filed on Nov. 8, 2011, now abandoned.

(30) Foreign Application Priority Data
Jan. 29, 2009 (AU) 2009900328

(51) Int. Cl. B01J 20/26; B01D 15/38; B01J 20/30; C07C 37/82; C08F 226/06

(52) U.S. Cl. CPC B01J 20/268; C07C 37/82; C08F 226/06; B01J 20/3057; B01D 15/3852; A23L 1/0156; A23V 2002/00

(57) ABSTRACT

The present invention provides methods of designing molecularly imprinted polymers (MIPs) which have applications in extracting bioactive compounds from a range of bioprocessing feedstocks and wastes. The present invention is further directed to MIPs designed by the methods of the present invention.

### CROSS-REFERENCE TO RELATED APPLICATIONS

The present application claims priority from Australian Provisional Application No. 2009900328 the content of which is incorporated herein by reference.

### FIELD OF THE INVENTION

The present invention is directed to methods of designing molecularly imprinted polymers (MIPs) which have applications in extracting bioactive compounds from a range of bioprocessing feedstocks and wastes. The present invention is further directed to MIPs designed by the methods of the present invention.

### BACKGROUND OF THE INVENTION

Molecularly imprinted polymers (MIPs) can be designed with variable levels of cross-linking to produce polymers with controlled rigidity or flexibility, dependent upon the functional requirement, which contain cavities (binding sites) that can be tailor made to be specific for any target analyte. Using a pure sample of templates, MIPs can be prepared that are specific for the template compound or selective for other molecules having a similar chemical structure. MIPs are a polymeric network having high selectivity and specificity, which can be likened to that of an antibody for its antigen. MIPs offer the benefits of enhanced resistance to temperature, extremes of pH, solvents, proteases, and degradation or denaturation processes, which makes them reusable materials for the pre-concentration, extraction and separation of potential value-add biomolecules from complex feedstocks.

Strong epidemiological evidence exists for the health benefits of fruit and vegetables on cardiovascular disease, gastrointestinal tract cancers, cataracts and other diseases and ailments. Phytochemicals are candidates for contributing to a major part of this effect. Although knowledge of the mechanisms of uptake, metabolism and how these molecules exert their biological effects in vivo is uncertain, the health benefits afforded by these bioactive compounds is widely recognised and accepted.

One class of phytochemicals which are of considerable interest are the polyphenols, of which resveratrol is an example. Other interesting classes are the chalcones, indoles, coumarins, flavanones and flavanols, anthocyanins and the phytosterols and phytostanols.

With regard to resveratrol and its analogues or phytosterols and their analogues, there has been little published literature examining ways to produce MIPs of the required specificity. The literature that does exist predominately emphasises only the analytical mode of separation. Where attempts have been made to use the produced MIP in a preparative separation context, no attempt has been made to optimise the MIP performance or address some of the key challenges for larger scale use. For example, the work of Xiang et al.¹ is based on the concept that non-covalent molecular imprinting techniques are unsuitable for templates, such as resveratrol, dissolved in polar solvents, and hence it was necessary for acrylamide to be used as the monomer, whilst in the work of Ma et al.² no attention was given to control the selectivity of the derived MIP, with the consequence that the resveratrol derivatives, trans-polydatin and emodin, bound more strongly to the MIP despite their glucosidated or modified structures. The work of Cao et al.³ to a large extent simply restates the same conclusions of Xiang et al.¹ or Ma et al.², a situation which is not surprising in view of the use of very similar methods and an identical set of test compounds. There is thus a need for specific MIPs to be developed, which have been tailored and optimised for a particular template or class of templates with the derived molecularly imprinted polymers suitable for industrial-type applications. Specifically, there is a need to make suitable MIPs that are compatible with systems currently employed by the manufacturing industries, permitting the isolation of target compounds of commercial importance within the food and similar industries.

### SUMMARY OF THE INVENTION

The present inventors have designed a resveratrol-selective MIP (described as MIP 8 in this document) using resveratrol as template. They have also designed a number of other molecularly imprinted polymers with resveratrol analogue templates, including an imine analogue of resveratrol (called “green resveratrol” by the inventors), and an amide analogue. They have demonstrated the application of their MIP 8 in a solid phase extraction technique, allowing the selective enrichment of resveratrol from grape seed and peanut meal extract. This outcome can be contrasted to the low purification factors obtained with the MIP systems of the prior art, such as those described by Ma et al.², Xiang et al.¹ or Cao et al.³. In addition, they have demonstrated the ability to scale up the molecularly imprinted solid phase technique, thereby illustrating the scalability of the process.

In a first aspect, the present invention provides a method of preparing a molecularly imprinted polymer (MIP) having a desired level of specificity for a compound, the method comprising the steps of polymerizing a monomer comprising one or more non-covalent bonding sites and a cross-linking agent in the presence of a template molecule and porogen and subsequently removing the template, wherein the template is structurally analogous to the compound or comprises a moiety which is structurally analogous to the compound, and wherein the template comprises one or more non-covalent bonding sites wherein said non-covalent bonding sites are complementary to the non-covalent bonding sites of the monomer, and further wherein the template has either more or less non-covalent bonding sites than the compound, whereby the MIP has a different level of specificity for the compound than if the compound itself was used as the template.

In one embodiment, the present invention provides a method of preparing a molecularly imprinted polymer (MIP) having a desired level of specificity for a compound, the method comprising the steps of polymerizing a monomer comprising one or more hydrogen-bonding sites and a cross-linking agent in the presence of a template and porogen and subsequently removing the template, wherein the template is structurally analogous to the compound or comprises a moiety which is structurally analogous to the compound, and wherein the template comprises one or more hydrogen-bonding sites complementary to the one or more hydrogen-bonding sites of the monomer, and further wherein the template has either more or less hydrogen-bonding sites than the compound, whereby the MIP has a different level of specificity for the compound than if the compound itself was used as the template.

In a second aspect, the present invention provides a method of guiding the selection of a monomer for use in a molecularly imprinted polymer (MIP) which is to be imprinted with a template comprising one or more non-covalent bonding sites, wherein the MIP is to be prepared by polymerizing the selected monomer with a cross-linking agent in the presence of a template and porogen and subsequently removing the template, said method comprising the steps of providing a group of monomers having one or more non-covalent bonding sites which are complementary to the non-covalent bonding sites of the template, assessing the energy of formation of the complex formed between each monomer of the group of monomers and the template, and selecting the selected monomer from the number of monomers using the energy of formation of the complex as a factor in the selection.

In one embodiment, the present invention provides a method of guiding the selection of a monomer for use in a molecularly imprinted polymer (MIP) which is to be imprinted with a template comprising one or more hydrogen-bonding sites, wherein the MIP is to be prepared by polymerizing the selected monomer with a cross-linking agent in the presence of a template and porogen and subsequently removing the template, said method comprising the steps of providing a group of monomers having one or more hydrogen-bonding sites which are complementary to the hydrogen-bonding sites of the template, assessing the energy of formation of the hydrogen-bonded complex formed between each monomer of the group of monomers and the template, and selecting the selected monomer from the number of monomers using the energy of formation of the hydrogen-bonded complex as a factor in the selection.

In a third aspect, the present invention provides a method of selecting the ratio of monomers to template in the preparation of a molecularly imprinted polymer (MIP) which is to be imprinted with the template, wherein the MIP is to be prepared by polymerizing the monomer with a cross-linking agent in the presence of the template and porogen and subsequently removing the template, said method comprising the step of assessing the energy of formation of the complex formed between the template and a varying number of the monomers, and selecting the ratio of monomers to template using the energy of formation of the complex as a factor in the selection.

In a fourth aspect, there is provided a pre-polymerisation complex for use in preparing a MIP comprising one or more monomers each comprising one or more non-covalent bonding sites and a template wherein the template comprises one or more non-covalent bonding sites complementary to the one or more non-covalent bonding sites of the monomer.

In one embodiment, there is provided a pre-polymerisation hydrogen-bonded complex for use in preparing a MIP comprising one or more monomers each comprising one or more hydrogen-bonding sites and a template wherein the template comprises one or more hydrogen-bonding sites complementary to the one or more hydrogen-bonding sites of the monomer.

In a fifth aspect, there is provided a MIP prepared according to the method of the first aspect.

In one embodiment, the monomer comprising one or more non covalent-bonding sites is selected by the process of the second aspect.

In a sixth aspect, there is provided a MIP prepared by polymerizing a monomer with a cross-linking agent in the presence of a template and porogen and subsequently removing the template wherein the selection of the monomer is guided by the process of the second aspect or the ratio of monomer to template is selected by the process of the third aspect.

In a seventh aspect, there is provided a method of designing an analogue of a compound comprising a trans-ethylene linker, the method comprising replacing the trans-ethylene linker with an imine, amide or secondary amine linker.

Preferably, the trans-ethylene link is replaced with an imine link. The imine link is structurally equivalent to the ethylene link but is far simpler to synthesise.

In an eighth aspect, there is provided a method of preparing a MIP which is specific for a compound having a trans-ethylene linker, the method comprising the steps of polymerizing a monomer and a cross-linking agent in the presence of a template and porogen and subsequently removing the template, wherein the template is an analogue of the compound.

In a ninth aspect, the present invention provides a molecularly imprinted polymer (MIP) imprinted with a polyphenol or an analogue thereof wherein the MIP comprises polymerised 4-vinylpyridine together with a polymerised cross-linking agent.

In a tenth aspect, the present invention provides a method of preparing a MIP according to the ninth aspect, said method comprising the steps of
(i) polymerising the MIP in the presence of the polyphenol(s) or analogue(s) thereof and a porogen; and
(ii) removing the polyphenol(s) or analogue(s) thereof from the MIP.

In a eleventh aspect, the present invention provides a method of extracting one or more polyphenols from a sample by exposing the sample to a MIP according to the first aspect.

In a preferred form, the sample is plant based, and sourced from foodstuffs such as grapes and their seeds, skins, and juice (and wine), apples, pears, berries, and other fruits, tea, peanuts (or peanut meal), and cereals such as wheat, corn, rice and their oils, and byproducts thereof.

In one embodiment, the MIP is encased in a permeable mesh.

In an twelfth aspect, the present invention provides a method of at least partially separating the constituents of a sample by chromatography, the method comprising the step of (i) preparing a chromatographic column comprising a MIP according to the first aspect; (ii) passing the sample through the column; and (iii) collecting fractions of the sample from the column.

In a thirteenth aspect, the present invention provides a MIP imprinted with one or more compounds selected from the group consisting of sterols and stanols, and analogues or derivatives thereof, wherein said MIP comprises a polymerised monomer.

Preferably, the derivative is a polyphenol ester such as a ferulic acid ester or a gallic acid ester. The polyphenol provides hydrogen-bonding and π-π bonding sites.

In a fourteenth aspect, the present invention provides a method of preparing a MIP according to the thirteenth aspect, said method comprising the steps of

(i) polymerising the MIP in the presence of the sterol(s) or stanol(s), or analogue(s) or derivative(s) thereof, and a porogen; and
(ii) removing the sterol(s) or stanol(s), or analogue(s) or derivative(s) thereof, thereof from the MIP.

In a fifteenth aspect, the present invention provides a method of extracting one or more sterol(s) or stanol(s), or analogue(s) or derivative(s) thereof, from a sample by exposing the sample to a MIP according to the thirteenth aspect.

In one embodiment, the MIP is encased in a permable mesh.

In a sixteenth aspect, the present invention provides a method of at least partially separating the constituents of a sample by chromatography, the method comprising the step of (i) preparing a chromatographic column comprising a MIP according to the thirteenth aspect; (ii) passing the sample through the column; and (iii) collecting fractions of the sample from the column.

In the course of investigations into the functional polymers of this invention, the present inventors have prepared a number of novel and inventive compounds. The seventeenth aspect of this invention is directed to these novel compounds.

In an eighteenth aspect, there is provided a method of at least partially separating components of a sample comprising two or more of said components, said method comprising sequentially exposing the sample to at least two MIPs wherein each MIP has been imprinted with a different template.

In a nineteenth aspect, there is provided a MIP encased in a permeable mesh.

In a twentieth aspect, there is provided a method of extracting a component from a sample comprising exposing the sample to a MIP according to the nineteenth aspect.

In a twenty first aspect, there is provided a MIP imprinted with (E)-5-[(4-hydroxy-phenylimino)-methyl]-benzene-1,3-diol or 3,5-dihydroxy-N-(4-hydroxyphenyl)benzamide.

In a twenty second aspect, there is provided a method of extracting resveratrol from a sample, said method comprising exposing the sample to a MIP according to the twenty first aspect.

### BRIEF DESCRIPTION OF THE FIGURES

FIG. 1. Structure of resveratrol-3-O-β-D-glucuronide.

FIG. 2. Production of (E)-5-(4-(methacryloyloxy)styryl)-1,3-phenylene bis(2-methylacrylate) for forming “covalent” MIPS.

FIGS. 3A and 3B. Structure of reseveratrol (FIG. 3A) and computer generated model of resveratrol surface using PM3 calculations displaying electronic character and likely interaction sites (FIG. 3B).

FIG. 4. Molecular modelling titration results showing estimated interaction energy between resveratrol and 4VP clusters using PM3 geometry optimisation calculations showing a distinct minimum at three eq 4VP.

FIG. 5. Space filled image of a resveratrol complex with three 4VP units interacting through each of the stilbene hydroxyl and pyridinyl nitrogen head groups. Calculations did not include solvent considerations and should only be used as a guide.

FIGS. 6A-6D. Modelling titration data for the estimated net interaction energies (ΔE_{f}) for the complexes between resveratrol and the functional monomers acrylamide (AAM) (FIG. 6A) and 4-vinylbenzoic acid (4VBA) (FIG. 6B) using semi-empirical calculations using a PM3 force field. In addition to this the static binding data for 50 mg of polymer in a solution of 0.5 min resveratrol in acetonitrile is shown (FIGS. 6C and 6D).

FIG. 7. Downfield movement of the aromatic —OH shift of resveratrol upon addition of increasing molar equivalents of the functional monomer 4VP. Indicates that a pre-polymerisation complex is forming which is likely through H bonding interactions with the pyridinyl N group.

FIG. 8. Rebinding data for the polymers MIP 1, MIP 2, MIP 3 and MIP 4 and corresponding NIPs displaying resveratrol affinity from a resveratrol solution (0.05 min) in acetonitrile.

FIG. 9. Resveratrol binding affinity to polymers MIP 1, MIP 2, MIP 3, MIP 4 NIP 1 and NIP 2 in a binding assay using 0.5 min resveratrol solution in acetonitrile.

FIG. 10. Static rebinding data for MIP and their respective NIP control materials prepared with the following T:FM:XL ratios: 1:3:15 (MIP 8), 1:3:17 (MIP 10), 1:3:20 (MIP 9), 1:3:30 (MIP 2) and 1:3:40 (MIP 5).

FIG. 11. Static Binding data for multiple MIP 8 batches A-J (LS3-37p84-17-4-08a-j), K (LS3-26p62-1-4-08a), L (LS3-26p62-1-4-08b) and M (1s2-6p10-7-2-07) indicating the reproducibility in MIP production and binding performance in acetonitrile. All measurements were conducted in duplicate.

FIG. 12. Resveratrol binding capacity under static conditions to MIP 8 (30 mg) over a range of resveratrol concentrations in ACN up to 5 min. Samples were mixed overnight (18 hours) using a suspension mixer at approximately 40 rpm. Samples were then centrifuged, and an aliquot of the supernatant analysed by HPLC to determine the free resveratrol concentration.

FIGS. 13A-13C. Cross-reactivity binding data for MIP 8 under static conditions for a range of resveratrol analogues (0.5 min) in acetonitrile without competition.

FIG. 14. Static binding data comparing resveratrol affinity between a resveratrol imprinted polymer (MIP 8) and MIPs templated with resveratrol analogues.

FIG. 15. Results from MISPE binding assay comparing both the effect of cross-linking amount and influence of ethanol in the porogen (MIP_{E}) under dynamic SPE conditions after removal of non-specific binding via extensive washing with acetonitrile.

FIGS. 16A and 16B. MISPE binding of resveratrol (0.5 min) under aqueous conditions with resveratrol bound (pmole/g polymer) after washing with: (FIG. 16A) loading solution; and (FIG. 16B) 2×20% (v/v) ethanol in water.

FIGS. 17A and 17B. The effect of ethanol and water content on the retention/binding of resveratrol under aqueous conditions. Binding was assessed using resveratrol solutions (0.5 min) in 20, 50 and 80% ethanol (v/v), respectively, to MIP 8. The data shown refers to percentage of the initial resveratrol solution bound after (FIG. 17A) loading and (FIG. 17B) clean up washes comprising 1 mL loading solution (i.e 20, 50 and 80% ethanol) and three consecutive washes of 50% aqueous ethanol (v/v).

FIGS. 18A-18D. Demonstrate that MIP 8 is capable of concentrating resveratrol from a complex feedstock containing multiple potential competitors for available binding sites. The diagram in FIG. 18A shows the HPLC trace of spiked grape seed extract with large amounts of unknown species masking resveratrol; FIG. 18B shows the resveratrol (RT 2.735 min) eluted after applying a resveratrol standard solution (0.5 min); FIG. 18C shows resveratrol (RT 2.735 min) eluted from MISPE column after application of spiked grape seed extract; and FIG. 18D shows the results after application of spiked grape seed to the respective NISPE column resulting in no observable peak at 2.735 min, indicating that no resveratrol was retained.

FIGS. 19A-19C. Chromatograms of (FIG. 19A) untreated peanut meal, (FIG. 19B) MISPE treated peanut meal and (FIG. 19C) peanut meal extract after SPE treatment using a non-imprinted control polymer (NIP) as stationary phase.

FIGS. 20A-20C. Chromatograms of (FIG. 20A) untreated peanut meal with resveratrol concentration of approximately 0.98 μg/mL of peanut meal extract, (FIG. 20B) MISPE treated peanut meal resulting in resveratrol concentration of approximately 19.5 μg/mL and (FIG. 20C) peanut meal extract after SPE treatment using a non-imprinted control polymer (NIP) as stationary phase.

FIG. 21. The chemical structures of cholesterol and the commonly found phytosterols β-sitosterol, stigmasterol, campesterol and brassicasterol and the phytostanols β-sitostanol and campestanol.

FIG. 22. The chemical structures of the main components present in γ-Oryzanol.

FIG. 23. Performance of MIP19 (black) and NIP19 (grey) (Ch:4-VP:Crosslinker)

FIG. 24. Performance of MIP20 (black) and NIP20 (grey) (Ch:MMA:Crosslinker)

FIG. 25. Performance of MIP20 (black) and NIP20 (grey) with stigmasterol as rebinding molecule.

FIG. 26. Performance of MIP19 (black) and NIP19 (grey) with stigmasterol as rebinding molecule.

FIG. 27. Performance of MIP22 (black) and NIP22 (grey) with stigmasterol as rebinding molecule.

FIG. 28. Performance of MIP21 (black) and NIP21 (grey) with stigmasterol as rebinding molecule.

FIG. 29. Performance of MIP21 (black) and NIP21 (grey) with cholesterol as rebinding molecule.

FIG. 30. Performance of MIP20 (black) and NIP20 (grey) with cholesterol as rebinding template and 2 cycles of rebinding.

FIG. 31. Conversion of phytosterols to the corresponding ferulate esters.

FIG. 32. Performance of MIP23 (black) and NIP23 (grey) with cholesteryl ferrulate as a rebinding template (4-VP as a functional monomer).

FIG. 33. Performance of MIP24 (black) and NIP24 (grey) with cholesteryl ferrulate as a rebinding template (methacrylic acid as a functional monomer).

FIG. 34. Performance of covalent polymers (1:10 (FM: EDGMA): MIP7 (black) and NIP7 (grey)

FIG. 35. Performance of hybrid polymer MIP 26 (black) and NIP 26 (grey) with cholesterol as a rebinding substrate previously described.

FIG. 36. Modelling titration data for (E)-resveratrol against the functional monomers 4-Vinylpyridine (4VP, ), acrylamide (AAM, ♦), methacrylic acid (MAA, ▪), methylmethacrylate (MMA, +) and styrene (Sty, Δ) showing predicted ΔEᵢ values for monomer equivalents ranging from 1-6.

FIGS. 37A and 37B. (FIG. 37A) Static binding isotherms multiple batches for the binding of (E)-resveratrol with multiple batches of P1. Measurements were determined in duplicate with a minimum of 3 replicates. Error bars indicate the standard error of the mean (SEM). (FIG. 37B) Selective capacity of the MIP for (E)-resveratrol: the inset shows the binding data in Scatchard (34) format.

FIG. 38. Binding performance of imprinted and non-imprinted polymers towards (E)-resveratrol under static conditions.

FIG. 39. Static binding from single analyte assays showing the amount of bound analyte per gram of polymer for (E)-resveratrol 1 and seven polyhydroxy stilbene structural analogues by the MIP P1 (black) and the NIP control N1 (grey).

FIG. 40. Single analyte selectophore binding of alkene 1, amide 2, and imine 3, with MIP_{RES}.

FIG. 41. Recognition of (E)-Resveratrol by MIP_{RES}, MIP_{AMIDE} and MIP_{IMINE}.

FIG. 42. Relative binding capacity of MIP_{RES} for (E)-resveratrol. (E)-resveratrol standard (0.5 μmol) was load onto MIP_{RES} (100 mg) in either acetonitrile (black) or EtOH/H₂O (1:1, v/v) (grey) respectively. Each column was subsequently washed using the loading solvent and the amount of (E)-resveratrol remaining on-column determined from a 5 point calibration curve.

FIG. 43. RP-HPLC chromatograms of peanut meal extract and MISPE eluates: untreated peanut meal extract (front line), eluate from MIP_{RES} MISPE column (middle line) and eluate from NISPE column (back line). Chromatograms were obtained at 321 nm (E)-resveratrol elutes at Rₜ=12.2 mins.

FIGS. 44A and 44B. (FIG. 44A) Static binding isotherms for the binding of (E)-resveratrol to MIP_{AMIDE} (▴) and NIP_{AMIDE} (▴); (FIG. 44B) comparison of the selective affinity of both MIP_{AMIDE} (▴) and MIP_{RES} (♦) for (E)-resveratrol, expressed as the selectivity (MIP-NIP).

FIG. 45. Cross-reactivity studies on MIP_{RES}, MIP_{AMIDE} and their respective NIP control polymers for (E)-resveratrol 1, 3,5-dihydroxy-N-(4-hydroxyphenyl)benzamide 2, catechin 3 and (E)-piceid 4.

FIGS. 46A-46D. RP-HPLC chromatograms using EtOH/H₂O (8:2 v/v) as mobile phase solvent B with UV-Vis detection at 321 nm for (FIG. 46A) peanut meal extract and eluates from (FIG. 46B) MIP_{RES}, (FIG. 46C) MIP_{AMIDE}, and (FIG. 46D) NIP_{RES} control columns. (E)-resveratrol elutes at Rₜ=12.2 min. The identity of (E)-resveratrol was confirmed by LC-ESI-MS.

FIG. 47. Schematic showing the concept for sequential MISPE treatment of extracts obtained from complex food sources, whereby multiple bioactive components may be separated from a single source.

FIGS. 48A-48C. RP-HPLC chromatograms of (FIG. 48A) the peanut meal extract (10 g/500 mL) in EtOH/H₂O (1:1 v/v) and acid elution of molecules captured by (FIG. 48B) MIP_{RES} then (FIG. 48C) MIP_{AMIDE}. Chromatograms were obtained by UV-Vis detection at λ=321 nm (E)-resveratrol elutes at Rₜ=17 minutes.

FIGS. 49A-49C. RP-HPLC chromatograms of (FIG. 49A the resveratrol-depleted peanut meal extract resulting from the first round of MISPE processing and acid elution of molecules captured by (FIG. 49B) MIP_{RES} then (FIG. 49C) MIP_{AMIDE}. Chromatograms were obtained by UV-Vis detection at λ=321 nm. A-type procyanidin elutes at R_{f}=15 minutes.

FIG. 50. Schematic depiction of a sequential SPE format MIP column configuration for the isolation of multiple target compounds from a feed extract. All undesirable species pass through each MIP column leaving multiple target species bound to their respective MISPE columns, which can then be eluted to yield each of the respective target compounds in high purity.

### DETAILED DESCRIPTION OF THE INVENTION

The formation of a MIP typically involves the following steps: a template is mixed together with a monomer (typically in excess) to form pre-polymerisation complexes comprising a single template associated with a number of monomer molecules; these pre-polymerisation complexes are then cross-linked into a polymer by polymerisation of the monomers with a cross-linking agent; and the template is subsequently removed from the cross-linked complexes. The cross-linked monomers of the complexes comprise surfaces and cavities which are complementary to the shape of the template. The cross-linking reaction is generally carried out in the presence of porogen which ensures that the MIP has an open structure comprising a number of pores. These pores allow the molecules of a solution to move through the MIP so that the molecules are able to interact with the surfaces and cavities. An analyte which has the same or similar shape to the template will more strongly interact with the MIP than other molecules in the solution, so that the MIP can be used to capture, concentrate and/or separate the analyte.

Various different types of manufacture can be employed, e.g. (a) monolith type preparation, (b) particles made by grinding and sizing, (c) precipitation polymerization procedures.

The present inventors have synthesised a series of compounds based on resveratrol to generate a library of compounds, which may be present in crude bioprocessing feedstocks, and which have been used as templates for creating and investigating MIPs as tailor-made affinity adsorbents for bioactives derived from plant and other biological sources. All of these molecules can be synthesised to incorporate variable functionality characteristics, based on hydrophobicity, hydrophilicity and other physicochemical parameters, to produce molecules with defined composition-of-matter. These chemical compounds can be designed, synthesised and derivatised with a variety of different functionalities to include inter alia amino, carboxyl, hydroxyl, alkyl or aryl substituents distributed throughout the points of diversity on the respective scaffolds to create a subset of compound analogues which can be used as templates for the design and characterisation of MIPs. These functionalities may be selected on the basis of both steric and electronic considerations. Compounds with an increased number of polar functional groups or “points” are more likely to be instructive in probing and defining the created MIP cavities. Increasing the number of points available for interaction with functional monomer molecules during the MIP templating process potentially increases the specificity of the cavity formed for any defined template.

As an illustration of the generality of methods of the present invention, the present inventors have also demonstrated that sterols and stanols esterified with polyphenol acids, such as gallic acid and ferulic acid, can be used as templates having additional “points” when compared to the sterol or stanol as such.

As would be clear to those skilled in the art, the techniques used are applicable to molecules other than resveratrol, the sterols and stanols.

Accordingly, in a first aspect, the present invention provides a method of preparing a molecularly imprinted polymer (MIP) having a desired level of specificity for a compound, the method comprising the steps of polymerizing a monomer comprising one or more non-covalent bonding sites and a cross-linking agent in the presence of a template and porogen and subsequently removing the template, wherein the template is structurally analogous to the compound or comprises a moiety which is structurally analogous to the compound, and wherein the template comprises one or more non-covalent bonding sites wherein said non-covalent bonding sites are complementary to the non-covalent bonding sites of the monomer, and further wherein the template has either more or less non-covalent bonding sites than the compound, whereby the MIP has a different level of specificity for the compound than if the compound itself was used as the template.

The term ‘non-covalent bonding site’ means a group or region of the molecule which is capable of a non-covalent bonding interaction. Examples of non-covalent bonding interactions include hydrogen bonding interactions, π-π bonding interactions, Lifshitz force interactions and van der Waals interactions.

Preferably, the non-covalent bonding site is a hydrogen-bonding site or a π-π bonding interaction site. More preferably, the non-covalent bonding site is a hydrogen-bonding site.

The term “complementary” means that a site on the template is able to bind via the non-covalent bonding interaction to a site on the monomer and vice versa.

In the circumstance where the template comprises a moiety which is structurally analogous to the compound, then the non-covalent bonding site(s) may be located (i) on the moiety, (ii) on the remaining part of the template, or (iii) if there are two or more non-covalent bonding sites, on both the moiety and on the remaining part of the template.

For instance, as will be seen in the Examples, the present inventors have used ferulic acid esters of sterols and stanols to prepare MIPs according to the present invention. These compounds comprise a moiety which is structurally analogous to the sterol or stanol (i.e. the sterol or stanol itself) with the remaining part of the molecule providing non-covalent bonding sites. The ferulic acid moiety provides two hydrogen-bonding sites and a π-π bonding interaction site.

In one embodiment, the present invention provides a method of preparing a molecularly imprinted polymer (MIP) having a desired level of specificity for a compound, the method comprising the steps of polymerizing a monomer comprising one or more hydrogen-bonding sites and a cross-linking agent in the presence of a template and porogen and subsequently removing the template, wherein the template is structurally analogous to the compound or comprises a moiety which is structurally analogous to the compound, and wherein the template comprises one or more hydrogen-bonding sites complementary to the one or more hydrogen-bonding sites of the monomer, and further wherein the template has either more or less hydrogen-bonding sites than the compound, whereby the MIP has a different level of specificity for the compound than if the compound itself was used as the template.

In one embodiment, the hydrogen-bonding site of the template is a phenolic hydroxyl group.

In another embodiment, the hydrogen-bonding site of the monomer is a pyridine nitrogen.

In a preferred form, the template further comprises one or more π-π bonding interaction sites and the monomer further comprises one or more complementary π-π bonding interaction sites.

In one embodiment, the π-π bonding interaction site of the template is the aromatic ring of a phenol. In another embodiment, the π-π bonding interaction site of the monomer is the aromatic ring of a pyridine.

The present inventors have found that a MIP formed using a template which is structurally analogous to the compound or comprises a moiety which is structurally analogous to the compound and further wherein the template comprises more hydrogen bonding sites than the compound will tend to be more specific for the compound (and for compounds with a very closely related structure) than if the compound itself is used as the template. This is because the additional hydrogen-bonding sites on the template will attract more monomer units to the pre-polymerisation complex so that the cavity formed by the template will conform more closely to the template and hence the compound and compensates for the loss of a potential binding site in cases where the opening of the cavity by chance is positioned such that one binding site of the template has no complementary binding site in the polymer.

Accordingly, in one embodiment, the template has more hydrogen-bonding sites than the compound, whereby the MIP has a greater level of specificity for the compound than if the compound itself was used as the template.

Further, if the MIP is formed using a template which is structurally analogous to the compound or comprises a moiety which is structurally analogous to the compound and the template comprises less hydrogen-bonding sites than the compound, then the MIP will tend to be less specific for the compound than if the compound itself is used as the template. Although the MIP is less specific for the compound itself, it will still possess a degree of specificity for the compound and also for compounds which have some structural similarities. This provides a means of extracting classes of potentially useful compounds from complex feed stocks. If desired, the extracted mixture comprising such a class of compounds can then be subjected to further purification techniques, such as extraction with more selective MIPs, to isolate sub-classes or individual molecules.

Accordingly, in another embodiment, the template has less hydrogen-bonding sites than the compound, whereby the MIP has a lower level of specificity for the compound than if the compound itself was used as the template.

As would be appreciated by the person skilled in the art, the methods of the present invention allows for the formation of MIPs which show improved levels of specificity for compounds which have no or fewer hydrogen-bonding sites by using, as templates, appropriately chosen structurally analogous compounds which do have a greater number of hydrogen-bonding sites. This provides a useful means of isolating compounds which are otherwise difficult to extract from complex mixtures.

In terms of relative abundance, a majority of compounds of interest as high value products within the food, fine chemical and pharmaceutical industries are likely to contain at least one hydrogen bonding site. Some contaminants, particularly toxic or hazardous compounds, such as polycyclic aromatics, are likely to fall into those families that lack hydrogen bonding sites and thus it will be essential in these cases to exploit other modes of molecular interaction. As would be understood to those skilled in the art, the approach taken with hydrogen-bonding is equally applicable to other types of non-covalent bonding and can be used to design MIPs which are selective for compounds such as polycyclic hydrocarbons.

In a second aspect, the present invention provides a method of guiding the selection of a monomer for use in a molecularly imprinted polymer (MIP) which is to be imprinted with a template comprising one or more non-covalent bonding sites, wherein the MIP is to be prepared by polymerizing the selected monomer with a cross-linking agent in the presence of a template and porogen and subsequently removing the template, said method comprising the steps of providing a group of monomers having one or more non-covalent bonding sites which are complementary to the non-covalent bonding sites of the template, assessing the energy of formation of the complex formed between each monomer of the group of monomers and the template, and selecting the selected monomer from the number of monomers using the energy of formation of the complex as a factor in the selection.

The design of molecularly imprinted polymers (MIPs) requires the selection of a monomer species that will interact favourably with the intended template species, such that a pre-polymerisation complex is formed between template and monomer. The use of tools such as molecular modelling and NMR spectroscopy of the pre-polymerisation complexes allow the selection of appropriate monomers by performing a ‘virtual screen’, which reduces the number of actual polymer preparations that are required to achieve optimised MIP development.

In one embodiment, the present invention provides a method of guiding the selection of a monomer for use in a molecularly imprinted polymer (MIP) which is to be imprinted with a template comprising one or more hydrogen-bonding sites, wherein the MIP is to be prepared by polymerizing the selected monomer with a cross-linking agent in the presence of a template and porogen and subsequently removing the template, said method comprising the steps of providing a group of monomers having one or more hydrogen-bonding sites which are complementary to the hydrogen-bonding sites of the template, assessing the energy of formation of the hydrogen-bonded complex formed between each monomer of the group of monomers and the template, and selecting the selected monomer from the number of monomers using the energy of formation of the hydrogen-bonded complex as a factor in the selection.

In a third aspect, the present invention provides a method of selecting the ratio of monomers to template in the preparation of a molecularly imprinted polymer (MIP) which is to be imprinted with the template, wherein the MIP is to be prepared by polymerizing the monomer with a cross-linking agent in the presence of the template and porogen and subsequently removing the template, said method comprising the step of assessing the energy of formation of the complex formed between the template and a varying number of the monomers, and selecting the ratio of monomers to template using the energy of formation of the complex as a factor in the selection.

In a preferred form of the second and third aspects, the energy of formation of the complex is assessed by molecular modeling techniques.

For instance, PM3 geometry optimization may be used to yield theoretical energy of formation values for the complex.

In another preferred form of the second and third aspects, the energy of formation is assessed by NMR-spectroscopy techniques. For instance, in the case of phenolic hydroxyl groups, the magnitude of the downfield shift of the ¹H NMR signal for that group is indicative of the strength of the hydrogen-bonding interactions. Typically, the downfield shift would be in the order of about 0.5 to about 1.5 ppm although, as would be understood by those skilled in the art, the choice of NMR solvent will be a important factor in determining the magnitude of the shift.

In a fourth aspect, there is provided a pre-polymerisation complex for use in preparing a MIP comprising one or more monomers, each comprising one or more non-covalent bonding sites, and a template wherein the template comprises one or more non-covalent bonding sites complementary to the one or more non-covalent bonding sites of the monomer.

In a preferred form, the monomers are selected by the method according to the second aspect.

In another preferred form, the ratio of monomer to template is selected by the process of the third aspect.

In one embodiment, there is provided a pre-polymerisation hydrogen-bonded complex for use in preparing a MIP comprising one or more monomers each comprising one or more hydrogen-bonding sites and a template wherein the template comprises one or more hydrogen-bonding sites complementary to the one or more hydrogen-bonding sites of the monomer.

In a fifth aspect, there is provided a MIP prepared according to the method of the first aspect.

In one embodiment, the monomer comprising one or more non-covalent bonding sites is selected by the process of the second aspect.

In a sixth aspect, there is provided a MIP prepared by polymerizing a monomer with a cross-linking agent in the presence of a template and porogen and subsequently removing the template wherein the selection of the monomer is guided by the process of the second aspect or the ratio of monomer to template is selected by the process of the third aspect.

The monomers used in the methods and MIPs of the present invention include those that promote or facilitate hydrogen-bonding interactions and/or π-π bonding interactions and include:

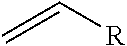

where R is selected from the group consisting of C₁₋₄alkyl, amide, nitrile, carboxylic acid, primary or secondary amine, CO₂C₁₋₄alkyl, C₁₋₄OH, hydroxyalkyl acrylate, benzene, benzyl amine, naphthalene, anthracene, pyridine, pyrimidine, purine, N-imidazole; and

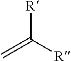

where R′ is selected from the group consisting of H and CH₃;
and R″ is selected from the group consisting of C₁₋₄alkyl, amide, nitrile, carboxylic acid, primary or secondary amine, CO₂C₁₋₄alkyl, C₁₋₄OH, and hydroxyalkyl methacrylate.

In a preferred form, the monomer is selected from the group consisting of:

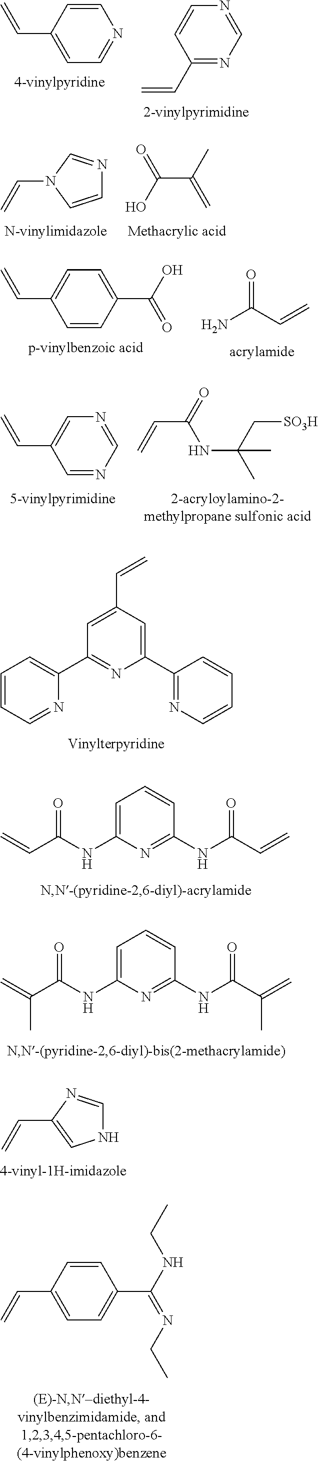

The person skilled in the art would be aware of a wide range of cross-linking agents that would be suitable for use in the methods and MIPs of the present invention. In a preferred embodiment, the cross-linking agent is selected from the group consisting of:

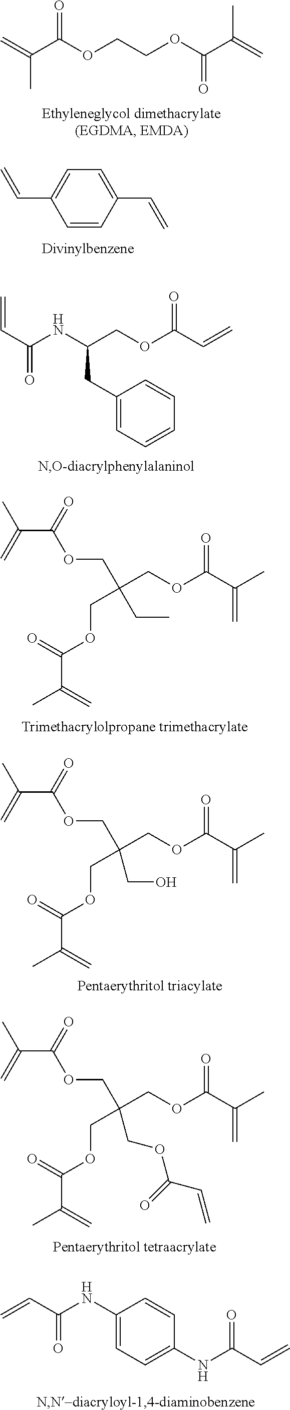

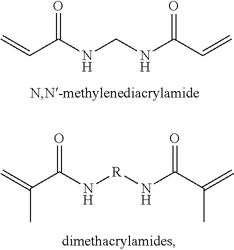

where R is an alkyl chain, preferably 1 to 4 carbons in length

In certain embodiments, the monomer and the cross-linking agent may be the same compound. Porogens suitable for use in the methods and MIPs of the present invention include those that promote or facilitate hydrogen-bonding interactions and those that promote or facilitate hydrophobic interactions or a combination of both.

Porogens that facilitate hydrogen bond formation include acetonitrile, acetone, ethyl acetate and dimethyl formamide (DMF) or a combination thereof, in addition to mixtures of the above with suitable quantities of ethanol, methanol or dimethyl sulfoxide (DMSO).

Porogens that facilitate hydrophobic interactions include aqueous mixtures of acetonitrile, acetone, ethyl acetate, DMF, ethanol, methanol, DMSO or a combination thereof.

With respect to phytosterols (or other classes or families of compounds) the above list can be extended with the inclusion also of water, optionally mixed with trifluoroacetic acid. Organic solvents such as chloroform, dichloromethane, hexane, toluene and the more polar organic solvents such as isopropanol, tertiary butyl alcohol and cyclohexanol may also be used.

In a seventh aspect, there is provided a method of designing an analogue of a compound comprising a trans-ethylene linker, the method comprising replacing the trans-ethylene linker with an imine, amide or secondary amine linker.

Preferably, the compound is resveratrol.

In an eighth aspect, there is provided a method of preparing a MIP which is specific for a compound having a trans-ethylene linker, the method comprising the steps of polymerizing a monomer and a cross-linking agent in the presence of a template and porogen and subsequently removing the template, wherein the template is an analogue of the compound and further wherein the analogue is designed according to the method of the seventh aspect.

In a ninth aspect, the present invention provides a molecularly imprinted polymer (MIP) imprinted with a polyphenol or an analogue thereof wherein the MIP comprises polymerised 4-vinylpyridine together with a polymerised cross-linking agent.

Preferably, the polyphenol or an analogue thereof is resveratrol or an analogue thereof. More preferably, the polyphenol or analogue thereof is an analogue of resveratrol where the trans-ethylene linker is replaced with an imine, amide or secondary amine linker. In certain embodiments, the analogue has more or less hydroxyl groups than resveratrol. In a more preferred form, the analogue of resveratrol is the imine (E)-5-[(4-hydroxy-phenylimino)-methyl]-benzene-1,3-diol or the amide 3,5-dihydroxy-N-(4-hydroxyphenyl)benzamide.

The imine and amide anlogues of resveratrol analogues are simpler and cleaner to synthesise than resvertrol itself. MIPs prepared using these templates have been shown to have a comparable binding affinity for resveratrol as MIPs prepared using resveratrol as a template.

In a preferred from, the MIP is for use in isolating resveratrol.

Preferably, when resveratrol is used as a template, the ratio of resveratrol to 4-vinylpyridine is 1:3. This ratio has been shown by modelling studies and by empirical results to be the optimum ratio for a resveratrol binding MIP. Similarly, the ratio of the imine (E)-5-[(4-hydroxy-phenylimino)-methyl]-benzene-1,3-diol and the amide 3,5-dihydroxy-N-(4-hydroxyphenyl)benzamide to 4-vinylpyridine is preferably 1:3.

Various classes of compounds can be used as templates for resveratrol. These include:

(a) the chalcones

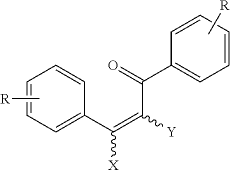

(b) the amide analogue linked by —NH—C(O)—;

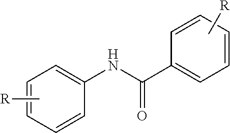

(c) cyclic systems such as the flavanols and coumarins, ie

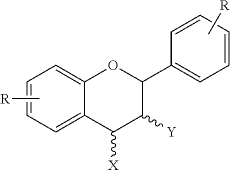

(d) alkaloids such as the nitrogen containing indoles, the rings of the indoles being substituted with R groups;
(e) other cyclic analogues are also relevant, such as the products from the following model reaction

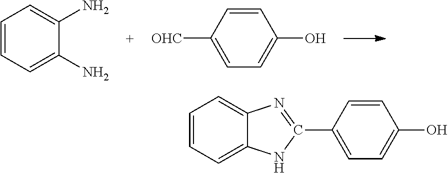

generating benzimidazoles, which are analogues of resveratrol. Each phenyl ring of the bezimidazole can additionally bear an R substituent.

In these compounds; each R may be zero (O) to three (3) substituents each of which is independently selected from the group consisting of: H, OH, CH₃, NH₂, SH, NO₂, COOH, C(O)NH₂, CHO, CN, NC, OCH₃, OC₁₋₄alkyl, SC₁₋₄alkyl, O-Sugar, N-Sugar, P(O)(OH)₂, S(O)₂(OH), OAr, NHAr, SAr, NHC₁₋₄alkylAr, OC(O)Ar, C(O)Oar, C(O)Ar, C(O)Nar, CF₃, OCF₃, Halogen, NHC₁₋₄alkyl, N(C₁₋₄alkyl)₂, SC₁₋₄alkyl, C(O)C₁₋₄alkyl, OC(O)C₁₋₄alkyl, C(O)OC₁₋₄alkyl, C(O)NHC₁₋₄alkyl, C(O)N(C₁₋₄alkyl)₂, C₁₋₄alkoxy, C₁₋₄alkylenedioxy, P(O)(OH)₂, P(O)(OC₁₋₄alkyl)₂, S(O)(OC₁₋₄alkyl)₂, C(NH₂)═C(NH₂)₂, C(NH₂)═C(CN)₂, C(CN)═C(CN)₂, C(CN)═C(NH₂)₂, C(NH₂)═C(C₁₋₄alkyl)₂, C(C₁₋₄alkyl)═C(NH₂)₂, C(CN)═C(C₁₋₄alkyl)₂, C(C₁₋₄alkyl)═C(CN)₂; and OR₁

wherein R₁ may be selected from the group consisting of H, Ac, glucose, galactose, gallate, ferulate.

X and Y indicate appropriate substituents. Typically, H or C₁₋₆alkyl. In the case of the coumarins, X or Y may be ═O and the oxygen containing ring may contain an additional double bond.

Conditions have been established and optimized for the synthesis of a small library of polyfunctionalized (E)-stilbene (resveratrol) analogues using a convergent methodology. The synthetic procedures have introduced various functional groups into the core scaffolds, which has resulted in the production of a library of low molecular weight compounds having unique compositions of matter. A summary of the molecules produced to date is shown in Table 1. These compounds, together with their saturated diphenethyl analogues, have been used as templates for the creation of molecularly imprinted polymers and as probes to investigate the binding characteristics of these MIPs.

TABLE 1
Structures of resveratrol analogues that have been synthesised with
0-4 ‘points’ for interaction with monomer during MIP formation.

| Molecule | | |
|---|---|---|
| Class | Structure | Comments |
| 0-Point | 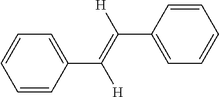 [SMILES: C(=C\c1ccccc1)/c1ccccc1] | 1. (E)-stilbene |
| | 1 | |
| | 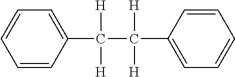 [SMILES: c1ccc(CCc2ccccc2)cc1] | 2. 1,2-diphenylethane |
| | 2 | |
| 1-Point | 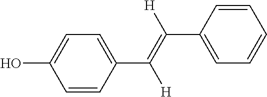 [SMILES: Oc1ccc(/C=C/c2ccccc2)cc1] | 3. (E)-4-hydroxystilbene |
| | 3 | |
| | 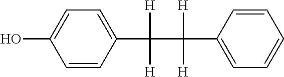 [SMILES: Oc1ccc(CCc2ccccc2)cc1] | 4. 4-phenethylphenol |
| | 4 | |
| | 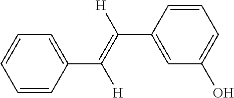 [SMILES: Oc1cccc(/C=C/c2ccccc2)c1] | 5. (E)-3-hydroxystilbene |
| | 5 | |
| | 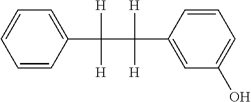 [SMILES: Oc1cccc(CCc2ccccc2)c1] | 6. 3-phenethylphenol |
| | 6 | |
| 2-Point | 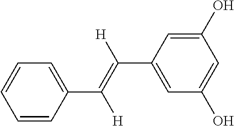 [SMILES: Oc1cc(O)cc(/C=C/c2ccccc2)c1] | 7. (E)-3,5- dihydroxystilbene |
| | 7 | |
| | 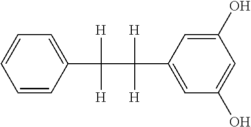 [SMILES: Oc1cc(O)cc(CCc2ccccc2)c1] | 8. 5-phenethylbenzene- 1,3-diol |
| | 8 | |
| | 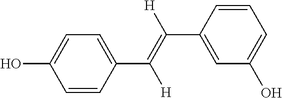 [SMILES: Oc1ccc(/C=C/c2cccc(O)c2)cc1] | 9. (E)-3,4′- dihydroxystilbene |
| | 9 | |
| | 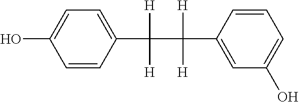 [SMILES: Oc1ccc(CCc2cccc(O)c2)cc1] | 10. 3,4′-(ethane-1,2- diyl)diphenol |
| | 10 | |
| 3-Point | 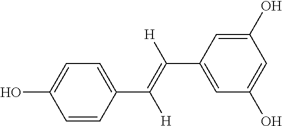 [SMILES: Oc1ccc(/C=C/c2cc(O)cc(O)c2)cc1] | 11. (E)-3,4′,5- trihydroxystilbene |
| | 11 | |
| | 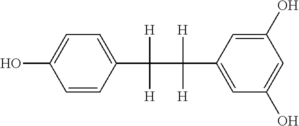 [SMILES: Oc1ccc(CCc2cc(O)cc(O)c2)cc1] | 12. 4-(3,5- dihydroxyphenethyl)- phenol |
| | 12 | |
| | 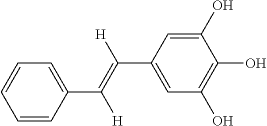 [SMILES: Oc1cc(/C=C/c2ccccc2)cc(O)c1O] | 13. (E)-3,4,5- trihydroxystilbene |
| | 13 | |
| | 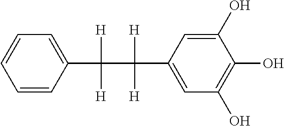 [SMILES: Oc1cc(CCc2ccccc2)cc(O)c1O] | 14. 5-phenethylbenzene- 1,2,3-triol |
| | 14 | |
| | 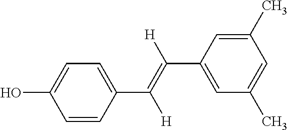 [SMILES: Cc1cc(C)cc(/C=C/c2ccc(O)cc2)c1] | 15 (E)-3,5-dimethyl-4′- hydroxystilbene |
| | 15 | |
| | 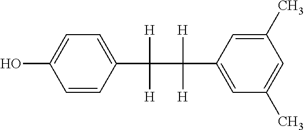 [SMILES: Cc1cc(C)cc(CCc2ccc(O)cc2)c1] | 16 4-(3,5- dimethylphenethyl)phenol |
| | 16 | |
| | 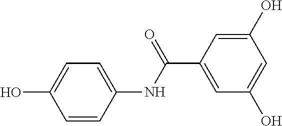 [SMILES: O=C(Nc1ccc(O)cc1)c1cc(O)cc(O)c1] | 17. 3,5-dihydroxy-N-(4- hydroxyphenyl)benzamide |
| | 17 | |
| | 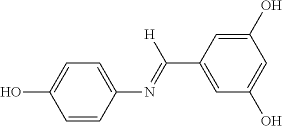 [SMILES: Oc1ccc(/N=C/c2cc(O)cc(O)c2)cc1] | 18. (E)-5[(4-hydroxy- phenylimino)-methyl]- benzene-1,3-diol |
| | 18 | |
| | 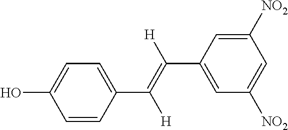 [SMILES: O=[N+]([O-])c1cc(/C=C/c2ccc(O)cc2)cc([N+](=O)[O-])c1] | 19. (E)-3,5-dinitro-4′- hydroxystilbene |
| | 19 | |
| | 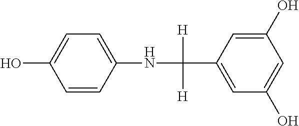 [SMILES: Oc1ccc(NCc2cc(O)cc(O)c2)cc1] | |
| | STILL TO PREPARE | |
| 4-Point | 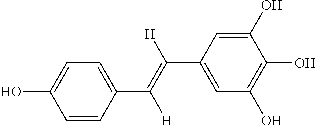 [SMILES: Oc1ccc(/C=C/c2cc(O)c(O)c(O)c2)cc1] | 20. (E)-3,4,4′,5- tetrahydroxystilbene |
| | 20 | |
| | 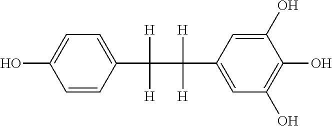 [SMILES: Oc1ccc(CCc2cc(O)c(O)c(O)c2)cc1] | 21. 5-(4-hydroxy- phenethyl)benzene- 1,2,3-triol |
| | 21 | |
| | 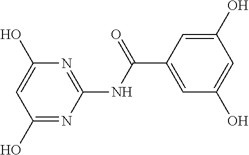 [SMILES: O=C(Nc1nc(O)cc(O)n1)c1cc(O)cc(O)c1] | |
| | STILL TO PREPARE | |

Additional compounds evaluated and templated are shown below. Examples have been used both as MIP templates and as MIP test compounds.

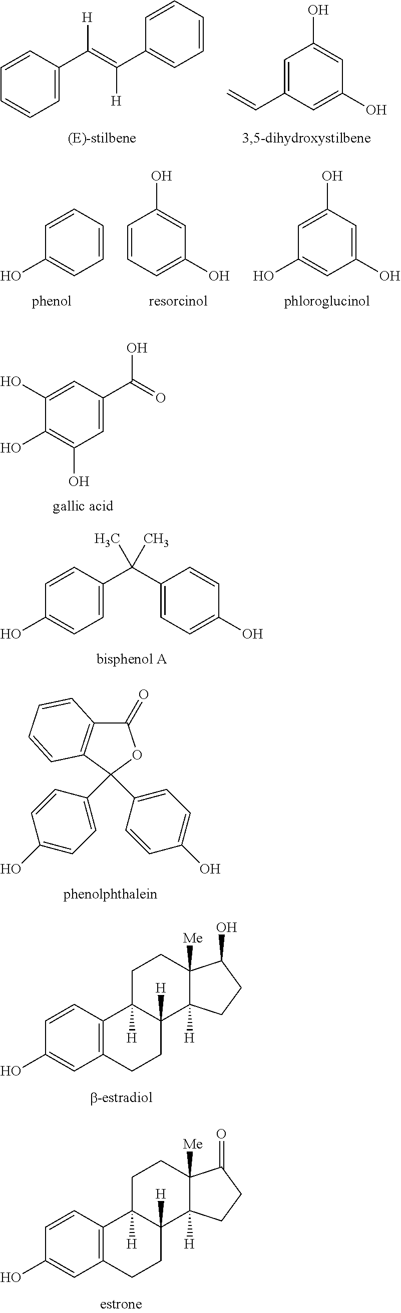

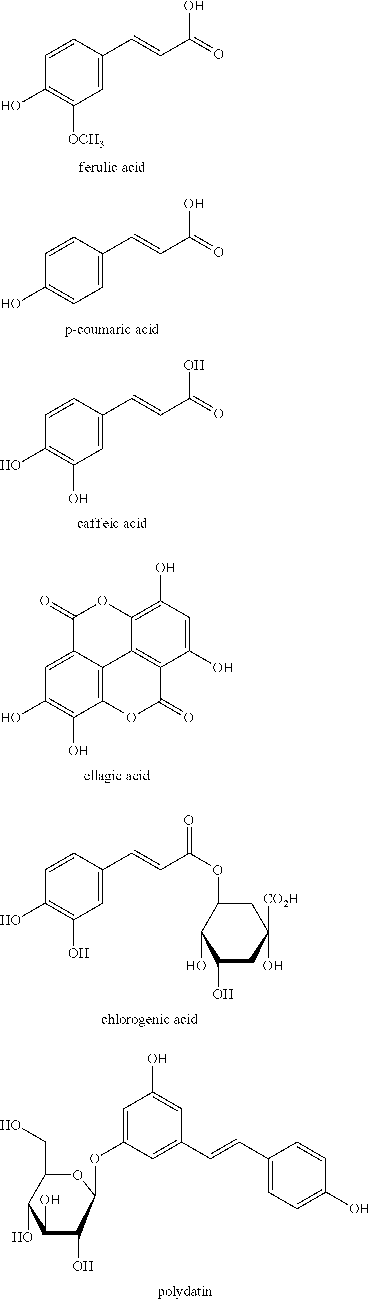

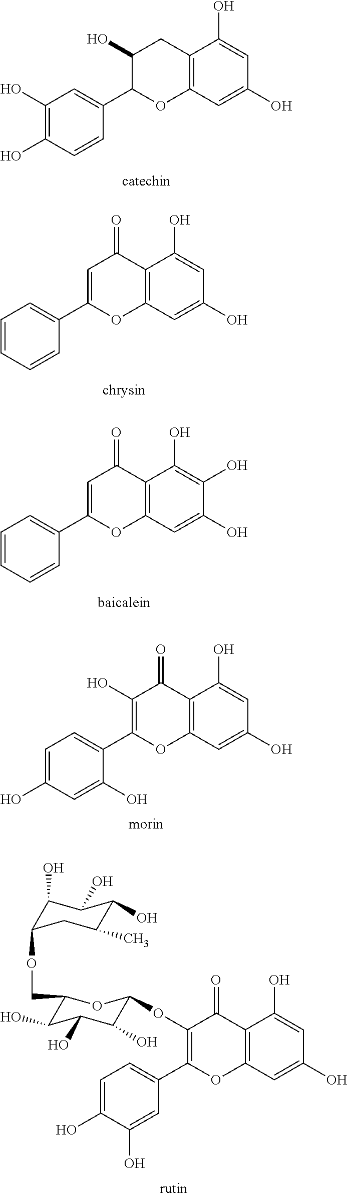

In a further preferred form, the analogue of resveratrol is a compound of Formula I or of Formula II wherein:

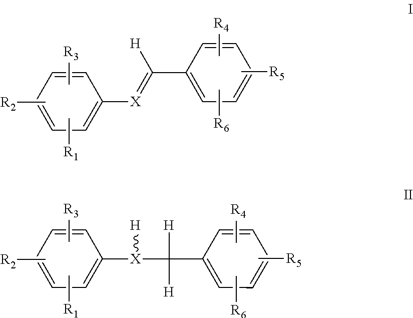

each of R₁, R₂, R₃, R₄, R₅ and R₆ is independently selected from: H, OH, CH₃, NH₂, SH, NO₂, COOH, C(O)NH₂, CHO, CN, NC, OCH₃, OC₁₋₄alkyl, SC₁₋₄alkyl, O-Sugar, N-Sugar, P(O)(OH)₂, S(O)₂(OH), OAr, NHAr, Sar, C₁₋₄alkylAr, NHC₁₋₄alkylAr, OC(O)Ar, C(O)Oar, C(O)Ar, C(O)Nar, CF₃, OCF₃, Halogen, NHC₁₋₄alkyl, N(C₁₋₄ alkyl)₂, SC₁₋₄alkyl, C(O)C₁₋₄alkyl, OC(O)C₁₋₄ alkyl, C(O)OC₁₋₄alkyl, C(O)NHC₁₋₄alkyl, C(O)N(C₁₋₄alkyl)₂, C₁₋₄alkoxy, C₁₋₄alkylenedioxy, P(O)(OH)₂, P(O)(OC₁₋₄alkyl)₂, S(O)(OC₁₋₄ alkyl)₂, C(NH₂)═C(NH₂)₂, C(NH₂)═C(CN)₂, C(CN)═C(CN)₂, C(CN)═C(NH₂)₂, C(NH₂)═C(C₁₋₄alkyl)₂, C(C₁₋₄alkyl)═C(NH₂)₂, C(CN)═C(C₁₋₄alkyl)₂, C(C₁₋₄alkyl)═C(CN)₂; and OR₁
   wherein R₁ may be selected from the group consisting of H, Ac, glucose, galactose, gallate, and ferulate.
X is selected from CH and N;
provided that at least one of R₁, R₂, R₃, R₄, R₅ and R₆ is OH.

Even more preferably, the compound is a compound of Formula I and X is N.

Suitable cross-linking agents are described above. Preferably, the cross-linking agent is EDGA.

In another preferred form, the ratio of polymerised 4-vinylpyridine to polymerised cross-linking agent is from three to fifteen. More preferably, five.

An expanded repertoire of templates used in MIP preparation could confer the potential to form a chemical class-selective MIP rather than a molecule-selective MIP.

Accordingly, in one embodiment, the MIP is imprinted with a mixture of two or more different polyphenols or analogues thereof.

In a tenth aspect, the present invention provides a method of preparing a MIP according to the ninth aspect, said method comprising the steps of
(i) polymerising the MIP in the presence of the polyphenol(s) or analogue(s) thereof and a porogen; and
(ii) removing the polyphenol(s) or analogue(s) thereof from the MIP.

In a preferred form, the ratio of the polyphenol(s) or analogue(s) thereof to 4-vinylpyridine to cross-linking agent is 1:3:15.

The temperature of the polymerisation and the period of time over which it occurs may be important. The present inventors have found that the preferred temperature range for preparing MIPs of the present invention is between 50-55° C., whereas the imprinted polymers prepared in the cited literature were at a single temperature of 45° C. (Xiang et al.¹) for excessive periods of time, such as 24 hours, or at a single temperature of 60° C. (Ma et al.² and Cao et al.³). In a preferred form, the porogen is selected from porogens that promote or facilitate hydrogen-bonding interactions.

More preferably, the porogen comprises one or more solvents selected from the group consisting of acetonitrile, acetone, ethyl acetate, and dimethylformamide. Even more preferably, the porogen further comprises one or more solvents selected from the group consisting of ethanol, methanol and dimethylsulfoxide.

In a preferred form, the porogen comprises a mixture of acetonitrile and ethanol. In an even more preferred form, the ratio of acetonitrile to ethanol is about 5 to 1.

In a eleventh aspect, the present invention provides a method of extracting one or more polyphenols from a sample by exposing the sample to a MIP according to the present invention or designed or prepared according to the methods of the present invention.

A sample could include, but not be limited to, agricultural waste, agricultural products, chemical reaction mixture, or a prepared mixture of compounds.

The extraction could be carried out using any suitable means including but not limited to a chromatographic column system, a batch adsorption (tank) system, a fluidised/expanded bed system, a membrane-related system and a “tea-bag” type of separation device.

In a preferred form, the sample is a foodstuff such as grape seed, grape skin, peanuts or peanut meal.

In a preferred form, the polyphenol is resveratrol.

In an twelfth aspect, the present invention provides a method of at least partially separating the constituents of a sample by chromatography, the method comprising the step of (i) preparing a chromatographic column comprising a MIP according to the first aspect; (ii) passing the sample through the column; and (iii) collecting fractions of the sample from the column.

In a thirteenth aspect, the present invention provides a MIP imprinted with one or more compounds selected from the group consisting of sterols and stanols, and analogues or derivatives thereof, wherein said MIP comprises a polymerised monomer.

Preferably, the monomer has one or more hydrogen-bonding sites and/or π-π bonding interaction sites. Suitable monomers are described above.

In one embodiment, the monomer is selected from the group consisting of 4-vinylpyridine, methylmethacrylic acid and other types of functional monomers, exemples of which are given above.

In another embodiment, the monomer is ethylene glycol dimethacrylate. Although often used as a cross-linking agent, ethylene glycol dimethacrylate has also been used by the present inventors as the sole monomer in certain MIPs.

In a preferred form, the MIP further comprises a polymerised cross-linking agent.

Suitable cross-linking agents are described above. A particularly preferred cross-linking agent is ethylene glycol dimethacrylate.

Preferably, the sterol and stanols are phytosterols and phytostanols.

Suitable sterols and stanols include: cholesterol, brassicasterol, beta-sitosterol, stigmasterol, campesterol, beta-sitostanol, and campestanol.

Preferably, the derivative is a ferulic acid ester of the sterol or stanol such as the components of γ-oryzanol. Suitable derivatives include: campersterylferulate, beta-sitosterylferulate, cycloartanylferulate, campestanylferulate, cycloartenylferulate and 24-methylen-cycloartanylferulate.

In a fourteenth aspect, the present invention provides a method of preparing a MIP according to the thirteenth aspect, said method comprising the steps of
(i) polymerising the MIP in the presence of the sterol(s) or stanol(s), or analogue(s) or derivative(s) thereof, and a porogen; and
(ii) removing the sterol(s) or stanol(s), or analogue(s) or derivative(s) thereof from the MIP.

Suitable porogens are described above. In a preferred form, the porogen is selected from the group consisting of chloroform, trifluoroacetic acid, water; and mixtures of trifluoroacetic acid and water.

In a fifteenth aspect, the present invention provides a method of extracting one or more sterol(s) or stanol(s), or analogue(s) or derivative(s) thereof, from a sample by exposing the sample to a MIP according to the present invention or prepared or designed according to the methods of the present invention.

A sample could include, but not be limited to, agricultural waste, agricultural products, chemical reaction mixture, or a prepared mixture of compounds.

The extraction could be carried out using any suitable means including but not limited to a chromatographic column system, a batch adsorption (tank) system, a fluidised/expanded bed system, a membrane-related system and a “tea-bag” type of separation device.

In a preferred form, the sample is a foodstuff such as avocado oil, sesame seed oil, wheat oil, or grapeseed oil.

In a sixteenth aspect, the present invention provides a method of at least partially separating the constituents of a sample by chromatography, the method comprising the step of (i) preparing a chromatographic column comprising a MIP according to the thirteenth aspect; (ii) passing the sample through the column; and (iii) collecting fractions of the sample from the column.

In the methods and MIPs of the present invention, the template may be incorporated in the polymer by non-covalent means, eg by hydrogen-bonding, π-π interactions, donor-acceptor interactions and van der Waals interactions or by covalent means such as by covalent attachment to monomers of the MIP.

Removal of the template from a covalent MIP can be achieved by any means known to be suitable to those in the art. The means include, but are not limited to, acid hydrolysis, base hydrolysis, reduction (using NaBH₄ or LiAIH₄), washing with a weak acid (to remove metal co-ordination bonds) and thermal cleavage to remove a reversible urethane bond. The latter procedure follows an adaptation of a method to cleave urethane bonds at elevated temperatures, such as 60° C. in DMSO, although the very harsh conditions of 180° C. in DMSO as describe by Ki and coworkers⁴ for the preparation of molecularly imprinted silica spheres, would not be suitable for the types of fully organic based monomers as detailed above. Additionally, enzymatic cleavage can be considered to be possible using e.g. esterases.

In the course of investigations into the functional polymers of this invention, the present inventors have prepared a number of novel and inventive compounds. The seventeenth aspect of this invention is directed to these novel compounds.

A list of various compounds used in the methods and compositions of the present invention are set out in Table 2 and Table 3 below. The novel compounds in the Table also form part of the present invention.

TABLE 2

| | STRUCTURE, NAME, | |
|---|---|---|
| | CHEMICAL FORMULA and | |
| No. | MOLECULAR WEIGHT | Comments |
| 1 | 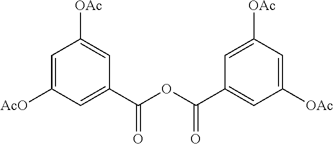 [SMILES: CC(=O)Oc1cc(OC(C)=O)cc(C(=O)OC(=O)c2cc(OC(C)=O)cc(OC(C)=O)c2)c1] | |
| | 3,5-diacetoxybenzoic anhydride | |
| | Chemical Formula: C₂₂H₁₈O₁₁ | |
| | Molecular Weight: 458.37 | |
| 2 | 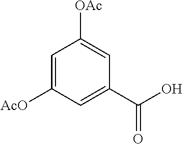 [SMILES: CC(=O)Oc1cc(OC(C)=O)cc(C(=O)O)c1] | |
| | 3,5-diacetoxybenzoic acid | |
| | Chemical Formula: C₁₁H₁₀O₆ | |
| | Molecular Weight: 238.19 | |
| 3 | 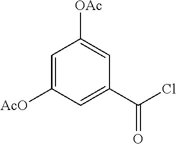 [SMILES: CC(=O)Oc1cc(OC(C)=O)cc(C(=O)Cl)c1] | |
| | 5-(chlorocarbonyl)-1,3-phenylene diacetate | |
| | Chemical Formula: C₁₁H₉ClO₅ | |
| | Molecular Weight: 256.64 | |
| 4 | 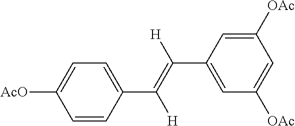 [SMILES: CC(=O)Oc1ccc(/C=C/c2cc(OC(C)=O)cc(OC(C)=O)c2)cc1] | |
| | (E)-5-(4-acetoxystyryl)-1,3-phenylene diacetate | |
| | Chemical Formula: C₂₀H₁₈O₆ | |
| | Molecular Weight: 354.35 | |
| | Acetate Protected Resveratrol | |
| 5 | 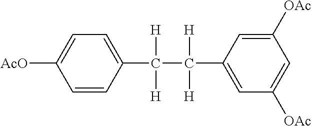 [SMILES: CC(=O)Oc1ccc(CCc2cc(OC(C)=O)cc(OC(C)=O)c2)cc1] | |
| | 5-(4-acetoxyphenethyl)-1,3-phenylene diacetate | |
| | Chemical Formula: C₂₀H₂₀O₆ | |
| | Molecular Weight: 356.37 | |
| | Saturated Protected | |
| | Resveratrol | |
| 6 | 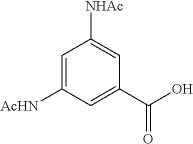 [SMILES: CC(=O)Nc1cc(NC(C)=O)cc(C(=O)O)c1] | |
| | 3,5-diacetamidobenzoic acid | |
| | Chemical Formula: C₁₁H₁₂N₂O₄ | |
| | Molecular Weight: 236.22 | |
| 7 | 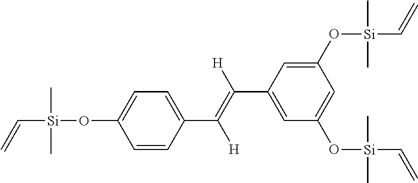 [SMILES: C=C[Si](C)(C)Oc1ccc(/C=C/c2cc(O[Si](C)(C)C=C)cc(O[Si](C)(C)C=C)c2)cc1] | NOVEL COMPOUND |
| | (E)-(5-(4-(dimethyl(vinyl)silyloxy)styryl)- | |
| | 1,3-phenylene)bis(oxy)bis(dimethyl(vinyl)silane) | |
| | Chemical Formula. C₂₆H₃₆O₃Si₃ | |
| | Molecular Weight: 480.82 | |
| 8 | 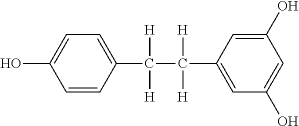 [SMILES: Oc1ccc(CCc2cc(O)cc(O)c2)cc1] | |
| | 5-(4-hydroxyphenethyl)benzene-1,3-dial | |
| | Chemical Formula: C₁₄H₁₄O₃ | |
| | Molecular Weight: 230.26 | |
| | Saturated Resveratrol | |
| | Or Saturated (2ₘₘ + 1ₚ) | |
| | Analogue | |
| 9 | 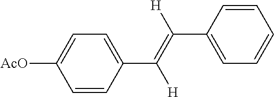 [SMILES: CC(=O)Oc1ccc(/C=C/c2ccccc2)cc1] | |
| | (E)-4-styrylphenyl acetate | |
| | Chemical Formula: C₁₆H₁₄O₂ | |
| | Molecular Weight: 238.28 | |
| 10 | 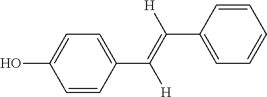 [SMILES: Oc1ccc(/C=C/c2ccccc2)cc1] | |
| | (E)-4-styrylphenol | |
| | Chemical Formula: C₁₄H₁₂O | |
| | Molecular Weight: 196.24 | |
| | (1ₚ + 0) Analogue | |
| 11 | 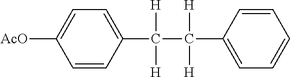 [SMILES: CC(=O)Oc1ccc(CCc2ccccc2)cc1] | NOVEL COMPOUND |
| | 4-phenethylphenyl acetate | |
| | Chemical Formula: C₁₆H₁₆O₂ | |
| | Molecular Weight: 240.30 | |
| 12 | 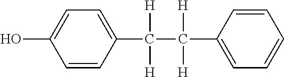 [SMILES: Oc1ccc(CCc2ccccc2)cc1] | |
| | 4-phenethylphenol | |
| | Chemical Formula: C₁₄H₁₄O | |
| | Molecular Weight: 198.26 | |
| | Saturated (1ₚ + 0) analogue | |
| 13 | 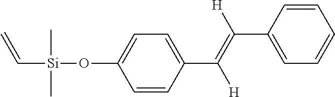 [SMILES: C=C[Si](C)(C)Oc1ccc(/C=C/c2ccccc2)cc1] | NOVEL COMPOUND |
| | (E)-dimethyl(4-styrylphenoxy)(vinyl)silane | |
| | Chemical Formula: C₁₈H₂₀OSi | |
| | Molecular Weight: 280.44 | |
| 14 | 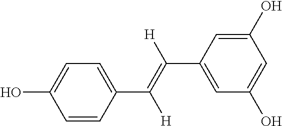 [SMILES: Oc1ccc(/C=C/c2cc(O)cc(O)c2)cc1] | |
| | (E)-5-(4 hydroxystyrybbenzene-1,3-diol | |
| | Chemical Formula: C₁₄H₁₂O₃ | |
| | Molecular Weight: 228.24 | |
| | Resveratrol | |
| 15 | 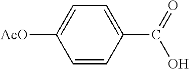 [SMILES: CC(=O)Oc1ccc(C(=O)O)cc1] | |
| | 4-acetoxybenzoic acid | |
| | Chemical Formula: C₉H₈O₄ | |
| | Molecular Weight: 180.16 | |
| 16 | 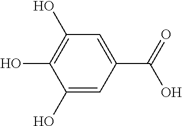 [SMILES: O=C(O)c1cc(O)c(O)c(O)c1] | |
| | 3,4,5-trihydroxybenzoic acid | |
| | Chemical Formula: C₇H₆O₅ | |
| | Molecular Weight: 170.12 | |
| | Gallic acid | |
| 17 | 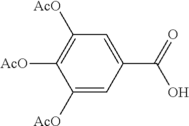 [SMILES: CC(=O)Oc1cc(C(=O)O)cc(OC(C)=O)c1OC(C)=O] | |
| | 3,4,5-triacetoxybenzoic acid | |
| | Chemical Formula: C₁₃H₁₂O₈ | |
| | Molecular Weight: 296.23 | |
| 18 | 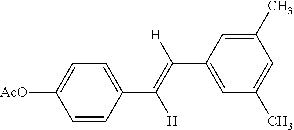 [SMILES: CC(=O)Oc1ccc(/C=C/c2cc(C)cc(C)c2)cc1] | NOVEL COMPOUND |
| | (E)-4-(3,5-dimethylstyryl)phenyl acetate | |
| | Chemical Formula: C₁₈H₁₈O₂ | |
| | Molecular Weight: 266.33 | |
| 19 | Acetone extract of 10.0 g of | MIXTURE |
| | Werribee supplied grape seeds | |
| | BATCH:02VIN03 | |
| 20 | 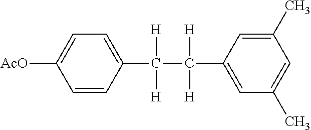 [SMILES: CC(=O)Oc1ccc(CCc2cc(C)cc(C)c2)cc1] | NOVEL COMPOUND |
| | 4-(3,5-dimethylphenethyl)phenyl acetate | |
| | Chemical Formula: C₁₈H₂₀O₂ | |
| | Molecular Weight: 268.35 | |
| 21 | 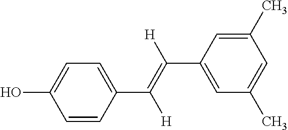 [SMILES: Cc1cc(C)cc(/C=C/c2ccc(O)cc2)c1] | 0 |
| | (E)-4-(3,5-dimethylstyryl)phenol | |
| | Chemical Formula: C₁₆H₁₆O | |
| | Molecular Weight: 224.30 | |
| | Dimethylₘₘ Resveratrol | |
| | Analogue | |
| 22 | 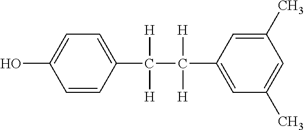 [SMILES: Cc1cc(C)cc(CCc2ccc(O)cc2)c1] | NOVEL COMPOUND |
| | 4-(3,5-dimethylphenethyl)phenol | |
| | Chemical Formula: C₁₆H₁₈O | |
| | Molecular Weight: 226.31 | |
| 23 | 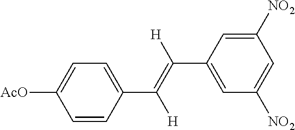 [SMILES: CC(=O)Oc1ccc(/C=C/c2cc([N+](=O)[O-])cc([N+](=O)[O-])c2)cc1] | NOVEL COMPOUND |
| | (E)-4-(3,5-dinitrostyryl)phenyl acetate | |
| | Chemical Formula: C₁₆H₁₂N₂O₆ | |
| | Molecular Weight: 328.28 | |
| 24 | 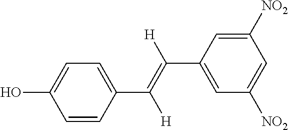 [SMILES: O=[N+]([O-])c1cc(/C=C/c2ccc(O)cc2)cc([N+](=O)[O-])c1] | NOVEL COMPOUND |
| | (E)-4-(3,5-dinitrostyryl)phenol | |
| | Chemical Formula: C₁₄H₁₀N₂O₅ | |
| | Molecular Weight: 286.24 | |
| | Dinitroₘₘ Resveratrol | |
| | Analogue | |
| 25 | 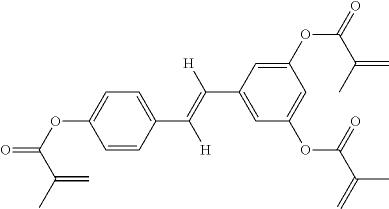 [SMILES: C=C(C)C(=O)Oc1ccc(/C=C/c2cc(OC(=O)C(=C)C)cc(OC(=O)C(=C)C)c2)cc1] | NOVEL COMPOUND |
| | (E)-5-(4-(methacryloyloxy)styryl)- | |
| | 1,3-phenylene bis(2-methylacrylate) | |
| | Chemical Formula: C₂₆H₂₄O₆ | |
| | Molecular Weight: 432.47 | |
| | A Covalent (Methacrylate) | |
| | Resveratrol Template | |
| 26 | 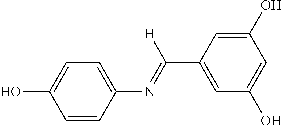 [SMILES: Oc1ccc(/N=C/c2cc(O)cc(O)c2)cc1] | |
| | (E)-5-((4-hydroxyphenylimino) | |
| | methyl)benzene-1,3-diol | |
| | Chemical Formula: C₁₃H₁₁NO₃ | |
| | Molecular Weight: 229.23 | |
| | “Green” (imine) Resveratrol | |
| 27 | 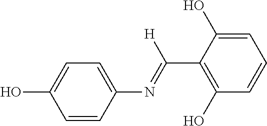 [SMILES: Oc1ccc(/N=C/c2c(O)cccc2O)cc1] | NOVEL COMPOUND |
| | (E)-2-((4-hydroxyphenylimino)methyl) | |
| | benzene-1,3-diol | |
| | Chemical Formula: C₁₃H₁₁NO₃ | |
| | Molecular Weight: 229.23 | |
| 28 | 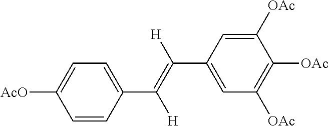 [SMILES: CC(=O)Oc1ccc(/C=C/c2cc(OC(C)=O)c(OC(C)=O)c(OC(C)=O)c2)cc1] | NOVEL COMPOUND |
| | (E)-5-(4-acetoxystyryl)benzene- | |
| | 1,2,3-triyl triacetate | |
| | Chemical Formula: C₂₂H₂₀O₈ | |
| | Molecular Weight: 412.39 | |
| 29 | 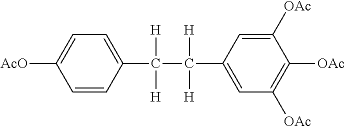 [SMILES: CC(=O)Oc1ccc(CCc2cc(OC(C)=O)c(OC(C)=O)c(OC(C)=O)c2)cc1] | NOVEL COMPOUND |
| | 5-(4-acetoxyphenethyl)benzene- | |
| | 1,2,3-triyl triacetate | |
| | Chemical Formula: C₂₂H₂₂O₈ | |
| | Molecular Weight: 414.41 | |
| 30 | 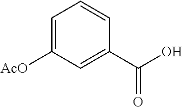 [SMILES: CC(=O)Oc1cccc(C(=O)O)c1] | |
| | 3-acetoxybenzoic acid | |
| | Chemical Formula: C₉H₈O₄ | |
| | Molecular Weight: 180.16 | |
| 32 | 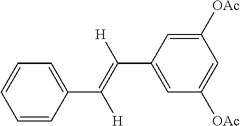 [SMILES: CC(=O)Oc1cc(/C=C/c2ccccc2)cc(OC(C)=O)c1] | |
| | (E)-5-styryl-1,3-phenylene diacetate | |
| | Chemical Formula: C₁₈H₁₆O₄ | |
| | Molecular Weight: 296.32 | |
| 33 | 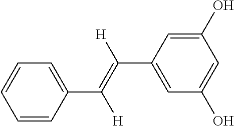 [SMILES: Oc1cc(O)cc(/C=C/c2ccccc2)c1] | |
| | (E)-5-styrylbenzene-1,3-diol | |
| | Chemical Formula: C₁₄H₁₂O₂ | |
| | Molecular Weight: 212.24 | |
| | (2ₘₘ + 0) Analogue | |
| 34 | 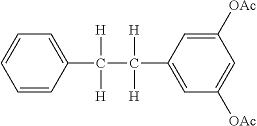 [SMILES: CC(=O)Oc1cc(CCc2ccccc2)cc(OC(C)=O)c1] | NOVEL COMPOUND |
| | 5-phenethy1-1,3-phenylene diacetate | |
| | Chemical Formula: C₁₈H₁₈O₄ | |
| | Molecular Weight: 298.33 | |
| 35 | 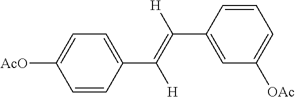 [SMILES: CC(=O)Oc1ccc(/C=C/c2cccc(OC(C)=O)c2)cc1] | |
| | (E)-3-(4-acetoxystyryl)phenyl acetate | |
| | Chemical Formula: C₁₈H₁₆O₄ | |
| | Molecular Weight: 296.32 | |
| 36 | 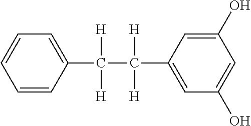 [SMILES: Oc1cc(O)cc(CCc2ccccc2)c1] | |
| | 5-phenethylbenzene-1,3-diol | |
| | Chemical Formula: C₁₄H₁₄O₂ | |
| | Molecular Weight: 214.26 | |
| 37 | 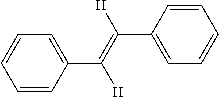 [SMILES: C(=C\c1ccccc1)/c1ccccc1] | |
| | (E)-1,2-diphenylethene | |
| | Chemical Formula: C₁₄H₁₂ | |
| | Molecular Weight: 180.25 | |
| | (E)-Stilbene | |
| 38 | 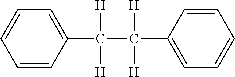 [SMILES: c1ccc(CCc2ccccc2)cc1] | |
| | 1,2-diphenylethane | |
| | Chemical Formula: C₁₄H₁₄ | |
| | Molecular Weight: 182.26 | |
| 39 | 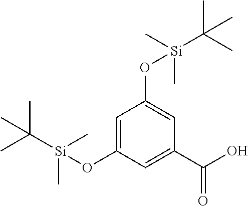 [SMILES: CC(C)(C)[Si](C)(C)Oc1cc(O[Si](C)(C)C(C)(C)C)cc(C(=O)O)c1] | |
| | 3,5-bis(tert-butyldimethylsilyloxy) | |
| | benzoic acid | |
| | Chemical Formula: C₁₉H₃₄O₄Si₂ | |
| | Molecular Weight: 382.64 | |
| 40 | 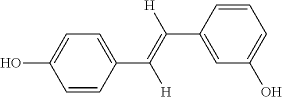 [SMILES: Oc1ccc(/C=C/c2cccc(O)c2)cc1] | |
| | (E)-3-(4-hydroxystyryl)phenol | |
| | Chemical Formula: C₁₄H₁₂O₂ | |
| | Molecular Weight: 212.24 | |
| | (1ₚ + 1ₘ) analogue | |
| 41 | 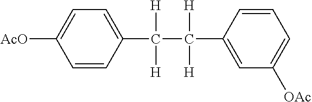 [SMILES: CC(=O)Oc1ccc(CCc2cccc(OC(C)=O)c2)cc1] | |
| | 3-(4-acetoxyphenethyl)phenyl acetate | |
| | Chemical Formula: C₁₈H₁₈O₄ | |
| | Molecular Weight: 298.33 | |
| 42 | 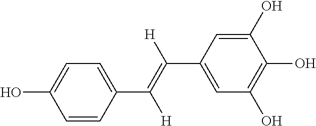 [SMILES: Oc1ccc(/C=C/c2cc(O)c(O)c(O)c2)cc1] | |
| | (E)-5-(4-hydroxystyryl)benzene-1,2,3-triol | |
| | Chemical Formula: C₁₄H₁₂O₄ | |
| | Molecular Weight: 244.24 | |
| | (3ₘₚₘ + 1ₚ) analogue | |
| 43 | 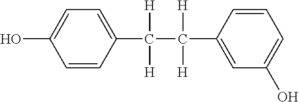 [SMILES: Oc1ccc(CCc2cccc(O)c2)cc1] | |
| | 3-(4-hydroxyphenethyl)phenol | |
| | Chemical Formula: C₁₄H₁₄O₂ | |
| | Molecular Weight: 214.26 | |
| 44 | 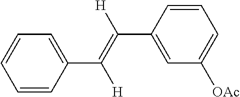 [SMILES: CC(=O)Oc1cccc(/C=C/c2ccccc2)c1] | |
| | (E)-3-styrylphenyl acetate | |
| | Chemical Formula: C₁₆H₁₄O₂ | |
| | Molecular Weight: 238.28 | |
| 45 | 8:2(v/v) EtOH/water extract of | MIXTURE |
| | 10.007 g of Werribee supplied | |
| | peanut meal | |
| 46 | 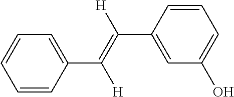 [SMILES: Oc1cccc(/C=C/c2ccccc2)c1] | |
| | (E)-3-styrylphenol | |
| | Chemical Formula: C₁₄H₁₂O | |
| | Molecular Weight: 196.24 | |
| | (1ₘ + 0) analogue | |
| 47 | 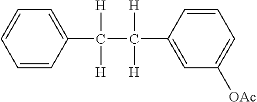 [SMILES: CC(=O)Oc1cccc(CCc2ccccc2)c1] | |
| | 3-phenethylphenyl acetate | |
| | Chemical Formula: C₁₆H₁₆O₂ | |
| | Molecular Weight: 240.30 | |
| 48 | 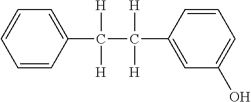 [SMILES: Oc1cccc(CCc2ccccc2)c1] | |
| | 3-phenethylphenol | |
| | Chemical Formula: C₁₄H₁₄O | |
| | Molecular Weight: 198.26 | |
| 49 | 8:2(v/v) EtOH/water extract of | MIXTURE |
| | 200.012 g of Werribee supplied | |
| | peanut meal | |
| 50 | 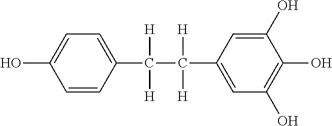 [SMILES: Oc1ccc(CCc2cc(O)c(O)c(O)c2)cc1] | NOVEL COMPOUND |
| | 5-(4-hydroxyphenethyl)benzene-1,2,3-triol | |
| | Chemical Formula: C₁₄H₁₄O₄ | |
| | Molecular Weight: 246.26 | |
| | (3ₘₚₘ + 1ₚ) saturated analogue | |
| 51 | 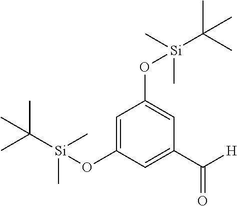 [SMILES: CC(C)(C)[Si](C)(C)Oc1cc(C=O)cc(O[Si](C)(C)C(C)(C)C)c1] | |
| | 3,5-bis(tert-butyldimethylsilyloxy) | |
| | benzaldehyde | |
| | Chemical Formula: C₁₉H₃₄O₃Si₂ | |
| | Molecular Weight: 366.64 | |
| 52 | 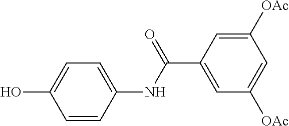 [SMILES: CC(=O)Oc1cc(OC(C)=O)cc(C(=O)Nc2ccc(O)cc2)c1] | |
| | 5-(4-hydroxyphenylcarbamoyl)- | |
| | 1,3-phenylene diacetate | |
| | Chemical Formula: C₁₇H₁₅NO₆ | |
| | Molecular Weight: 329.30 | |
| 53 | 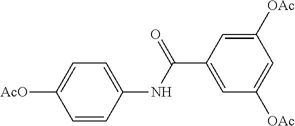 [SMILES: CC(=O)Oc1ccc(NC(=O)c2cc(OC(C)=O)cc(OC(C)=O)c2)cc1] | NOVEL COMPOUND |
| | 5-(4-acetoxyphenylcarbamoyl)- | |
| | 1,3-phenylene diacetate | |
| | Chemical Formula: C₁₉H₁₇NO₇ | |
| | Molecular Weight: 371.34 | |
| 54 | 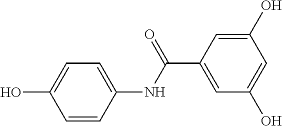 [SMILES: O=C(Nc1ccc(O)cc1)c1cc(O)cc(O)c1] | |
| | 3,5-dihydroxy-N-(4-hydroxyphenyl) | |
| | benzamide | |
| | Chemical Formula: C₁₃H₁₁NO₄ | |
| | Molecular Weight: 245.23 | |
| | “Amide” Resveratrol | |
| | Analogue | |
| 55 | 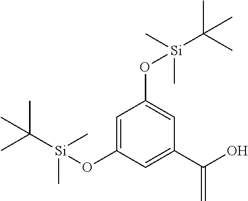 [SMILES: C=C(O)c1cc(O[Si](C)(C)C(C)(C)C)cc(O[Si](C)(C)C(C)(C)C)c1] | |
| | (5-vinyl-1,3-phenylene)bis(oxy) | |
| | bis(tert-butyldimethylsilane) | |
| | Chemical Formula: C₂₀H₃₆O₂Si₂ | |
| | Molecular Weight: 364.67 | |
| 56 | 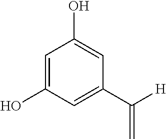 | |
| | 5-vinylbenzene-1,3-diol | |
| | Chemical Formula: C₈H₈O₂ | |
| | Molecular Weight: 136.15 | |
| 57 | 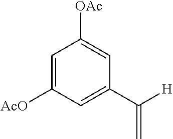 [SMILES: C=Cc1cc(OC(C)=O)cc(OC(C)=O)c1] | |
| | 5-vinyl-1,3-phenylene diacetate | |
| | Chemical Formula: C₁₂H₁₂O₄ | |
| | Molecular Weight: 220.22 | |
| 58 | 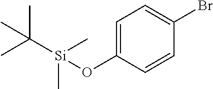 | |
| | (4-bromophenoxy) | |
| | (tert-butyl)dimethylsilane | |
| | Chemical Formula: C₁₂H₁₉BrOSi | |
| | Molecular Weight: 287.27 | |
| 59 | 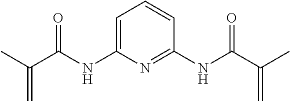 [SMILES: C=C(C)C(=O)Nc1cccc(NC(=O)C(=C)C)n1] | NOVEL COMPOUND |
| | N,N′-(pyridine-2,6-diyl)bis(2-methylacrylamide) | |
| | Chemical Formula: C₁₃H₁₅N₃O₂ | |
| | Molecular Weight: 245.28 | |
| 60 | 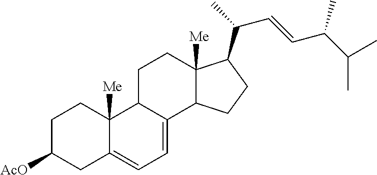 [SMILES: CC(=O)O[C@H]1CC[C@@]2(C)C(=CC=C3C2CC[C@@]2(C)C3CC[C@@H]2[C@H](C)/C=C/[C@H](C)C(C)C)C1] | |
| | (3S,10R,13R,17R)-17-((2R,5R,E)-5,6-dimethylhept-3-en-2-yl)- | |
| | 10,13-dimethyl-2,3,4,9,10,11,12,13,14,15,16,17-dodecahydro- | |
| | 1H-cyclopenta[a]phenanthren-3-yl acetate | |
| | Chemical Formula: C₃₀H₄₆O₂ | |
| | Molecular Weight: 438.69 | |
| | Ergosteryl acetate | |
| 61 | 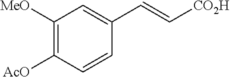 [SMILES: COc1cc(/C=C/C(=O)O)ccc1OC(C)=O] | |
| | (Z)-3-(4-acetoxy-3-methoxyphenyl) | |
| | acrylic acid | |
| | Chemical Formula: C₁₂H₁₂O₅ | |
| | Molecular Weight: 236.22 | |
| 62 | 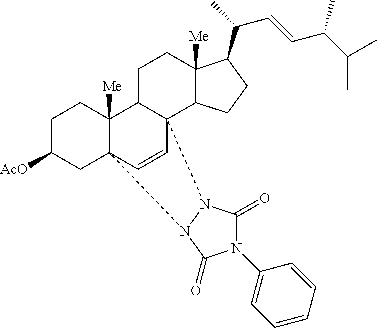 | |
| | A name could not be generated for this structure. | |
| | Chemical Formula: C₃₈H₅₁N₃O₄ | |
| | Molecular Weight: 613.83 | |
| | Diels Alder 1,4-adduct of | |
| | protected Ergisterol | |
| 63 | 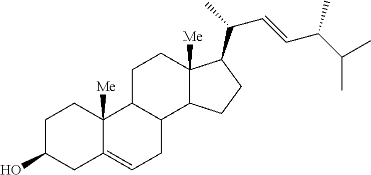 [SMILES: CC(C)[C@@H](C)/C=C/[C@@H](C)[C@H]1CCC2C3CC=C4C[C@@H](O)CC[C@]4(C)C3CC[C@@]21C] | |
| | (3S,10R,13R,17R)-17-((2R,5R,E)-5,6-dimethylhept-3-en-2-yl)- | |
| | 10,13-dimethyl-2,3,4,7,8,9,10,11,12,13,14,15,16,17- | |
| | tetradecahydro-1H-cyclopenta[a]phenanthren-3-ol | |
| | Chemical Formula: C₂₈H₄₆O | |
| | Molecular Weight: 398.66 | |
| | Δ⁵ Brassicasterol | |
| 64 | 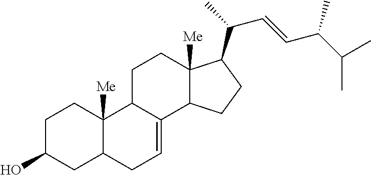 [SMILES: CC(C)[C@@H](C)/C=C/[C@@H](C)[C@H]1CCC2C3=CCC4C[C@@H](O)CC[C@]4(C)C3CC[C@@]21C] | |
| | (3S,10S,13R,17R)-17-((2R,5R,E)-5,6-dimethylhept-3-en-2-yl)- | |
| | 10,13-dimethyl-2,3,4,5,6,9,10,11,12,13,14,15,16,17- | |
| | tetradecahydro-1H-cyclopenta[a]phenanthren-3-ol | |
| | Chemical Formula: C₂₈H₄₆O | |
| | Molecular Weight: 398.66 | |
| | Unwanted Δ⁷ isomer of | |
| | Brassicasterol (Δ⁵) | |
| 65 | 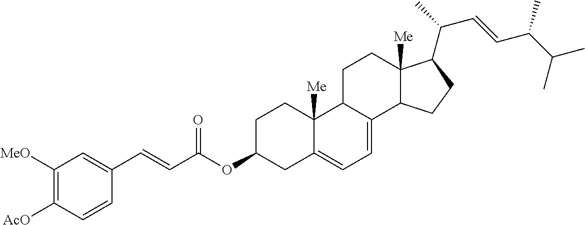 | NOVEL COMPOUND |
| | (E)-((3S,10R,13R,17R)-17-((2R,5R,E)-5,6-dimethylhept-3-en-2-yl)- | |
| | 10,13-dimethyl-2,3,4,9,10,11,12,13,14,15,16,17-dodecahydro- | |
| | 1H-cyclopenta[a]phenanthren-3-yl)-3-(4-acetoxy-3-methoxyphenyl)acrylate | |
| | Chemical Formula: C₄₀H₅₄O₅ | |
| | Molecular Weight: 614.85 | |
| 66 | 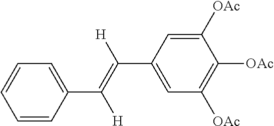 [SMILES: CC(=O)Oc1cc(/C=C/c2ccccc2)cc(OC(C)=O)c1OC(C)=O] | |
| | (E)-5-styrylbenzene-1,2,3-triyltriacetate | |
| | Chemical Formula: C₂₀H₁₈O₆ | |
| | Molecular Weight: 354.35 | |
| 67 | 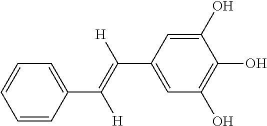 [SMILES: Oc1cc(/C=C/c2ccccc2)cc(O)c1O] | |
| | (E)-5-styrylbenzene-1,2,3-triol | |
| | Chemical Formula: C₁₄H₁₂O₃ | |
| | Molecular Weight: 228.24 | |
| | (3ₘₚₘ + 0) analogue | |
| 68 | 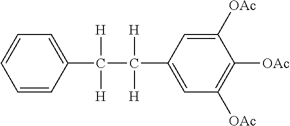 [SMILES: CC(=O)Oc1cc(CCc2ccccc2)cc(OC(C)=O)c1OC(C)=O] | NOVEL COMPOUND |
| | 5-phenethylbenzene-1,2,3-triyl triacetate | |
| | Chemical Formula: C₂₀H₂₀O₆ | |
| | Molecular Weight: 356.37 | |
| 69 | 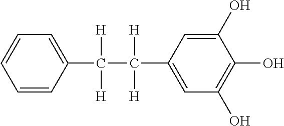 [SMILES: Oc1cc(CCc2ccccc2)cc(O)c1O] | NOVEL COMPOUND |
| | 5-phenethylbenzene-1,2,3-triol | |
| | Chemical Formula: C₁₄H₁₄O₃ | |
| | Molecular Weight: 230.26 | |
| | Saturated (3ₘₚₘ + 0) analogue | |
| 70 | 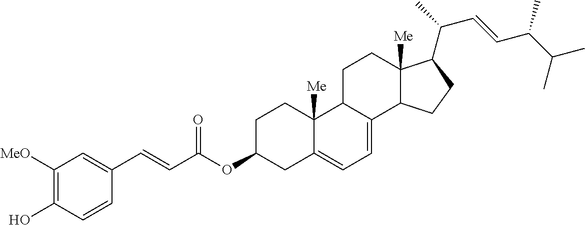 | |
| | (E)-((3S,10R,13R,17R)-17-((2R,5R,E)-5,6-dimethylhept-3-en-2-yl)- | |
| | 10,13-dimethyl-2,3,4,9,10,11,12,13,14,15,16,17-dodecahydro- | |
| | 1H-cyclopenta[a]phenanthren 3 yl) 3-(4-hydroxy-3-methoxyphenyl)acrylate | |
| | Chemical Formula: C₃₈H₅₂O₄ | |
| | Molecular Weight: 572.82 | |
| | Ergosteryl ferulate adduct | |
| 71 | 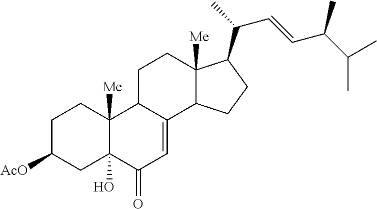 [SMILES: CC(=O)O[C@H]1CC[C@]2(C)C3CC[C@@]4(C)C(CC[C@@H]4[C@H](C)/C=C/[C@@H](C)C(C)C)C3=CC(=O)[C@@]2(O)C1] | |
| | (35,5R,10R,13R,17R)-7-(12R,5R,E)-5,6-thmethylhept-3-en-2-yl)- | |
| | 5-hydroxy-10,13-dimethyl-6-oxo-2,3,4,5,6,9,10,11,12,13,14,15,16,17- | |
| | tetradecahydro-1H-cyclopenta[a]phenanthren-3-yl acetate | |
| | Chemical Formula: C₃₀H₄₆O₄ | |
| | Molecular Weight: 470.68 | |
| | 5-ol, 6-one analogue of | |
| | protected ergosterol | |

TABLE 3
Additional commercial sourced compounds.

| STRUCTURE, NAME, | |
|---|---|
| CHEMICAL FORMULA and | |
| MOLECULAR WEIGHT | ID/NAME |
| 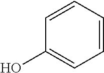 | Phenol |
| phenol | |
| Chemical Formula: C₆H₆O | |
| Molecular Weight: 94.11 | |
| 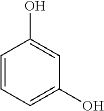 | Resorcinol |
| resorcinol | |
| Chemical Formula: C₆H₆O₂ | |
| Molecular Weight: 110.11 | |
| 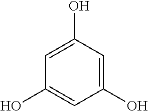 | Phloroglucinol |
| benzene-1,3,5-triol | |
| Chemical Formula: C₆H₆O₃ | |
| Molecular Weight: 126.11 | |
| 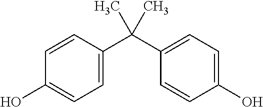 [SMILES: CC(C)(c1ccc(O)cc1)c1ccc(O)cc1] | Bisphenol A |
| 4,4′-(propane-2,2-diyl)diphenol | |
| Chemical Formula: C₁₅H₁₆O₂ | |
| Molecular Weight: 228.29 | |
| 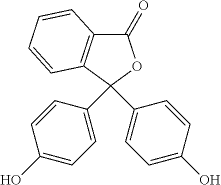 [SMILES: O=C1OC(c2ccc(O)cc2)(c2ccc(O)cc2)c2ccccc21] | Phenol- phthalein |
| 3,3-bis(4-hydroxyphenyl)isobenzofuran-1(3H)-one | |
| Chemical Formula: C₂₀H₁₄O₄ | |
| Molecular Weight: 318.32 | |
| 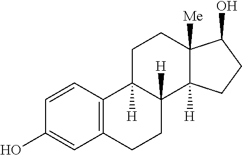 | β-Estradiol |
| (8R,9S,13S,14S,17S)-13-methyl-7,8,9,11,12,13,14,15,16,17- | |
| decahydro-6H-cyclopenta[a]phenanthrene-3,17-diol | |
| Chemical Formula: C₁₈H₂₄O₂ | |
| Molecular Weight: 272.38 | |
| 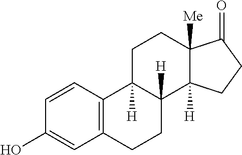 | Estrone |
| (8R,9S,13S,14S)-3-hydroxy-13-methyl-7,8,9,11,12,13,15,16- | |
| octahydro-6H-cyclopenta[a]phenanthren-17(14H)-one | |
| Chemical Formula: C₁₈H₂₂O₂ | |
| Molecular Weight: 270.37 | |
| 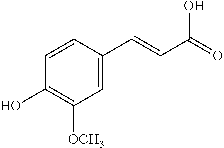 [SMILES: COc1cc(/C=C/C(=O)O)ccc1O] | Ferulic acid |
| (E)-3-(4-hydroxy-3-methoxyphenyl)acrylic acid | |
| Chemical Formula: C₁₀H₁₀O₄ | |
| Molecular Weight: 194.18 | |
| 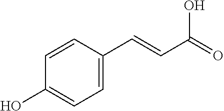 [SMILES: O=C(O)/C=C/c1ccc(O)cc1] | p-Coumaric acid |
| (E)-3-(4-hydroxyphenyl)acrylic acid | |
| Chemical Formula: C₉H₈O₃ | |
| Molecular Weight: 164.16 | |
| 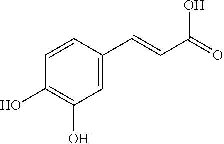 [SMILES: O=C(O)/C=C/c1ccc(O)c(O)c1] | Caffeic acid |
| (E)-3-(3,4-dihydroxyphenyl)acrylic acid | |
| Chemical Formula: C₉H₈O₄ | |
| Molecular Weight: 180.16 | |
| 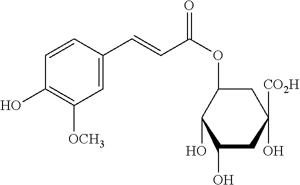 | Chlorogenic acid |
| (1R,3S,4S,5S)-3-((E)-3-(3,4-dihydroxyphenyl)acryloyloxy)- | |
| 1,4,5-trihydroxycyclohexanecarboxylic acid | |
| Chemical Formula: C₁₆H₁₈O₉ | |
| Molecular Weight: 354.31 | |
| 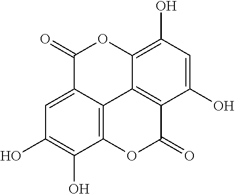 [SMILES: O=c1oc2c(O)cc(O)c3c(=O)oc4c(O)c(O)cc1c4c23] | Ellagic acid |
| 1,3,7,8-tetrahydroxychromeno[5,4,3-cde] | |
| chromene-5,10-dione | |
| Chemical Formula: C₁₄H₆O₈ | |
| Molecular Weight: 302.19 | |
| 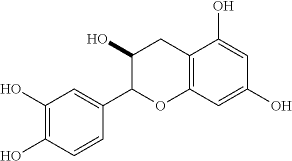 [SMILES: Oc1cc(O)c2c(c1)OC(c1ccc(O)c(O)c1)[C@@H](O)C2] | Catechin |
| 2-(3,4-dihydroxyphenyl)chroman-3,5,7-triol | |
| Chemical Formula: C₁₅H₁₄O₆ | |
| Molecular Weight: 290.27 | |
| 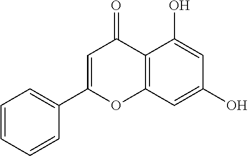 [SMILES: O=c1cc(-c2ccccc2)oc2cc(O)cc(O)c12] | Chrysin |
| 5,7-dihydroxy-2-phenyl-4H-chromen-4-one | |
| Chemical Formula: C₁₅H₁₀O₄ | |
| Molecular Weight: 254.24 | |
| 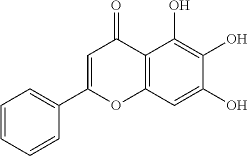 [SMILES: O=c1cc(-c2ccccc2)oc2cc(O)c(O)c(O)c12] | Baicalein |
| 5,6,7-trihydroxy-2-phenyl-4H-chromen-4-one | |
| Chemical Formula: C₁₅H₁₀O₅ | |
| Molecular Weight: 270.24 | |
| 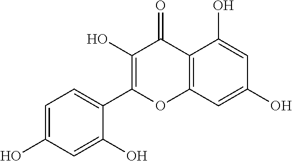 [SMILES: O=c1c(O)c(-c2ccc(O)cc2O)oc2cc(O)cc(O)c12] | Morin |
| 2-(2,4-dihydroxyphenyl)-3,5,7-trihydroxy-4H-chromen-4-one | |
| Chemical Formula: C₁₅H₁₀O₇ | |
| Molecular Weight: 302.24 | |
| 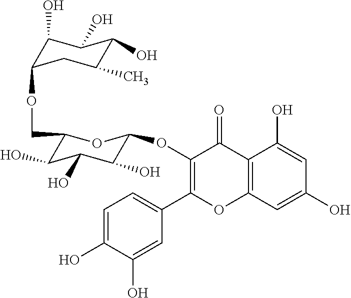 | Rutin |
| 2-(3,4-dihydroxyphenyl)-5,7-dihydroxy-3-((2R,3R,4R,5R, | |
| 6S)-3,4,5-trihydroxy-6-(((1S,2S,3S,4R,5R)-2,3,4-trihydroxy- | |
| 5-methylcyclohexyloxy)methyl)tetrahydro- | |
| 2H-pyran-2-yloxy)-4H-chromen-4-one | |
| Chemical Formula: C₂₈H₃₂O₁₅ | |
| Molecular Weight: 608.54 | |
| 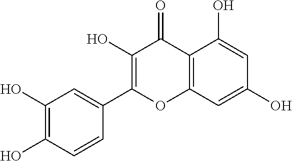 [SMILES: O=c1c(O)c(-c2ccc(O)c(O)c2)oc2cc(O)cc(O)c12] | Quercetin |
| 2-(3,4-dihydroxphenyl)-3,5,7-trihydroxy-4H-chromen-4-one | |
| Chemical Formula: C₁₅H₁₀O₇ | |
| Molecular Weight: 302.24 | |

In a preferred form, the present invention provides novel compounds of Formula I and Formula II wherein:

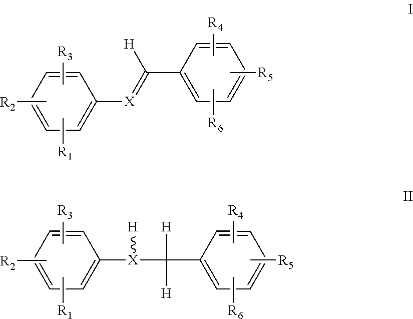

each of R₁, R₂, R₃, R₄, R₅ and R₆ is independently selected from: H, OH, CH₃, NH₂, SH, NO₂, COOH, C(O)NH₂, CHO, CN, NC, OCH₃, OC₁₋₄alkyl, SC₁₋₄alkyl, O-Sugar, N-Sugar, P(O)(OH)₂, S(O)₂(OH), OAr, NHAr, Sar, C₁₋₄alkylAr, NHC₁₋₄alkylAr, OC(O)Ar, C(O)Oar, C(O)Ar, C(O)NAr, CF₃, OCF₃, Halogen, NHC₁₋₄alkyl, N(C₁₋₄alkyl)₂, SC₁₋₄alkyl C(O)C₁₋₄alkyl, OC(O) C₁₋₄alkyl, C(O)OC₁₋₄alkyl, C(O)NHC₁₋₄alkyl, C(O)N(C₁₋₄alkyl)₂, C₁₋₄alkoxy, C₁₋₄alkylenedioxy, P(O)(OH)₂, P(O)(OC₁₋₄alkyl)₂, S(O)(OC₁₋₄alkyl)₂, C(NH₂)═C(NH₂)₂, C(NH₂)═C(CN)₂, C(CN)═C(CN)₂, C(CN)═C(NH₂)₂, C(NH₂)═C(C₁₋₄alkyl)₂, C(C₁₋₄alkyl)=C(NH₂)₂, C(CN)═C(C₁₋₄alkyl)₂, C(C₁₋₄alkyl)═C(CN)₂; and OR₁
wherein R₁ may be selected from the group consisting of H, Ac, glucose, galactose, gallate, ferulate.
X is selected from CH and N;
provided that at least one of R₁, R₂, R₃, R₄, R₅ and R₆ is OH.

Even more preferably, the compound is a compound of Formula I and X is N.

In an eighteenth aspect, there is provided a method of at least partially separating components of a sample comprising two or more of said components, said method comprising sequentially exposing the sample to at least two MIPs wherein each MIP has been imprinted with a different template.

The present inventors have further shown that MIPs can be used to extract components from samples by using MIPs encased in “teabags” ie a permeable mesh that allows the MIP to be readily inserted or “dipped” into the sample and then removed. Suitable meshes include cotton-based materials, Gilson® 63 μm sieve mesh and Sigma Aldrich dialysis tubing cellulose membrane (12 kDa MWCO).

Accordingly, in a nineteenth aspect, there is provided a MIP encased in a permeable mesh. The MIO can include, but need not be limited to, the MIPs of the present invention or prepared or designed according to the methods of the present invention.

In a twentieth aspect, there is provided a method of extracting a component from a sample comprising exposing the sample to a MIP according to the nineteenth aspect.

In a twenty first aspect, there is provided a MIP imprinted with (E)-5-[(4-hydroxy-phenylimino)-methyl]-benzene-1,3-diol or 3,5-dihydroxy-N-(4-hydroxyphenyl)benzamide, wherein said MIP comprises a polymerised monomer.

In one embodiment, the MIP comprises a cross-linking agent.

Preferably, the MIP is for use in the extraction of resveratrol.

The monomer, cross-linking agent and porogen used in preparation of the MIP may be selected from those described in relation to the other aspects of the invention.

Preferably, the monomer is 4-vinylpyridine and the cross-linking agent is EDGA. More preferably, the ratio of template to 4-vinylpyridine used in preparation of the MIP is 1:3.

Preferably, the MIP comprises one or more features of one or more of the other aspects of the invention.

In a twenty second aspect, there is provided a method of extracting resveratrol from a sample, said method comprising exposing the sample to a MIP according to the twenty first aspect.

Preferably, the resveratrol is subsequently washed or elute from the MIP.

It would be understood by those skilled in the art that the aspects of the present invention are closely interrelated and that therefore the features of one aspect of the present invention may also be relevant to another aspect of the present invention.

In order that the nature of the present invention may be more clearly understood preferred forms thereof will now be described by reference to the following non-limiting Examples.

### EXAMPLES

### A Synthesis of Resveratrol and Resveratrol Analogues

A number of polyfunctionalized (E)-stilbene analogues 1 have been synthesised as templates around which new molecularly imprinted polymers (MIPs) can be generated. These compounds, along with their “saturated” diphenethyl analogues 2, were also used as probes to investigate the characteristics of these MIPs. These compounds were synthesized using a convergent methodology where the key step was the palladium catalysed coupling of a functionalized benzoyl chloride 3 with a functionalized styrene 4. Best results were obtained when the acid chlorides were freshly generated from the parent benzoic acids 5, which were then immediately on-reacted. This synthetic methodology is described in Scheme 1.

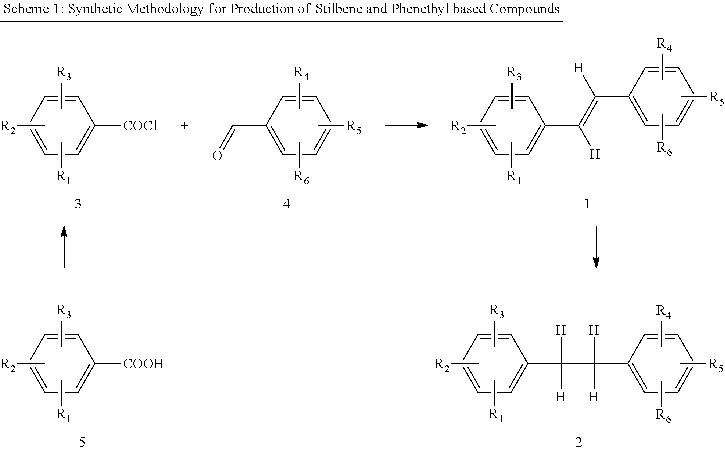

The key coupling step for generating the nitrogen isosteres is shown below in Scheme 2.

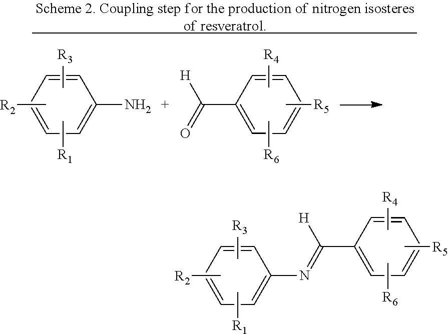

Most AR solvents were used as purchased from the manufacturer except for dimethylformamide (DMF), which was dried over 4 Å molecular sieves and toluene which was dried over sodium wire. Milli-Q distilled water was used for aqueous manipulations. Saturated aqueous solutions of reagents were written, for example, as sat. NaHCO₃. Solvent extracts of aqueous solutions were dried over anhydrous sodium sulfate, filtered and then rotary evaporated to dryness at low pressure (10-400 mbar) and 30-35° C. in a temperature-controlled water bath.

Analytical thin layer chromatography (TLC) was performed using aluminium sheets (Merck) coated with silica gel 60 F₂₅₄. The components were visualised by (i) fluorescence at 254 nm and (ii) exposure to iodine vapour or dipping into an ethanolic phosphomolybdic acid solution and heating until charred.

Column chromatography was conducted using silica gel 60 (Merck), 0.040-0.063 mm (230-400 mesh): eluent mixtures are expressed as volume/volumes.

Melting points were determined using a Büchi B-545 melting point apparatus.

Proton nuclear magnetic resonance (¹H NMR) spectra were recorded at (i) 200 MHz on a Bruker AC-200 spectrometer, (ii) 300 MHz with a Bruker DPX-300 spectrometer, or (iii) 400 MHz with a Bruker DRX-400 spectrometer. The ¹H NMR spectra refer to solutions in deuterated solvents as indicated. The residual solvent peaks have been used as an internal reference. Resonances were assigned according to the following convention: chemical shift (δ) measured in parts per million (ppm) relative to the residual solvent peak, multiplicity, number of protons, coupling constants (J Hz), and assignment. Multiplicities are denoted as (s) singlet, (d) doublet, (dd) doublet of doublets, (ddd) doublet of doublet of doublets, (t) triplet, (dt) doublet of triplets, (td) triplet of doublets, (q) quartet, or (m) multiplet and prefixed (b) broad where appropriate.

Carbon nuclear magnetic resonance (¹³C NMR) spectra were recorded at (i) 50 MHz on a Bruker AC-200 spectrometer, (ii) 75 MHz on a Bruker DPX-300 spectrometer, or (iii) 100 MHz on a Bruker DRX-400 spectrometer with the spectra referring to deuterated solutions in solvents indicated.

Low resolution electrospray ionisation mass spectra (ESI) were recorded on a Micromass Platform II API QMS Electrospray mass spectrometer. Analyses were conducted in both positive (ESI+) and negative (ESI−) polarity. Principle ion peaks (m/z) are reported with their intensities expressed as percentages of the base peak in brackets. High-resolution electrospray mass spectra (HRMS) were recorded on a Brucker BioApex 47e Fourier Transform mass spectrometer.

The following examples serve to illustrate these synthetic methodologies.

### Compounds Synthesised

### Example 1

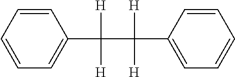 [SMILES: c1ccc(CCc2ccccc2)cc1]

### 1,2-Diphenylethane (1)

Trans-stilbene and 10% Pd/C in methanol was hydrogenated overnight at 95 psi. The reaction was then filtered through a Celite pad and the clear filtrate concentrated by rotary evaporation. Purification of the resultant product with column chromatography (isocratically eluted with hexane) gave an absolute yield of 1,2-diphenylethane as a clear oil. This solidified upon standing at room temperature. R_{f} 0.95 (4:1 hexane/EtOAc), 0.30 (hexane); mp 49.5-50.0° C., ¹H NMR (CDCl₃): δ 2.95 (s, 4H, 2×CH₂), 7.18-7.23 (m, 6H, H-2, H-4, H-6, H-2′, H-4′, H-6′), 7.28-7.33 ((m, 4H, H-3, H-5, H-3′, H-5′); ¹³C JMOD NMR (CDCl₃): δ 37.18 (2×CH₂), 125.18 (4,4′), 127.59 (2,6,2′,6′), 127.71 (3,5,3′,5′), 141.01 (1,1′); LREI mass spectrum; m/z 182 (M., 100%), 183 (16%), 91 (51%).

### Example 2

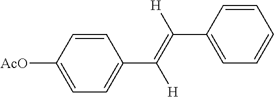 [SMILES: CC(=O)Oc1ccc(/C=C/c2ccccc2)cc1]

### (E)-4-Acetoxystilbene, [(E)-4-Acetoxyphenethene Benzene] (2)

Freshly distilled benzoyl chloride, 4-acetoxystyrene, N-ethylmorpholine and palladium diacetate (2.00 mole %) were added to toluene and this mixture heated overnight at 120° C. After cooling to room temperature, ethyl acetate was added and the solution washed twice each with 0.1M HCl and water. The organic layer was then dried, filtered and rotary evaporated to give a brown solid. Recrystallization from EtOAc/hexane produced (E) 4-acetoxyphenethene benzene as beige coloured fine needles. R_{f} 0.53 (2:1 hexane/EtOAc); mp 150-151° C.; ¹H NMR (CDCl₃): δ 2.28 (s, 3H, OAc), 6.99-7.10 (m, 4H), 7.21-7.26 (m, 1H), 7.30-7.36 (m, 2H), 7.46-7.50 (m, 4H); ¹H NMR (CD₃OD): δ 2.24 (s, 3H, OAc), 7.03-7.23 (m, 5H, J=13.5 Hz, Hₜᵣₐₙₛ), 7.28-7.34 (m, 2H), 7.49-7.57 (m, 4H); ¹³C JMOD NMR (CDCl₃): δ 20.13 (OCOCH₃), 120.81 (3,5), 125.55 (2′,6′), 126.44 (3′,5′), 126.72 (2×Cₜᵣₐₙₛ), 127.72 (2,6), 127.99 (4′), 134.18 (1), 136.22 (1′), 149.13 (4′), 168.390 (OCOCH₃); LRESI positive ion mass spectrum; m/z 261 (MNa⁺, 100%), 293 (MNa⁺⁺MeOH, 95%).

### Example 3

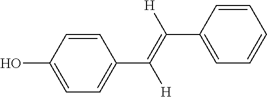 [SMILES: Oc1ccc(/C=C/c2ccccc2)cc1]

### (E)-4-Hydroxystilbene, [(E)-4-Hydroxyphenethene benzene] (3)

A solution of potassium hydroxide in methanol was added to 4-acetoxyphenethene benzene dissolved in methanol. This reaction was heated under an argon atmosphere to 65° C. for 60 minutes. The solution was poured into water and then acidified to pH 4 with dilute HCl. Sodium chloride was added and the solution extracted with EtOAc. The organic layer was then separated, washed three times with water, then dried, filtered and rotary evaporated to return a pale pink coloured solid. This was purified with column chromatography (isocratically eluted with 2:1 hexane/EtOAc) to give 4-hydroxyphenethene benzene as a white solid. R_{f} 0.42 (2:1 hexane/EtOAc); mp: 189.6-190.0° C.; ¹H NMR (CDCl₃): δ 4.71 (s, 1H, OH), 6.79-6.82 (m, 2H, Jₒᵣₜₕₒ=8.7 Hz, H2, H6), 6.93 (d, 1H, Jₜᵣₐₙₛ=16.4 Hz, Hₐₗₖₑₙₑ), 7.02 (d, 1H, Jₜᵣₐₙₛ=16.4 Hz, Hₐₗₖₑₙₑ), 7.18-7.23 (m, 1H, H4′), 7.29-7.32 (m, 2H, H3′, H5′), 7.37-7.40 (m, 2H, H3, H5), 7.44-7.47 (m, 2H, H2′, H6′); LRESI negative ion mass spectrum; m/z 195 ([M−H]−, 100%), 196 (16%).

### Example 4

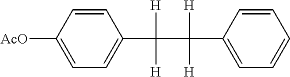 [SMILES: CC(=O)Oc1ccc(CCc2ccccc2)cc1]

### 4-Phenethyl-acetoxybenzene (4)

(E) 4-Acetoxyphenethene benzene and 10% Pd/C in methanol was hydrogenated overnight at 95 psi. Filtration through a Celite pad gave a clear filtrate that was rotary evaporated to a viscous oil. Gradient elution column chromatography (4:1 hexane/EtOAc to 2:1 hexane/EtOAc) gave 4-phenethyl-acetoxybenzene as a clear viscous oil. R_{f} 0.53 (2:1 hexane/EtOAc); ¹H NMR (CDCl₃): δ 2.26 (s, 3H, OAc), 2.89 (s, 4H, 2×CH₂), 6.95-6.99 (m, 2H), 7.13-7.20 (m, 5H), 7.23-7.29 (m, 2H); ¹³C JMOD NMR (CDCl₃): δ 20.15 (CH₃), 36.35, 36.93 (2×CH₂), 120.45 (2,6), 125.11 (4′), 127.48 (2′,6′), 127.56 (3′,5′), 128.46 (3,5), 138.37 (4), 140.64 (1′), 148.04 (1), 168.56 (C═O); LRESI positive ion mass spectrum; m/z 263 (MNa⁺, 100%), 264 (18%).

### Example 5

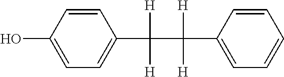 [SMILES: Oc1ccc(CCc2ccccc2)cc1]

### 4-Phenethyl-phenol (5)

A solution of potassium hydroxide in methanol was added to 4-phenethyl-acetoxybenzene dissolved in methanol. This reaction was heated to 65° C. for 2 hours under an argon atmosphere. The clear solution was then poured into water, acidified to pH 2 with 1M HCl, and extracted with EtOAc. The organic layer was separated and washed three times with water, then dried, filtered and rotary evaporated to give a pale pink coloured solid. Purification by isocratic elution on column chromatography using 2:1 EtOAc/hexane gave 4-phenethyl-phenol as a white solid. R_{f} 0.55 (2:1 EtOAc/hexane); ¹H NMR (CDCl₃): δ 2.84 (s, 4H, 2×CH₂), 6.69-6.74 (m, 2H, J=8.6 Hz, ArHₒᵣₜₕₒ), 6.99-7.04 (m, 2H, J=8.6 Hz, ArHₒᵣₜₕₒ), 7.12-7.19 (m, 3H), 7.22-7.28 (m, 2H); ¹³C JMOD NMR (CDCl₃): δ 36.09, 37.24 (2×CH₂), 114.29 (2,6), 124.99 (4′), 127.42 (2′,6′), 127.59 (3′,5′), 128.67 (3,5), 133.20 (4), 140.90 (1′), 152.55 (1); LRESI negative ion mass spectrum; m/z 197 ([M−H]⁻, 100%), 198 (16%).

### Example 6

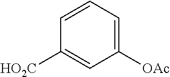 [SMILES: CC(=O)Oc1cccc(C(=O)O)c1]

### 3-Acetoxybenzoic acid (6)

A suspension of 3-hydroxybenzoic acid in ethyl acetate was cooled in an ice-bath. Acetic anhydride and pyridine were added and the reaction allowed proceeding for 60 minutes. The resultant homogenous solution was then stirred at room temperature overnight. Formic acid and further ethyl acetate were then added and the reaction poured onto ice-water. The organic phase was separated and washed six times with water, then dried, filtered and rotary evaporated to give a white solid. This solid was recrystallized from EtOAc/hexane (1:1) to produce 3-acetoxybenzoic acid as a white powder. R_{f} 0.46 (2:1 EtOAc/hexane); mp 133.0-133.5° C.; ¹H NMR (CDCl₃): δ 2.33 (s, 3H, OAc), 7.34-7.38 (m, 1H, H-4), 7.49 (t, 1H, Jₒᵣₜₕₒ=7.8 Hz, H-4), 7.84 (t, 1H, Jₘₑₜₐ=2.0 Hz, H-2), 8.00 (dt, 1H, H-6); ¹³C JMOD NMR (CDCl₃): δ 20.00 (OC(O)CH₃), 122.42 (2), 126.21 (4), 126.60 (6), 128.56 (5), 129.84 (1), 149.70 (3), 168.19 (COCH₃), 170.27 (COOH); LRESI positive ion mass spectrum; m/z 203 (MNa⁺, 100%), 204 (11%).

### Example 7

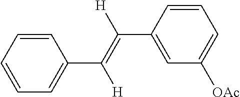 [SMILES: CC(=O)Oc1cccc(/C=C/c2ccccc2)c1]

### (E)-4-Acetoxystilbene, [(E)-3-Acetoxyphenethene Benzene] (7)

A suspension of 3-acetoxybenzoic acid in dry toluene N,N-DMF and thionyl chloride was heated at 100° C. for three hours under an argon atmosphere. The solvents were removed by vacuum distillation to give a yellow oil. This material was dissolved in dry toluene and the solution was sonicated under vacuum for 30 minutes to remove dissolved gases. Styrene, N-ethylmorpholine and palladium diacetate (2 mole %) were added and this mixture was heated at 120° C. for 22 hours under an argon gas atmosphere. The solution was cooled to room temperature and ethyl acetate was added. The solution was then washed three times with 0.1 M HCl and twice with water then dried, filtered and rotary evaporated to give a brown solid. Gradient elution column chromatography (9:1 hexane/EtOAc to 4:1 hexane/EtOAc) gave a white solid. This was recrystallized from 2:3 EtOAc/hexane (50 mL) to return (E)-3-acetoxystilbene as white fine needles. R_{f} 0.45 (hexane/EtOAc 9:1), 0.75 (hexane/EtOAc 4:1); mp 106.5-107.0° C.; ¹H NMR (CDCl₃): δ 2.30 (s, 3H, OAc), 6.94-7.00 (m, 1H, H-4), 7.04 (d, 1H, Jₜᵣₐₙₛ=16.4 Hz, Hₐₗₖₑₙₑ)₉ 7.08 (d, 1H, Hₐₗₖₑₙₑ), 7.23-7.28 (m, 2H), 7.30-7.37 (m, 4), 7.45-7.50 (m, 2H, H-2′, H-6′); ¹³C JMOD NMR (CDCl₃): δ 20.16 (OC(O)CH₃), 118.30 (4), 119.68 (2), 123.21 (6), 125.67 (2′,6′), 126.76, 126.92 (2×Cₐₗₖₑₙₑ), 127.75 (3′,5′), 128.61 (4′), 128.84 (5), 136.05 (1), 138.11 (1′), 150.17 (3), 168.41 (C═O); LRESI positive ion mass spectrum; m/z 261 (MNa⁺, 100%), 293 ((MNa⁺+MeOH, 74%); HRESI positive ion mass spectrum; C₁₆H₁₄O₂Na⁺; calc. 239.1072.

### Example 8

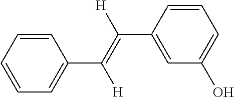 [SMILES: Oc1cccc(/C=C/c2ccccc2)c1]

### (E)-3-Hydroxystilbene, [(E)-3-Hydroxyphenethene benzene] (8)

A solution of potassium hydroxide in methanol was added to a suspension of 3-acetoxystilbene in methanol. This solution was heated to 70° C. for 2 hours under an argon atmosphere. The clear solution was then acidified to pH 4 with dilute hydrochloric acid. Water was added, and the volume reduced by rotary evaporation until the first appearance of a precipitate. Further water was added and the solution then extracted four times with EtOAc. The combined organic extracts were dried, filtered and rotary evaporated to give a pale yellow coloured solid. This was purified by gradient elution column chromatography (4:1 hexane/EtOAc to 2:1 hexane/EtOAc) to give (E)-3-hydroxystilbene as a white solid. R_{f} 0.36 (4:1 hexane/EtOAc), 0.74 (2:1 hexane/EtOAc); mp: 125.0-125.5° C., ¹H NMR (CD₃OD): δ 6.70-6.74 (m, 1H, H-4), 7.00-7.05 (m, 2H), 7.12 (s, 2H), 7.16-7.28 (m, 2H), 7.33-7.39 (m, 2H), 7.53-7.56 (m, 2H, H-2′, H-6′); ¹³C JMOD NMR (CD₃OD): δ 114.77 (2), 116.60 (4), 120.17 (6), 128.34 (2′,6′), 129.37, 130.45, 130.59 (2×Cₐₗₖₑₙₑ, 4′), 130.50 (3′,5′), 131.49 (5), 139.61 (1′), 141.05 (1), 159.54 (3); LRESI negative ion mass spectrum; m/z 19504-Hr, 100%), 196 (16%), 391 ([2M−H]⁻);

HRESI negative ion mass spectrum; [M−H]⁻ calc. 195.0810.

### Example 9a and 9b

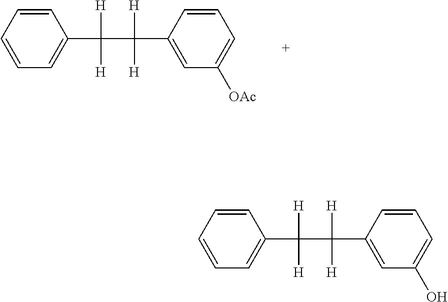 [SMILES: CC(=O)Oc1cccc(CCc2ccccc2)c1 ; Oc1cccc(CCc2ccccc2)c1]

### 3-Phenethyl-acetoxybenzene (9a) and 3-phenethyl phenol (9b)

A mixture of (E)-3-acetoxtstilbene and 10% Pd/C in methanol was hydrogenated overnight at 90 psi. Filtration through a Celite pad gave a solution which was rotary evaporated to return a grey coloured oil. This was purified by gradient elution column chromatography (9:1 EtOAc/hexane to 4:1 EtOAc/hexane) to return 2 major products comprising 3-phenethyl-acetoxybenzene as a white powder and 3-phenethyl phenol as a white solid.

### 3-Phenethyl-Acetoxybenzene

R_{f} 0.32 (9:1 hexane/EtOAc); ¹H NMR (CDCl₃): δ 2.30 (s, 3H, OAc), 2.96 (s, 4H, 2×CH₂), 6.94-6.98 (m, 2H), 7.05-7.08 (m, 1H), 7.19-7.25 (m, 3H), 7.27-7.34 (m, 3H).

### 3-Phenethyl Phenol

R_{f} 0.16 (9:1 hexane/EtOAc); ¹H NMR (CDCl₃): δ 2.83-2.95 (m, 4H, 2×CH₂), 4.84 (bs, 1H, OH), 6.64-6.68 (m, 2H), 6.75-6.78 (m, 1H), 7.12-7.31 (m, 6H).

### Example 10

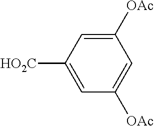 [SMILES: CC(=O)Oc1cc(OC(C)=O)cc(C(=O)O)c1]

### 3,5-Diacetoxybenzoic acid (10)

A suspension of 3,5-dihydroxybenzoic acid in ethyl acetate was cooled in an ice-bath. Acetic anhydride and pyridine were added and the reaction allowed proceeding for 60 minutes. The homogenous solution was stirred overnight at room temperature. Formic acid was added and the solution then poured onto ice-water. Further ethyl acetate was added and the organic phase separated and successively washed twice each with sat. NaHCO₃ and water, then dried, filtered and rotary evaporated to give a white solid. Recrystallization from EtOAc/hexane produced 3,5-diacetoxybenzoic acid as a white powder. R_{f} 0.20 (1:1 hexane/EtOAc), 0.39 (3:1 hexane/EtOAc); mp 161-162° C., lit mp: 157-159° C. Turner et al, Macromolecules, 1993, 26, 4617-4623; ¹H NMR (CDCl₃): δ 2.29 (s, 6H, 2×OAc), 7.18 (pseudo t, 1H, J=2.1 Hz, H4), 7.70 (pseudo d, 2H, J=2.1 Hz, H2, H6); ¹³C JMOD NMR (CD3OD): δ 18.43 (2×CH3), 118.94 (C4), 119.02 (C2,6), 131.70 (C1), 150.19 (C5), 165.46 (COOH), 168.17 (2×OCOCH3); LRESI positive ion mass spectrum; m/z 261 (MNa⁺, 100%).

### Example 11

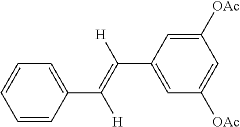 [SMILES: CC(=O)Oc1cc(/C=C/c2ccccc2)cc(OC(C)=O)c1]

### (E)-3,5-Diacetoxystilbene, [(E)-5-styryl-1,3-phenylene diacetate] (11)

3,5-Diacetoxybenzoic acid was suspended in dry toluene. N,N-DMF and thionyl chloride were added and the reaction heated under an argon atmosphere to 100° C. for 3 hours. The solvents were removed by vacuum distillation to give a pale yellow solid, which was suspended in dry toluene and sonicated under vacuum for 15 minutes. Styrene, N-ethylmorpholine and palladium diacetate (2 mole %) were added and the reaction mixture heated overnight at 120° C. under an argon gas atmosphere. Upon cooling to room temperature, ethyl acetate was added and the solution washed successively three times with 0.1 M HCl and twice with water, then dried, filtered and rotary evaporated to give a brown solid. Purification with column chromatography (gradient elution starting with 6:1 hexane/EtOAc and finished with 2:1 hexane/EtOAc) produced (E)-5-styryl-1,3-phenylene diacetate as a white powder. Further recrystallisation from EtOAc/hexane produced the compound as white fine needles. R_{f} 0.44 (4:1 hexane/EtOAc); mp 90.0-90.5° C., ¹H NMR (CDCl₃): δ 2.31 (s, 6H, 2×OAc), 6.83 (pseudo t, 1H, J=2.1 Hz, H-4), 7.04 (d, 1H, J=16.3 Hz, Hₜᵣₐₙₛ), 7.09 (d, 1H, J=trans, 16.3 Hz, Hₜᵣₐₙₛ), 7.12-7.14 (m, 2H, H-2, H-6), 7.25-7.30 (m, 1H, H-4′), 7.34-7.39 (m, 2H, H-3′, H-5′), 7.47-7.50 (m, 2H, H-2′, H-6′); ¹³C JMOD NMR (CDCl3): δ 20.09 (3×CH₃), 113.32 (4), 115.89 (2,6), 125.73 (2′,6′), 125.98, 127.13 (2×Cₜᵣₐₙₛ), 127.75 (3′,5′), 129.72 (4′), 135.69 (1′), 138.72 (1), 150.35 (3, 5), 167.93 (2×C═O); LRESI positive ion mass spectrum; m/z 319 (MNa⁺, 100%); HRESI positive ion mass spectrum; C18H16O4Na⁺; calc. 319.0946. measured 319.0943.

### Example 12

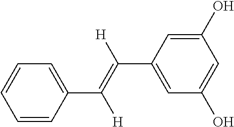 [SMILES: Oc1cc(O)cc(/C=C/c2ccccc2)c1]

### (E)-3,5-Dihydroxystilbene, [(E)-5-styrylbenzene-1,3-diol] (12)

A solution of potassium hydroxide in methanol was added to (E)-5-styryl-1,3-phenylene diacetate suspended in methanol. The solution was heated under a nitrogen atmosphere to 70° C. for 100 minutes. Upon cooling to room temperature, the solution was acidified to pH 3 by addition of 1 M HCl. Water was added, and the volume reduced by rotary evaporation and stopped at the first appearance of a precipitate. Ethyl acetate was added and the organic layer was separated and washed four times with water until the aqueous washings were neutral. The organic material was then dried, filtered and rotary evaporated to give an orange oil. Purification by gradient elution column chromatography (2:1 hexane/EtOAc to 1:1 hexane/EtOAc) gave (E)-5-styrylbenzene-1,3-diol as a white solid. R_{f} 0.17 (hexane/EtOAc 4:1), 0.62 (hexane/EtOAc 1:1); mp 157.0-157.5° C., ¹H NMR (CD₃OD): δ 6.18 (pseudo t, 1H, J=2.2 Hz, H-4), 6.46 (d, 2H, J=2.2 Hz, H-2, H-6), 6.95 (d, 1H, J=16.3 Hz, Hₜᵣₐₙₛ), 7.01 (d, 1H, J=16.3 Hz, Hₜᵣₐₙₛ), 7.15-7.21 (m, 1H, H-4′), 7.26-7.32 (m, 2H, H-3′, H-5′), 7.44-7.48 (m, 2H, 2′,6′); ¹³C JMOD NMR (CD₃OD): δ 100.91 (4), 103.92 (2,6), 125.13 (2′,6′), 126.19, 127.27, 127.48 (2×Cₜᵣₐₙₛ, 4′), 127.31 (3′,5′), 136.32 (1′), 138.52 (1), 157.28 (3, 5); LRESI positive ion mass spectrum; m/z 213 (MH⁺, 100%), 214 (18%); HRESI positive ion mass spectrum; C₁₄H₁₂O₂Na⁺; calc. 235.0735. measured 235.0729.

### Example 13a and 13b

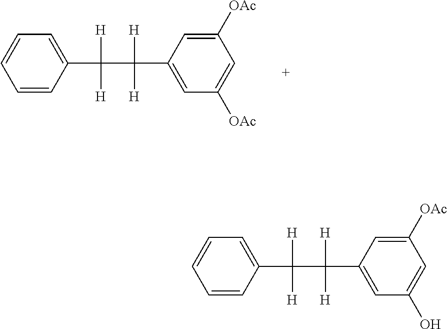 [SMILES: CC(=O)Oc1cc(CCc2ccccc2)cc(OC(C)=O)c1 ; CC(=O)Oc1cc(O)cc(CCc2ccccc2)c1]

### 5-Phenethyl-1,3-phenylene diacetate (13a) and 3-hydroxy-5-phenethylphenyl acetate (13b)

A mixture of (E)-5-styryl-1,3-phenylene diacetate and 10% Pd/C in methanol was hydrogenated overnight at 90 psi. Filtration through a Celite pad gave a clear solution which was rotary evaporated to return a clear gum, which was subsequently subjected to gradient elution column chromatography (4:1 EtOAc/hexane to 2:1 EtOAc/hexane). This procedure resulted in two major products (i) 5-phenethyl-1,3-phenylene diacetate as a clear oil, that solidified on standing and (ii) 3-hydroxy-5-phenethylphenyl acetate as a clear viscous oil.

### 5-Phenethyl-1,3-phenylene diacetate

R_{f} 0.61 (2:1 hexane/EtOAc); mp 48.0-48.5° C., ¹H NMR (CDCl₃): δ 2.25 (s, 6H, 2×OAc), 2.90 (s, 4H, 2×CH₂), 6.75 (pseudo t, 1H, J=2.1 Hz, H-4), 6.80 (pseudo d, 2H, J=2.1 Hz, H-2, H-6), 7.13-7.30 (m, 5H, H-2′, H-3′, H-4′, H-5′, H-6′); ¹³C JMOD NMR (CDCl₃): δ 20.07 (OCOCH₃), 36.26, 36.59 (2×CH₂), 112.04 (4), 118.01 (2,6), 125.13 (4′), 127.43, 127.47 (2′,3′,5′,6′), 140.16 (1′), 143.21 (1), 150.04 (3,5), 167.99 (OCOCH3); LRESI positive ion mass spectrum; m/z 321 (MNa⁺, 100%), 322 (20%); HRESI positive ion mass spectrum; C₁₈H₁₈O₄Na⁺; calc. 321.1103. measured 321.1095.

### 3-hydroxy-5-phenethylphenyl acetate

R_{f} 0.42 (2:1 hexane/EtOAc); ¹H NMR (CDCl₃): δ 2.25 (s, 3H, OAc), 2.79-2.91 (m, 4H, 2×CH₂), 6.41 (pseudo t, 1H, J=2.2 Hz, H-4), 6.48 (pseudo d, 2H, J=2.3 Hz, H-2, H-6), 7.13-7.20 (m, 3H, H-2′, H-4′, H-6′), 7.23-7.29 (m, 2H, H-3′, H-5′); ¹³C JMOD NMR (CDCl₃): δ 20.19 (OCOCH₃), 36.26, 36.59 (2×CH₂), 105.82 (2), 112.47 (4), 112.50 (6), 125.04 (4′), 127.42, 127.45 (2′,3′,5′,6′), 140.48 (1′), 143.61 (5), 150.32 (1), 155.66 (3), 169.43 (OCOCH3); LRESI positive ion mass spectrum; m/z 279 (MNa⁺, 100%), 280 (20%).

### Example 14

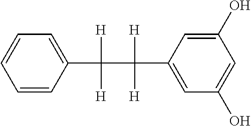 [SMILES: Oc1cc(O)cc(CCc2ccccc2)c1]

### 5-Phenethylbenzene-1,3-diol (14)

Potassium hydroxide dissolved in methanol was added to a solution of 5-phenethyl-1,3-phenylene diacetate in methanol and heated to 70° C. for 150 minutes under an argon atmosphere. Upon return to room temperature, the solution was acidified to pH 3 by addition of 1M HCl. Water was then added and the volume reduced by rotary evaporation. The solution was then extracted four times with ethyl acetate and the combined extracts dried, filtered and rotary evaporated to give a brown oil. Purification with column chromatography (gradient elution starting with 4:1 hexane/EtOAc and finishing with 2:1 hexane/EtOAc) gave 5-phenethylbenzene-1,3-diol as a clear oil. R_{f} 0.36 (2:1 hexane/EtOAc); ¹H NMR (CDCl₃): δ 2.75-2.81 (m, 2H, CH₂), 2.84-2.90 (m, 2H, CH₂), 4.70 (bs, 2H, 2×OH)), 6.17 (pseudo t, 1H, J=2.2 Hz, H-4), 6.22 (d, 2H, J=2.2 Hz, H-2, H-6), 7.13-7.19 (m, 3H, H-2′, H-4′, H-6′), 7.23-7.29 (m, 2H, H-3′, H-5′); ¹³C JMOD NMR (CDCl₃): δ 36.25, 36.61 (2×CH₂), 99.85 (4), 107.41 (2,6), 125.00 (4′), 127.41 (2′,6′), 127.49 (3′,5′), 140.72 (1′), 144.16 (1), 155.47 (3,5); LRESI negative ion mass spectrum m/z 213 ([M−H]⁻, 100%), 214 (14%), 427 ([2M−H]⁻, 13%).

### Example 15

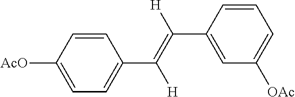 [SMILES: CC(=O)Oc1ccc(/C=C/c2cccc(OC(C)=O)c2)cc1]

### (E)-3,4′-Diacetoxystilbene, [(E)-3-(4-acetoxystyryl)phenyl acetate] (15)

A suspension of 3-acetoxybenzoic acid in dry toluene, N,N-DMF and thionyl chloride was heated to 100° C. under an argon atmosphere and maintained for 3 hours. The solvents were removed by vacuum distillation and the resultant solid redissolved in dry toluene, then this solution was sonicated under vacuum for 20 minutes. 4-Acetoxystyrene, N-ethylmorpholine and palladium diacetate (2 mole %) were added and the mixture heated overnight at 120° C. under an argon gas atmosphere. Upon cooling to room temperature, ethyl acetate was added, which was subsequently washed three times with 0.1 M HCl and twice with water, then dried, filtered and rotary evaporated to give of a brown liquid. Purification by column chromatography (gradient elution starting with 4:1 hexane/EtOAc and finishing with 2:1 hexane/EtOAc) gave a white solid. ¹H NMR showed this to be mostly (E)-3,4′-diacetoxystilbene and a small amount of unreacted 4-acetoxystyrene. Recrystallization from EtOAc/hexane gave (E)-3-(4-acetoxystyryl)phenyl acetate exclusively as white mica plates. R_{f} 0.32 (4:1 hexane/EtOAc), 0.59 (2:1 hexane/EtOAc); mp 124.5-125.0° C., ¹H NMR (CDCl₃): δ 2.28 (s, 3H, OAc), 2.29 (s, 3H, OAc), 6.93-7.09 (m, 5H, H-4, 2×Hₜᵣₐₙₛ, H-3′, H-5′), 7.21-7.22 (m, 1H, H-2), 7.30-7.34 (m, 2H, H-5, H-6), 7.45-7.50 (m, 2H, H-2′, H-6′); ¹³C JMOD NMR (CDCl₃): δ 20.11 (2×OC(O)CH₃), 118.27 (4), 119.74 (2), 120.86 (3′,5′), 123.19 (6), 126.55 (2′,6′), 126.96, 127.76 (2×Cₜᵣₐₙₛ), 128.61 (5), 133.79 (1′), 137.92 (1), 149.32 (4′), 150.15 (3), 168.35 (2×C═O).

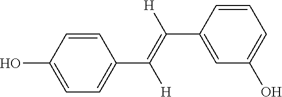 [SMILES: Oc1ccc(/C=C/c2cccc(O)c2)cc1]

### Example 16

### (E)-3,4′-Dihyroxystilbene, [(E)-3,4′-(ethene-1,2-diyl)diphenol] (16)

A solution of potassium hydroxide in methanol was added to (E)-3-(4-acetoxystyryl)phenyl acetate suspended in methanol and heated to 70° C. for 100 minutes under an argon atmosphere. Upon cooling to room temperature, the solution was acidified to pH 3 by addition of 1M HCl. Water was then added and the volume reduced by rotary evaporation until the first appearance of a precipitate. Further water and ethyl acetate were added and the 2 phases separated. The organic phase was washed three times with water until the aqueous washings were neutral, then dried, filtered and rotary evaporated to return a cream coloured solid. Purification by column chromatography (gradient elution starting with 2:1 hexane/EtOAc and finishing with 1:1 hexane/EtOAc) gave (E)-3,4′-(ethene-1,2-diyl)diphenol as a cream coloured powder. R_{f} 0.35 (2:1 hexane/EtOAc); mp 213.0-213.5° C., ¹H NMR (CD₃OD): δ 6.60-6.63 (m, 1H, H-2), 6.74 (d, 2H, Jₒᵣₜₕₒ=8.7 Hz, H-3′, H-3′), 6.85 (d, 1H, J=16.3 Hz, Hₜᵣₐₙₛ), 6.89-6.94 (m, 2H, H-4, H-6), 6.98 (d, 1H, J=16.3 Hz, Hₜᵣₐₙₛ), 7.09 (pseudo t, 1H, Jₒᵣₜₕₒ=7.9 Hz, H-5), 7.33 (d, 2H, H-2′, H-6′); ¹³C JMOD NMR (CD₃OD): δ 111.21 (2), 112.79 (4), 114.15 (3′,5′), 116.68 (6), 124.51 (5), 126.49 (2′,6′), 127.14 (Cₜᵣₐₙₛ), 128.12 (1′), 128.22 (Cₜᵣₐₙₛ), 138.35 (1), 155.90 (4′), 156.20 (3); LRESI negative ion mass spectrum; m/z 211 ([M−H]⁻, 100%), 212 (13%); HRESI positive ion mass spectrum; C₁₄H₁₂O₂Na⁺; calc. 235.0735. measured 235.0730.

### Example 17

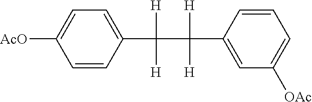 [SMILES: CC(=O)Oc1ccc(CCc2cccc(OC(C)=O)c2)cc1]

### 3-(4-Acetoxyphenylethyl)phenyl acetate (17)

A mixture of (E)-3,4′-(ethene-1,2-diyl)diphenol and 10% Pd/C in methanol was hydrogenated overnight at 85 psi. Filtration through a Celite plug gave a clear solution which was rotary evaporated to return a clear viscous oil. This material was purified by isocratic elution from column chromatography with 2:1 hexane/EtOAc to give 3-(4-acetoxyphenylethyl)phenyl acetate as a clear viscous oil. This material slowly solidified upon standing overnight at room temperature. R_{f} 0.69 (2:1 hexane/EtOAc); mp 69.5-70.0° C., ¹H NMR (CDCl₃): δ 2.259 (s, 3H, OAc), 2.263 (s, 3H, OAc), 2.89 (s, 4H, 2×CH₂), 6.88-6.92 (m, 2H, H-4, H-6), 6.94-7.02 (m, 3H, Jₒᵣₜₕₒ=8.6 Hz, H-2, H-3′, H-5′), 7.12-7.16 (m, 2H, H-2′, H-6′), 7.22-7.28 (m, 1H, H-5); ¹³C JMOD NMR (CDCl₃): δ 20.07 (2×OCOCH₃), 35.91, 36.55 (2×CH₂), 118.23 (4), 120.38 (3′,5′), 120.96 (2), 124.99 (6), 128.30 (5), 128.38 (2′,6′), 137.96 (1′), 142.25 (1), 148.03 (4′), 149.85 (3), 168.43, 168.51 (2×OCOCH3); LRESI positive ion mass spectrum; m/z 321 (MNa⁺, 100%), 299 (MH⁺, 10%).

### Example 18

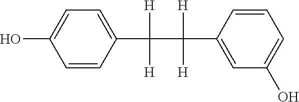 [SMILES: Oc1ccc(CCc2cccc(O)c2)cc1]

### 3,4′-(Ethane-1,2-diyl)diphenol (18)

Potassium hydroxide dissolved in methanol was added to 3-(4-acetoxyphenylethyl)phenyl acetate suspended in methanol and heated to 80° C. under an argon atmosphere for 2 hours. Upon cooling to room temperature, the solution was acidified to pH 3 by addition of 1 M HCl. Water was then added and the volume reduced by rotary evaporation, The solution was then extracted four times with ethyl acetate and the combined extracts dried, filtered and rotary evaporated to give a viscous yellow oil. Purification by isocratic elution from column chromatography with 2:1 hexane/EtOAc) gave 3,4′-(ethane-1,2-diyl)diphenol as a white solid. R_{f} 0.41 (2:1 hexane/EtOAc); mp 108.0-108.5° C., ¹H NMR (CDCl₃): δ 2.84 (s, 4H, 2×CH₂), 4.60 (s, 1H, OH), 4.65 (s, 1H, OH), 6.63-6.78 (m, 5H, H-2, H-4, H-6, H-3′, H-5′), 7.00-7.07 (m, 2H, H-2′, H-6′), 7.10-7.18 (m, 1H, H-5); ¹H NMR (CD₃OD): δ 2.79 (s, 4H, 2×CH₂), 6.58-6.73 (m, 5H, H-2, H-4, H-6, H-3′, H-5′), 6.97-7.02 (m, 2H, H-2′, H-6′), 7.15-7.10 (m, 1H, H-5); ¹³C JMOD NMR (CD₃OD): δ 34.19, 35.45 (2×CH₂), 109.81 (4), 112.13 (3′,5′), 112.52 (2), 117.17 (5), 126.38 (6), 126.56 (2′,6′), 130.24 (1′), 140.94 (1), 152.24 (4′), 154.13 (3); LRESI negative ion mass spectrum m/z 213 ([M−H]⁻, 100%), 427 ([2M−H]⁻, 66%)

### Example 19

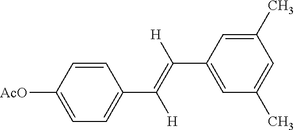 [SMILES: CC(=O)Oc1ccc(/C=C/c2cc(C)cc(C)c2)cc1]

### (E)-3,5-Dimethyl-4′-acetoxystilbene, [(E)-4-(3,5-Dimethylstyryl)phenyl acetate] (19)

3,5-Dimethylbenzoic acid was suspended in dry toluene. N,N-DMF and thionyl chloride were added and the reaction heated at 100° C. under an argon atmosphere for 3 hours. After cooling to room temperature the solvents were removed by vacuum distillation. The residual yellow oil was dissolved in dry toluene and the solution sonicated under vacuum. 4-Acetoxystyrene, N-ethylmorpholine and palladium diacetate (2 mole %) were added and the mixture heated overnight at 120° C. under an argon atmosphere. Upon cooling to room temperature, ethyl acetate was added and the solution washed successively three times with 0.1 M HCl and twice with water, then dried, filtered and rotary evaporated to give a brown oil. Purification by gradient elution column chromatography (9:1 hexane/EtOAc to 7:1 hexane/EtOAc) gave 4-acetoxystyrene as a major product and a cream coloured solid as a secondary product. This solid was subsequently triturated with hexane to give (E)-4-(3,5-dimethylstyryl)phenyl acetate as a white solid. Recrystallization of this product from hexane produced translucent fine needles. R_{f} 0.34 (9:1 hexane/EtOAc), 0.59 (2:1 hexane/EtOAc); mp 84.9-85.0° C., ¹H NMR (CDCl₃): δ 2.28 (s, 3H, OAc), 2.31 (s, 6H, 2×CH₃), 6.89 (bs, 1H, H4), 6.97 (d, 1H, J=16.4 Hz, Hₜᵣₐₙₛ), 7.02-7.07 (m, 3H, H3′, H5′, Hₜᵣₐₙₛ), 7.10 (bs, 2H, 2,6), 7.45-7.50 (m, 2H, Jₒᵣₜₕₒ=8.7 Hz, H2′, H6′); ¹³C JMOD NMR (CDCl₃): δ 20.12 (OCOCH₃), 20.32 (2×CH₃), 120.79 (3′,5′), 123.49 (2,6), 128.20, 128.55 (4, 2×Cₐₗₖₑₙₑ), 134.38 ((1′), 136.14 (1), 137.14 (3,5), 149.02 (4′), 168.40 (3×C═O); LRESI positive ion mass spectrum; m/z 555 (2M+Na⁺, 14%), 321 (MNa⁺+MeOH, 53%), 289 (MNa⁺, 100%), 267 (MH⁺, 12%).

### Example 20

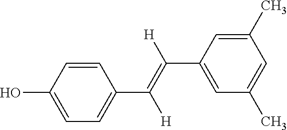 [SMILES: Cc1cc(C)cc(/C=C/c2ccc(O)cc2)c1]

### (E)-3,5-Dimethyl-4′-hydroxystilbene, [(E)-4-(3,5-Dimethylstyryl)phenol] (20)

A solution of potassium hydroxide in methanol was added to (E)-4-(3,5-dimethylstyryl)phenyl acetate dissolved in methanol and heated to 65° C. under a nitrogen atmosphere for 2 hours. The solution was then poured onto ice-water, then acidified to pH 3 with 1 M HCl. Sodium chloride was added and the solution then extracted three times with ethyl acetate. The combined extracts were dried, filtered and rotary evaporated to give a yellow solid. Purification by flash chromatography (gradient elution starting with 9:1 hexane/EtOAc and finishing with 4:1 hexane/EtOAc) gave (E)-4-(3,5-dimethylstyryl)phenol as a white amorphous powder. R_{f} 0.29 (4:1 hexane/EtOAc); mp: 142.0-142.2° C., ¹H NMR (CDCl₃): δ 2.31 (s, 6H, 2×CH₃), 4.66 (s, 1H, OH), 6.77-6.81 (m, 2H, Jₒᵣₜₕₒ=8.7 Hz, 3′,5′), 6.86 (bs, 1H, 4), 6.88 (d, 1H, Tₜᵣₐₙₛ=16.1 Hz, alkene), 7.00 (d, 1H, alkene), 7.08 (s, 2H, 2,6), 7.34-7.39 (m, 2H, 2′,6′); ¹³C JMOD NMR (CDCl₃): δ 19.25 (2×CH₃), 114.22 (3′,5′), 122.82 (2,6), 124.88 (Cₐₗₖₑₙₑ), 126.55 (2′,6′), 126.72 (Cₐₗₖₑₙₑ), 127.43 (4), 128.39 (1′), 136.58 (3,5), 136.66 (1), 155.74 (4′); LRESI negative ion mass spectrum; m/z 223 ([M−H]⁻, 100%), 224 (18%); HRESI negative ion mass spectrum; m/z [M−H]⁻ calc. 223.1123. measured 223.1126.

### Example 21

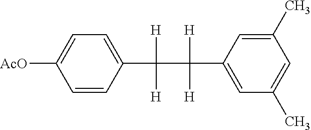 [SMILES: CC(=O)Oc1ccc(CCc2cc(C)cc(C)c2)cc1]

### 4-(3,5-Dimethylphenethyl)phenyl-acetate (21)

A mixture of (E)-4-(3,5-dimethylstyryl)phenyl acetate and 10% Pd/C in methanol was hydrogenated overnight at 90 psi. Filtration through a Celite pad gave a clear solution which was rotary evaporated to a viscous oil. Purification by gradient elution column chromatography (9:1 EtOAc/hexane to 4:1 EtOAc/hexane) gave 4-(3,5-dimethylphenethyl)phenyl-acetate as a clear viscous oil. R_{f} 0.64 (2:1 hexane/EtOAc), 0.32 (4:1 hexane/EtOAc), 0.18 (9:1 hexane/EtOAc); ¹H NMR (CDCl₃): δ 2.267, 2.270, 2.271 (3×s, 9H, 2×CH₃, OAc), 2.77-2.90 (m, 4H, 2×CH₂), 6.79 (bs, 2H, 2,6), 6.82 (bs, 1H, 4), 6.95-7.00 (m, 2H, Jₒᵣₜₕₒ=8.6 Hz, 2′,6′), 7.14-7.19 (m, 2H, 3′,5′); ¹³C JMOD NMR (CDCl₃): δ 20.13 (OCOCH₃), 20.40 (2×CH₃), 36.52, 36.92 (2×CH₂), 120.47 (3′,5′), 125.44 (2, 6), 126.78 (4), 136.89 (3,5), 138.65 (1′), 140.64 (1), 148.06 (4′), 168.57 (OCOCH₃); LRESI positive ion mass spectrum; m/z 291 (MNa⁺, 100%), 292 (20%), 269 (MH⁺, 5%).

### Example 22

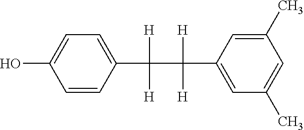 [SMILES: Cc1cc(C)cc(CCc2ccc(O)cc2)c1]

### 4-(3,5-Dimethylphenethyl)phenol (22)

A solution of potassium hydroxide in methanol was added to 4-(3,5-dimethylphenethyl)phenyl-acetate dissolved in methanol and heated for 3 hours at 65° C. under an argon atmosphere. The volume was reduced by rotary evaporation and the concentrated solution was poured into water, then acidified to pH 3 with 1M HCl and extracted three times with EtOAc. The combined extracts were then dried, filtered and rotary evaporated to give a clear viscous oil. Purification by column chromatography (gradient elution starting with 9:1 hexane/EtOAc and finishing with 4:1 hexane/EtOAc) gave 4-(3,5-dimethylphenethyl)phenol as a clear viscous oil. R_{f} 0.71 (2:1 EtOAc/hexane), 0.59 (2:1 hexane/EtOAc), 0.39 (4:1 hexane/EtOAc); ¹H NMR (CD₃OD): δ 2.28 (s, 4H, 2×CH₃), 2.78-2.84 (m, 1H, 2×CH₂), 4.59 (bs, 1H, OH), 6.71-6.76 (m, 2H, Tₒᵣₜₕₒ=8.5 Hz, 2,6), 6.80 (bs, 2H, 2′,5′), 6.82 (bs, 1H, 4′), 7.03-7.07 (m, 2H, 3,5); ¹³C JMOD NMR (CDCl₃): δ 20.57 (2×CH₃), 36.42, 37.36 (2×Cₐₗₖₑₙₑ), 114.64 (3,5), 125.65 (2′,6′), 126.88 (4′), 128.86 (2,6), 133.72 (1), 137.06 (3′,5′), 141.14 (1′), 152.67 (4); LRESI negative ion mass spectrum; m/z 225 ([M−H]⁻, 100%), 226 (17%); HRESI positive ion mass spectrum; m/z MH⁺ calc. 227.1436. measured 227.1438.

### Example 23

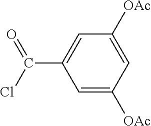 [SMILES: CC(=O)Oc1cc(OC(C)=O)cc(C(=O)Cl)c1]

### 3,5-Diacetoxy Benzoyl Chloride (23)

A suspension of 3,5-diacetoxybenzoic acid in dry toluene, N,N-DMF and thionyl chloride was heated at 100° C. for three hours under a nitrogen atmosphere. The solvents were removed by vacuum distillation and the remaining viscous liquid treated with hexane to precipitate a yellow solid. Hexane was removed by rotary evaporation to give a pale yellow powder. This compound was immediately on-reacted. R_{f} 0.88 (2:1 EtOAc/hexane). R_{f} 0.88 (2:1 EtOAc/hexane).

### Example 24

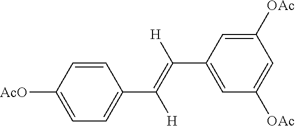 [SMILES: CC(=O)Oc1ccc(/C=C/c2cc(OC(C)=O)cc(OC(C)=O)c2)cc1]

### (E)-3, 4′,5-Triacetoxystilbene, [(E)-5-(4-acetoxystyryl)-1,3-phenylene diacetate] (24)

3,5-diacetoxybenzoyl chloride, 4-acetoxystyrene, N-ethylmorpholine and palladium diacetate (0.45 mole %) were dissolved in dry toluene (20 mL) and heated overnight at 120° C. under a nitrogen atmosphere. Upon cooling to room temperature, ethyl acetate was added and the reaction washed twice each with 0.1 M HCl and water, then dried, filtered and rotary evaporated to give a brown solid. Purification by isocratic elution from column chromatography with 1:1 hexane/Et₂O gave a white solid. This material was purified by gradient elution chromatography (4:1 hexane/EtOAc to 2:1 hexane/EtOAc) to give E-5-(4-acetoxystyryl)-1,3-phenylene diacetate as a white solid. R_{f} 0.29 (2:1 hexane/EtOAc); mp 112.5-113.0° C., ¹H NMR (CDCl₃): δ 2.27 (s, 9H, 3×OAc), 6.80 (pseudo t, 1H, J=2.1 Hz, 4′), 6.93 (d, 1H, J=16.3 Hz, Hₜᵣₐₙₛ), 7.03 (d, 1H, J=16.3 Hz, Hₜᵣₐₙₛ), 7.04-7.09 (m, 4H, 3,5,2′,6′), 7.44-7.47 (m, 2H, 2,6); ¹³C JMOD NMR (CDCl₃): δ 20.07 (3×CH₃), 113.39 (4′), 115.88 (2′,6′), 120.88 (3,5), 126.19, 126.64, 128.64 (2×Cₜᵣₐₙₛ, 2, 6), 133.45 (1), 138.53 (1′), 149.46 (4), 150.34 (3′,5′), 167.91, 168.30 (3×C═O); LRESI positive ion mass spectrum; m/z 377 (MNa⁺, 100%), 378 (21%).

### Example 25

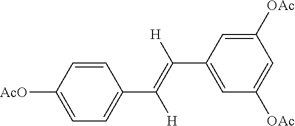 [SMILES: CC(=O)Oc1ccc(/C=C/c2cc(OC(C)=O)cc(OC(C)=O)c2)cc1]

### (E)-3, 4′,5-Triacetoxystilbene, (E)-5-(4-acetoxystyryl)-1,3-phenylene diacetate (25)

A mixture of 3,5-diacetoxybenzoic acid, N,N-DMF and thionyl chloride in dry toluene was heated to 100° C. under an argon atmosphere for 3 hours. The solvents were removed by vacuum distillation and the solid white residue subsequently suspended in dry toluene and sonicated under vacuum. 4-acetoxystyrene, N-ethylmorpholine and palladium diacetate (2.0 mole %) were added and this mixture was heated overnight at 120° C. Upon cooling to room temperature, ethyl acetate was added and the solution washed three times with 0.1 M HCl, then, dried, filtered and rotary evaporated to give a brown solid. Purification by gradient elution column chromatography (2:1 hexane/EtOAc to 100% EtOAc) gave (E)-5-(4-acetoxystyryl)-1,3-phenylene diacetate as a white solid. R_{f} 0.29 (2:1 hexane/EtOAc); mp 112.5-113.0° C., ¹H NMR (CDCl₃): δ 2.27 (s, 9H, 3×OAc), 6.80 (pseudo t, 1H, J=2.1 Hz, 4′), 6.93 (d, 1H, J=16.3 Hz, Hₜᵣₐₙₛ), 7.03 (d, 1H, J=16.3 Hz, Hₜᵣₐₙₛ), 7.04-7.09 (m, 4H, 3,5,2′,6′), 7.44-7.47 (m, 2H, 2,6); ¹³C JMOD NMR (CDCl₃): δ 20.07 (3×CH₃), 113.39 (4′), 115.88 (2′,6′), 120.88 (3,5), 126.19, 126.64, 128.64 (2×Cₜᵣₐₙₛ, 2, 6), 133.45 (1), 138.53 (1′), 149.46 (4), 150.34 (3′,5′), 167.91, 168.30 (3×C═O); LRESI positive ion mass spectrum; m/z 377 (MNa⁺, 100%), 378 (21%).

### Example 26

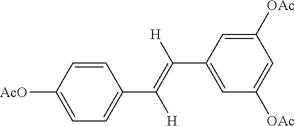 [SMILES: CC(=O)Oc1ccc(/C=C/c2cc(OC(C)=O)cc(OC(C)=O)c2)cc1]

### (E)-3, 4′,5-Triacetoxystilbene, (E)-5-(4-acetoxystyryl)-1,3-phenylene diacetate (26)

3,5-diacetoxybenzoyl chloride, 4-acetoxystyrene, N-ethylmorpholine and palladium diacetate (0.45 mole %) were dissolved in dry toluene and heated overnight at 120° C. under a nitrogen atmosphere. Upon cooling to room temperature, ethyl acetate was added and the reaction washed twice with 0.1 M HCl, then water and dried, filtered and rotary evaporated to a brown solid. Purification by isocratic elution from column chromatography (1:1 hexane/Et₂O) gave a white solid. This material was purified by gradient elution chromatography (4:1 hexane/EtOAc to 2:1 hexane/EtOAc) to give (E)-5-(4-acetoxystyryl)-1,3-phenylene diacetate as a white solid. R_{f} 0.29 (2:1 hexane/EtOAc); mp 112.5-113.0° C., ¹H NMR (CDCl₃): δ 2.27 (s, 9H, 3×OAc), 6.80 (pseudo t, 1H, J=2.1 Hz, 4′), 6.93 (d, 1H, J=16.3 Hz, Hₜᵣₐₙₛ), 7.03 (d, 1H, J=16.3 Hz, Hₜᵣₐₙₛ), 7.04-7.09 (m, 4H, 3,5,2′,6′), 7.44-7.47 (m, 2H, 2,6); ¹³C JMOD NMR (CDCl₃): δ 20.07 (3×CH₃), 113.39 (4′), 115.88 (2′,6% 120.88 (3,5), 126.19, 126.64, 128.64 (2×Cₜᵣₐₙₛ, 2, 6), 133.45 (1), 138.53 (1′), 149.46 (4), 150.34 (3′,5′), 167.91, 168.30 (3×C═O); LRESI positive ion mass spectrum; m/z 377 (MNa⁺, 100%), 378 (21%).

### Example 27

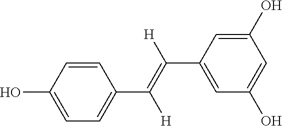 [SMILES: Oc1ccc(/C=C/c2cc(O)cc(O)c2)cc1]

### (E)-5-(4-hydroxystyryl)benzene-1,3-diol (27)

Potassium hydroxide dissolved in methanol was added to (E)-5-(4-acetoxystyryl)-1,3-phenylene diacetate suspended in methanol and heated to 65° C. under a nitrogen atmosphere for 1 hour. The volume was reduced by rotary evaporation and the solution then acidified to pH 3 with 1 M HCl. Ethyl acetate was added and the solution then washed three times with saturated brine, then dried, filtered and rotary evaporated to give a dark red solid. Purification by isocratic elution from column chromatography (0,040-0,063 mm SiO₂) with EtOAc gave (E)-5-(4-hydroxystyryl)benzene-1,3-diol as a pale beige coloured solid. R_{f} 0.65 (EtOAc); ¹H NMR (CD₃OD): δ 6.13 (pseudo t, 1H, J=2.2 Hz, 4), 6.41-6.42 (m, 2H, 2,6), 6.71-6.79 (m, 3H, Hₜᵣₐₙₛ, 3%5% 6.93 (d, 1H, J=16.3 Hz, Hₜᵣₐₙₛ), 7.29-7.36 (m, 2H, Jₒᵣₜₕₒ=8.6 Hz, 2′,6′). ¹³C JMOD NMR (CD₃OD): δ. LRESI positive ion mass spectrum; m/z 229 (MH⁺, 100%), 230 (23%).

### Example 28

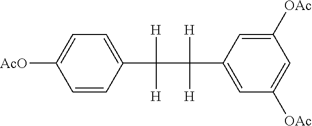 [SMILES: CC(=O)Oc1ccc(CCc2cc(OC(C)=O)cc(OC(C)=O)c2)cc1]

### 4-(3,5-Diacetoxyphenethyl)-acetoxybenzene

A mixture of (E)-4-(3,5-diacetoxyphenethylene)-acetoxybenzene and 10% Pd/C in methanol was hydrogenated overnight at 95 psi. Filtration through a Celite pad gave a clear solution which was rotary evaporated to give a viscous oil. Purification by isocratic elution from column chromatography (0,040-0,063 mm SiO₂) with 2:1 EtOAc/hexane) gave 4-(3,5-diacetoxyphenethyl)-acetoxybenzene as a clear viscous oil, which solidified to a white solid after standing at room temperature. R_{f} 0.68 (2:1 EtOAc/hexane), 0.52 (2:1 Et₂O/hexane); mp 52.0-52.5° C., ¹H NMR (CDCl₃): δ 2.25 (s, 6H, 2×OAc), 2.26 (s, 3H, OAc), 2.88 (s, 4H, 2×CH₂), 6.75-6.77 (m, 3H), 6.95-6.98 (m, 2H, Jₒᵣₜₕₒ=8.4 Hz, ArH), 7.12-7.14 (m, 2H, ArH); ¹³C JMOD NMR (CDCl₃): δ 20.03 (3×CH₃), 35.57, 36.47 (2×CH₂), 112.08 (4′), 118.00 (3,5), 120.46 (2′,6′), 128.33 (2, 6), 137.64 (1), 142.91 (1′), 148.02 (4), 150.01 (3′,5′), 167.98, 168.53 (3×C═O); LRESI positive ion mass spectrum; m/z 379 (MNa⁺, 100%), 380 (21%).

### Example 29

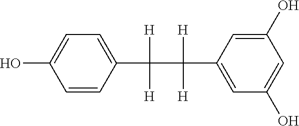 [SMILES: Oc1ccc(CCc2cc(O)cc(O)c2)cc1]

### 4-(3,5-Dihydroxyphenethyl)-phenol (29)

A solution of potassium hydroxide in methanol was added to 4-(3,5-diacetoxyphenethyl)-acetoxybenzene in methanol then heated to 65° C. for 30 minutes under an argon atmosphere. The solution was then poured into water, acidified to pH 4 with 1 M HCl. NaCl was added and the solution extracted with EtOAc. The organic layer was separated and washed twice with saturated brine, then dried over anhydrous Na₂SO₄, filtered and rotary evaporated to give an orange solid. Purification by isocratic elution from column chromatography (0,040-0,063 mm SiO₂) with 2:1 EtOAc/hexane gave 4-(3,5-diahydroxyphenethyl)-phenol as a white solid. R_{f} 0.44 (2:1 EtOAc/hexane); Mp: 160.5-161.0° C., ¹H NMR (CD₃OD): δ 2.61-2.75 (m, 4H, 2×CH₂), 6.05 (pseudo t, 1H, J=2.2 Hz, 4′), 6.09-6.10 (m, 2H, 2′,6′), 6.61-6.66 (m, 2H, Jₒᵣₜₕₒ8.6 Hz, 3,5), 6.91-6.96 (m, 2H, 2,6); ¹³C JMOD NMR (CD₃OD): δ 35.56, 37.08 (2×CH₂), 98.81 (4′), 105.82 (2′,6′), 113.66 (3,5), 128.01 (2, 6), 131.82 (1), 143.33 (1′), 153.85 (4), 156.80 (3′,5′); LRESI positive ion mass spectrum; m/z 231 (MH⁺, 100%), 232 (12%), 253 (MNa⁺, 11%); HRESI positive ion mass spectrum; MH⁺ calc. 231.1021. measured 231.1014.

### Example 30

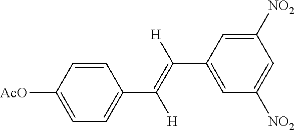 [SMILES: CC(=O)Oc1ccc(/C=C/c2cc([N+](=O)[O-])cc([N+](=O)[O-])c2)cc1]

### (E)-3,5-dinitro-4′-acetoxystilbene, (E)-4-(3,5-dinitrostyryl)phenyl acetate (30)

3,5-Dinitrobenzoyl chloride, 4-acetoxystyrene, N-ethylmorpholine and palladium diacetate (2 mole %) were added to dry toluene and heated overnight at 120° C. under an argon gas atmosphere. Upon cooling to room temperature, ethyl acetate was added and the solution washed four times with 0.1M HCl and twice with water. The organic layer was then dried over anhydrous Na₂SO₄, filtered and rotary evaporated to give a dark brown solid. This material was suspended in CH₂Cl₂ and silica added before removing the solvent by rotary evaporation. The remaining powder was then loaded as a narrow band onto a column and purified by isocratic elution chromatography (0.040-0,063 mm SiO₂) with CH₂Cl₂ to give (E)-4-(3,5-dinitrostyryl)phenyl acetate as a bright yellow solid. R_{f} 0.42 (CH₂Cl₂); mp, 217.5-218.0° C., ¹H NMR (CDCl₃): δ 2.30 (s, 3H, OAC), 7.08-7.17 (m, 3H, H_{3′}, H_{5′}), 7.32 (d, 1H, Jₜᵣₐₙ=16.3 Hz, Hₐₗₖₑₙₑ), 7.54-7.58 (m, 2H, Jₒᵣₜₕₒ=8.5 Hz, H_{2′}, H_{6′}), 8.60-8.61 (m, 2H, Jₘₑₜₐ=2.0 Hz, H₂, H₆), 8.87 (pseudo t, 1H, H₄); ¹³C JMOD NMR (CDCl₃): δ 20.31 (CH₃), 115.92 (4); 121.75 (3′,5′), 124.22 (Cₐₗₖₑₙₑ), 125.48 (2,6), 127.66 (2′,6′), 132.42 (Cₐₗₖₑₙₑ), 133.15 (1′), 140.39 (1), 148.05 (3,5), 150.28 (4′), 168.55 (CO); EI MS: m/z 328 (M+, 6%), 286 (100%), 147 (30%).

### Example 31

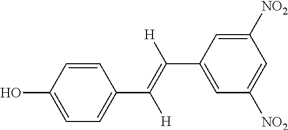 [SMILES: O=[N+]([O-])c1cc(/C=C/c2ccc(O)cc2)cc([N+](=O)[O-])c1]

### (E)-3,5-dinitro-4′-hydroxystilbene, (E)-4-(3,5-dinitrostyryl)phenol) (31)

A solution of potassium hydroxide in methanol was added to (E)-4-(3,5-dinitrostyryl)phenyl acetate suspended in methanol and heated to 65° C. under an argon atmosphere for 2 hours. The reaction solution volume was halved by rotary evaporation, then poured onto ice-water and then acidified to pH 3 with 1M HCl. The resultant bright yellow solid was then extracted with ethyl acetate and the organic layer was removed and washed three times with water. The extract was dried over anhydrous Na₂SO₄, then filtered and rotary evaporated to give a bright orange solid. Purification by gradient elution chromatography (absolute CH₂Cl₂ to 9:1 CH₂Cl₂/MeOH) gave (E)-4-(3,5-dinitrostyryl)phenol as a bright orange powder. R_{f} 0.31 (CH₂Cl₂); ¹H NMR (CD₃OD): δ 6.79 (m, 2H, Jₒᵣₜₕₒ=8.5 Hz, H_{3′}, H_{5′}), 7.16 (d, 1H, Jₜᵣₐₙ=16.2 Hz, Hₐₗₖₑₙₑ), 7.40-7.49 (m, 3H, Hₐₗₖₑₙₑ, H_{2′}, H_{6′}), 8.67-8.69 (m, 2H, H₂, H₆), 8.73 (m, 1H, H₄); ¹³C JMOD NMR (CD₃OD): δ. ESI MS: m/z 286 (100%), 147 (53%).

### Example 32

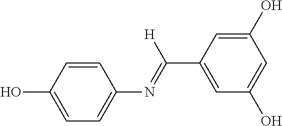 [SMILES: Oc1ccc(/N=C/c2cc(O)cc(O)c2)cc1]

### (E)-5-[(4-Hydroxy-phenylimino)-methyl]-benzene-1,3-diol (32)

A mixture of 3,5-dihydroxybenzaldehyde, 4-aminophenol and anhydrous sodium sulphate in dichloromethane was vigorously stirred at room temperature for 3 hours. Further anhydrous sodium sulphate was added and stirring continued for 1 hour. Dichloromethane was removed by rotary evaporation. The remaining white powder was resuspended in boiling ethanol, then removed by vacuum filtration. The recovered solid was rinsed with further boiling ethanol. The clear filtrates were combined and rotary evaporated to dryness to give (E)-5-[(4-hydroxy-phenylimino)-methyl]-benzene-1,3-diol as a pale pink solid. Lit (WO 2006/108864 A2) 162° C. (dec); ¹H NMR (d₆-DMSO): δ 6.36 (pseudo t, 1H, J=2.2 Hz, H-4), 6.80-6.85 (m, 4H, H-2, H-6, H-3′, H-5′), 7.16-7.22 (m, 2H, Jₒᵣₜₕₒ=8.8 Hz, H-2′, H-6′), 8.43 (s, 1H, imine-H), 9.47 (bs, 3H, 3×phenolic-OH); ¹³C JMOD NMR (d₆-DMSO): δ 106.27 (4), 107.41 (2,6), 116.70 (3′,5′), 123.42 (2′,6′), 139.35 (1), 143.61 (1′), 157.16 (4′), 158.38 (imine-C), 159.62 (3,5); LRESI positive ion mass spectrum; m/z 230 (MH⁺, 100%), 231 (13%); HRESI positive ion mass spectrum; C₁₃H₁₁NO₃Na⁺; calc. 252.0637. measured 252.0633.

### Example 33

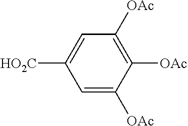 [SMILES: CC(=O)Oc1cc(C(=O)O)cc(OC(C)=O)c1OC(C)=O]

### 3,4,5-Triacetoxybenzoic acid (33)

A suspension of 3,4,5-trihydroxybenzoic acid in ethyl acetate was cooled in an ice-bath, then acetic anhydride and pyridine were added. The reaction was allowed to proceed for 45 minutes and then the solution was then heated to reflux for 3 hours. Further acetic anhydride was then added and the solution stirred overnight at room temperature. Formic acid was added and the solution poured onto ice-water. The organic layer was removed, then washed four times with sat. NaHCO₃ and twice with water, dried, filtered and rotary evaporated to give a white solid. Recrystallization from 1:1 EtOAc/hexane gave 3,4,5-triacetoxybenzoic acid as a white powder. R_{f} 0.47 (EtOAc); mp 167.5-168.0° C., ¹H NMR (CDCl₃): δ 2.279 (s, 6H, 2×OAc), 2.284 (s, 3H, OAc), 7.84 (s, 2H, arom); ¹³C JMOD NMR (CDCl₃): δ. LRESI positive ion mass spectrum; m/z 319 (M+Na⁺, 100%), negative ion mass spectrum; m/z 295 ([M−H]⁻, 100%).

### Example 34

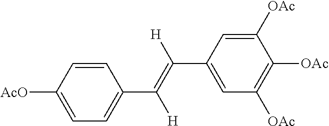 [SMILES: CC(=O)Oc1ccc(/C=C/c2cc(OC(C)=O)c(OC(C)=O)c(OC(C)=O)c2)cc1]

### (E)-3,4,5,4′-Tetraacetoxystilbene[(E)-5-(4-acetoxystyryl)benzene-1,2,3-triyltriacetate] (34)

3,4,5-Triacetoxybenzoic acid was suspended in dry toluene. N,N-DMF and thionyl chloride were added and the reaction heated to 100° C. for 3 hours under an argon atmosphere. Solvents were removed by vacuum distillation to give a pale yellow solid. This acid chloride was suspended in dry toluene and the mixture sonicated under vacuum for 30 minutes. 4-Acetoxystyrene, N-ethylmorpholine and palladium diacetate (2 mole %) were added and the mixture heated overnight at 120° C. under an argon atmosphere. Upon cooling to room temperature, ethyl acetate was added and the solution washed successively once with water, three times with 0.1 M HCl, and again with water, then dried, filtered and rotary evaporated to give a dark brown viscous oil. Purification by gradient elution column chromatography (absolute hexane to absolute EtOAc) separated and recovered 4-acetoxystyrene and (E)-3,4,5,4′-tetra acetoxystilbene. Recrystallisation of (E)-3,4,5,4′-tetra acetoxystilbene from hexane:ethyl acetate (3:2) gave (E)-3,4,5,4′-tetra acetoxystilbene as fine white needles. R_{f} 0.49 (1:1 hexane/EtOAc), 0.23 (2:1 hexane/EtOAc); mp 162.5-163.0° C., ¹H NMR (CDCl₃): δ 2.26 (s, 3H, OAc), 2.27 (s, 6H, 2×OAc), 2.28 (s, 3H, OAc), 6.91 (d, 1H, J=16.2 Hz, Hₜᵣₐₙₛ), 6.99 (d, 1H, Hₜᵣₐₙₛ), 7.04-7.08 (m, 2H, Jₒᵣₜₕₒ=8.6 Hz, Jₘₑₜₐ=1.9 Hz, H-3′, H-5′), 7.20 (bs, 2H, H-2, H-6), 7.42-7.47 (m, 2H, H-2′, H-6′); ¹³C JMOD NMR (CDCl₃): δ 19.12, 19.62, 20.08 (OCOCH₃), 117.45 (2,6), 120.87 (3′,5′), 125.65 (Cₐₗₖₑₙₑ), 126.64 (2′,6′), 128.70 (Cₐₗₖₑₙₑ), 132.76 (1), 133.39 (4), 134.89 ((1′), 142.61 (3,5), 149.47 (4′), 165.99, 166.82, 168.30 (OCOCH₃); LRESI positive ion mass spectrum; m/z 435 (MNa⁺, 100%); HRESI positive ion mass spectrum; calculated for m/z C₂₂H₂₀O₈Na⁺, 435.1056. measured 435.1061.

### Example 35

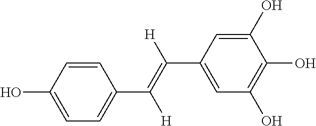 [SMILES: Oc1ccc(/C=C/c2cc(O)c(O)c(O)c2)cc1]

### (E)-3,4,5,4′-Tetrahydroxystilbene[(E)-5-(4-hydroxystyryObenzene-1,2,3-triol] (35)

A solution of p-toluene sulphonic acid monohydrate (4 mg, 0.021 mmol) in methanol (1.0 mL) was added to (E)-3,4,5,4′-tetraacetoxystilbene (150 mg, 0.3623 mmol) suspended in methanol (9 mL). The reaction was heated to reflux for 4 hours under an argon atmosphere. The solvent was removed by rotary evaporation and the remaining pink solid then triturated with hexane. The solid was filtered off and air dried to give a pink coloured powder. Purification by gradient elution column chromatography (2:1 EtOAc/hexane to absolute EtOAc) gave (E)-3,4,5,4′-tertahydroxystilbene as a pale yellow powder. R_{f} 0.34 (2:1 EtOAc/hexane); ¹H NMR (CD₃OD): δ 6.50 (bs, 2H, H-2, H-6), 6.70 (d, 1H, Jₜᵣₐₙₛ=16.2 Hz, Hₐₗₖₑₙₑ), 6.69-6.76 (m, 2H, Jₒᵣₜₕₒ=8.7 Hz, H-3′, H-5′), 6.77 (d, 1H, Hₐₗₖₑₙₑ), 7.25-7.30 (m, 2H, H-2′, H-6′); ¹³C JMOD NMR (CD₃OD): δ 104.12 (2,6), 114.07 (3′,5′), 124.55, 124.90 (2×Cₐₗₖₑₙₑ), 126.01 (2′,6′), 128.45 (1′), 128.56 (1), 131.62 (4), 144.63 (3, 5), 155.42 (4′); LRESI negative ion mass spectrum; m/z 243 ([M−H]⁻, 100%), 244 (17%).

### Example 36

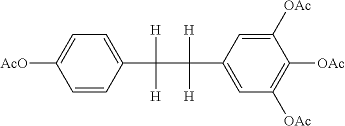 [SMILES: CC(=O)Oc1ccc(CCc2cc(OC(C)=O)c(OC(C)=O)c(OC(C)=O)c2)cc1]

### 5-(4-Acetoxyphenethyl)benzene-1,2,3-triyltriacetate (36)

A mixture of (E)-3,4,5,4′-tetraacetoxystilbene and 10% Pd/C in methanol was hydrogenated overnight at 90 psi. The solution was filtered through a Celite pad, then rotary evaporated to a grey oil. This oil was then dissolved in ethyl acetate, then cooled in an ice-bath before addition of acetic anhydride and pyridine. The reaction was allowed to proceed for 60 minutes before addition of further acetic anhydride and subsequent heating at 80° C. for 3 hours. This mixture was then stirred at room temperature overnight. The reaction solution was then poured onto ice-water, and extracted with ethyl acetate. The organic layer was subsequently removed, washed four times with water, then dried, filtered and rotary evaporated to give an orange oil. This oil was purified by gradient elution chromatographically (4:1 hexane/EtOAc to 1:1 hexane/EtOAc) to give 5-(4-acetoxy phenethyl)benzene-1,2,3-triyltriacetate as a white powder. R_{f} 0.36 (1:1 hexane/EtOAc); mp 121.5-122.0° C., ¹H NMR (CDCl₃): δ 2.24 (s, 6H, 2×OAc), 2.25 (s, 3H, OAc), 2.26 (s, 3H, OAc), 2.87 (bs, 4H, 2×CH₂), 6.89 (bs, 2H, H-2, H-6), 6.95-6.99 (m, 2H, Jₒᵣₜₕₒ=8.4 Hz, Jₘₑₜₐ=2.0 Hz, H-3′, H-5′), 7.11-7.16 (m, 2H, H-2′, H-6′); ¹³C JMOD NMR (CDCl₃): δ 19.11, 19.59, 20.06 (4×OCOCH₃), 35.54, 36.23 (2×CH₂), 119.56 (2,6), 120.49 (3′,5% 128.32 (2′,6′), 131.72 (4), 137.54 (1′), 139.02 (1), 142.19 (3, 5), 148.04 (4′), 166.08, 166.86, 168.51 (4×OCOCH3); LRESI positive ion mass spectrum; m/z 437 (MNa⁺, 100%), 438 (22%); HRESI positive ion mass spectrum; C₂₂H₂₂O₈Na⁺; calc. 437.1212. measured 437.1196.

### Example 37

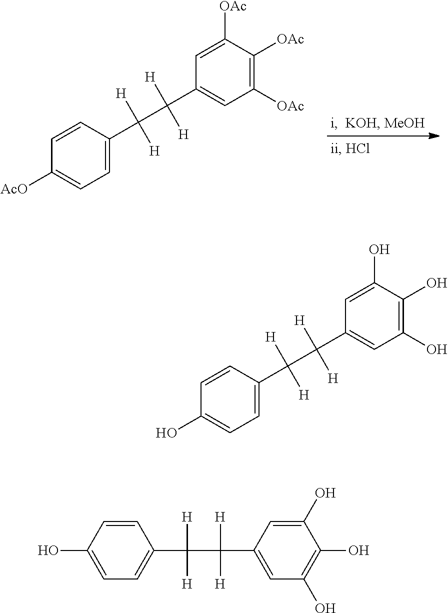 [SMILES: CC(=O)Oc1ccc(CCc2cc(OC(C)=O)c(OC(C)=O)c(OC(C)=O)c2)cc1 ; Oc1ccc(CCc2cc(O)c(O)c(O)c2)cc1 ; Oc1ccc(CCc2cc(O)c(O)c(O)c2)cc1]

### 5-(4-hydroxyphenethyl)benzene-1,2,3-triol (37)

A solution of p-toluene sulphonic acid monohydrate in methanol was added to a suspension of 1-(3,4,5-tri-O-acetylphenethyl)-4-O-acetylbenzene in methanol. The reaction was heated to 85° C. overnight under an argon gas atmosphere. The solvent was removed by rotary evaporation and the oil purified with column chromatography (isocratically eluted with 2:1 EtOAc/hexane) to return 5-(4-hydroxyphenethyl)benzene-1,2,3-triol as a white solid. R_{f} 0.55 (2:1 EtOAc/hexane); mp 180.0-188.5° C., ¹H NMR (CD₃OD): δ 2.55-2.61 (m, 2H, CH₂), 2.66-2.72 (m, 2H, CH₂), 6.13 (bs, 2H, H-2, H-6), 6.61-6.65 (m, 2H, Jₒᵣₜₕₒ=8.5 Hz, H-3′, H-5′), 6.90-6.93 (m, 2H, H-2′, H-6′); ¹³C JMOD NMR (CD₃OD): δ 39.03 (CH₂), 39.75 (CH₂), 109.36 (2,6), 116.74 (3′,5′), 131.14 (2′,6′), 132.74 (4), 135.11 (1′), 131.37 (1), 147.43 (3,5), 156.83 (4′); LRESI negative ion mass spectrum; m/z 245 ([M−H]⁻, 100%), 246 (18%); HRESI negative ion mass spectrum; calculated m/z for C₁₄H₁₄O₄—H⁻ 245.0814. measured 245.0814.

### Example 38

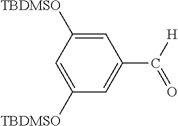 [SMILES: CC(C)(C)[Si](C)(C)Oc1cc(C=O)cc(O[Si](C)(C)C(C)(C)C)c1]

### 3,5-Di(tert-butyldimethylsilyloxy)benzaldehyde (38)

The reaction was conducted under an argon gas atmosphere. Diisopropylethylamine was added to a solution of 3,5-dihydroxybenzaldehyde dissolved in dried N,N-DMF. After The solution was well agitated at room temperature for 20 minutes and then tert-butyldimethylsilylchloride dissolved in dry N,N-DMF was added drop-wise over 15 minutes and the mixture stirred at room temperature overnight. The reaction was then poured into ice-water, extracted with dichloromethane, and the combined extracts dried (anhyd. Na₂SO₄) and rotary evaporated to return a brown oil. Purification with column chromatography (gradient eluted beginning with hexane (100%) and finishing with 9:1 hexane/EtOAc) 3,5-di(tert-butyldimethylsilyloxy)benzaldehyde as a clear oil. R_{f} 0.80 (4:1 hexane/EtOAc), 0.50 (19:1 hexane/EtOAc); ¹H NMR (CDCl₃): δ 0.22 (s, 12H, 2×Si(CH₃)₂), 0.99 (s, 18H, 2×SiC(CH₃)₃), 6.59 (pseudo t, 1H, J=2.3 Hz, H₄), 6.96 (pseudo d, 2H, J=2.3 Hz, H₂, H₆), 9.86 (s, 1H, CHO); ¹³C JMOD NMR (CDCl₃): δ −4.15 (SiCH₃). 18.46 (C(CH₃)₃), 25.90 (C(CH₃)₃), 114.61 (2,6), 118.59 (4), 138.73 (1), 157.55 (3,5), 191.80 (CHO).

### Example 39a

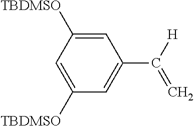 [SMILES: C=Cc1cc(O[Si](C)(C)C(C)(C)C)cc(O[Si](C)(C)C(C)(C)C)c1]

### (5-Vinyl-1,3-phenylene)bis(oxy)bis(tert-butyldimethylsilane (39a)

The reactions were conducted under an argon gas atmosphere. Methylenetriphenylphosphorane was first generated in situ by heating to reflux a mixture of methyltriphenylphosphonium bromide (34.787 g, 97.443 mmol, Aldrich Chem. Co.) and potassium tert-butoxide (9.010 g, 73.856 mmol, Aldrich Chem. Co.) in anhydrous THF. On return to room temperature, a solution of 3,5-di(tert-butyldimethylsilyloxy)benzaldehyde in anhydrous THF was added drop wise and the reaction heated to reflux overnight. Ethyl acetate (600 mL) was then added and the solution washed with water, dried (anhyd. Na₂SO₄), filtered and rotary evaporated to a brown oil. Purification with column chromatography (gradient eluted beginning with hexane (100%) and finishing with 7:1 hexane/EtOAc) gave (5-vinyl-1,3-phenylene)bis(oxy)bis(tert-butyldimethylsilane) as a pale yellow oil; R_{f} 0.44 (50:1 hexane/EtOAc), 0.75 (25:1 hexane/EtOAc); ¹H NMR (CDCl₃): δ 0.21 (s, 12H, 2×Si(CH₃)₂), 0.99 (s, 18H, 2×SiC(CH₃)₃), 5.02 (dd, 1H, J=12.8 Hz, J=1.5 Hz, Hₐₗₖ), 5.66 (dd, 1H, J=13.2 Hz, J=0.7 Hz, Hₐₗₖ), 6.59 (pseudo t, 1H, J=1.6 Hz, H₂), 6.96 (pseudo d, 2H, J=1.6 Hz, H₄, H₆), 6.58 (dd, 1H, Hₐₗₖ); ¹³C JMOD NMR (CDCl₃): δ −8.03 (SiCH₃). 18.56 (C(CH₃)₃), 26.06 (C(CH₃)₃), 111.80 (4,6), 112.02 (2), 114.20 (═CH₂), 137.12 (CH═), 139.78 (5), 156.97 (1,3); LRESI positive ion mass spectrum; m/z 365 (MH⁺, 100%), 366 (37).

### Example 39b

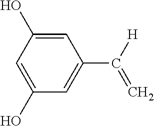

### 5-Vinylbenzene-1,3-diol, [3,5-Dihdroxy styrene] (39)

The reaction was conducted under an argon gas atmosphere. Tetrabutylammonium fluoride dissolved in THF was added over 15 minutes to (5-vinyl-1,3-phenylene)bis(oxy)bis(tert-butyldimethylsilane) in anhydrous THF, and the reaction left at room temperature for 90 minutes. The volume was then reduced by rotary evaporation and the volume replaced with ethyl acetate. The solution was then washed with water, dried (anhyd. Na₂SO₄), filtered and rotary evaporated to a brown oil. Purification with column chromatography (isocratically eluted with 1:1 hexane/EtOAc) gave a viscous pale yellow oil. Further column chromatography (gradient eluted beginning with 4:1 hexane/EtOAc and finishing with 1:1 hexane/EtOAc) returned 5-vinylbenzene-1,3-diol as a pale yellow oil; R_{f} 0.58 (1:1 hexane/EtOAc), 0.76 (1:2 hexane/EtOAc); ¹H NMR (CD₃OD): δ 5.16 (dd, 1H, J=10.8 Hz, J=1.1 Hz, Hₐₗₖ), 5.66 (dd, 1H, J=17.6 Hz, J=1.2 Hz, Hₐₗₖ), 6.22 (pseudo t, 1H, J=2.2 Hz, H₂), 6.40 (pseudo d, 2H, J=2.2 Hz, H₄, H₆), 6.58 (dd, 1H, Hₐₗₖ); ¹³C JMOD NMR (CD₃OD): δ 102.24 (2), 104.92 (4,6), 112.95 (═CH₂), 137.11 (CH═), 140.07 (5), 158.31 (1,3); LRESI negative ion mass spectrum; m/z 13504-Hr, 100%), 136 (17%).

### Example 40

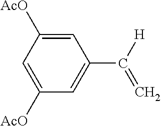 [SMILES: C=Cc1cc(OC(C)=O)cc(OC(C)=O)c1]

### 5-Vinyl-1,3-phenylene diacetate, [3,5-Diacetoxystyrene] (40)

The reaction was conducted under an argon gas atmosphere. 5-Vinylbenzene-1,3-diol was dissolved in ethyl acetate. Pyridine and acetic anhydride were added and the reaction heated to reflux for 4 hours and left overnight at room temperature. The volume was increased with further ethyl acetate and the solution washed with water, dried (anhyd. Na₂SO₄), filtered and rotary evaporated to a yellow semisolid. Purification with column chromatography (isocratically eluted with 4:1 hexane/EtOAc) gave 5-vinyl-1,3-phenylene diacetate as a clear oil; R_{f} 0.29 (4:1 hexane/EtOAc); ¹H NMR (CDCl₃): δ 2.30 (s, 6H, 2×OAc), 5.33 (d, 1H, J=10.9 Hz, Hₐₗₖ), 5.75 dd, 1H, J=17.5 Hz, Hₐₗₖ), 6.66 (dd, 1H, Hₐₗₖ), 6.84 (pseudo t, 1H, J=2.1 Hz, H₂), 7.03 (pseudo d, 2H, J=2.1 Hz, H₄, H₆); ¹³C JMOD NMR (CDCl₃): δ 21.37 (2×CH₃), 114.93 (2), 116.18 (═CH₂), 117.04 (4,6), 135.66 (CH═), 140.27 (5), 151.54 (1,3), 169.22 (2×CO);

LRESI positive ion mass spectrum; m/z 243 (MH⁺, 100%), 244 (13%).

### Example 41

### (E)-3,3′,5,5′-Tetraacetoxy-stilbene (41)

Attempted preparation of the (2+2) adduct; (E)-3,5,3′,5′-Tetraacetoxystilbene. Systematically named as (E)-5,5′(ethene-1,2-diyl)bis(benzene-5,3, 1-triyl)tetraacetate.

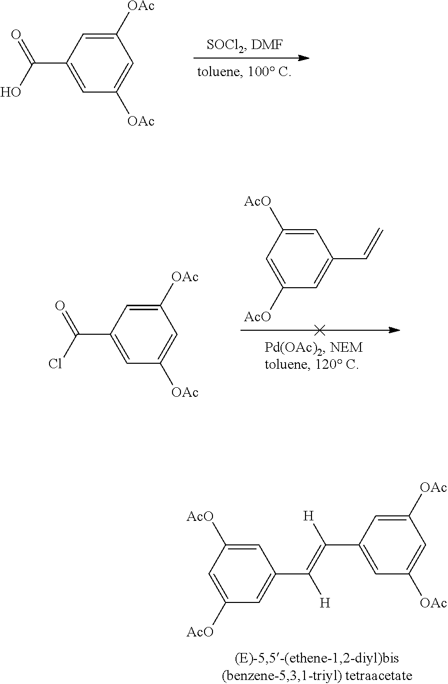 [SMILES: CC(=O)Oc1cc(OC(C)=O)cc(C(=O)O)c1 ; CC(=O)Oc1cc(OC(C)=O)cc(C(=O)Cl)c1 ; C=Cc1cc(OC(C)=O)cc(OC(C)=O)c1 ; CC(=O)Oc1cc(/C=C/c2cc(OC(C)=O)cc(OC(C)=O)c2)cc(OC(C)=O)c1]

3,5-Diacetoxybenzoic acid (1.841 g, 7.735 mmol, batch BDp125-21-9-07) was suspended in sodium wire dried toluene (50 mL). Dry N,N-DMF (0.5 mL) and thionyl chloride (5.0 mL, 69 mmol) were added and the reaction was heated to reflux for three hours under an Argon gas atmosphere. All the material dissolved within 20 minutes. The solvents were removed by vacuum distillation (0.1 mm/60° C.) and the resultant yellow solid then redissolved in dry toluene (25 mL) and sonicated under vacuum for 15 minutes to remove dissolved gases. 3,5-Diacetoxystyrene (1.547 g, 7.032 mmol, batch BDp143-18-10-07), N-ethylmorpholine (983 μL, 7.735 mmol) and palladium diacetate (35 mg, 0.0155 mmol, 2 mole %) were added and the mixture heated to reflux overnight under an Argon gas atmosphere. On return to room temperature, ethyl acetate (200 mL) was added and the solution washed successively with water (5×50 mL), dried (anhyd. Na₂SO₄), filtered and rotary evaporated to give 3.269 g of dark brown gum. TLC of this crude material showed a number of products. ¹H NMR (300 MHz, CDCl₃) showed no obvious trans-stilbene product, and styrene starting material. ESI mass spectroscopy suggested no penta-O-acetate or partially deacetylated adduct(s). The positive mode showed a m/z 243 [MNa⁺] consistent for the styrene starting material. The negative ion mode showed a m/z 237 [M−H]⁻ consistent for the acid starting material.

The crude product was then re-acetylated. The whole 3.269 g was dissolved in ethyl acetate (150 mL) and pyridine (40 mL) and acetic anhydride (40 mL) added. This reaction was heated to reflux for 5 hours and left overnight at room temperature. Water (50 mL) was added and the solution reduced to half volume with rotary evaporation. Dichloromethane (500 mL) was then added and the solution washed with water (6×400 mL), dried (anhyd. Na₂SO₄), filtered and rotary evaporated to give 5.253 g of dark brown viscous oil (This still had a residual pyridine smell). Purification with column chromatography (0,040-0,063 mm SiO₂, gradient eluted starting with 4:1 hexane/EtOAc and finished with 2:1 hexane/EtOAc EtOAc) returned as major product recovered 3,5-diacetoxystyrene (1.506 g) as a clear oil

¹H NMR (CDCl₃): δ 2.30 (s, 6H, 2×OAc), 5.32 (d, 1H, J=10.8 Hz, Hₐₗₖ), 5.74 dd, 1H, J=17.5 Hz, Hₐₗₖ), 6.65 (dd, 1H, Hₐₗₖ), 6.77 (pseudo t, 1H, J=2.1 Hz, H₂), 7.03 (pseudo d, 2H, J=2.1 Hz, H₄, H₆).

### Prophetic Example 42

### (E)-3,5-3′,5′-Tetrahydroxy-stilbenes (42)

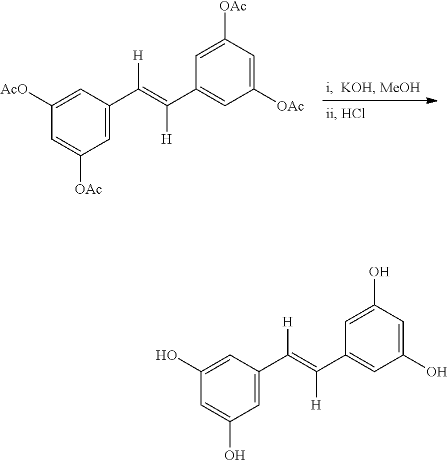 [SMILES: CC(=O)Oc1cc(/C=C/c2cc(OC(C)=O)cc(OC(C)=O)c2)cc(OC(C)=O)c1 ; Oc1cc(O)cc(/C=C/c2cc(O)cc(O)c2)c1]

### Prophetic Example 43

### 3,5-Diacetoxy-5-(3,5-diacetoxy-phenethyl)-benzene-(43)

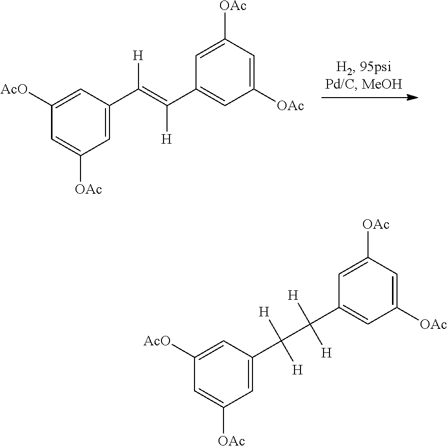 [SMILES: CC(=O)Oc1cc(/C=C/c2cc(OC(C)=O)cc(OC(C)=O)c2)cc(OC(C)=O)c1 ; CC(=O)Oc1cc(CCc2cc(OC(C)=O)cc(OC(C)=O)c2)cc(OC(C)=O)c1]

### Prophetic Example 44

### 3,5-Dihydroxy-5-(3,5-diahydroxy-phenethyl)-benzene-(44)

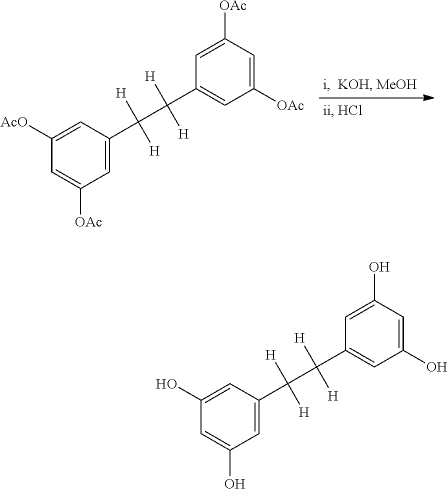 [SMILES: CC(=O)Oc1cc(CCc2cc(OC(C)=O)cc(OC(C)=O)c2)cc(OC(C)=O)c1 ; Oc1cc(O)cc(CCc2cc(O)cc(O)c2)c1]

### Example 45

### (E)-3,4,5-3′,5′-Pentacetoxystilbene (45)

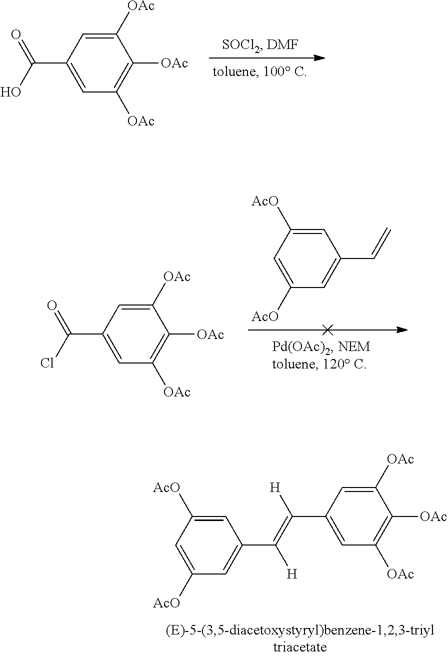 [SMILES: CC(=O)Oc1cc(C(=O)O)cc(OC(C)=O)c1OC(C)=O ; CC(=O)Oc1cc(C(=O)Cl)cc(OC(C)=O)c1OC(C)=O ; C=Cc1cc(OC(C)=O)cc(OC(C)=O)c1 ; CC(=O)Oc1cc(/C=C/c2cc(OC(C)=O)c(OC(C)=O)c(OC(C)=O)c2)cc(OC(C)=O)c1]

The general methodology was sourced from Spencer, A., “Selective Preparation of Non-Symmetrically Substituted Divinylbenzenes by Palladium Catalysed Arylations of Alkenes with Bromobenzoic Acid Derivatives”, J. Organomet. Chem., 1984, 265, 323-331⁵. An increased amount of catalyst was used because of the failure of the (2+2) preparation BDp145-22-10-07.

3,4,5-Triacetoxybenzoic acid (2.294 g, 7.75 mmol, batch BDp79-1-8-07) was suspended in sodium wire dried toluene (500 mL) and thionyl chloride (10.0 mL, 138 mmol) added. The reaction was heated to reflux under an Argon gas atmosphere for three hours. All the material dissolved within 30 minutes. The solvents were removed by vacuum distillation (0.1 mm/60° C.) to give a white solid, which was redissolved in dry toluene (20 mL) and sonicated under vacuum for 30 minutes to remove dissolved gases. 3,5-Diacetoxystyrene (1.365 g, 6.205 mmol, batch BDp143-18-10-07), N-ethylmorpholine (985 μL, 7.775 mmol) and palladium diacetate (87 mg, 0.3875 mmol, 5 mole % with respect to the acid chloride) were added and the mixture heated to reflux overnight under an Argon gas atmosphere. Further palladium diacetate (87 mg, 0.3875 mmol, 5 mole %) was added and the heating continued for an additional 4 hours. On return to room temperature, ethyl acetate (500 mL) was added and the solution washed successively with 0.1M HCl (300 mL) and water (3×300 mL), dried (anhyd. Na₂SO₄), filtered and rotary evaporated to give 3.371 g of dark brown gum. TLC of this crude material showed a number of products. ¹H NMR (400 MHz, CDCl₃) showed no obvious trans-stilbene product, and styrene starting material. ESI mass spectroscopy: both positive and negative ion modes, suggested no penta-O-acetate or partially deacetylated adduct(s). 3,4,5-Triacetoxybenzoic acid (2.294 g, 7.75 mmol, batch BDp79-1-8-07) was suspended in sodium wire dried toluene (500 mL) and thionyl chloride (10.0 mL, 138 mmol) added. The reaction was heated to reflux under an Argon gas atmosphere for three hours. All the material dissolved within 30 minutes. The solvents were removed by vacuum distillation (0.1 mm/60° C.) to give a white solid, which was redissolved in dry toluene (20 mL) and sonicated under vacuum for 30 minutes to remove dissolved gases. 3,5-Diacetoxystyrene (1.365 g, 6.205 mmol, batch BDp143-18-10-07), N-ethylmorpholine (985 μL, 7.775 mmol) and palladium diacetate (87 mg, 0.3875 mmol, 5 mole % with respect to the acid chloride) were added and the mixture heated to reflux overnight under an Argon gas atmosphere. Further palladium diacetate (87 mg, 0.3875 mmol, 5 mole %) was added and the heating continued for an additional 4 hours.

¹H NMR (CDCl₃): δ 2.28 (s, 6H, 2×OAc), 5.32 (d, 1H, J=10.8 Hz, Hₐₗₖ), 5.73 dd, 1H, J=17.5 Hz, Hₐₗₖ), 6.64 (dd, 1H, Hₐₗₖ), 6.82 (pseudo t, 1H, J=2.1 Hz, H₂), 7.01 (pseudo d, 2H, J=2.1 Hz, H₄, HO.

### Prophetic Example 46

### (E)-3,4,5-3′,5′-Pentacetoxystilbene (46)

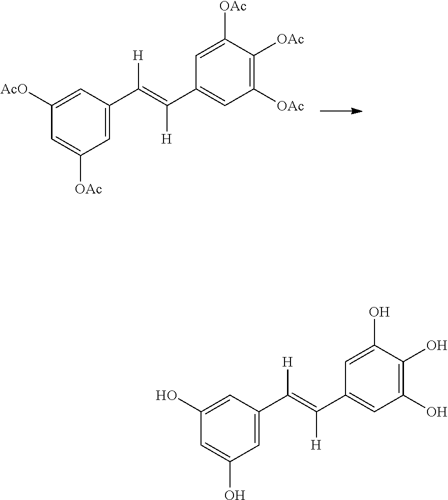 [SMILES: CC(=O)Oc1cc(/C=C/c2cc(OC(C)=O)c(OC(C)=O)c(OC(C)=O)c2)cc(OC(C)=O)c1 ; Oc1cc(O)cc(/C=C/c2cc(O)c(O)c(O)c2)c1]

### Prophetic Example 47

### 1,2,3,Triacetoxy-5-(3,5-diacetoxy-phenethyl)-benzene-(47)

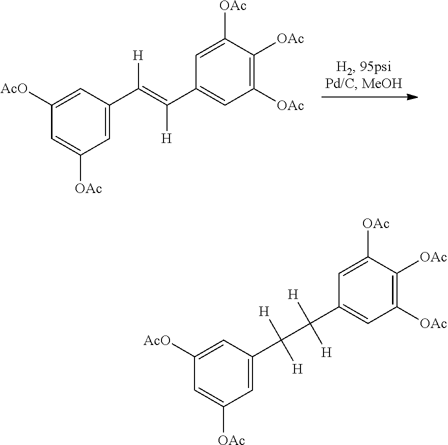 [SMILES: CC(=O)Oc1cc(/C=C/c2cc(OC(C)=O)c(OC(C)=O)c(OC(C)=O)c2)cc(OC(C)=O)c1 ; CC(=O)Oc1cc(CCc2cc(OC(C)=O)c(OC(C)=O)c(OC(C)=O)c2)cc(OC(C)=O)c1]

### Prophetic Example 48

### 1,2,3,Trihydroxy-5-(3,5-diahydroxy-phenethyl)-benzene-(48)

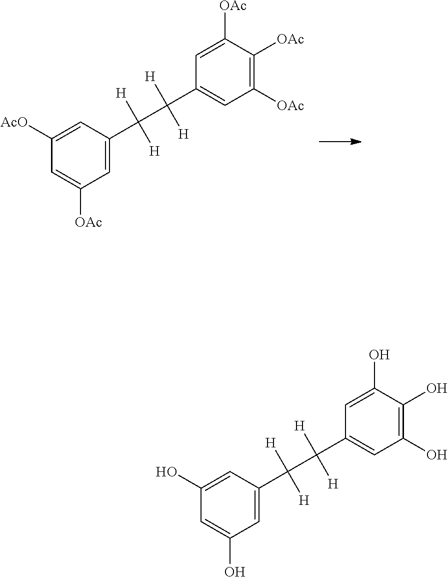 [SMILES: CC(=O)Oc1cc(CCc2cc(OC(C)=O)c(OC(C)=O)c(OC(C)=O)c2)cc(OC(C)=O)c1 ; Oc1cc(O)cc(CCc2cc(O)c(O)c(O)c2)c1]

### Example 49

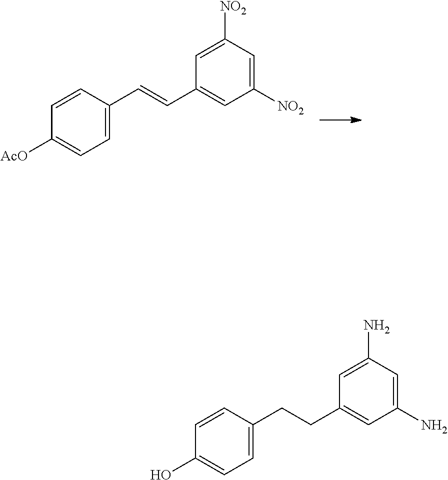 [SMILES: CC(=O)Oc1ccc(/C=C/c2cc([N+](=O)[O-])cc([N+](=O)[O-])c2)cc1 ; Nc1cc(N)cc(CCc2ccc(O)cc2)c1]

### 4-(3,5-diaminophenethyl)-phenol (49)

A mixture of (E)-3,5-dinitro-4′-acetoxystilbene and 10% Pd/C in methanol was hydrogenated overnight at 90 psi. Filtration through a Celite pad gave a pale pink coloured solution which was rotary evaporated to dryness to give a pale pink solid.

### Example 50

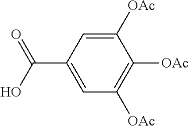 [SMILES: CC(=O)Oc1cc(C(=O)O)cc(OC(C)=O)c1OC(C)=O]

### 3,4,5-Triacetoxybenzoic acid (50)

A suspension of 3,4,5-trihydroxybenzoic acid in ethyl acetate was cooled in an ice-bath and acetic anhydride and pyridine added. After 1 hour, the resultant solution was then heated to reflux for 3 hours. Further acetic anhydride was added and the solution stirred overnight at room temperature. Formic acid was then added, and the solution poured onto ice-water. The organic layer was separated and washed with saturated aqueous sodium bicarbonate and water, dried (anhyd. Na₂SO₄), filtered and rotary evaporated to a white solid. Recrystallization from a 1:1 mixture of EtOAc/hexane gave two crops of 3,4,5-triacetoxybenzoic acid; R_{f} 0.47 (EtOAc); mp 167.5-168.0° C.; ¹H NMR (300 MHz, CDCl₃): δ 2.279 (s, 6H, 2×OAc), 2.284 (s, 3H, OAc), 7.84 (s, 2H, arom); ¹³C JMOD NMR (75 MHz, CDCl₃): δ 20.48, 20.87 (3×OCOCH₃), 123.16 (2,3), 127.75 (1), 139.71 (4), 143.89 (3,5), 166.72, 167.93 (3×OCOCH₃), 169.96 (CO₂H); LRESI positive ion mass spectrum; m/z 319 (M+Na⁺, 100%), negative ion mass spectrum; m/z 295 ([M−H]⁻, 100%).

### Example 51

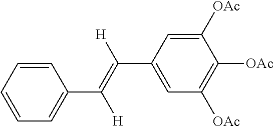 [SMILES: CC(=O)Oc1cc(/C=C/c2ccccc2)cc(OC(C)=O)c1OC(C)=O]

### (E)-3,4,5-Triacetoxystilbene (51)

3,4,5-Triacetoxybenzoic acid was suspended in dried toluene and N,N-DMF and thionyl chloride added. The reaction was heated to 100° C. under a nitrogen atmosphere for three hours and the solvents removed by vacuum distillation to return a yellow solid. This was suspended in dry toluene and sonicated under vacuum for 30 minutes to remove dissolved gases. Styrene, N-ethylmorpholine, and palladium (II) diacetate 2 mole %, Aldrich) were added and the mixture heated to reflux overnight under a nitrogen atmosphere. On return to room temperature, ethyl acetate was added and the solution washed successively with water, 0.1M HCl and water, dried (anhyd. Na₂SO₄), filtered and rotary evaporated to a dark brown viscous oil. Purification with column chromatography (gradient eluted starting with 3:1 hexane/EtOAc and finished with 2:1 hexane/EtOAc) returned (E)-3,4,5-triacetoxy stilbene as a cream coloured solid; R_{f} 0.86 (1:1 hexane/EtOAc), 0.34 (2:1 hexane/EtOAc); mp 119.0-119.5° C.; ¹H NMR (300 MHz, CDCl₃): δ 2.30 (s, 3H, OAc), 2.31 (s, 6H, 2×OAC), 7.02 (d, 1H, J=16.3 Hz, Hₜᵣₐₙₛ), 7.05 (d, 1H, Hₜᵣₐₙₛ), 7.26 (bs, 2H, H-2, H-6), 7.26-7.32 (m, 1H, H-4′), 7.34-7.40 (m, 2H, H-3′, H-5′), 7.47-7.52 (m, 2H, Jₒᵣₜₕₒ=8.6 Hz, H-2′, H-6′); ¹³C JMOD NMR (75 MHz, CDCl₃): δ 20.38, 20.87 (OCOCH₃), 118.70 (2,6), 126.68 (Cₐₗₖₑₙₑ), 126.96 (2′,6′), 128.34 (Cₐₗₖₑₙₑ), 128.99 (3′,5′), 131.02 (4′), 133.97 (4), 136.32 (1), 136.88 (1′), 143.87 (3,5), 167.25 (OCOCH₃), 168.09 (2×OCOCH₃); LRESI positive ion mass spectrum; m/z 377 (MNa⁺, 100%), 393 (MK⁺, 41%).

### Example 52

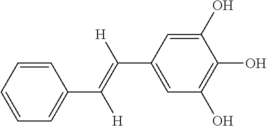 [SMILES: Oc1cc(/C=C/c2ccccc2)cc(O)c1O]

### (E)-3,4,5-Trihydroxystilbene [(E)-5-styrylbenzene-1,2,3-triol] (52)

A methanolic solution of p-toluene sulphonic acid monohydrate was added to (E)-3,4,5-triacetoxystilbene suspended in methanol (50 mL) and the reaction then heated to 85° C. overnight under a positive pressure nitrogen gas atmosphere The solvent was removed by rotary evaporation to give a salmon pink coloured solid which was purified with column chromatography (isocratically eluted with 2:1 EtOAc/hexane) to return (E)-3,4,5-trihydroxystilbene as a pale beige coloured powder. R_{f} 0.63 (2:1 EtOAc/hexane); mp 172.0-172.5° C., ¹H NMR (300 MHz, CD₃OD): δ 6.61 (bs, 2H, H-2, H-6), 6.90 (d, 1H, Jₜᵣₐₙₛ=16.3 Hz, Hₐₗₖₑₙₑ), 6.96 (d, 1H, Hₐₗₖₑₙₑ), 7.18-7.23 (m, 1H, H-4′), 7.30-7.35 (m, 2H, H-3′, H-5′), 7.46-7.50 (m, 2H, H-2′, H-6′); ¹³C JMOD NMR (75 MHz, CD₃OD): δ 107.71 (2,6), 127.82 (Cₐₗₖₑₙₑ), 127.91 (2′,6% 128.78 (Cₐₗₖₑₙₑ), 130.39 (3′,5′), 130.83 (4′), 131.11 (1), 135.32 (4), 139.95 (1′), 147.82 (3,5); LRESI negative ion mass spectrum; m/z 227 ([M−H]⁻, 100%), 228 (15%).

### Example 53

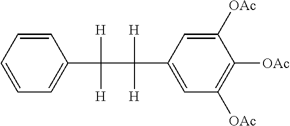 [SMILES: CC(=O)Oc1cc(CCc2ccccc2)cc(OC(C)=O)c1OC(C)=O]

### 5-Phenethylbenzene-1,2,3-triyl triacetate (53)

(E)-3,4,5-Triacetoxystilbene and 10% Pd/C in methanol was hydrogenated overnight at 90 psi. The reaction was then filtered through a Celite pad and the filtrate rotary evaporated to a grey solid. This was dissolved in ethyl acetate, cooled in an ice-bath and acetic anhydride and pyridine added and the reaction heated to 80° C. for 3 hours. Further acetic anhydride was added and solution stirred overnight at room temperature. The reaction was then poured onto ice-water and extracted with ethyl acetate. The extract was washed with water, dried (anhyd. Na₂SO₄), filtered and rotary evaporated to return a yellow oil. This was chromatographed (isocratically eluted with 2:1 hexane/EtOAc) to give 5-phenethylbenzene-1,2,3-triyl triacetate as a cream coloured solid. R_{f} 0.49 (1:1 hexane/EtOAc), 0.66 (1:2 hexane/EtOAc); mp 79.5-80.0° C.; ¹H NMR (300 MHz, CDCl₃): δ 2.28 (s, 6H, 2×OAc), 2.29 (s, 3H, OAc), 2.93 (bs, 4H, 2×CH₂), 6.95 (bs, 2H, H-2, H-6), 7.16-7.24 (m, 3H, H-2′, H-4′, H-6′), 7.26-7.33 (m, 2H, H-3′, H-5′); ¹³C JMOD NMR (75 MHz, CDCl₃): δ 20.23 (OCOCH₃), 20.70 (2×OCOCH₃), 37.30, 37.44 (2×CH₂), 120.70 (2,6), 126.26 (4′), 128.53 (2′,6′), 128.60 (3′,5′), 132.83 (4), 140.40 (1), 141.16 (1′), 143.33 (3, 5), 167.22 (OCOCH₃), 168.00 (2×OCOCH₃); LRESI positive ion mass spectrum; m/z 379 (MNa⁺, 100%).

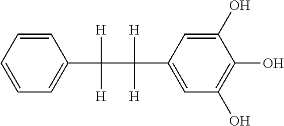 [SMILES: Oc1cc(CCc2ccccc2)cc(O)c1O]

### Example 54

### 5-Phenethylbenzene-1,2,3-triol (54)

p-Toluene sulphonic acid monohydrate dissolved in methanol was added to a solution of 5-phenethylbenzene-1,2,3-triyl triacetate in methanol. The reaction was heated to 85° C. overnight under a positive pressure nitrogen gas atmosphere and the solvent then removed by rotary evaporation. The remaining salmon pink coloured solid was purified with column chromatography (isocratically eluted with 1:1 EtOAc/hexane) to return 5-phenethylbenzene-1,2,3-triol as a cream coloured solid. R_{f} 0.50 (1:1 EtOAc/hexane); mp 124.0-124.5° C.; ¹H NMR (300 MHz, CD₃OD): δ 2.67-2.76 (m, 2H, CH₂), 2.79-2.87 (m, 2H, CH₂), 6.23 (bs, 2H, H-2, H-6), 7.12-7.17 (m, 3H, H-2′, H-4′, H-6′), 7.23-7.28 (m, 2H, H-3′, H-5′); ¹³C JMOD NMR (75 MHz, CD₃OD): δ 39.39 (CH₂), 39.82 (CH₂), 109.32 (2,6), 127.44 (4′), 129.93 (2′,6′), 130.17 (3′,5′), 132.80 (4), 135.15 (1), 143.95 (1′), 147.46 (3,5); LRESI negative ion mass spectrum; m/z 229 ([M−H]⁻, 100%).

A further “three point” exemplar has been generated with the synthesis of 3,5-dihydroxy-N-(4-hydroxyphenyl)benzamide (17). The procedure used was that described by S. J. Kim et al., (Amorepacific Corporation; Applicant), “Hydroxybenzamide derivatives, the method for preparing thereof and the cosmetic composition containing the same”, WO2007/021067 A1. The below scheme summarizes this preparation, starting from commercially available 3,5-dihydroxybenzoic acid (25) and 4-aminophenol.

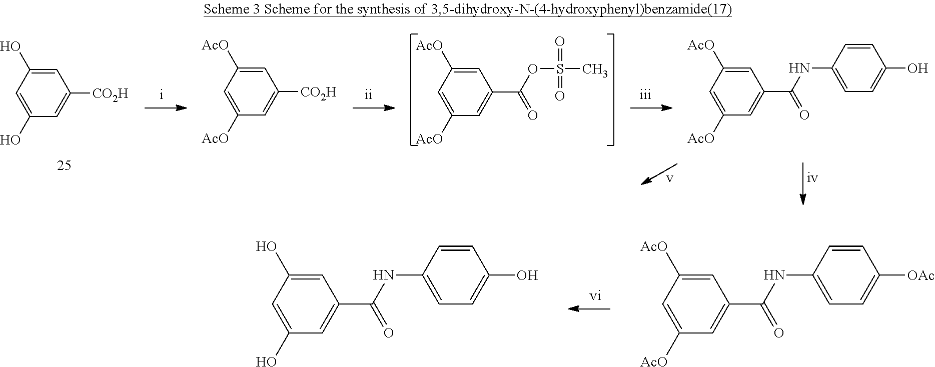

Reagents and conditions: (i) Ac₂O, Et₃N, EtOAc, reflux, 4 hours, 34% yield; (ii) Et₃N, THF, CH₃SO₂Cl, 30 minutes; (iii) 4-aminophenol, 0° C., 4 hours, 82% yield; (iv) Ac₂O, pyridine, DMAP, EtOAc, reflux, 2 hours, 76% yield; (v) (a) KOH_{(aq)}, reflux, 75 minutes. (b) HCl_{(aq)}, 55% yield; (vi) (a) KOH_{(aq)}, reflux, 2 hours. (b) HCl_{(aq)}, 60% yield

### Example 55

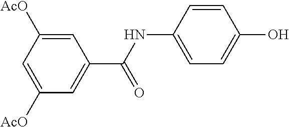 [SMILES: CC(=O)Oc1cc(OC(C)=O)cc(C(=O)Nc2ccc(O)cc2)c1]

### 5-(4-Hydroxyphenylcarbamoyl)-1,3-phenylene diacetate (55)

The reaction was conducted under a positive pressure of Argon gas. Triethylamine was added to a solution of 3,5-diacetoxybenzoic acid in anhydrous THF cooled to 0° C. Methane sulphonyl chloride was then slowly added by syringe and the reaction stirred for a further 25 minutes. 4-Aminophenol was then added, and the reaction kept at 0° C. for a further 4 hours before being stored overnight at −20° C. The reaction was then acidified (pH 2) with 1M HC and the solvents removed by rotary evaporation. The residual gum was dissolved in EtOAc and washed with water. dried (anhyd. Na₂SO₄), filtered and rotary evaporated to return a white solid. ¹H NMR (CD₃OD) showed this to be at best 75% pure 5-(4-hydroxyphenylcarbamoyl)-1,3-phenylene diacetate. This material was used as is for ongoing reactions. R_{f} 0.56 (2:1 EtOAc/hexane).

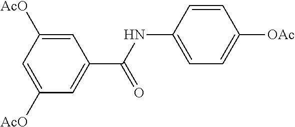 [SMILES: CC(=O)Oc1ccc(NC(=O)c2cc(OC(C)=O)cc(OC(C)=O)c2)cc1]

### Example 56

### 5-(4-Acetoxyphenylcarbamoyl)-1,3-phenylene diacetate (56)

“Crude” 5-(4-hydroxyphenylcarbamoyl)-1,3-phenylene diacetate was suspended in ethyl acetate. 4-(Dimethylamino)pyridine, pyridine and acetic anhydride were then added and the solution then heated to reflux for 2 hours and left to stand at room temperature overnight. Further ethyl acetate was added and the reaction washed with 0.1M HCl and water, dried (anhyd. Na₂SO₄), filtered and rotary evaporated to return a white solid. This was re-dissolved in ethyl acetate, silica added and the solvent rotary evaporated off. The remaining powder was loaded onto a silica column as a dry plug and chromatographed (gradient eluted beginning with 2:1 EtOAc/hexane and finishing with 5:1 EtOAc/hexane) to give 5-(4-acetoxyphenylcarbamoyl)-1,3-phenylene diacetate as a white powder. ¹H NMR (d₆-DMSO) showed this to be ca. 80% pure. This material was used as is for the ongoing hydrolysis reaction. R_{f} 0.63 (2:1 EtOAc/hexane).

### Example 57

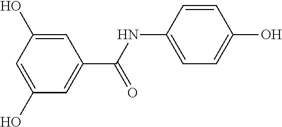 [SMILES: O=C(Nc1ccc(O)cc1)c1cc(O)cc(O)c1]

### 3,5-Dihydroxy-N-(4-Hydroxyphenyl)Benzamide (57)

### METHOD 1; Hydrolysis of the Diacetate.

The reaction was conducted under a positive pressure of Argon gas. An aqueous solution of potassium hydroxide was added to “crude” 5-(4-hydroxyphenylcarbamoyl)-1,3-phenylene diacetate. The reaction was heated to reflux for 75 minutes, returned to room temperature and 1M HCl added until a precipitate formed (pH approx. 3). This was vacuum filtered off and the solid repeatedly washed in the funnel with water, and then dried under vacuum to return 3,5-dihydroxy-N-(4-hydroxyphenyl)benzamide as fluffy small white needles. R_{f} 0.39 (4:1 EtOAc/hexane); mp 266.0-266.5° C.; ¹H NMR (CD₃OD): δ 6.47 (pseudo t, 1H, Jₘₑₜₐ=2.2 Hz, H-4), 6.77-6.82 (m, 4H, H-3′, H-5′, H-2, H-6), 7.43-7.46 (m, 2H, Jₒᵣₜₕₒ=8.9 Hz, H-2′, H-6′); ¹H NMR (d₆-DMSO): δ 6.42 (pseudo t, 1H, Jₘₑₜₐ=2.2 Hz, H-4), 6.72-6.78 (m, 4H, H-3′, H-5′, H-2, H-6), 7.51-7.56 (m, 2H, Jₒᵣₜₕₒ=8.9 Hz, H-2′, H-6′), 9.20 (s, 1H), 9.51 (s, 2H), 9.83 (s, 1H); ¹³C JMOD NMR (CD₃OD): δ 104.41 (4), 104.67 (2,6), 113.92 (3′,5′), 122.13 (2′,6′), 129.10 (1′), 136.08 (1), 153.26 (4′), 157.41 (3,5), 166.70 (C═O); LRESI negative ion mass spectrum; m/z 244 ([M−H]⁻, 100%), 489 ([2M−H]⁻, 29%); HRESI positive ion mass spectrum; m/z for C₁₃H_{11N}O₄, calculated [MH]⁺ 246.0766. measured 246.0764.

### METHOD 2; Hydrolysis of the Triacetate.

The reaction was conducted under a positive pressure of Argon gas. A solution of potassium hydroxide in water was added to “crude” 5-(4-acetoxyphenylcarbamoyl)-1,3-phenylene diacetate. The reaction was heated to reflux for 2 hours and on return to room temperature, 1M HCl was then added until a precipitate formed (pH approx. 3). This was vacuum filtered off and repeatedly washed with water. The white solid then dried under vacuum to return 3,5-dihydroxy-N-(4-hydroxyphenyl)benzamide as fluffy small white needles. Melting point and spectroscopic data were identical to the previously prepared material.

### Example 58

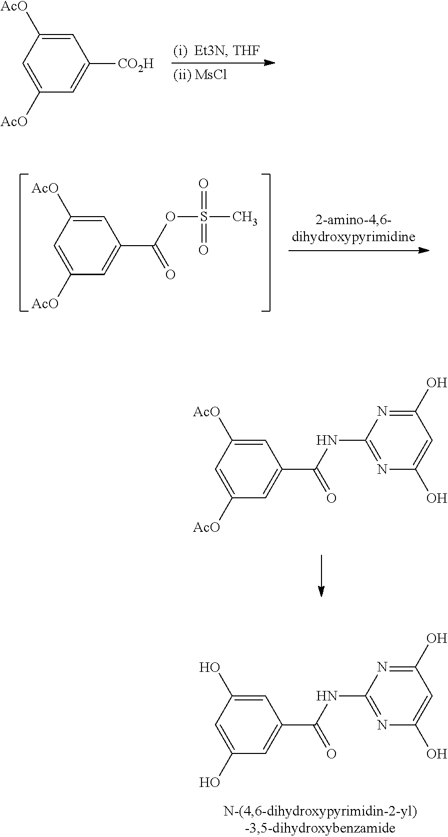

Additional compounds sourced/evaluated/templated are shown below. Examples have been used both as MIP templates and as MIP test analytes.

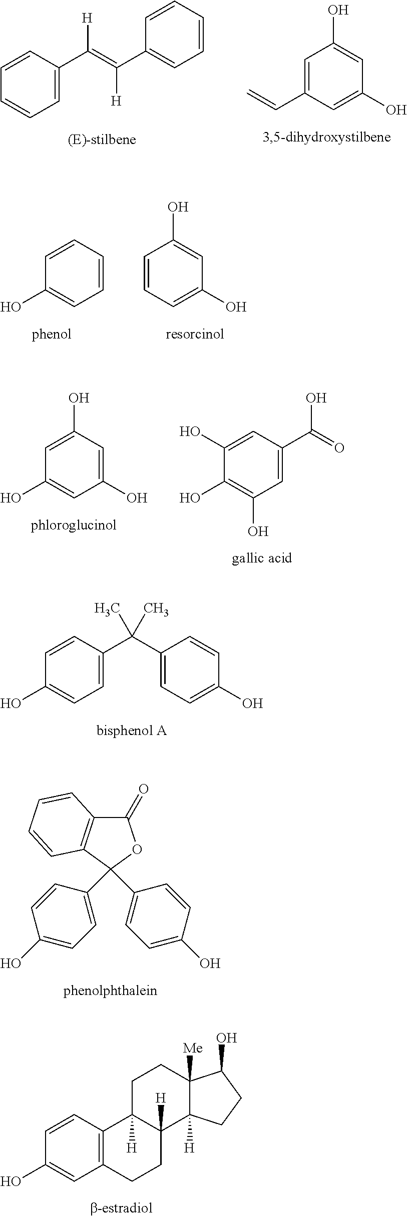

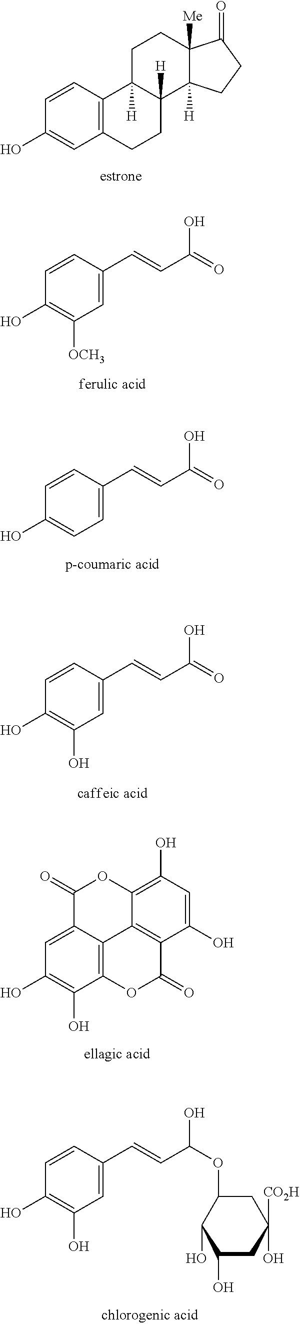

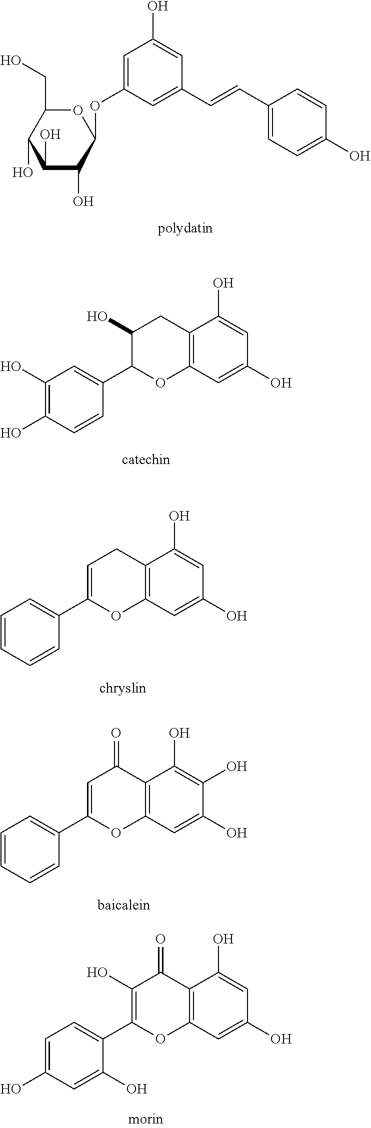

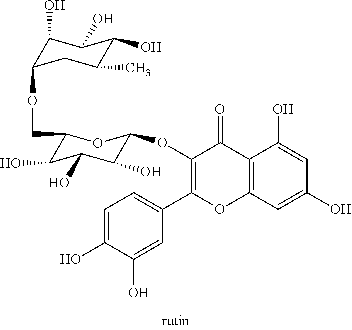

The compounds which have been synthesized and sourced externally provide useful templates for (i) constructing MIPs around defined templates, (ii) assessment of binding using pure and complex mixtures of compounds, and (iii) full characterisation of the selectivity of said MIPs for target compounds. with the synthesised templates as test compounds and with real bioprocess waste materials.

Development of MIP systems centred around more complex resveratrol analogues as templates, such as the flavone class of molecules, and different classes of polyphenols.

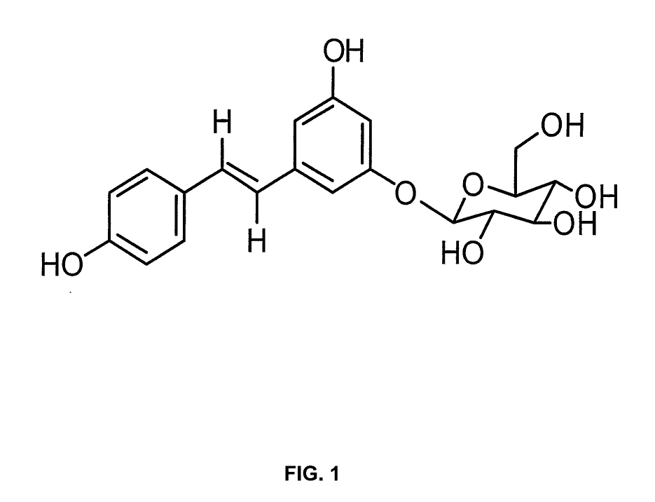

Other molecules, and their analogues, currently being investigated as potential templates for MIPs include inter alia cacatechin, morin, ellagic acid, procyanidin which is being targeted with MIP_{Amide}. Another possible class of molecules for MIP templating are naturally occurring resveratrol derivatives such as the glucuronides. Romero-Peréz et al⁶ have reported significant amounts of these glucuronides in grape berry skins, quantities that typically exceed that of resveratrol, (J. Agric. Food Chem., Vol. 49, No. 1, 2001, 210-215). Development of a MIP specific for such compounds could have considerable value for concentrating, separating and extracting these bioactive molecules as secondary downstream products from the same feedstock used to isolate resveratrol. If required, these glucuronides could then be either chemically or enzymatically converted to resveratrol. D. A. Learmonth (Bioconjugate Chem., 2003, 14, 262-267)⁷ has reported a synthetic methodology for preparing these glucuronide templates (FIG. 1).

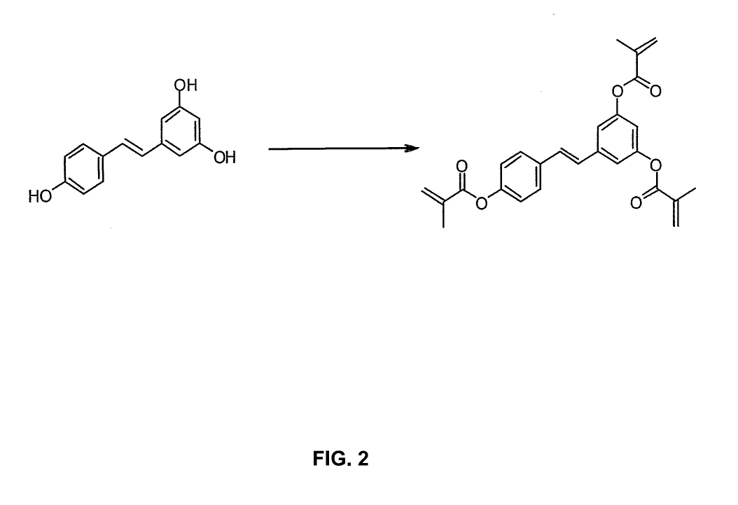

We have also explored the development of a new class of MIPs, called “covalently bound” MIPs, which are expected to have both greater selectivity and capacity for individual target molecules. Trial experiments have already generated a small quantity of (E)-5-(4-(methacryloyloxy)styryl)-1,3-phenylene bis(2-methylacrylate), shown in FIG. 2, as a likely candidate for initial exploratory MIP studies.

### (E)-3,4′,5-Tri(methacryloyl)oxystilbene (BDp47-5-12-2006). Systematically named as (E)-5-(4-(methacryloyloxy)styryl)-1,3-phenylene bis(2-methylacrylate)

The reaction was conducted under an Argon gas atmosphere. Resveratrol was dissolved in dry acetonitrile, to which anhydrous sodium sulphate was added. The solution was vigorously stirred for 30 minutes and the solid material was filtered off. Triethylamine was then added to the filtrate and immediately cooled in an ice-bath. Methacryloyl chloride dissolved in acetonitrile was slowly added and the reaction solution was allowed to cool to room temperature, then stirred overnight. The reaction was then cooled again in an ice-bath and further triethylamine added, followed by ice-water and the solution stirred for a further 20 minutes. Ethyl acetate was added and the phases separated. The organic layer was washed four times with water, then dried, filtered and rotary evaporated to give a brown gum. This gum was purified by gradient elution chromatography (9:1 hexane/EtOAc to 4:1 hexane/EtOAc) to give pure (E)-3,4′,5-tri(methacryloyl)oxystilbene as a clear oil. R_{f} 0.47 (4:1 hexane/EtOAc); ¹H NMR (CDCl₃): δ 2.04 (s, 9H, 3×CH₃), 5.72-5.76 (m, 3H, 3×vinyl), 6.33-6.34 (m, 3H, 3×vinyl), 6.88 (pseudo t, 1H, Jₘₑₜₐ=2.1 Hz, H-4), 6.97 (d, 1H, J=16.3 Hz, Hₜᵣₐₙₛ), 7.10 (d, 1H, Hₜᵣₐₙₛ), 7.10-7.15 (m, 4H, H-2, H-6, H-3′, H-5′), 7.47 (dt, 2H, Jₒᵣₜₕₒ=8.6 Hz, H-2′, H-6′). LRESI positive ion mass spectrum; m/z 455 (MNa⁺, 100%), 456 (31%).

### Example S1

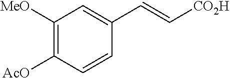 [SMILES: COc1cc(/C=C/C(=O)O)ccc1OC(C)=O]

### trans-4-O-Acetylferulic acid [(E)-3-(4-acetoxy-3-methoxyphenyl)acrylic acid.] (S1)

### Method 1.

Ferulic acid was added to an aqueous solution of sodium hydroxide cooled in an ice-bath. The suspension was well stirred until all the solid had dissolved, and then acetic anhydride was then added. After 5 minutes, the cooling bath was removed and the solution stirred at room temperature for a further 90 minutes. The reaction was returned back to 0° C., 2M aq. HCl added to precipitate a white solid (pH ca. 4), and this vacuum filtered and repeatedly washed in the funnel with further water. Recrystallization from hexane/ethyl acetate collected 2 crops of trans-4-O-acetylferulic acid as a white powder. R_{f} 0.52 (4:1 EtOAc/hexane); mp 197.5-198.0° C.; lit mp 192° C. (Roberts et al, Eur. J. Med. Chem., (1994), 29, 841-854⁸); ¹H NMR (300 MHz, CDCl₃): δ 2.33 (s, 3H, OAc), 3.88 (s, 3H, OMe), 6.40 (d, 1H, Jₜᵣₐₙₛ=15.9 Hz, Hₐₗₖₑₙₑ), 7.06-7.17 (m, 3H, arom), 7.73 (d, 1H, Jₜᵣₐₙₛ 15.9 Hz, Hₐₗₖₑₙₑ).

### Method 2.

Ferulic acid was dissolved in pyridine. Acetic anhydride (15.0 mL) was added and the reaction stirred overnight at room temperature overnight. The solution was then poured onto ice and acidified with 2M HCl (pH 2). The resultant white solid was filtered off, washed in the funnel with further water and then dried. Recrystallized from boiling dichloromethane and ethanol gave two crops of trans-4-0-acetylferulic acid as a white powder. Mp 201.0-201.5° C.; lit mp 192° C. (Roberts et al, Eur. J. Med. Chem., (1994), 29, 841-854⁸); R_{f} and ¹H NMR (300 MHz, CDCl₃) were identical to the earlier prepared material.

### Example S2

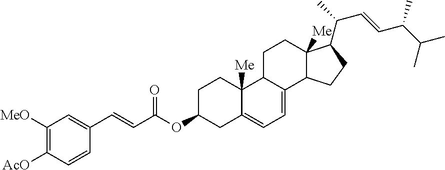

### 3-O-(trans-4-O-Acetylferuloyl)-ergasterol (Systematically named as (E)-((3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl) 3-(4-acetoxy-3-methoxyphenyl)acrylate) (S2)

trans-4-O-Acetylferulic acid was dissolved in dry dichloromethane and 4-dimethylaminopyridine and N,N-dicyclohexylcarbodiimide added. The reaction was stirred at room temperature for 60 minutes to generate the activated acid, and ergosterol was then added and washed in with further dichloromethane. The solution was stirred at room temperature overnight and the resultant white solid removed by filtration. The filtrate was rotary evaporated to return a white solid which was purified with column chromatography (gradient eluted starting with 50:1 CHCl₃/MeOH and finished with 50:4 CHCl₃/MeOH) to give 3-O-(trans-4-O-acetylferuloyl)-ergasterol as a white solid. R_{f} 0.58 (2:1 hexane/EtOAc), 0.90 (49:1 CHCl₃/MeOH); mp 181.5-182.05° C.; ¹H NMR (300 MHz, CDCl₃): δ 0.64 (s, 3H, CH₃), 0.83 (d, 3H, J=6.8 Hz, CH₃), 0.85 (d, 3H, J=6.8 Hz, CH₃), 0.93 (d, 3H, J=6.8 Hz, CH₃), 0.99 (s, 3H, CH₃), 1.04 (d, 3H, J=6.6 Hz, CH₃), 1.20-2.60 (broad multiplet with an indeterminate number of protons), 2.32 (s, 3H, OAc), 3.86 (s, 3H, OCH₃), 4.80-4.90 (m, 1H, H-3), 5.14-5.28 (m, 2H, H-22, H-23), 5.38-5.42 (m, 1H, H-6/5), 5.59-5.61 (m, 1H, H-5/6), 6.40 (d, 1H, Jₜᵣₐₙₛ=15.9 Hz, Hₐₗₖₑₙₑ), 7.03-7.14 (m, 3H, arom), 7.65 (d, 1H, Jₜᵣₐₙₛ=15.9 Hz, Hₐₗₖₑₙₑ); ¹³C JMOD NMR (75 MHz, CDCl₃): δ 12.30 (18), 16.43, 17.84, 19.89, 20.18, 20.82 (5×CH₃), 21.28 (11), 21.35 (CH₃), 23.24 (15), 28.48, 28.50 (2,16), 33.33 (25), 37.01, 37.37, 38.19, 39.29 (10,4,13,1,12), 40.64 (20), 43.07 (24), 46.31 (9), 54.77, 55.99, 56.10 (14, OCH₃, 17), 73.22 (3), 111.47 (2ₐᵣₒₘ), 116.59 (7), 119.08 (6), 120.55 (Cₐₗₖₑₙₑ), 121.41 (6ₐᵣₒₘ), 123.44 (5ₐᵣₒₘ), 132.23 (23), 133.70 (1ₐᵣₒₘ), 135.80 (22), 138.72 (5), 141.65 (8), 141.71 (4ₐᵣₒₘ), 143.97 (Cₐₗₖₑₙₑ), 151.63 (C—OCH₃), 166.36 (C3-OCOCH═), 168.89 (Ar—OCOCH₃).

### Example S3

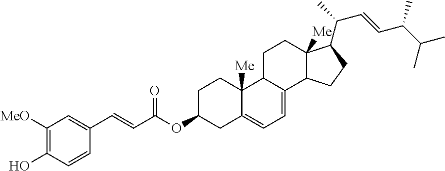

### 3-O-(trans-feruloyl)-ergasterol (Systematically named as (E)-((3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl) 3-(4-hydroxy-3-methoxyphenyl)acrylate) (S3)

3-O-(trans-4-O-Acetylferuloyl)-ergasterol was dissolved in a 2:1 mixture of chloroform and methanol and potassium carbonate added and the reaction heated to 65° C. for 6 hours. On return to room temperature, saturated aqueous ammonium chloride was added and the solution repeatedly extracted with dichloromethane. The combined extracts were washed with water, dried (anhyd. Na₂SO₄), filtered and rotary evaporated to return a white solid. Purification with column chromatography (gradient eluted started with CH₂Cl₂ and finished with 98:2 CH₂Cl₂/MeOH) returned 3-O-(trans-feruloyl)-ergasterol as a white solid. R_{f} 0.31 (4:1 hexane/EtOAc), 0.49 (100% CH₂Cl₂), 0.84 (98:2 CH₂Cl₂/MeOH); mp 182.0-182.5° C.; ¹H NMR (400 MHz, CDCl₃): δ 0.64 (s, 3H, CH₃), 0.84-0.85 (m, 6H, 2×CH₃), 0.92 (d, 3H, J=6.8 Hz, CH₃), 0.99 (s, 3H, CH₃), 1.04 (d, 3H, J=6.6 Hz, CH₃), 1.26-2.08 (broad multiplet with an indeterminate number of protons), 2.41-2.48 (m, 1H), 2.56-2.61 (m, 1H), 3.93 (s, 3H, OCH₃), 4.82-4.86 (m, 1H, H-3), 5.15-5.26 (m, 2H, H-22, H-23), 5.39-5.40 (m, 1H, H-6/5), 5.59-5.60 (m, 1H, H-5/6), 5.83 (s, 1H, OH), 6.28 (d, 1H, Jₜᵣₐₙₛ=15.9 Hz, Hₐₗₖₑₙₑ), 6.91 (d, 1H, Jₒᵣₜₕₒ=8.2 Hz, H6ₐᵣₒₘ), 7.03-7.10 (m, 2H, Hₐᵣₒₘ), 7.60 (d, 1H, Jₜᵣₐₙₛ=15.9 Hz, Hₐₗₖₑₙₑ); ¹³C JMOD NMR (100 MHz, CDCl₃): δ 12.41 (18), 16.55, 17.95, 19.99, 20.29, 21.46 (5×CH₃), 21.40 (11), 23.35 (15), 28.62 (2,16), 33.44 (25), 37.17, 37.49, 38.32, 39.41 (10,4,13,1,12), 40.75 (20), 43.17 (24), 46.42 (9), 54.88, 56.10, 56.26 (14, OCH₃, 17), 73.09 (3), 109.70 (2ₐᵣₒₘ), 115.08 (7), 116.36 (Cₐₗₖₑₙₑ), 116.69 (6), 120.59 (5ₐᵣₒₘ), 123.36 (6ₐᵣₒₘ), 127.44 (1ₐᵣₒₘ), 132.34 (23), 135.92 (22), 138.96 (5), 141.83 (8), 144.93 (Cₐₗₖₑₙₑ), 147.13 (4ₐᵣₒₘ), 148.28 (C—OCH₃), 166.99 (C═O).

### Example S4

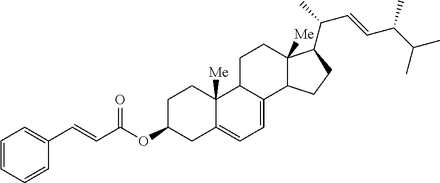

### 3-O-ergasteryl cinnamate (Systematically named as (3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl cinnamate) (S4)

To the best of the inventor's knowledge, this compound is novel.

trans-Cinnamic acid was dissolved in dry dichloromethane. 4-Dimethylaminopyridine and N,N-dicyclohexylcarbodiimide were added and the reaction was stirred at room temperature for 15 minutes. Ergosterol was then added along with further dry dichloromethane and the reaction stirred at room temperature overnight. The resultant white solid was filtered off and the solid washed with further dichloromethane The combined filtrates were rotary evaporated and the resultant white solid then purified with column chromatography (gradient eluted starting with CH₂Cl₂ (100%) and finished with 97:3 (v/v) CH₂Cl₂/MeOH) to return a white solid. ¹H NMR (300 MHz, CDCl₃) showed this to be ca. 95% pure title compound. A sample was taken and recrystallized from a 2:1 hexane and ethyl acetate mixture to give 3-O-ergasteryl cinnamate as “glistening” white mica plates. R_{f} 0.68 (4:1 hexane/EtOAc), 0.89 (99:1 CH₂Cl₂/MeOH); mp 171.0-171.1° C., ¹H NMR (300 MHz, CDCl₃): δ 0.64 (s, 3H, CH₃), 0.83 (d, 3H, J=6.8 Hz, CH₃), 0.85 (d, 3H, J=6.7 Hz, CH₃), 0.93 (d, 3H, J=6.8 Hz, CH₃), 0.99 (s, 3H, CH₃), 1.04 (d, 3H, J=6.6 Hz, CH₃), 1.23-2.62 (broad multiplet with an indeterminate number of protons), 4.80-4.91 (m, 1H, H-3), 5.14-5.28 (m, 2H, H-22, H-23), 5.38-5.42 (m, 1H, H-6/5), 5.59-5.61 (m, 1H, H-5/6), 6.43 (d, 1H, Jₜᵣₐₙ=16.0 Hz, Hₐₗₖₑₙₑ), 7.36-7.40 (m, 3H, 3ₐᵣₒₘ, 4ₐᵣₒₘ, 5ₐᵣₒₘ), 7.51-7.55 (m, 2H, 2ₐᵣₒₘ, 6ₐᵣₒₘ), 7.68 (d, 1H, Hₐₗₖₑₙₑ); ¹³C JMOD NMR (75 MHz, CDCl₃): δ 12.42 (18), 16.56, 17.97, 20.01, 20.31, 21.48 (5×CH₃), 21.41 (11), 23.36 (15), 28.60, 28.63 (2,16), 33.45 (25), 37.14, 37.50, 38.33, 39.42 (10,4,13,1,12), 40.76 (20), 43.19 (24), 46.44 (9), 54.90 (14), 56.11 (17), 73.26 (3), 116.72 (7), 119.02 (6), 120.64 (Cₐₗₖₑₙₑ), 128.38 (2ₐᵣₒₘ,6ₐᵣₒₘ), 129.20 (3ₐᵣₒₘ,5ₐᵣₒₘ), 130.50 (4ₐᵣₒₘ), 132.35 (23), 134.90 (1ₐᵣₒₘ), 135.93 (22), 138.92 (5), 141.81 (8), 144.81 (Cₐₗₖₑₙₑ), 166.68 (C═O).

### Example S5

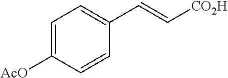 [SMILES: CC(=O)Oc1ccc(/C=C/C(=O)O)cc1]

### (E)-4-O-Acetoxycinnamic acid (Systematically named as (E)-3-(4-acetoxyphenyl)acrylic acid) (S5)

p-Coumaric acid was suspended in pyridine and acetic anhydride and DMAP were added. All the solids rapidly dissolved and the solution was stirred overnight at room temperature. The reaction was then poured onto ice and acidified (to pH 3) by addition of 2M HCl. The resultant white solid was filtered off, washed in the funnel with further water, and then dried under vacuum over desiccant. The dry material was recrystallized from ethyl acetate to give (E)-4-O-acetoxycinnamic acid as white needles, and a second crop was obtained from the volume reduced filtrate. R_{f} 0.52 (4:1 EtOAc/hexane); mp 201.1-201.2° C.; lit mp 205-208° C. (Roberts et al, Eur. J. Med. Chem., (1994), 29, 841-854⁷); ¹H NMR (300 MHz, CD₃OD): δ 2.31 (s, 3H, OAC), 6.48 (d, 1H, Jₜᵣₐₙₛ=16.0 Hz, Hₐₗₖₑₙₑ), 7.17-7.20 (m, 2H, Jₒᵣₜₕₒₛ=8.6 Hz, 3ₐᵣₒₘ, 5ₐᵣₒₘ) 7.64-7.73 (m, 3H, Hₐₗₖₑₙₑ 2ₐᵣₒₘ, 6ₐᵣₒₘ); LRESI negative ion mass spectrum; m/z 205 ([M−H]⁻, 100%), 206 (12%).

### Example S6

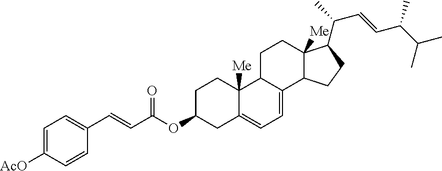

### 3-O-(trans-4-O-Acetoxycinnamoyl)-ergasterol (Systematically named as (E)-((3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl) 3-(4-acetoxyphenyl)acrylate) (S6)

A mixture of (E)-4-O-acetoxycinnamic acid, 4-dimethylaminopyridine and N,N-dicyclohexylcarbodiimide in dry dichloromethane was stirred at room temperature for 20 minutes. Ergosterol was then added and washed in with further dry dichloromethane and the reaction stirred at room temperature overnight. The resultant white solid was filtered off, washed well in the funnel with further dichloromethane and the combined filtrates rotary evaporated to return a yellow solid. Purification with column chromatography (gradient eluted starting with CH₂Cl₂ and finished with 97:3 CH₂Cl₂/MeOH) returned 3-O-(trans-4-O-acetoxycinnamoyl)-ergasterol as a white solid. R_{f} 0.60 (4:1 hexane/EtOAc), 0.56 (CH₂Cl₂ 100%); mp 181.1-181.2° C.; ¹H NMR (300 MHz, CDCl₃): δ 0.64 (s, 3H, CH₃), 0.82-0.86 (m, 6H, 2×CH₃), 0.92 (d, 3H, J=6.8 Hz, CH₃), 0.99 (s, 3H, CH₃), 1.04 (d, 3H, J=6.6 Hz, CH₃), 1.22-2.57 (broad multiplet with an indeterminate number of protons), 2.31 (s, 3H, OAc), 4.79-4.90 (m, 1H, H-3), 5.10-5.28 (m, 2H, H-22, H-23), 5.38-5.41 (m, 1H, H-6/5), 5.58-5.61 (m, 1H, H-5/6), 6.38 (d, 1H, Jₜᵣₐₙₛ=16.0 Hz, Hₐₗₖₑₙₑ), 7.10-7.14 (m, 2H, Jₒᵣₜₕₒ=8.6 Hz, 3ₐᵣₒₘ,5ₐᵣₒₘ), 7.51-7.56 (m, 2H, 2ₐᵣₒₘ,6ₐᵣₒₘ), 7.67 (d, 1H, Hₐₗₖₑₙₑ); ¹³C JMOD NMR (75 MHz, CDCl₃): δ 12.34 (18), 16.46, 17.90, 19.94, 20.24, 21.34, 21.41 (5×CH₃, OC(O)CH₃), 21.32 (11), 23.29 (15), 28.52, 28.55 (2,16), 33.37 (25), 37.05, 37.40, 38.23, 39.34 (10,4,1,12), 40.69 (20), 43.09 (13), 43.11 (24), 46.35 (9), 54.81 (14), 56.03 (17), 73.24 (3), 116.66 (7), 119.08 (6), 120.59 (C_{cinn alkene}), 122.34 (3ₐᵣₒₘ,5ₐᵣₒₘ), 129.39 (2ₐᵣₒₘ,6ₐᵣₒₘ), 132.27 (23), 132.49 (1ₐᵣₒₘ), 135.85 (22), 138.78 (5), 141.69 (8), 143.58 (C_{cinn alkene}), 152.32 (4ₐᵣₒₘ), 166.42 (C3-OCOCH₃), 169.22 (Ar—OCOCH₃).

### Example S7

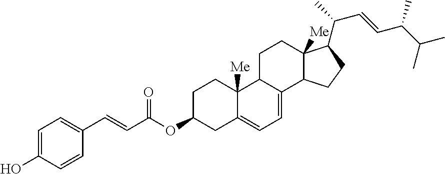

### 3-O-(trans-coumaroyl)-ergasterol (Systematically named as (E)-((3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl) 3-(4-hydroxyphenyl)acrylate) (S7)

Potassium carbonate was added to 3-O-(trans-4-O-acetylferuloyl)-ergasterol dissolved in a 2:1 mixture of chloroform and methanol. This was then heated to 70° C. for 6 hours, further 2:1 chloroform (10.0 mL) and methanol (5.0 mL) added, and the reaction left to stir at room temperature overnight. Saturated aqueous ammonium chloride and additional dichloromethane were added and the two phases separated. The organic material was repeatedly washed with water, dried (anhyd. Na₂SO₄), filtered and rotary evaporated to return a cream coloured solid. Recrystallization from ethyl acetate gave pure 3-O-(trans-coumaroyl)-ergasterol as a white solid. The filtrate was reduced in volume to return a second crop. R_{f} 0.28 (100% CH₂Cl₂); mp 213.1-213.2° C.; ¹H NMR (400 MHz, CDCl₃): δ 0.64 (s, 3H, CH₃), 0.82-0.85 (m, 6H, 2×CH₃), 0.92 (d, 3H, J=6.8 Hz, CH₃), 0.99 (s, 3H, CH₃), 1.04 (d, 3H, J=6.6 Hz, CH₃), 1.24-2.09 (broad multiplet with an indeterminate number of protons), 2.41-2.47 (m, 1H), 2.56-2.60 (m, 1H), 4.81-4.85 (m, 1H, H-3), 5.15-5.26 (m, 3H, H-22, H-23, OH), 5.39-5.41 (m, 1H, H-6/7), 5.59-5.60 (m, 1H, H-7/6), 6.29 (d, 1H, Jₜᵣₐₙₛ=15.9 Hz, Hₐₗₖₑₙₑ), 6.80-6.86 (d, 2H, Jₒᵣₜₕₒ=8.6 Hz, H3ₐᵣₒₘ, H5ₐᵣₒₘ), 7.41-7.45 (m, 2H, H2ₐᵣₒₘ, H6ₐᵣₒₘ), 7.62 (d, 1H, Hₐₗₖₑₙₑ); ¹³C JMOD NMR (75 MHz, CDCl₃): δ 12.43 (18), 16.58, 17.96, 20.01, 20.30, 21.47 (5×CH₃), 21.42 (11), 23.34 (15), 28.62 (2,16), 33.46 (25), 37.17, 37.51, 38.33, 39.43 (10,4,1,12), 40.76 (20), 43.19 (24), 43.21 (13), 46.45 (9), 54.91 (14), 56.12 (17), 73.23 (3), 116.24, 116.44, 116.69, (7, C_{cinn alkene},3ₐᵣₒₘ,5ₐᵣₒₘ), 120.61 (6), 127.73 (1ₐᵣₒₘ), 130.29 (2ₐᵣₒₘ,6ₐᵣₒₘ), 132.37 (23), 135.94 (22), 138.98 (5), 141.89 (8), 144.62 (C_{cinn alkene}), 158.02 (4ₐᵣₒₘ), 167.28 (C3-OCOCH₃); LRESI negative ion mass spectrum; m/z 541 ([M−H]⁻, 100%), 542 (51%); HR EI negative ion mass spectrum; calculated for [C₃₇H₄₉O₃—H]⁻¹ 541.36817. measured 541.36801.

### Example S8

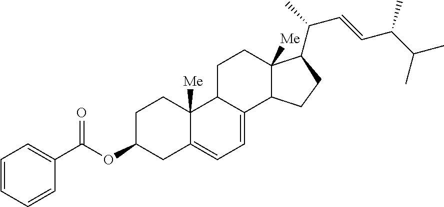

### Ergosteryl benzoate (Systematically named as 3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl benzoate) (S8)

The reaction was done under a positive pressure nitrogen gas atmosphere. Ergosterol was dissolved in dry pyridine and this solution cooled in an ice-bath. Freshly distilled benzoyl chloride was added and the reaction allowed to return back to room temperature, and left to stir overnight. The reaction was then poured onto ice-water and the white solid extracted with dichloromethane. The organic extract was then washed successively with water and saturated aqueous NaHCO₃, dried (anhyd. Na₂SO₄), filtered and rotary evaporated to return a pale brown solid. Purification with column chromatography (isocratically eluted with CH₂Cl₂, 100%) gave ergosteryl benzoate as a white solid. R_{f} 0.69 (100% CH₂Cl₂); mp 157.1-157.2° C.; lit mp 169-171° C. [Done, R. E.; Kruse, L. I. J. Org. Chem. 1985, 51, 4047-4053⁹]; ¹H NMR (400 MHz, CDCl₃): δ 0.65 (s, 3H, CH₃), 0.83 (d, 3H, J=6.6 Hz, CH₃), 0.85 (d, 3H, J=6.6 Hz, CH₃), 0.93 (d, 3H, J=6.8 Hz, CH₃), 1.01 (s, 3H, CH₃), 1.05 (d, 3H, J=6.6 Hz, CH₃), 1.26-2.67 (broad multiplet with an indeterminate number of protons), 4.92-5.01 (m, 1H, H-3), 5.16-5.27 (m, 2H, H-22, H-23), 5.40-5.42 (m, 1H, H-6/5), 5.60-5.62 (m, 1H, H-5/6), 7.39-7.45 (m, 2H, 3ₐᵣₒₘ, 5ₐᵣₒₘ), 7.53-7.57 (m, 1H, 4ₐᵣₒₘ), 8.01-8.06 (m, 2H, 2ₐᵣₒₘ, 6ₐᵣₒₘ); ¹³C JMOD NMR (100 MHz, CDCl₃): δ 12.43 (18), 16.59, 17.96, 20.01, 20.30, 21.47 (5×CH₃), 21.42 (11), 23.36 (15), 28.59, 28.63 (2,16), 33.45 (25), 37.13, 37.27, 37.52, 38.32 (10,4,12), 40.76 (20), 43.19 (24), 43.20 (13), 46.44 (9), 54.91 (14), 56.11 (17), 73.74 (3), 116.72 (7), 120.68 (6), 128.61 (2ₐᵣₒₘ,6ₐᵣₒₘ), 129.89 (3ₐᵣₒₘ,5ₐᵣₒₘ), 131.17 (1ₐᵣₒₘ), 132.36 (23), 133.07 (4ₐᵣₒₘ), 135.92 (22), 138.88 (5), 141.87 (8), 166.31 (C═O).

### Example S9

### Ergosteryl-3,4-dimethoxy cinnamate (BDp145-19-6-2008; 5.478 g crude that still requires final purification)

### Systematically named as (E)-((3S,10R,13R,17R)-17-((2R,5R,E)-5,6-dimethylhept-3-en-2-yl)-10,13-dimethyl-2,3,4,9,10,11,12,13,14,15,16,17-dodecahydro-1H-cyclopenta[a]phenanthren-3-yl) 3-(3,4-dimethoxyphenyl)acrylate

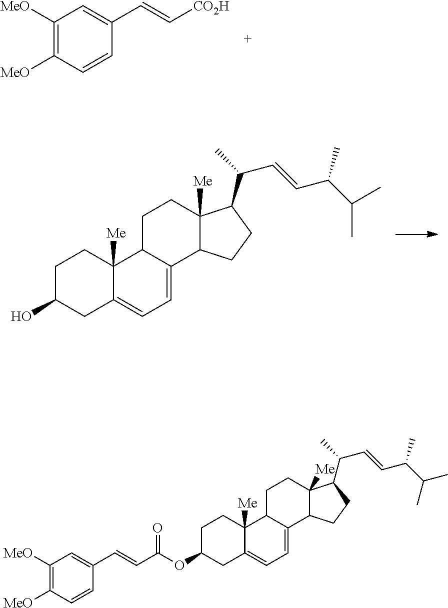

The procedure used was that reported for β-sitostanol described by Condo Jr, A. M.; Baker, D. C.; Moreau, R. A.; and Hicks, K. B., J. Agric. Food Chem., (2001), 49, 4961-4964, “Improved Method for the Synthesis of trans-Feruloyl-β-sitostanol.”¹⁰

3,4-Dimethoxy cinnamic acid (1.765 g, 8.475 mmol, Aldrich Chem. Co.) was dissolved in dichloromethane (30.0 mL, dried by passage through alumina) and 4-dimethylaminopyridine (105 mg, 0.861 mmol, Sigma) and N,N-dicyclohexylcarbodiimide (1.925 g, 9.345 mmol, Aldrich) added. The reaction was stirred at room temperature for 20 minutes to generate the activated acid. Ergosterol (3.360 g, 8.470 mmol, Aldrich) was then added along with further dry dichloromethane (10.0 mL) and the solution stirred at room temperature overnight. The resultant white solid was filtered off and washed with further dichloromethane (3×30 mL). The filtrates were combined and rotary evaporated to return 5.478 g of a pale yellow solid

TLC showed this to be predominantly a mixture of higher R_{f} new product and ergosterol starting material.

R_{f} 0.45 (4:1 hexane/EtOAc), 0.58 (CH₂Cl₂ 100%);

### B Molecularly Imprinted Polymers: Resveratrol

### Design of Molecularly Imprinted Polymers

The design of molecularly imprinted polymers (MIPs) requires the selection of a monomer species that will interact favourably with the intended template species, such that a pre-polymerisation complex is formed between template and monomer. The use of tools such as molecular modelling and NMR spectroscopy assist in the selection of appropriate monomers by performing a ‘virtual screen’, which reduces the number of polymer preparations required in MIP development.

### Molecular Modelling

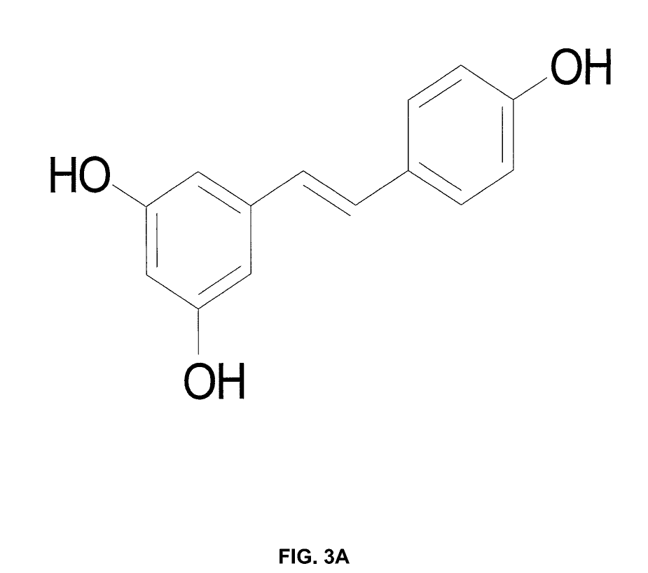

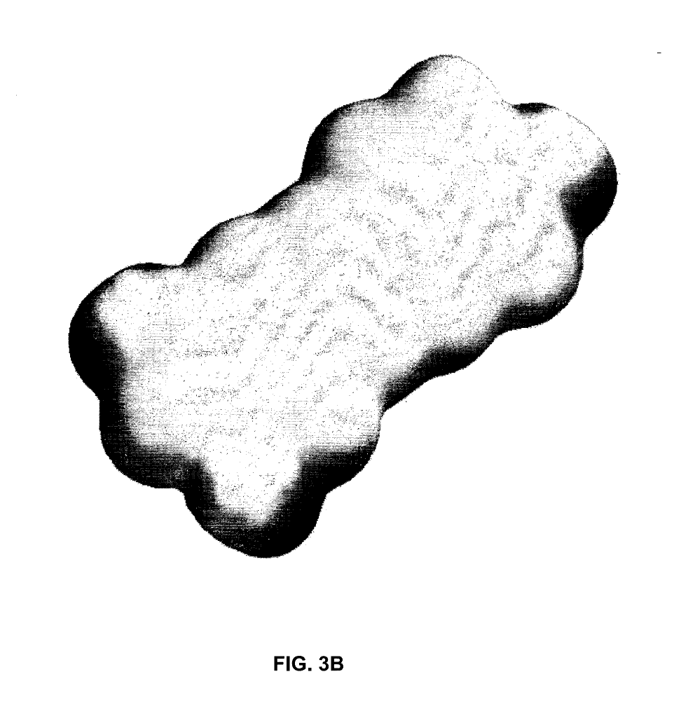

Modelling calculations were performed using PM3 geometry optimisation without solvent considerations to yield theoretical energy of formation values, ΔH_{f}. The geometry optimisation and surface electron potential of resveratrol was generated (FIGS. 3A and 3B), giving some insight into how resveratrol may interact with potential functional monomers.

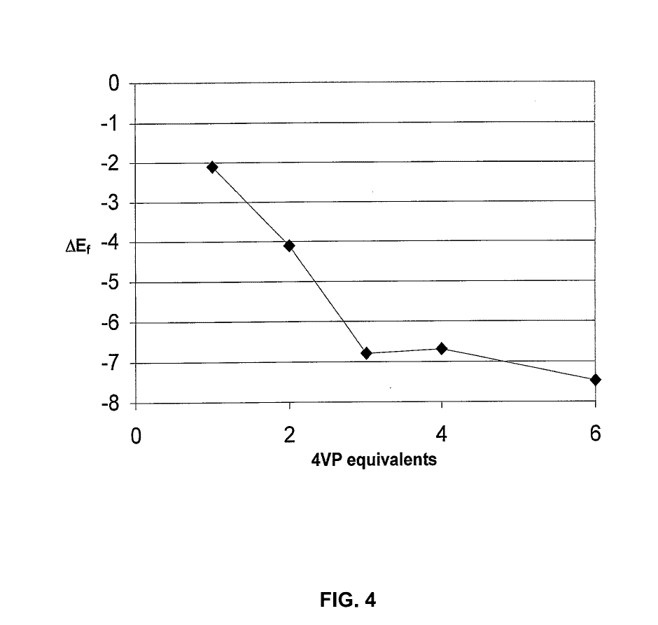

Monomer clusters were modelled, with clusters sizes ranging from 1 to 6 monomer units. These calculations yielded predictive ΔH_{f} values for the interaction of the monomer with itself at these cluster sizes (Table 4). Introduction of the theoretical geometry optimisation and surface electron parameters for resveratrol parameters permit estimation of the average energy of formation for the complex (FIG. 4) determined using the following equation:

4Eᵢ=(ΔH_{f}_{_}_{Complex}−(ΔH_{f}_{_}_{Template}+ΔH_{f}_{_}_{Monomer})).

The presence of cross-linking monomers were not included in these modelling calculations.

### 4-Vinylpyridine Based Complexes

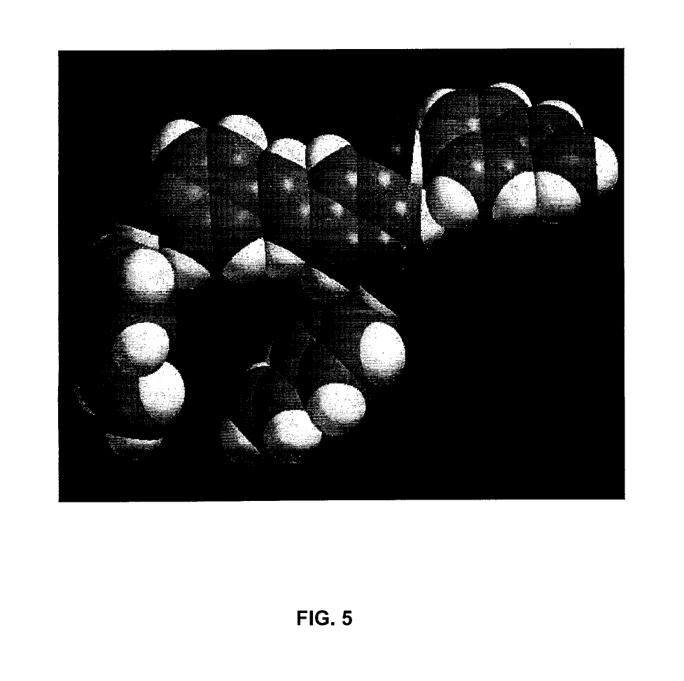

4VP is expected to interact through both hydrogen bonding and aromatic π-π interactions (FIG. 5).

TABLE 4
PM3 calculations of ΔEᵢ and ΔH_{f} for Resveratrol - 4VP modelling titration.

| 4VP | Av. ΔH_{f} | Av. ΔH_{f} | | Av. ΔEᵢ |
|---|---|---|---|---|
| Monomer | Kcal/mol | Kcal/mol | Av. Kcal/mol | interaction |
| eq | monomer cluster | Resveratrol | ΔE complex | Kcal/mol |
| 1 | 46.1118 | −74.5499 | −30.5636 | −2.1255 |
| 2 | 87.7339 | −74.5499 | 9.0896 | −4.0944 |
| 3 | 132.8645 | −74.5499 | 51.4990 | −6.8156 |
| 4 | 175.7882 | −74.5499 | 94.5293 | −6.7089 |
| 6 | 261.6890 | −74.5499 | 179.6830 | −7.4561 |

Table 4 shows that the optimum ratio of resveratrol to 4VP in the formation of a MIP should theoretically be 1:3. Similar studies for the imine (E)-5-[(4-hydroxy-phenylimino)-methyl]-benzene-1,3-diol or the amide 3,5-dihydroxy-N-(4-hydroxyphenyl)benzamide also show 1:3 ratios.

Both Ma et al and Zhuang et al (J. Chromatographic Sci. (2008), 46 (8), pp 739-742, have reported the preparation of MIPs from reseveratrol and 4-vinylpyridine. However, they did not identify the optimal 1:3 resveratrol:4-VP ratio either by empirical or modelling techniques.

### Acrylamide and 4-Vinylbenzoic Acid Complexes

Modelling titration experiments were conducted using various other functional monomers, including acrylamide (AAM) and 4-vinylbenzoic acid (4VBA) for which the data are shown below, as well as with 4-vinylpyridine (4VPyr). Because AAM has been employed by Xiang et al.¹ in a unsystematic experiment based on a resveratrol:AAM:EGDMA ratio of 1:6:30, we replicated this preparation for comparison purposes, despite the fact that our modelling studies indicating the requirement for only 3 equivalents AAM were needed for optimal MIP construction.

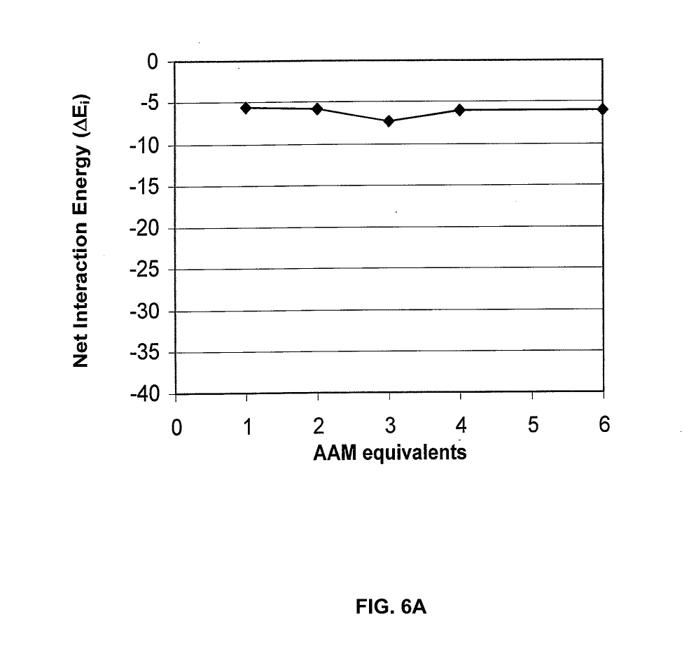

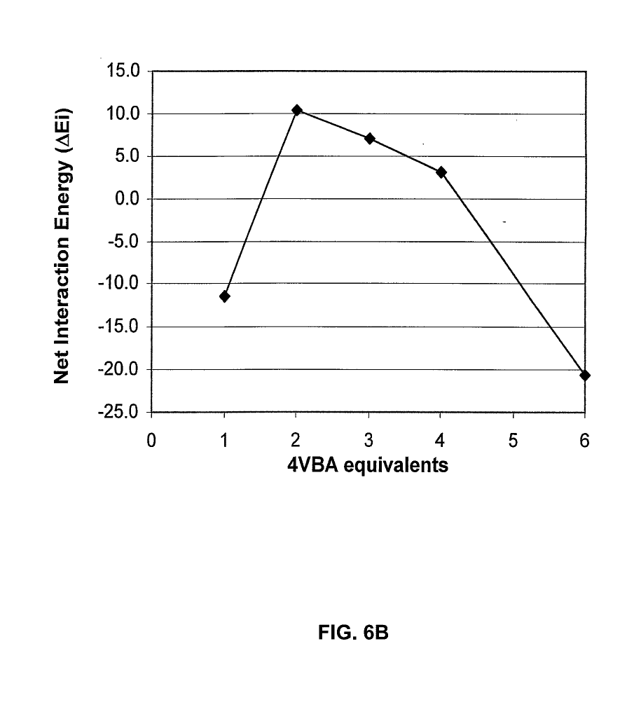

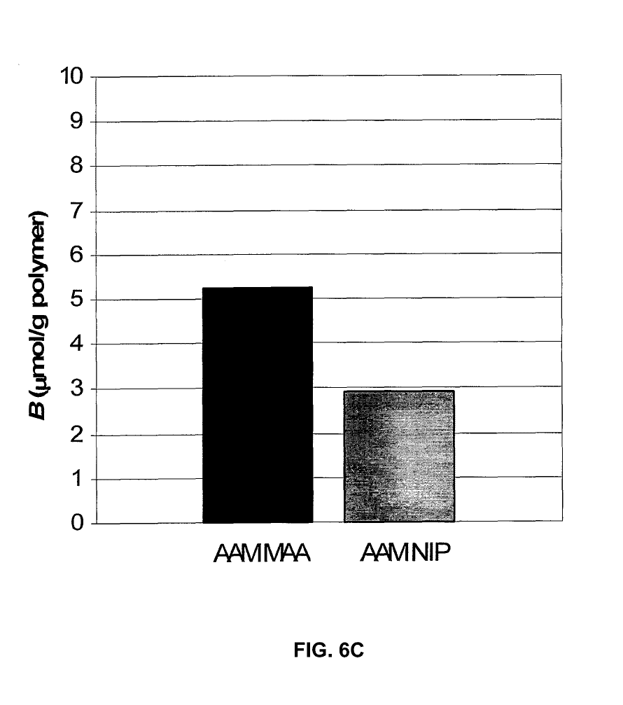

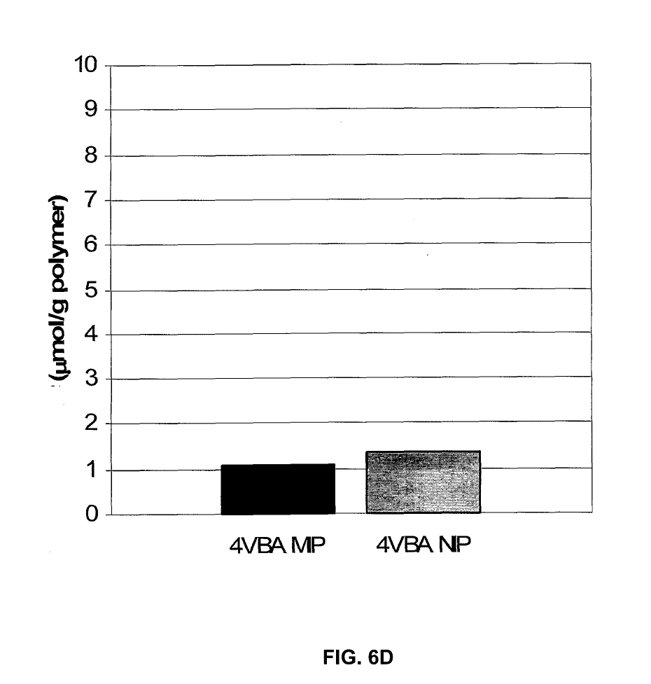

Static binding evaluation data for the AAM-based MIP derived with the 1:6:30 ratio confirmed our conclusion from the modelling studies, with the results indicating that although some low affinity for resveratrol was observed, the selective performance was significantly poorer than for MIPs employing 4-vinylpyridine. A molecularly imprinted polymer was also prepared using 4VBA as functional monomer based on the preparation conditions used to make MIP 8 (the most successful 4VP-based MIP). In this case, the modelling data failed to show any reliable minimum for the monomer ratio and in most cases predicted unfavourable interactions (for 2, 3 and 4 equivalents 4VBA). The static binding evaluation data supported this prediction with no recognition of resveratrol and negligible affinity observed (FIGS. 6A-6D). This result may be explained by the lack of suitable donor-acceptor interactions between the electron deficient aromatic groups of resveratrol and 4VBA, thus preventing any favourable interactions from taking place.

| | Initial | Resveratrol | | | |
|---|---|---|---|---|---|
| Polymer | Reference | Template | Functional | Cross-linker | |
| Code | Code | (T) | Monomer (FM) | (XL) | Porogen |
| AAM MIP | LS1-9 | 1 mmol | AAM, 6 mmol | EGDMA, 30 mmol | ACN |
| AAM NIP1 | LS1-9b | — | AAM, 6 mmol | EGDMA, 30 mmol | ACN |
| 4VBAMIP | LS2-7p12 | 1 mmol | 4VBA, 3 mmol | EGDMA, 15 mmol | ACN/EtOH |
| | | | | | (5/1) |
| 4VBA NIP | LS2-7p12b | — | 4VBA, 3 mmol | EGDMA, 15 mmol | ACN/EtOH |
| | | | | | (5/1) |

### ¹H NMR Spectroscopy Titrations

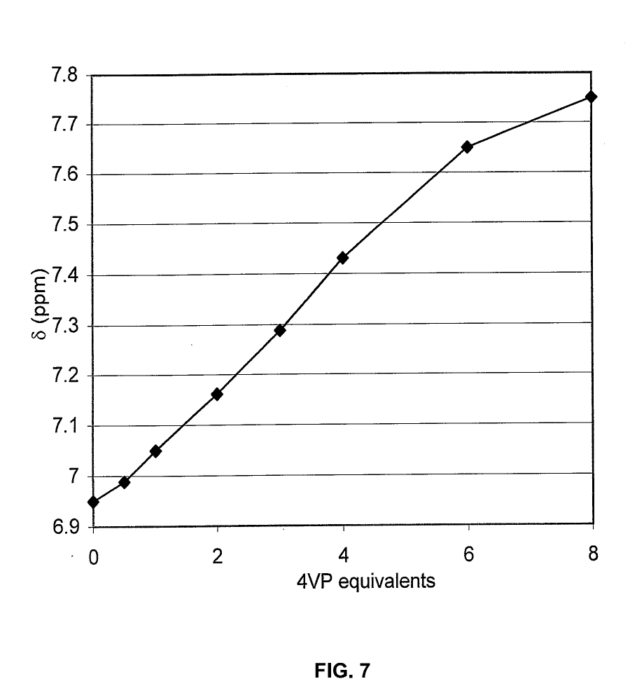

Resveratrol was dissolved in CD₃CN, and was titrated with increasing molar equivalents of 4VP. Upon each addition, ¹H NMR spectra were recorded, and the change in aromatic —OH shifts followed. The presence of H bonding interactions was evidenced by the consistent downfield movement of the aromatic —OH shift (Table 5 and FIG. 7). After 8 equivalents, the OH peak became unobservable.

TABLE 5
¹H NMR titration for Resveratrol against increasing amounts of 4VP.

| Resveratrol | 4VP molar | 4VP vol. (μL) | 4VP total vol. | Δ |
|---|---|---|---|---|
| (mmol) | eq | additions | (μL) | (ppm) |
| 0.1 | 0 | 0 | 0 | 6.95 |
| 0.1 | 0.5 | 5.1 | 5.1 | 6.99 |
| 0.1 | 1.0 | 5.1 | 10.2 | 7.05 |
| 0.1 | 2.0 | 5.1 | 20.4 | 7.16 |
| 0.1 | 3.0 | 10.2 | 30.6 | 7.29 |
| 0.1 | 4.0 | 10.2 | 40.8 | 7.43 |
| 0.1 | 6.0 | 20.4 | 61.2 | 7.65 |
| 0.1 | 8.0 | 20.4 | 81.6 | 7.75 |

Resveratrol imprinted monoliths were prepared using 4VP and EGDMA as functional and cross-linking monomers respectively in various porogenic mixtures (Table 6) After polymerisation monoliths were ground, sieved to a particle size of 60-90 μm, followed by repeated washing in methanol containing acetic acid (10% v/v) to remove the template.

Molecularly Imprinted Polymers (MIPs) Templated with Resveratrol

First and Second generation molecularly imprinted polymers (MIPs) were prepared as described in Table 6, using the following procedure. In a glass test tube fitted with a suba seal, the template (resveratrol) and functional monomer (4VP) were sonicated for 10 minutes prior to the addition of cross-linker (ethylene glycol dimethacrylate, EGDMA) and free radical initiator (AIBN) followed by N_{2(g)} purge for 10 minutes. Thermal polymerization for 1^{st} generation MIPs was carried out at 45° C. for 12 hours, then at 50° C. for 24 hours, and for 2^{nd} generation MIPs thermal polymerization was performed at 50° C. for 16 hours, then 55° C. for 5 hours and 60° C. for 4 hours, resulting in bulk monolith polymers. Non-imprinted control polymers (NIPs) were prepared in exactly the same manner, but in the absence of the template. Monolith polymers were removed from reaction tubes and ground using a Reutsh 200 ball mill, then sieved to size (60-90 μm). Fines were removed by repeated suspension of polymer particles in acetone and filtering through a 20 μm glass frit. The resveratrol template was removed by repeated washing in methanol/acetic acid (10%) until no template was present as observed by UV-VIS spectrophotometry at vₘₐₓ of 235 nm, 307 nm and 321 nm UV-Vis analysis of the washing extracts showed that >99% of the template had been removed.

TABLE 6

| Polymer | Resveratrol | Functional | Cross-linker | |
|---|---|---|---|---|
| Code | Template (T) | Monomer (FM) | (XL) | Porogen |
| MIP 1 | 1 mmol | 4VP, 1 mmol | EGDMA, 10 mmol | ACN, 15 mL |
| NIP 1 | — | 4VP, 1 mmol | EGDMA, 10 mmol | ACN, 15 mL |
| MIP 2 | 0.33 mmol | 4VP, 1 mmol | EGDMA, 10 mmol | ACN, 15 mL |
| MIP 3 | 0.25 mmol | 4VP, 1 mmol | EGDMA, 10 mmol | ACN, 15 mL |
| MIP 4 | 0.17 mmol | 4VP, 1 mmol | EGDMA, 10 mmol | ACN, 15 mL |
| NIP 2-4 | — | 4VP, 1 mmol | EGDMA, 10 mmol | ACN, 15 mL |
| MIP 5 | 1.32 mmol | 4VP, 4 mmol | EGDMA, 55 mmol | ACN, 20 mL |
| NIP 5 | — | 4VP, 4 mmol | EGDMA, 55 mmol | ACN, 20 mL |
| MIP 6 | 0.33 mmol | 4VP, 1 mmol | EGDMA, 10 mmol | EtOH/H₂O (4:1 v/v), |
| | | | | 5 mL |
| NIP 6 | — | 4VP, 1 mmol | EGDMA, 10 mmol | EtOH/H₂O (4:1 v/v), |
| | | | | 5 mL |
| MIP 7 | 1 mmol | 4VP, 3 mmol | EGDMA, 15 mmol | ACN, 15 mL |
| NIP 7 | — | 4VP, 3 mmol | EGDMA, 15 mmol | ACN, 15 mL |
| MIP 8 | 1 mmol | 4VP, 3 mmol | EGDMA, 15 mmol | ACN/EtOH (5:1 |
| | | | | v/v), 6 mL |
| NIP 8 | — | 4VP, 3 mmol | EGDMA, 15 mmol | ACN/EtOH (5:1 |
| | | | | v/v), 6 mL |
| MIP 9 | 1 mmol | 4VP 3 mmol | EGDMA, 20 mmol | ACN/EtOH (5:1 |
| | | | | v/v), 6 mL |
| NIP 9 | — | 4VP 3 mmol | EGDMA, 20 mmol | ACN/EtOH (5:1 |
| | | | | v/v), 6 mL |
| MIP 10 | 1 mmol | 4VP 3 mmol | EGDMA, 17 mmol | ACN/EtOH (5:1 |
| | | | | v/v), 6 mL |
| NIP 10 | — | 4VP 3 mmol | EGDMA, 17 mmol | ACN/EtOH (5:1 |
| | | | | v/v), 6 mL |
| MIP 11 | 1 mmol | 4VP 3 mmol | TRIM, 3 mmol | ACN/EtOH (5:1 |
| | | | | v/v), 6 mL |
| NIP 11 | — | 4VP 3 mmol | TRIM, 3 mmol | ACN/EtOH (5:1 |
| | | | | v/v), 6 mL |
| MIP 12 | 1 mmol | NVIM 3 mmol | EGDMA | ACN/EtOH (5:1 v/v) |
| | | | 15 mmol | |
| NIP12 | — | NVIM 3 mmol | EGDMA | ACN/EtOH (5:1 v/v) |
| | | | 15 mmol | |
| MIP 13 | 1 mmol | NVIM 6 mmol | EGDMA | ACN/EtOH (5:1 v/v) |
| | | | 15 mmol | |
| NIP 13 | — | NVIM 6 mmol | EGDMA | ACN/EtOH (5:1 v/v) |
| | | | 15 mmol | |
| MIP 14 | 1 mmol | NVIM 6 mmol | TRIM 6 mmol | ACN/EtOH (5:1 v/v) |
| NIP 14 | — | NVIM 6 mmol | TRIM 6 mmol | ACN/EtOH (5:1 v/v) |

An example of a Typical MIP preparation is described below:

### Preparation of MIP 8 (LS3-54p118-1-7-08)

228.2 mg of resveratrol (1 mmol) and 321 μL of 4VP (3 mmol) were dissolved in 1 mL of EtOH and 5 mL of ACN in a 15 mL test tube and sealed with a suba seal. The solution was purged with N_{2(g)} for 2 minutes and then sonicated for 20 minutes. To the solution was added 2.83 mL of EGDMA (15 mmol) and 51 mg of AIBN (0.31 mmol) radical initiator. The reaction mixture was then purged with N_{2(g)} for a further 2 minutes prior to being submersed into a 50° C. water bath for 18 hours. The water temperature was then increased to 60° C. for 24 hours. As the polymerisation initiation liberates N_{2(g)}, the test tube was fitted with a needle and syringe barrel with plunger to act as a pressure valve. This was repeated for the NIP control polymer without the addition of the resveratrol template.

The bulk polymer monolith was then removed from the test tube, crushed and ground to a particle size of 60-90 μm using a Restch 200 ball mill. The resulting polymer particles were the washed repeatedly in MeOH/AcOH (10%) (v/v) to remove the template and any unreacted monomer species. The MIP and NIP materials were then washed with MeOH to remove any traces of AcOH and dried under reduced pressure.

### MIP Binding Evaluation

Initial MIP binding studies were performed using 4.5 mL fitted polypropylene reaction tubes fitted to a Mettler Toledo MiniBlock™ system. Using this apparatus, first generation MIPs were rapidly evaluated for resveratrol affinity after a short incubation period, followed by vacuum assisted filtration into fraction collection tubes. These evaluations were designed to identify the most suitable template to functional monomer ratio. The rebinding studies were performed as follows:

(1) 50 mg of MIP (or respective NIP control) was measured into a reaction tube fitted with a 25 μm polypropylene porous frit;
(2) a rebinding solution of resveratrol (0.05 min) in acetonitrile was added and the reaction mixture was incubated for 30 min at room temperature with continuous shaking;
(3) the filtrate was collected, diluted five-fold with acetonitrile—a 3 mL aliquot was analysed by UV-VIS spectroscopy at vₘₐₓ 235 nm, 307 nm and 321 nm using a quartz cuvette with a 1 cm path length and the concentration of free resveratrol calculated from an experimentally determined a five point calibration curve. The concentration of resveratrol bound to the MIP (or respective NIP) was then determined as the difference between the initial resveratrol concentration and the final free resveratrol concentration. This was then expressed as the amount bound, B, in μmoles/g polymer.
(4) MIPs were washed sequentially with acetonitrile methanol and methanol containing acetic acid (10% v/v) and again with methanol to remove bound resveratrol.

MIP negative control experiments for resveratrol binding were performed identically to the above procedure, with the exception that the rebinding solution did not contain resveratrol.

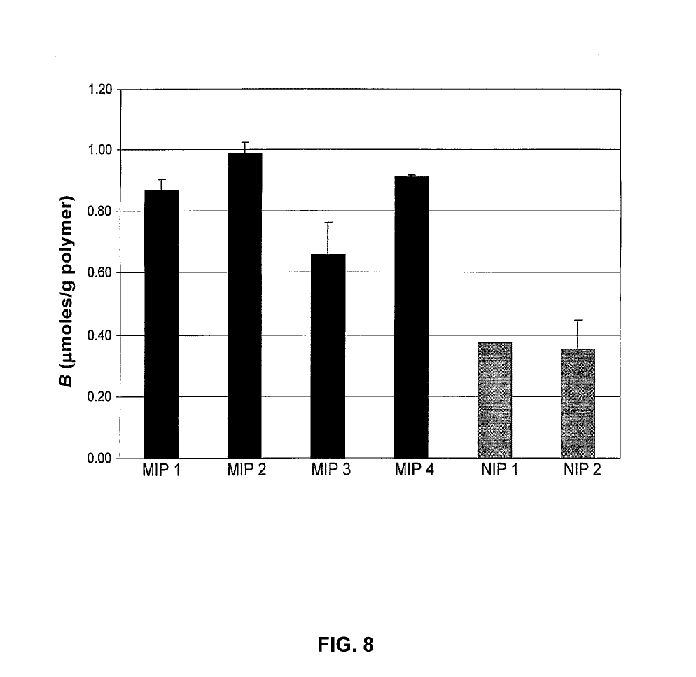

FIG. 8 illustrates the binding of resveratrol to MIPs prepared with the template resveratrol and using 4VP as the functional monomer. Multiple fast screening assays were performed using 1 mL of resveratrol solution (0.05 min) in acetonitrile to demonstrate the affinity of the MIP for the template resveratrol and the reproducibility of binding. Evidence of an imprinting effect is clearly apparent for each of the resveratrol imprinted polymers prepared and tested.

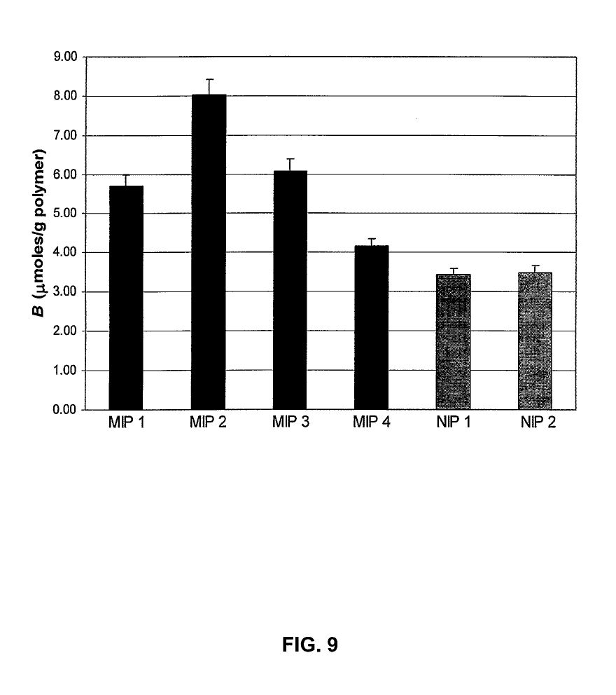

To further ascertain the most appropriate template to functional monomer ratio, an assay employing a 10 fold increase in resveratrol concentration (0.5 min) was employed. The results are set out in FIG. 9.

The relative imprinting factor (IF), relating the performance of the MIP with respect to the NIP control, was determined for each MIP, from

IF=B_{MIP}/B_{NIP},

where B_{MIP} and B_{NIP} is the amount of resveratrol bound to the MIP and NIP respectively.

TABLE 7
Relative binding performance with corresponding imprinting
factors (IF) for assays at both resveratrol concentrations of
0.05 and 0.5 mM.

| | 0.05 mM Resveratrol Assay | | | 0.5 mM Resveratrol Assay | | |
|---|---|---|---|---|---|---|
| | B_{MIP} | B_{NIP} | | B_{MIP} | B_{NIP} | |
| Polymer | μmoles/g | μmoles/g | IF | μmoles/g | μmoles/g | IF |
| MIP 1 | 0.904 | 0.376 | 2.40 | 5.706 | 3.411 | 1.67 |
| MIP 2 | 0.946 | 0.448 | 2.11 | 8.031 | 3.481 | 2.31 |
| MIP 3 | 0.746 | 0.448 | 1.70 | 6.080 | 3.481 | 1.75 |
| MIP 4 | 0.909 | 0.448 | 2.03 | 4.147 | 3.481 | 1.19 |

Thus, in accordance with the modeling data, the most suitable template:monomer ratio was identified to be 1:3 for the preparation of a resveratrol imprinted polymer using 4VP as the functional monomer (Table 7). MIPs prepared in this manner demonstrated maintained high performance levels (highest capacity and IF values) after consecutive repeated binding studies at both low and intermediate resveratrol concentrations (0.05 and 0.5 min respectively).

### Effect of Porogen and Cross-Linking Amount

MIPs based on this formulation were subsequently prepared with varying amounts of EGDMA to assess the effect of cross-linking on binding affinity. Example MIPs are MIP 2, MIP 5, MIP 7, MIP 8, MIP 9 and MIP 10).

However it was observed that MIP 7 resulted in a pseudo precipitation polymerisation that produced a talc-like polymer which was physically soft or weak and was reduced to fines or beads of a particle size <20 μm. This was due to the low amount of cross-linking and large porogen volume which was required to dissolve the resveratrol template. Although such sizes are useful for analytical techniques, this polymer was deemed unsuitable for future large scale or industry scale separation techniques, as the small particle size would result in unacceptably high back-pressures regardless of binding performance (data not shown).

Therefore a small amount of EtOH was included in the ACN porogen (MIP 8) which increased the solubility of the template resveratrol thus affording a smaller porogenic volume and the preparation of a monolithic polymer which could be ground and sieved to a suitable particle size.

An example preparation for a polymer containing an ACN/EtOH (5:1 v/v) porogenic mixture is described below for MIP 8 (LS2-6p10-7-2-07):

Static binding assays were employed to evaluate the effect of cross-linking. An example of an assay is detailed below:

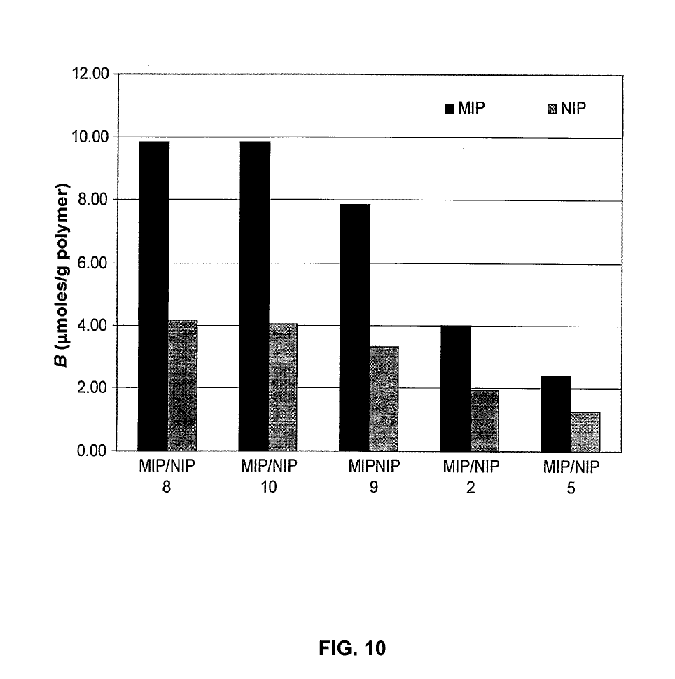

Into a 1.7 mL eppendorf tube was measured 30 mg of MIP, 1.5 mL of resveratrol (0.5 mM) in acetonitrile and the resulting sample mixture was mixed for 18 hours using a rotary suspension mixer at 28° C. Each sample was prepared in duplicate and repeated with NIP control samples. In addition to this blank control samples were prepared using 30 mg of the respective polymer with 1.5 mL of blank acetonitrile solution. The samples was then centrifuged at 13000 rpm for 15 minutes and a 200 μL aliquot removed for reverse phase HPLC analysis under isocratic conditions with UV-Vis detection at a wavelength of γ=321 nm. The area under the curve was determined and the concentration of free resveratrol determined using an experimental five point calibration curve. The concentration of resveratrol bound to the MIP was calculated by subtracting the free resveratrol concentration from the concentration of the binding solution. The results are set out in FIG. 10.

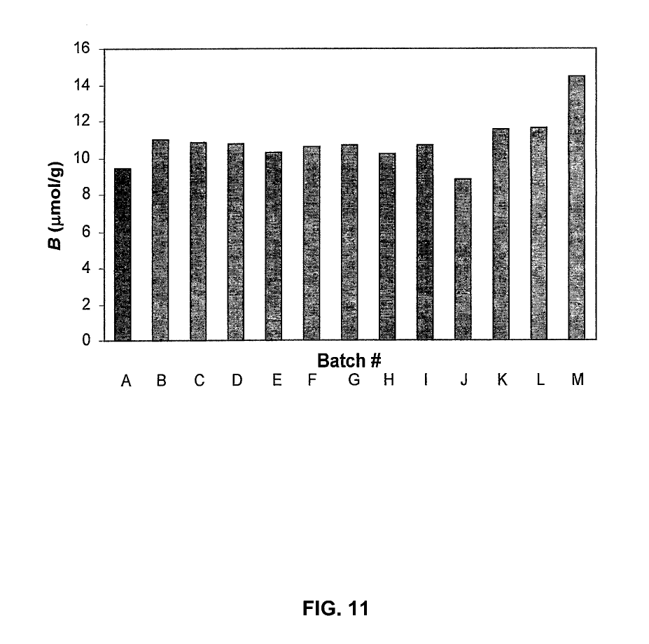

An important aspect of this invention is the ability to replicate the MIP preparation and subsequent binding affinity. As an example of this MIP 8 was prepared in multiple batches (FIG. 11), and the binding performance observed via static binding studies as described above. The results clearly show good reproducibility with consistent resveratrol binding performance of approximately 10-11 μmoles/g of polymer.

Further studies into binding characteristics of MIPs discussed within this invention include the static capacity and static cross-reactivity studies for which examples using MIP 8 are discussed below. Static Capacity Binding Assay: into 1.7 mL eppendorf tubes was weighed 30 mg of polymer (both MIP and NIP) to which was added increasing concentrations of resveratrol solution at constant volume (1.5 mL) in acetonitrile. The resulting mixture was incubated while mixing at approximately 40 rpm for 18 hours (unless stated otherwise), after which the mixture was centrifuged at 13000 rpm for 15 minutes. A 200 μL aliquot of the supernatant was removed and analysed by reverse phase HPLC analysis with UV detection, and the concentration of resveratrol in the supernatant determined via a 5 point calibration curve. This was subtracted from the initial binding solution to give the amount of resveratrol bound. Binding (B) was expressed as analyte bound, B in μmol/g polymer.

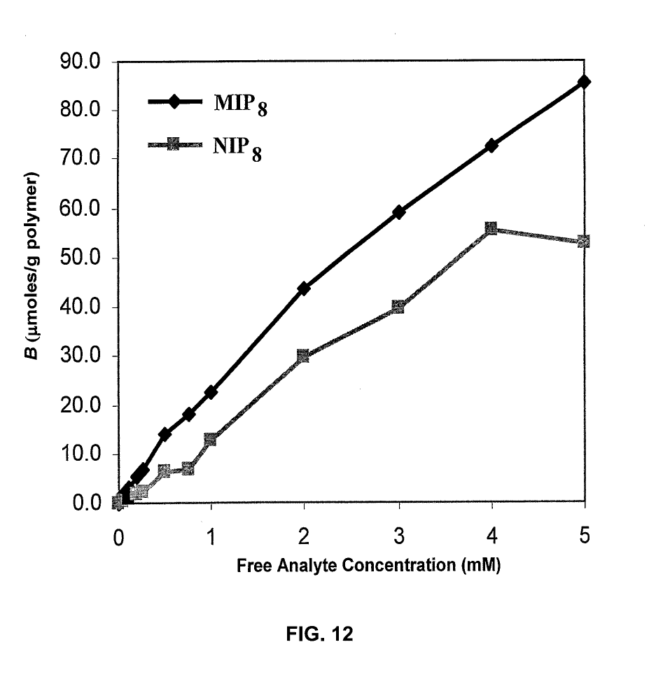

Preliminary data shows no clear capacity (as would be evidenced by a plateau in the curve) while the non-specific binding on the NIP control polymer was observed to be quite variable over repeat binding experiments (FIG. 12) and surprisingly high.

### Static Cross-Reactivity Binding Assay

For static cross-reactivity studies the binding procedure was the same as above with the exception that the concentration of analyte binding solutions was kept at 0.5 min.

Cross-reactivity studies on MIP_{E} (LS 2-6p10-7-1-07) were continued with a range of zero, one, two, three and four point compounds. Compounds included in the study were: Resveratrol (1), 3,5-dihydroxy-N-(4-hydroxyphenyl)benzamide (BDp105-21-8-07) (2), ‘Green’ Resveratrol (BDp55-11-12-2006) (3), (E)-5-(4-hydroxystyryl)benzene-1,2, 3-triol (BDp75-23-7-2007) (4), (E)-4-(3,5-dinitrostyryl)phenol (BDp15-2-11-06) (5), (E)-4-(3,5-dimethylstyryl)phenol (BDp127-22-9-06) (6), (E)-5-styrylbenzene-1, 3-diol (BDp123-8-3-07) (7), Caffeic acid (8), (E)-3,4′-(ethene-1,2-diyl)diphenol (BDp35-23-5-07) (9), (E)-4-styrylphenol (BDp81-5-10-2007) (10), (E)-3-styrylphenol (BDp47-15-6-07) (11), (E)-Stilbene (12), 5-(4-hydroxyphenethyl)benzene-1,3-diol (BDp55-12-7-2006) (13), 5-(4-hydroxyphenethyl)benzene-1,2,3-triol (BDp67-16-7-2007) (14), 3-(4-hydroxyphenethyl)phenol (BDp35-23-5-07) (15), Bisphenol A (16), 3-phenethylphenol (BDp55-27-6-07) (17), 4-phenethylphenol (BDp89-9-8-06) (18).

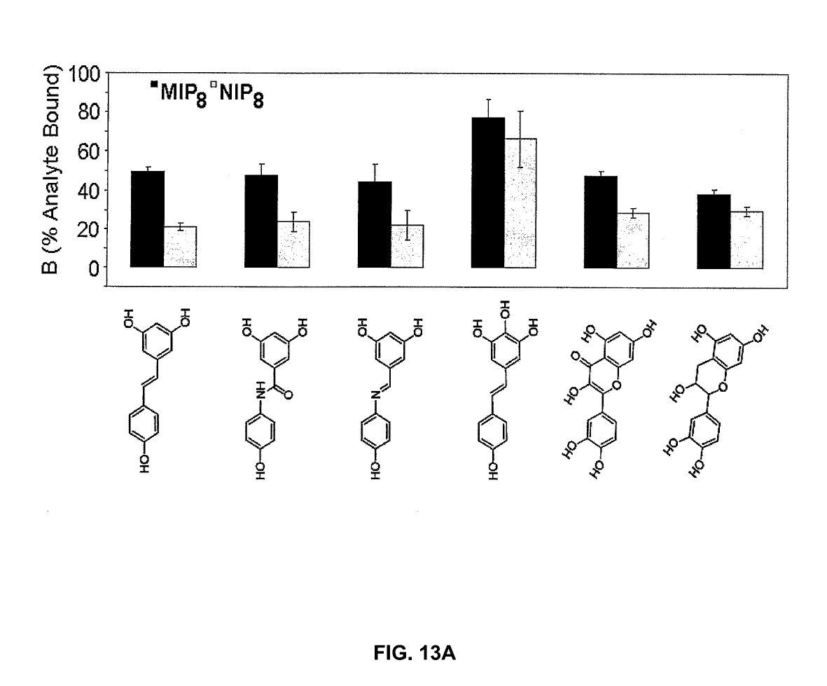

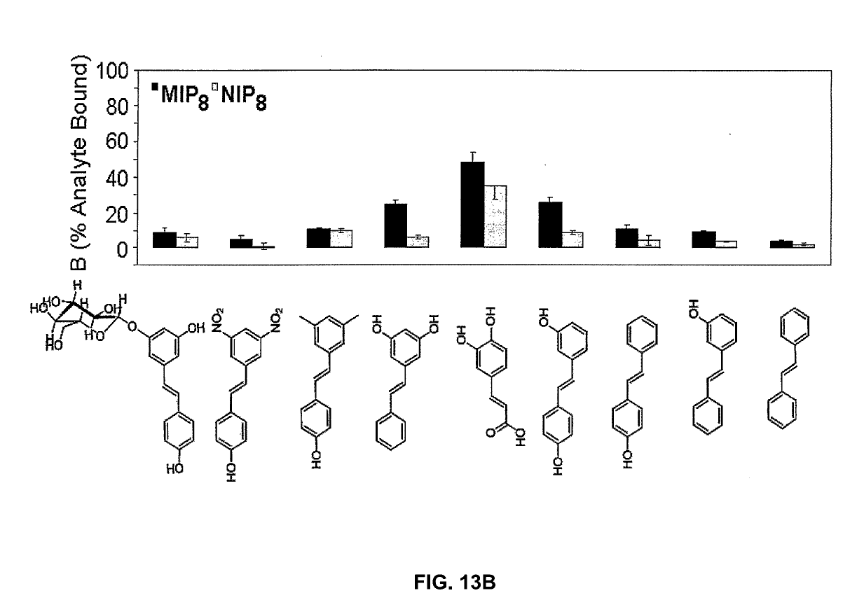

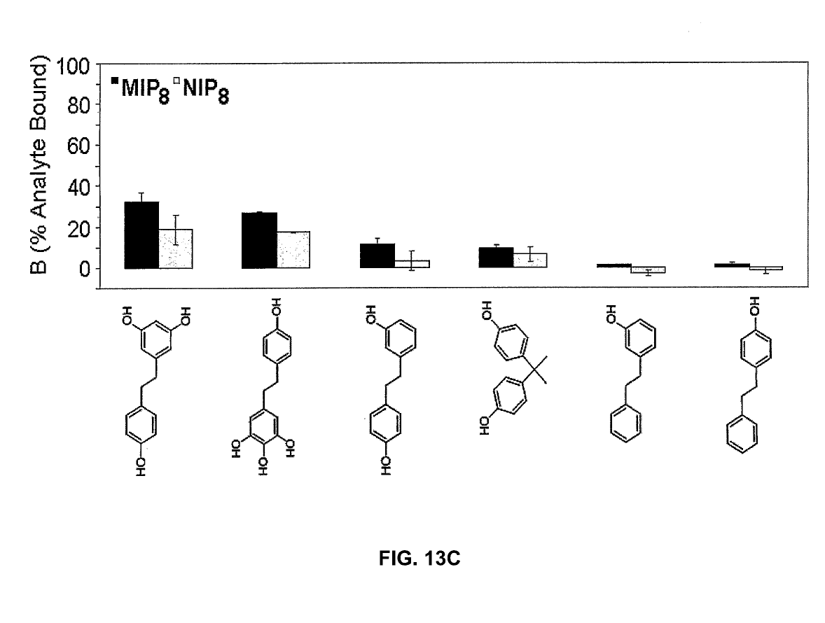

The numbering scheme used in this section corresponds to the numbering scheme in FIGS. 13A-13C.

Cross-reactivity binding data (FIGS. 13A-13C) highlights some of the structural requirements related to binding affinity onto MIP 8. It is clear that binding affinity is strongly influenced by the combined presence of multiple hydrogen bonding units and C═C, C═N or NH—C═O linker groups. The affinity and selectivity of the amide and imine analogues (2 and 3 respectively) is similar to that of resveratrol with approximately 55% of the applied analyte retained on MIP 8. Interestingly the removal of the C═C double bond (14) while retaining the extra —OH group, resulted in a decrease in sorption to both MIP 8 and NIP 8 by approximately 65%. This same trend was observed for all species, although to a lesser degree, indicating that either the increased electron density or rigidity afforded by the C═C linkage results in greater affinity and in many cases selectivity of MIP 8.

Other notable observations were the loss of affinity upon removal of two or more —OH units such as with the nitro (5), dimethyl (6) and stilbene (12) analogues for which minimal affinity was observed.

### MIPs Employing Resveratrol Analogues as Templates

Some of the resveratrol analogues that showed affinity towards MIP 8, such as the amide and the imine analogues (FIGS. 13A-13C), were also incorporated into MIP materials as templates in order to generate a cavity capable of binding resveratrol and/or other polyphenols, thus not requiring resveratrol as the template. These MIP materials were prepared as summarized in Table 8 using the procedure described above for MIP 8.

TABLE 8
Summary of some examples of MIP preparations employing resveratrol
analogues as the template species.

| | | Functional | | | |
|---|---|---|---|---|---|
| Polymer | | Monomer | Cross-linker | T:FM:XL | |
| Code | Template (T) | (FM) | (XL) | Ratio | Porogen |
| MIP 15 | (E)-5-((4- | 4VP, 1 mmol | EGDMA, 5 mmol | 1:3:15 | ACN/EtOH |
| | hydroxyphenylimino)methyl)benzene- | | | | (5:1), 2 mL |
| | 1,3-diol | | | | |
| | (BDp55-11-12- | | | | |
| | 2006), 0.33 mmol | | | | |
| MIP 16 | 3,5-dihydroxy- | 4VP, 1.5 mmol | EGDMA, 7.5 mmol | 1:3:15 | Acetone 3 mL |
| | N-(4- | | | | |
| | hydroxyphenyl)benzamide | | | | |
| | (BDp105-21-8- | | | | |
| | 07), 0.5 mmol | | | | |
| NIP 16 | — | 4VP, 1.5 mmol | EGDMA, 7.5 mmol | 0:3:15 | Acetone 3 mL |
| MIP 17 | (E)-4-(3,5- | 4VP, 0.75 mmole | EGDMA, | 1:3:15 | ACN/EtOH |
| | dinitrostyryl)phenol | | 3.75 mmole | | (5:1), |
| | (BDp15-2- | | | | 1.5 mL |
| | 11-06), 0.25 mmole | | | | |
| MIP 18 | (E)-5-(4- | 4VP, | EGDMA, 5 mmol | 1:4:20 | Acetone, |
| | hydroxystyryl)benzene- | 1 mmol | | | 2 mL |
| | 1,2,3-triol | | | | |
| | (BDp75-23-7- | | | | |
| | 2007), 0.25 mmole | | | | |
| NIP 18 | — | 4VP, | EGDMA, 5 mmol | 1:4:20 | Acetone, |
| | | 1 mmol | | | 2 mL |

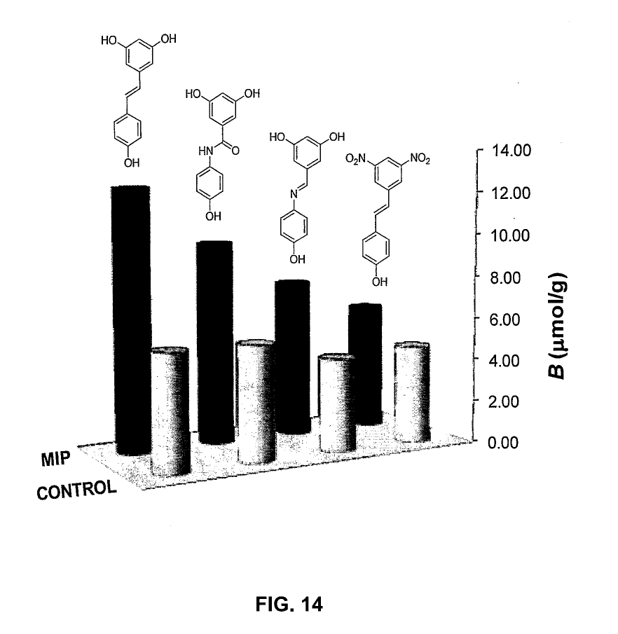

Examples of molecularly imprinted polymers templated with resveratrol analogues are shown in FIG. 14, which display their respective ability to bind resveratrol from an acetonitrile solution under static conditions. Polymers imprinted with the amide and the imine analogues exhibited good resveratrol affinity, with the amide imprinted polymers binding performance approaching that of MIP 8.

### Molecularly Imprinted Solid Phase Extraction (MISPE) Studies

In order to successfully apply MIP technology to the separation and enrichment of bioactives such as polyphenols at the industry scale, a format affording good separation and high flow is required. As such it was anticipated that an example of an appropriate format for this technology would be a scaleable liquid chromatography technique such as solid phase extraction.

### An Example of MISPE Column Preparation

Initial MISPE and non-imprinted solid phase extraction (NISPE) columns were prepared in 3.5 mL syringe barrels capped with 20 μm glass frits. 100 mg of dry MIP were slurry packed into the syringe barrels, allowed to settle overnight. A second glass frit cap was placed on the top of the wet packed column, which was then gently compressed using a plunger, with care taken to keep the column wet, thus preventing the formation of voids and cracks in the column packing. This was repeated for the respective NIP control materials. Columns were successively washed with methanol, methanol containing acetic acid (10% v/v), methanol and acetonitrile.

### General MISPE Assay Protocol

The MIP columns were pre-conditioned with, successively, methanol containing acetic acid (10% v/v), methanol and acetonitrile). After loading of 1 mL resveratrol (or analyte) solution (0.5 min) onto the MISPE columns, the eluate and solvent from subsequent washing steps were collected in a single fraction tube. Elution of the bound molecules was achieved by several washes with methanol containing acetic acid (10% v/v). Samples were clarified by centrifugation and an aliquot of the supernatant was analysed by reversed-phase HPLC. Samples comprising solvents other than acetonitrile or acetonitrile/H₂O were evaporated under vacuum at 30° C. then reconstituted in acetonitrile prior to analysis. All samples were chromatographed via isocratic elution in acetonitrile/H₂O (7:3) as mobile phase at a flow rate of 0.5 mL/min with UV-VIS detection at 321 nm for detection and quantification of resveratrol.

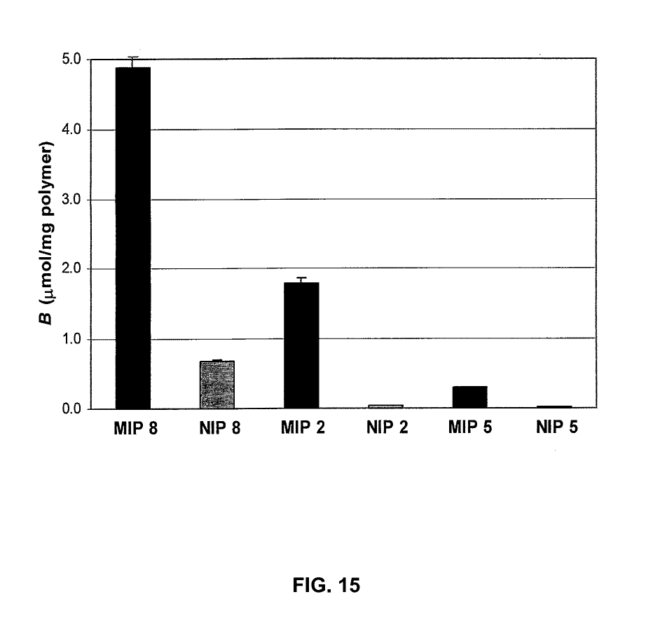

The preparation of MISPE materials have been further improved using a modified procedure and apparatus, which has resulted in MIPs that have demonstrated enhanced adsorption selectivity for resveratrol in multiple, reproducible MISPE assays. This procedure has been applied to several different MIPs prepared using various template:functional monomer:cross-linker (T:FM:XL) ratios and alternative porogens. The retention of resveratrol on these MISPE columns is shown in FIG. 15, which demonstrates that MIPs prepared using acetonitrile/ethanol (5:1 v/v) with a 1:3:15 T:FM:XL ratio clearly outperforms other MIP preparations with the imprinting factor approaching 10:1 after extensive washing with acetonitrile. The presence of ethanol in the porogenic solvent has a beneficial influence, which may result from the increased solubility of the template, greater structural rigidity due to a reduced porogenic volume or by influencing intermolecular interactions, such as π-π stacking, during the pre-polymerisation stage of preparation.

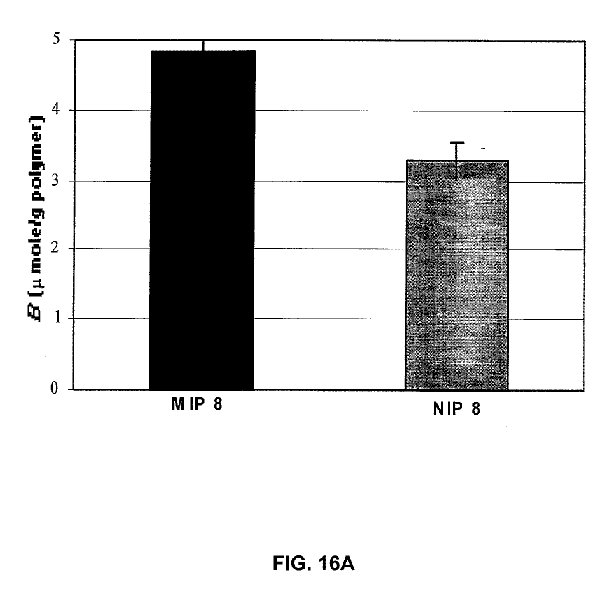

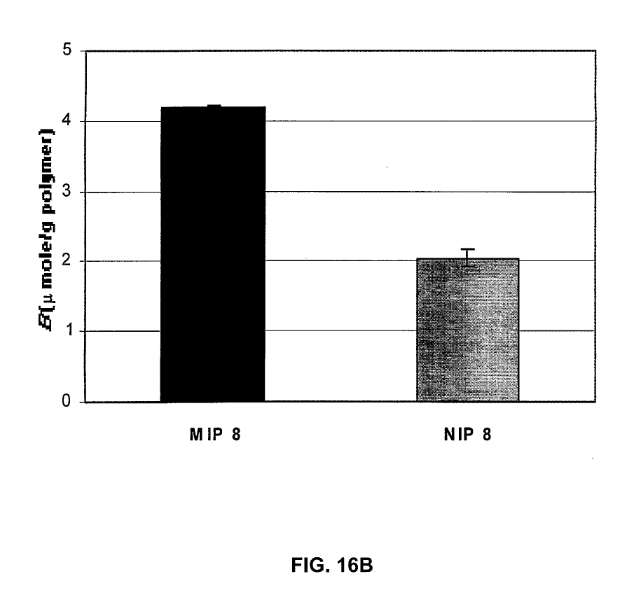

The performance of MIP 8 was subsequently examined under semi-aqueous conditions (ethanol (20% v/v in water) in MISPE format with a clean up step comprising two washes with 20% (v/v) ethanol in water, followed by successive washes with 1% (v/v) acetonitrile in methanol, and acetonitrile. FIGS. 16A and 16B display high adsorption onto MIP 8 with approximately 80% of the initial resveratrol solution retained and an imprinting factor approximately 2 after clean up with an aqueous ethanol rinse. However, selectivity between MIP and NIP was greatly increased while maintaining good levels of adsorption after washing with acetonitrile was applied to remove non-specific binding.

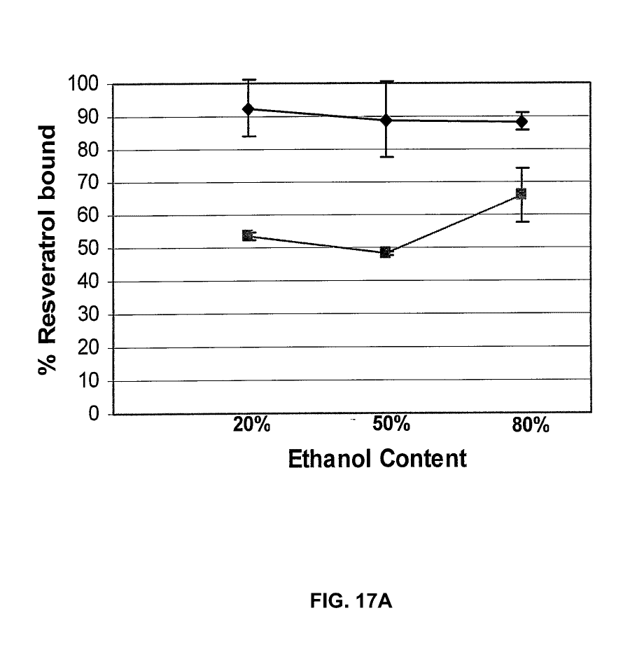

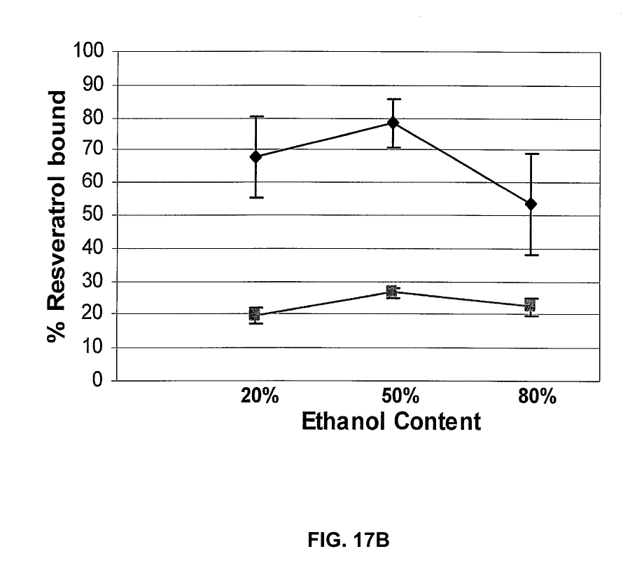

The effect of ethanol and water content on resveratrol affinity to MIP 8 was examined by increasing the ethanol content under aqueous binding conditions. The amount of resveratrol bound expressed as a percentage of the initial loading solution for ease of comparison (FIGS. 17A and 17B). In addition to this, FIGS. 17A and 17B illustrate the application of aqueous ethanol as an environmentally friendly alternative to acetonitrile as a clean up solvent to remove non-specific binding. Based on this data it appears that an EtOH content of between 20-50% results in preferential resveratrol recognition when under aqueous conditions.

Application of a Complex Sample from Agricultural Feedstocks

In order to assess the ability of MIP 8 to concentrate resveratrol from a complex feedstock such as grape press waste and thus be an effective value adding process to the Australian food industry, a grape seed sample was applied to a 100 mg MISPE column packed with MIP 8.

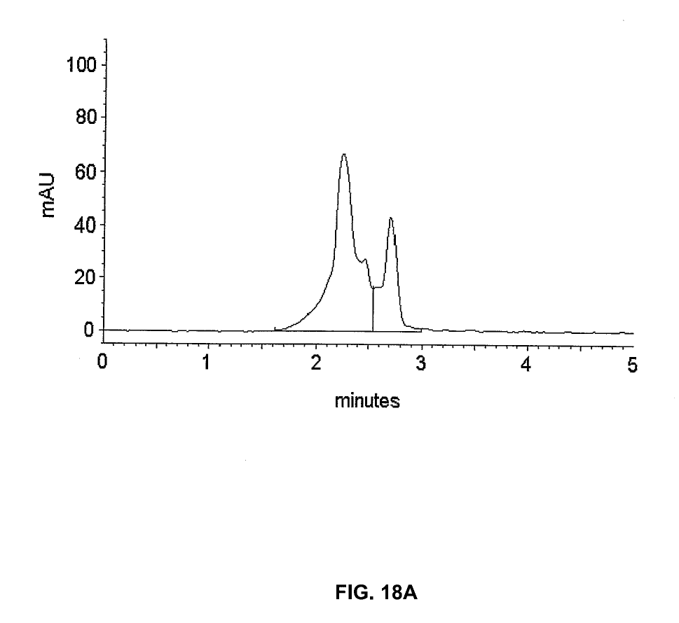

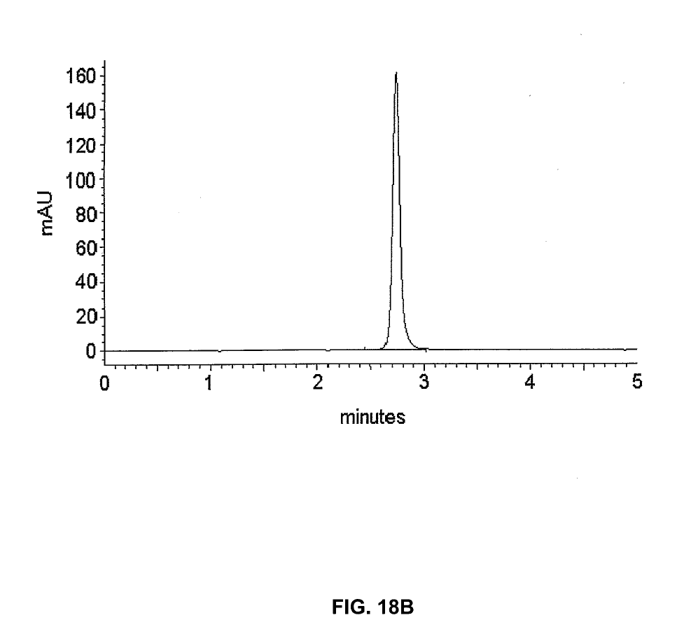

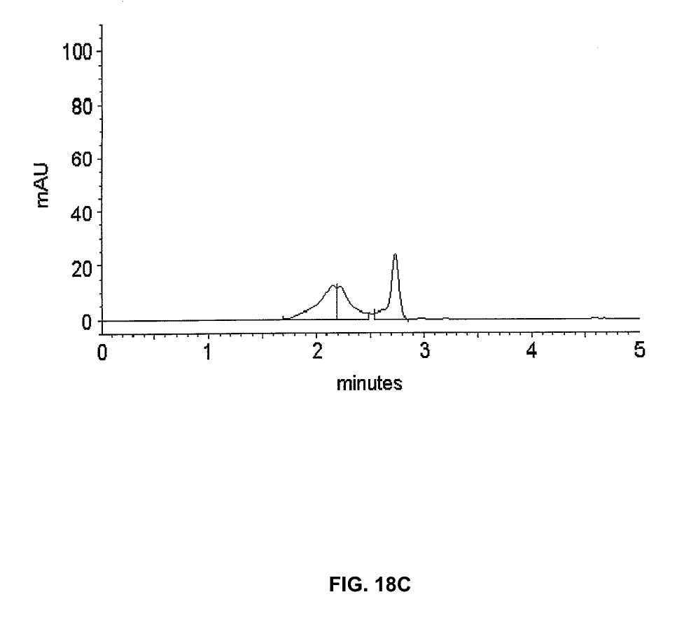

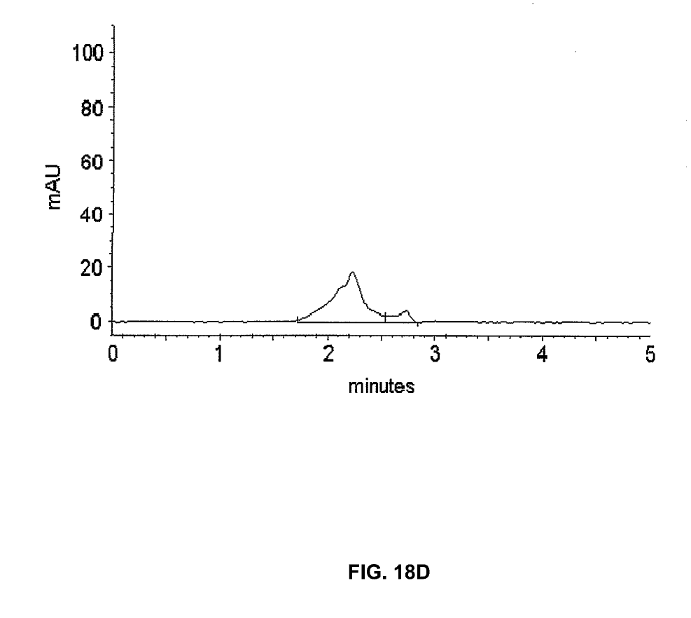

Raw grape seed was provided by CSIRO, Food Science Australia Werribee (Batch No. 02VIN03). 2×10 g samples were measured out, one of which was spiked with 200 μg of resveratrol, and continuously extracted in acetone via soxhlet based on procedure reported by Romero-Perez et al.⁶ yielding control BDp119-14-9-2006 and spiked BDp119-14-9-2006 extracts. 1 g of each extract were separately suspended in 80% (v/v) ethanol, then sonnicated for 30 minutes and centrifuged at 3000 rpm for 30 minutes. 1 mL aliquots of the respective soluble extracts were applied to off-line MISPE columns packed with 100 mg of MIP 8, the washed with 80% (v/v) ethanol followed by successive washes with acetonitrile and final elution with 10% (v/v) AcOH in methanol. A resveratrol standard solution in 80% (v/v) ethanol in water was applied to a separate MISPE column packed with MIP 8 and treated identically to the grape seed extracts. Non-imprinted control columns (NISPE) were treated exactly the same as their respective MIP counterparts. Aliquots of recovered bound material from MIPSE columns were analyzed by HPLC (see FIGS. 18A-18D).

FIG. 18B and FIG. 18C demonstrate that MISPE columns packed with MIP 8 were able to concentrate resveratrol from a complex feedstock such as grape seed extract, as evidenced by the peak consistent with resveratrol observed at 2.735 minutes after elution from the MISPE column, whereas no such peak was observed for the NISPE column.

### Demonstration of Molecularly Imprinted Solid Phase Extraction (MISPE) of Peanut Meal Extract on a 1 g of MIP Material

10 g of peanut meal extract (BDp63-9-7-07) made up in 500 mL EtOH/H₂O (50/50 v/v). Mixture was centrifuged and filtered after which 100 mL aliquot was treated by molecularly imprinted solid phase extraction (MISPE) using 1 g of MIP 8 material (1s3-26p62-1-4-08). The MIP column was then washed with 4 column volumes (4×10 mL) of aqueous ethanol (EtOH/H₂O 50/50 v/v), followed by a selective clean up wash using 3 column volumes of aqueous acetone (acetone/H₂O 50/50 v/v). The remaining bound analytes were eluted via 5×5 mL 10% AcOH in MeOH and 2×5 mL acetone. This procedure was repeated using a column packed with 1 g of non-imprinted control polymer (NIP). The elution fractions were combined, evaporated to dryness and made up to 1 mL in 50% aqueous EtOH of which 200 μL aliquots were analysed by reversed-phase HPLC employing isocratic elution at a flow rate of 0.5 mL/min (Table 9).

TABLE 9
Gradient profile employed for RP-HPLC analysis of untreated and MISPE
treated peanut meal extract. Solvent A = H₂O with 0.1% AcOH,
Solvent B = EtOH/H₂O (80:20 v/v) with 0.1% AcOH.

| Time (min) | % Solvent B |
|---|---|
| 0 | 25% |
| 2 | 25% |
| 6 | 37.5% |
| 9 | 37.5% |
| 12 | 62.5% |
| 15 | 62.5% |
| 18 | 100.0% |
| 22 | 100.0% |
| 25 | 25.0% |
| 29 | 25.0% |

### Results Summary

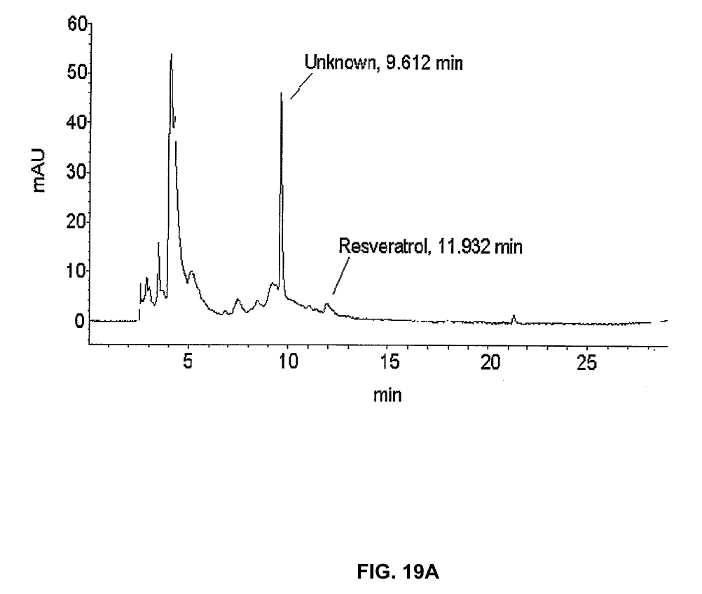

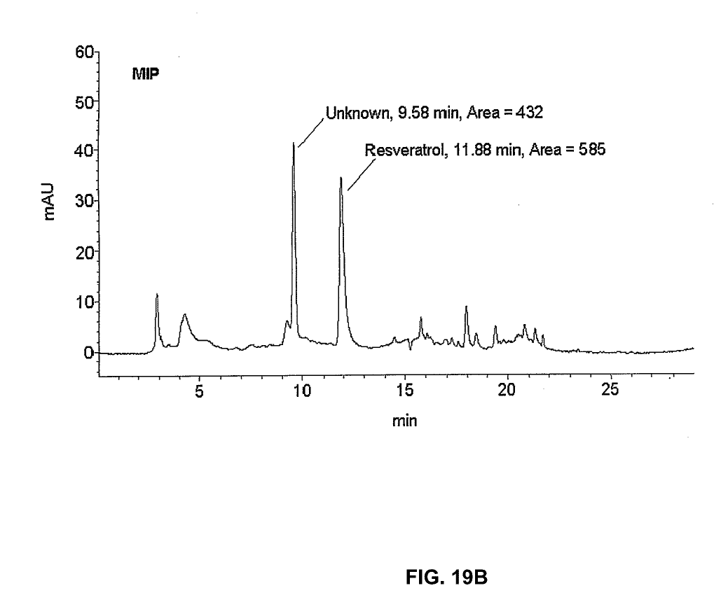

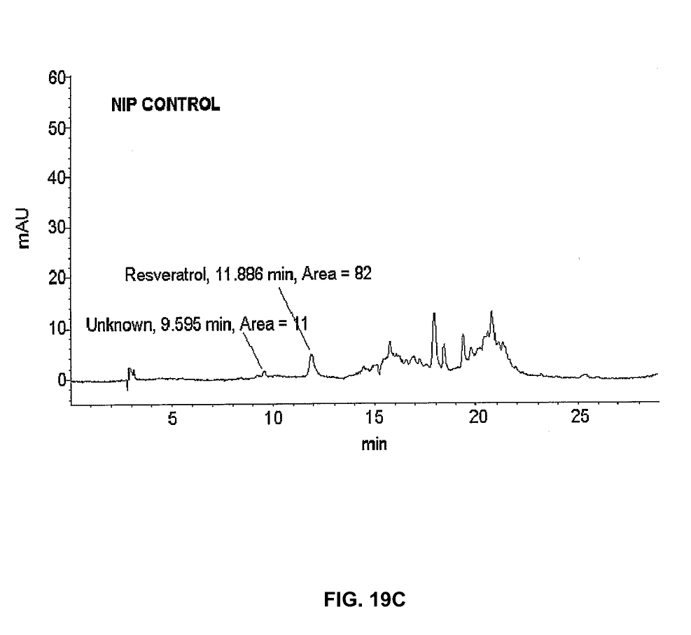

Reversed-phase HPLC generated chromatograms of untreated and MISPE treated peanut meal extract (FIGS. 19A-19C) demonstrate a selective enrichment of resveratrol (approximately 20 fold increase in concentration) and the retention (no enrichment observed) of an unidentified analyte (RT=9.6 min) It should be noted that the MISPE treatment procedure has yet to be optimised, and as such the enrichment of resveratrol and other analytes may yet be improved.

### Demonstration of Molecularly Imprinted Solid Phase Extraction (MISPE) of Peanut Meal Extract on a 1 g of MIP Material (40-Fold Enrichment)

### Experimental Summary

Peanut meal extract (10 g) was made up in 500 mL EtOH/H₂O (50/50 v/v). The mixture was centrifuged and filtered after which 100 mL aliquot was treated by molecularly imprinted solid phase extraction (MISPE) using 1 g of molecularly imprinted polymer (MIP) material. The MIP column was then washed with 4 column volumes (4×10 mL) of aqueous ethanol (EtOH/H₂O 50/50 v/v), followed by a selective clean up wash using 3 column volumes of aqueous acetone (acetone/H₂O 50/50 v/v). The remaining bound analytes were eluted via 5×5 mL 10% AcOH in MeOH and 2×5 mL acetone. This procedure was repeated using a column packed with 1 g of non-imprinted control polymer (NIP). The elution fractions were combined, evaporated to dryness and made up to 1 mL in 50% aqueous EtOH of which a 200 μL aliquots were analysed by reverse phase HPLC employing gradient elution at a flow rate of 0.5 mL/min (Table 10).

TABLE 10
Gradient profile employed for RP-HPLC analysis of
untreated and MISPE treated peanut meal extract.
Solvent A = H₂O with 0.1% AcOH, Solvent
B = EtOH/H₂O (80:20 v/v) with 0.1% AcOH.

| Time (min) | % Solvent B |
|---|---|
| 0 | 25.0 |
| 2 | 25.0 |
| 6 | 37.5 |
| 9 | 37.5 |
| 12 | 62.5 |
| 15 | 62.5 |
| 18 | 100.0 |
| 22 | 100.0 |
| 25 | 25.0 |
| 29 | 25.0 |

### Results Summary

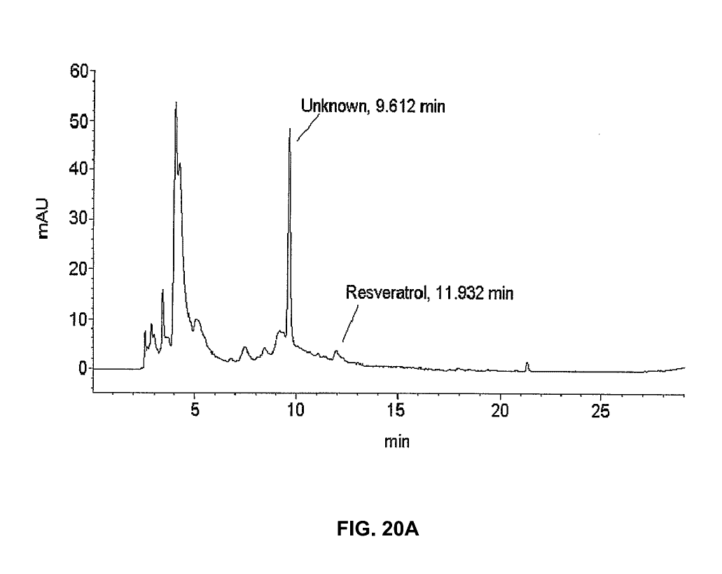

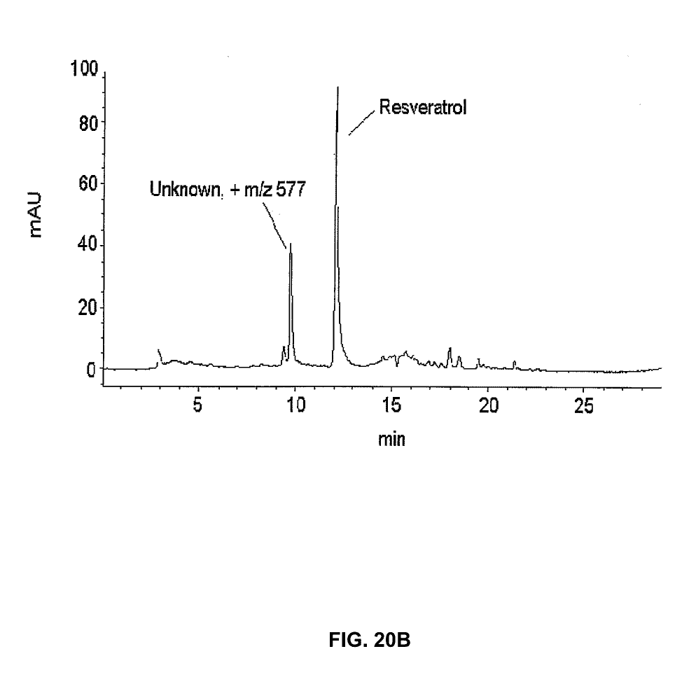

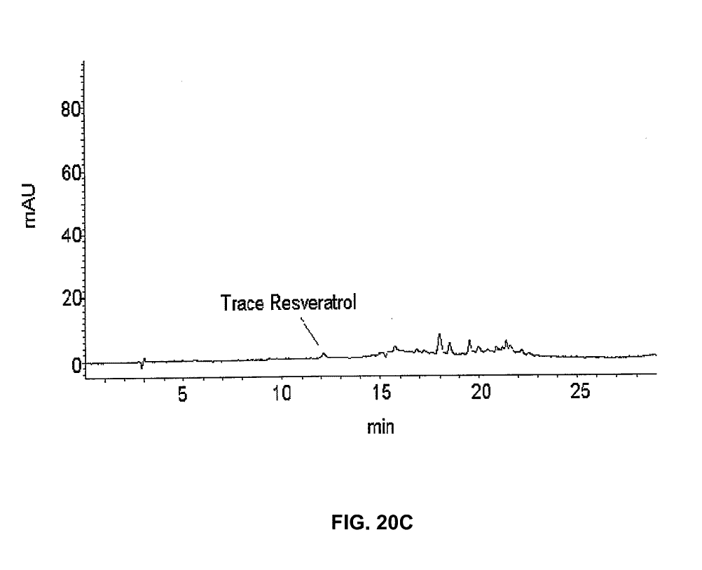

Reversed phase HPLC generated chromatograms of untreated and MISPE treated peanut meal extract (FIGS. 20A-20C) demonstrate a selective enrichment of resveratrol with an imprinting factor (I) of 66. It can also be noted that an unidentified analyte (RT=9.6 min) with a positive m/z of 577 amu was also captured but no significant enrichment was observed. It should be noted that this MISPE treatment procedure has yet to be fully optimised, and as such the enrichment of resveratrol and other analytes may be improved further.

### C Molecularly Imprinted Polymers: Phytosterols

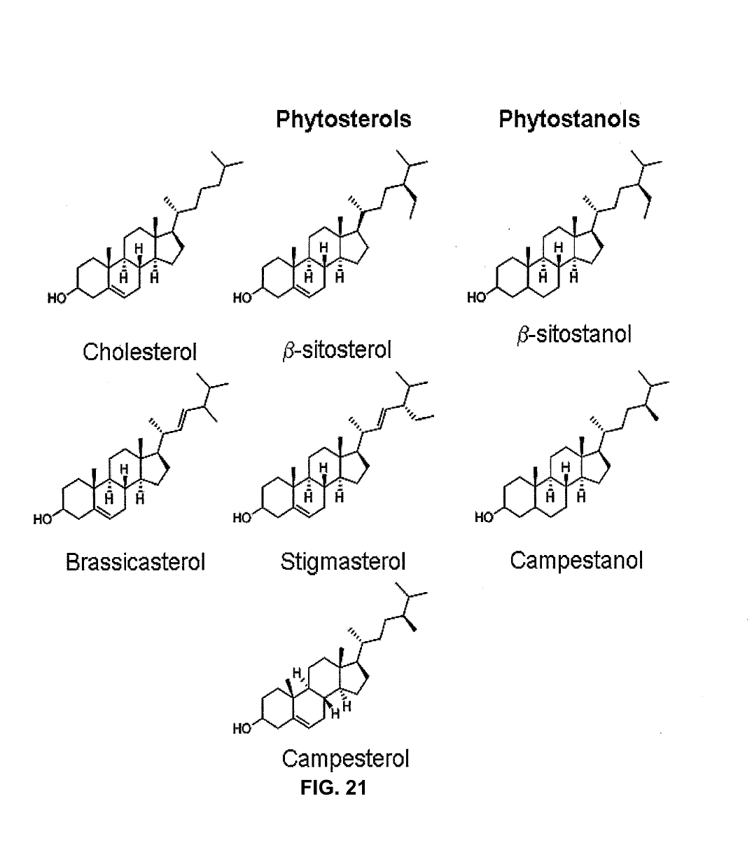

Phytosterols and Phytostanols are known to lower low density lipoprotein-cholesterol (LDL-C) levels in humans by up to 15%, and there are several products now on the market that are naturally derived fatty acid esters of phytostanols (stanol esters) and phytosterols. Phytosterols are plant fats that are structurally similar to the animal fat cholesterol. All plants including inter alia fruits, vegetables, grains, spices, seeds and nuts contain these sterol compounds or sterolins. Some of the most commonly found phytosterols include beta-sitosterol, stigmasterol and campesterol. Plant oils are a particularly rich source of phytosterols, however all sources are thought to be effective in the treatment or prevention of high cholesterol or hypercholesterolemia. FIG. 21 depicts the chemical structures of cholesterol and the commonly found phytosterols (β-sitosterol, stigmasterol, campesterol and brassicasterol) and phytostanols (β-sitostanol and campestanol).

### γ-Oryzanol.

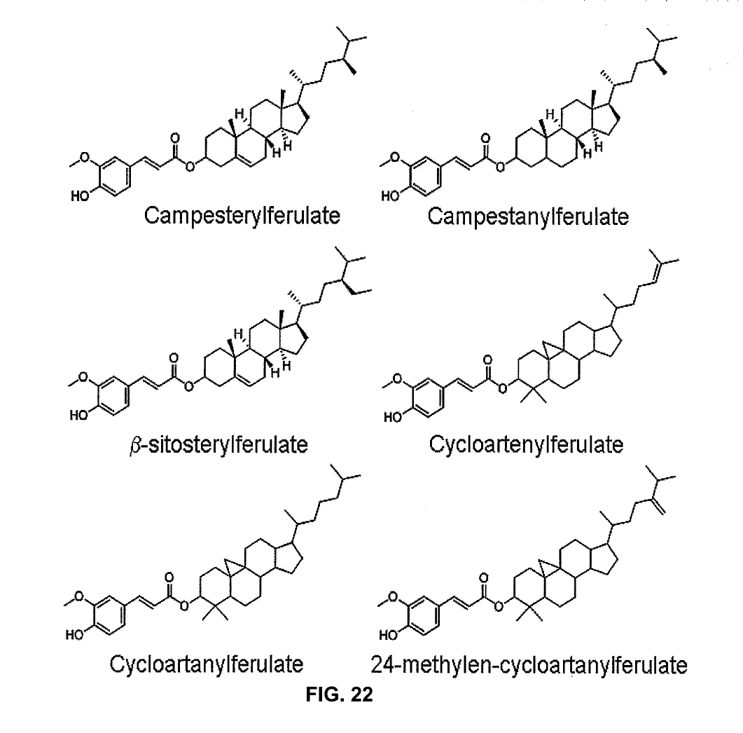

γ-Oryzanol is a mixture of ferulic acid esters of triterpene alcohols such as cycloartenol and 24-methylene cycloartanyl. γ-Oryzanol has been suggested to have potential functionality such as antioxidant activity, reduction of serum cholesterol, reduction of cholesterol absorption and decrease of early atherosclerosis, inhibition on platelet aggregation, inhibition of tumor promotion, menopausal syndrome treatment and antiulcerogenic activity. FIG. 22 depicts the six main components in γ-Oryzanol: campesterylferulate, campestanylferrulate, β-sitosterylferulate, cycloartenylferulate, cycloartanylferulate and 24-methylen-cycloartanylferulate.

Since the functionality of γ-Oryzanol is promising, rice bran or γ-Oryzanol may have great market potential and can be applied to a wide range of products and functional foods that may provide cholesterol-lowering and antioxidant effects.

### Preparation of Molecularly Imprinted Polymers

The various formulations of different noncovalent and covalent MIPs that have been prepared are summarized in Table 11.

TABLE 11
Composition of noncovalent MIPs prepared for assessment of sterol
binding.

| Polymer Type | Template | Configuration | Porogen | Name |
|---|---|---|---|---|
| Non-covalent | Cholesterol | T:4-VP:EDGMA | CHCl₃ | MIP 19 |
| | | 1:3:30 | | |
| Non-covalent | None | 4-VP:EDGMA | CHCl₃ | NIP 19 |
| | | 1:10 | | |
| Non-covalent | Cholesterol | T:MMA:EDGMA | CHCl₃ | MIP 20 |
| | | 1:3:30 | | |
| Non-covalent | None | MMA:EDGMA | CHCl₃ | NIP 20 |
| | | 1:10 | | |
| Non-covalent | Cholesterol | T:MMA:EDGMA | H₂O:TFA | MIP 21 |
| | | 1:3:30 | 9:1 | |
| Non-covalent | None | T:EDGMA | H₂O:TFA | NIP 21 |
| | | 1:10 | 9:1 | |
| Non-covalent | Stigmasterol | T:MMA:EDGMA | CHCl₃ | MIP 22 |
| | | 1:3:30 | | |
| Non-covalent | none | MMA:EDGMA | CHCl₃ | NIP 22 |
| | | 1:10 | | |
| Non-covalent | Cholesteryl | T:4-VP:EDGMA | CHCl₃ | MIP 23 |
| | ferulate | 1:3:30 | | |
| Non-covalent | none | 4-VP:EDGMA | CHCl₃ | NIP 23 |
| | | 1:10 | | |
| Non-covalent | Cholesteryl | T:MMA:EDGMA | CHCl₃ | MIP 24 |
| | ferulate | 1:3:30 | | |
| Non-covalent | none | MMA:EDGMA | CHCl₃ | NIP 24 |
| | | 1:10 | | |
| Covalent | Cholesteryl | T:EDGMA | CHCl₃ | MIP 25 |
| | ferulate | 1:10 | | |
| Covalent | none | EDGMA | CHCl₃ | NIP 25 |
| Hybrid | Cholesterol | T:M:C | CHCl₃ | MIP 26 |
| | | 1:3:30 | | |
| Hybrid | none | M:C | CHCl₃ | NIP 26 |
| | | 1:10 | | |

MMA—methylmethacrylate
M = (monomer)
C = (crosslinker, ie EDGMA)
TFA = Trifluoroacetic acid

### Polymerisation: Non-Covalent Approach (Template Cholesterol, Porogen Chloroform)

Molecularly imprinted polymers (MIPs) have been prepared using cholesterol and stigmasterol separately as the templating molecule. MIPs and their respective non-imprinted control polymers (NIPs) were prepared according to the following procedure. Template (cholesterol or stigmasterol: 1 eq), functional monomer (4VP or methylacrylic acid: 3 eq) were dissolved in porogen (CHCl₃), then sonicated for 10 minutes. The crosslinker (ethyleneglycol dimethacrylate, EGDMA: 30 eq) and free radical initiator (AIBN: 0.25 eq) were subsequently added and thermal polymerisation was performed at 60° C. for 24 hours. NIPS were prepared in an identical manner in the absence of template.

### Polymerisation: Non-Covalent Approach (Template Cholesterol, Porogen H₂O:TFA (9:1)

Molecularly imprinted polymers (MIPs) have been prepared using cholesterol as the templating molecule. MIPs and their respective NIPS were prepared according to the following: Template (cholesterol 1 eq), functional monomer (MMA: 3 eq) were dissolved in porogen (H2O:TFA, 9:1), then sonicated for 10 minutes. The crosslinker (ethyleneglycol dimethacrylate, EGDMA: 30 eq) and free radical initiator (AIBN: 0.25 eq) were subsequently added and thermal polymerisation was performed at 65° C. for 24 hours. NIPS were prepared in an identical manner in the absence of template.

### Performance of the Molecularly Imprinted Polymers:

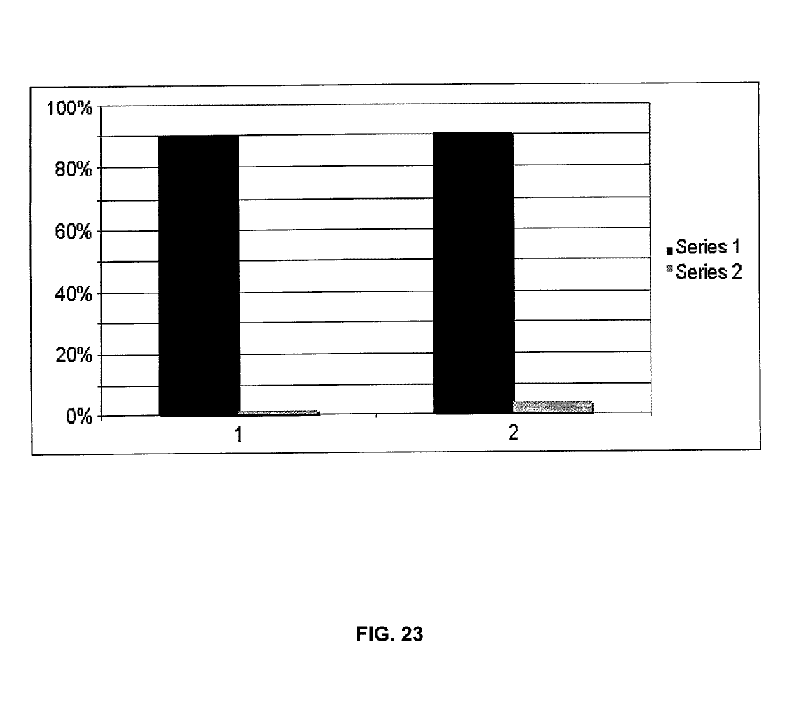

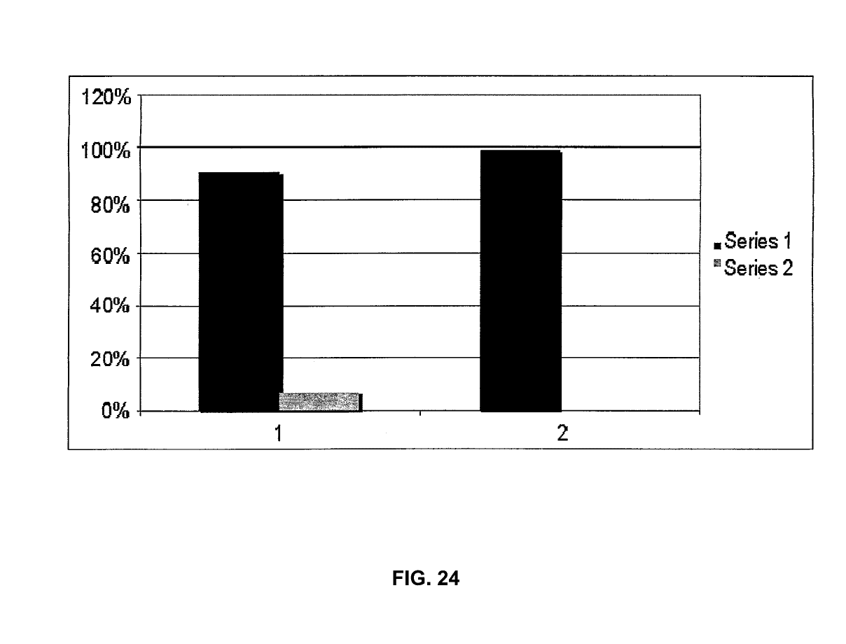

FIGS. 23 and 24 illustrate the performance of the two “non covalent models” of MIPS prepared using cholesterol as templating molecule, respectively. Binding of cholesterol solutions (0.5 min) was assessed using rebinding experiments described in the resveratrol section: the presence of cholesterol in eluates was quantified by measuring absorbance at 208 nm. The retention of cholesterol on these MISPE columns is shown and demonstrates that MIPs prepared using these methods specifically adsorbs cholesterol with the imprinting factor of at least 10:1.

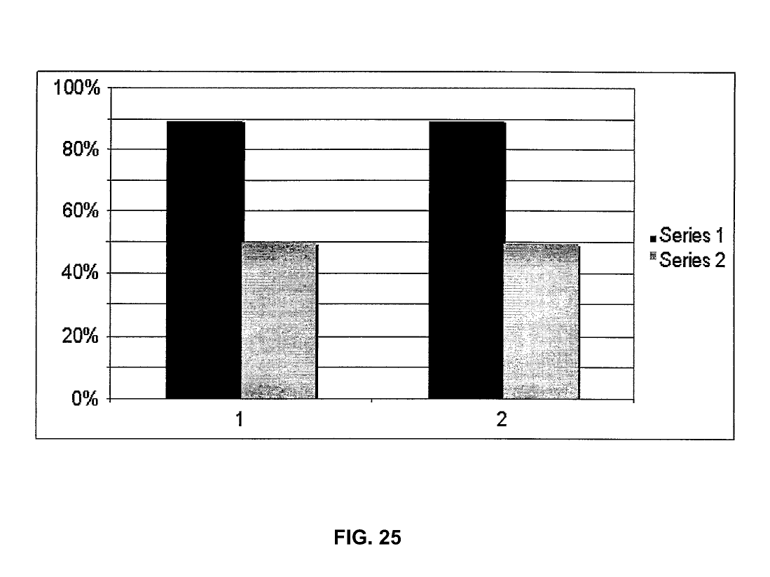

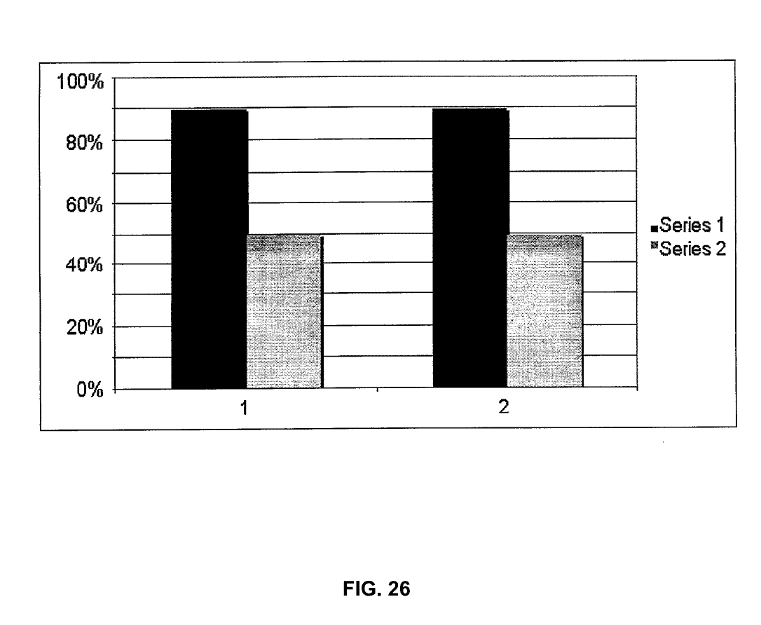

Similar rebinding experiments demonstrated that both of these MIPs were capable of binding stigmasterol as shown in FIGS. 25 and 26.

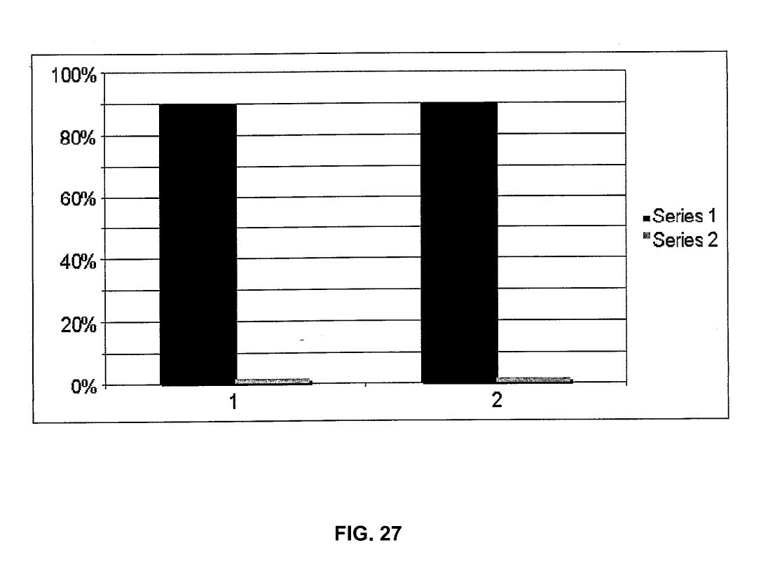

A second series of MIPs was and NIPs were prepared using stigmasterol as template to determine whether the chemical structure differences between stigmasterol and cholesterol affected the overall recognition of the MIP. MIPS were prepared as above using T:MMA:EDGMA in the ratio 1:3:30. The results of rebinding experiments are shown in FIG. 27.

Assessment of the “Green Polymer” (MIP21 and NIP21) with porogen 9:1 H₂O:TFA with cholesterol and stigmasterol in comparison to MIP19 and MIP20.

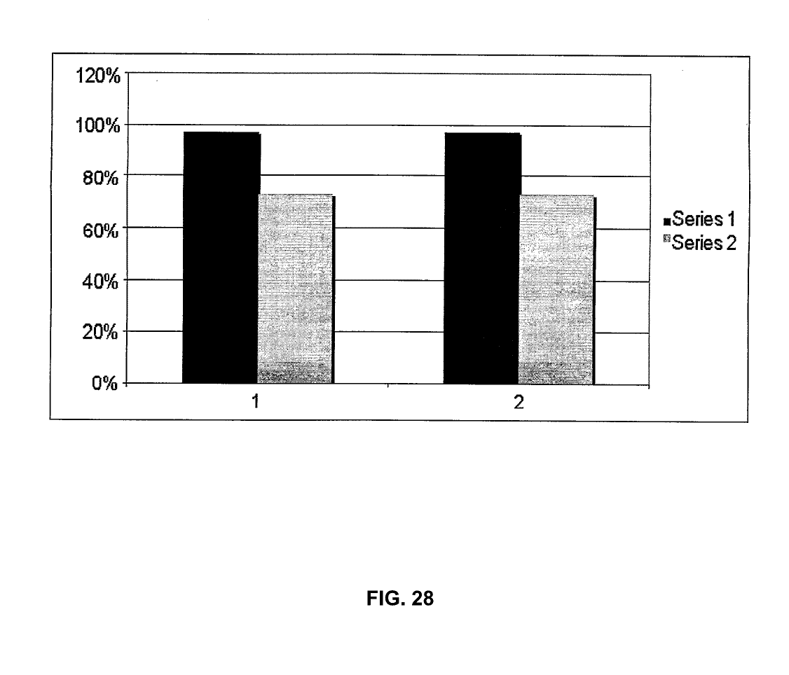

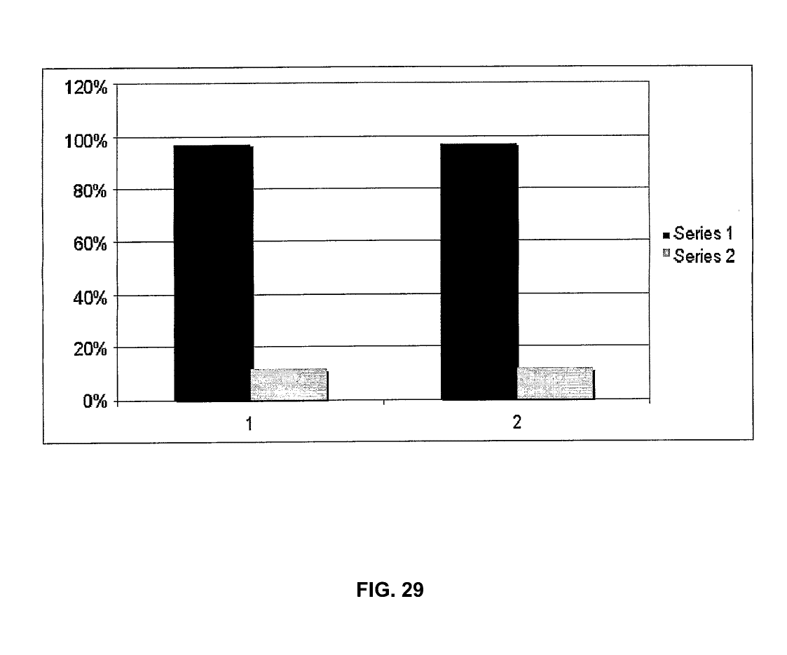

The preliminary conclusion which can be drawn is that the “Green Polymer” has a certain degree of recognition for both stigmasterol and cholesterol (see FIGS. 28 and 29), and therefore represents the most suitable model system for future investigations and applications in attempts to differentiate between the different classes of phytosterols and phytostanols, as bioactive targets.

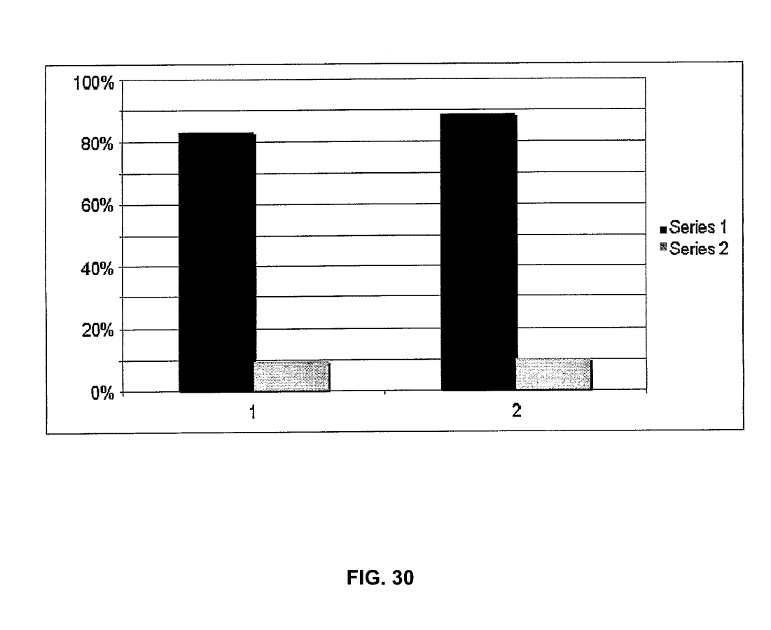

Attempts to recycle the polymer were promising and are summarized in FIG. 30 (chloroform as porogen). Results are encouraging due to the applicability and adaptability of the MIP/NIP technology in reusable form with no noticeable alteration to the functionality of the polymer.

### Non Covalent Polymerization Using Cholesteryl Ferrulate as Template.

### 1. Synthesis of Steryl Ferulates as Template for Non Covalent Polymerisation of MIPs.

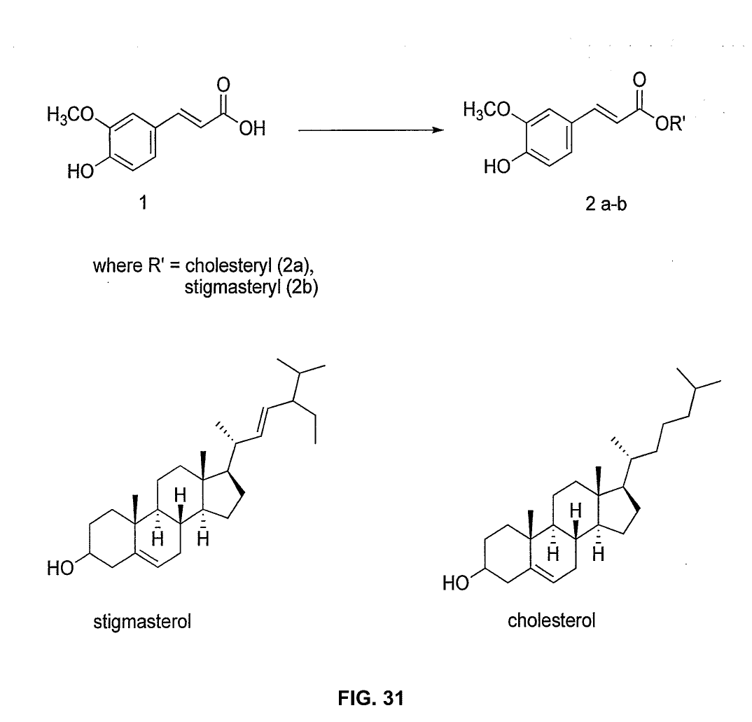

A mixture of the phytosterols containing stigmasterol, sitosterol, campesterol and β-sitosterol were derivatised to the corresponding ferrulate esters using readily available ferrulic acid (FIG. 31).

### Synthesis of trans-4-O-acetylferulic acid

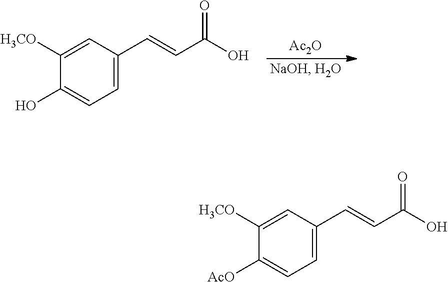

Ferullic acid was acetylated using Ac₂O/NaOH in water to produce trans-4-O-acetylferrulic acid. The product was purified by adjusting the solution pH to 4-5 with 1 M HCl to form a white precipitate, which was washed subsequently with H₂O, dried and collected as a crystalline solid.

¹H (300 MHz, CDCl₃) 7.0-7.5 (m, 3H, Ar), 6.2 (m, 1H), 3.70 (s, 3H, OCH₃), 2.0 (s, 3H, CH₃).

### Synthesis of 3-O-(trans-4-O-Acetylferuloyl) cholesterol

Trans-4-O-acetylferulic acid and cholesterol were dissolved in dry CH₂Cl₂. DCC in CH₂Cl₂ and DMAP were added and the mixture was stirred at room temperature for 18 hours. Solid by-product was removed by filtration and the solution was successively extracted once with H₂O, then twice with 10% HOAc and twice with H₂O. The organic extracts were combined and dried over anhydrous MgSO₄ and evaporated to yield a white solid. The solid was dissolved in minimal THF and chilled at 0° C. overnight to precipitate any residual by-product. The solution was filtered and the solvent evaporated to form a solid which was purified by isocratic elution in 25% EtOAc/Hexane by column chromatography to give the final product as an off white crystalline solid.

¹H (300 MHz, CDCl₃) 7.62 (d, 1H, J=16 Hz, Ar), 7.10 (m, 2H, Ar), 6.37 (d, 1H), 4.83 (m, 1H), 3.86 (s, 3H), 2.33 (s, 3H), 0.6-2.0 (m, 50H).

### Synthesis of 3-O-(trans-4-O-Acetylferuloyl) β-sitosterol

Trans-4-O-acetylferulic acid and β-sitosterol were dissolved in dry CH₂Cl₂. DCC in CH₂Cl₂ and DMAP were added and the mixture was stirred at room temperature for 18 hours. Solid by-product was removed by filtration and the solution was successively extracted once with H₂O, then twice with 10% HOAc and twice with H₂O. The organic extracts were combined and dried over anhydrous MgSO₄ and evaporated to yield a white solid. The solid was dissolved in minimal THF and chilled at 0° C. overnight to precipitate any residual by-product. The solution was filtered and the solvent evaporated to form a solid which was purified by isocratic elution in 25% EtOAc/Hexane by column chromatography to give the final product as an off white crystalline solid.

¹H (300 MHz, CDCl₃) 7.62 (d, 1H, J=16 Hz, Ar), 7.10 (m, 2H, Ar), 6.37 (d, 1H), 4.83 (m, 1H), 3.86 (s, 3H), 2.33 (s, 3H), 0.6-2.0 (m, 50H).

### Synthesis of 3-O-(trans-4-Feruloyl)-cholesterol

3-O-(trans-4-O-Acetylferuloyl) cholesterol was dissolved in 2:1 CHCl₃:MeOH and K₂CO₃ (0.2 eq) was added and the mixture refluxed for 6 hours. The reaction was subsequently quenched by the addition of satd aq. NH₄Cl and the layers organic layer was separated and washed twice with H₂O, then dried over MgSO₄. The final product was obtained as an off white crystalline solid after recrystallization from 3:1 CHCl₃: MeOH

¹H (300 MHz, CDCl₃) 7.62 (d, 1H, J=16 Hz, Ar), 7.10 (m, 2H, Ar), 6.91 (d, 1H), 6.2 (d, 1H), 5.96 (s, 1H), 4.83 (m, 1H), 3.86 (s, 3H), 0.6-2.0 (m, 50H).

### Polymerisation: Non-Covalent Approach (Template Cholesteryl Ferulate (2a), Porogen Chloroform)

Molecularly imprinted polymers (MIPs) have been prepared using cholesteryl ferrulate as the templating molecule. MIPs and their respective NIPs were prepared according to the following procedure. Template (cholesteryl ferrulate: 1 eq), functional monomer (4VP or methylacrylic acid: 3 eq) were dissolved in porogen (CHCl₃), then sonicated for 10 minutes. The crosslinker (ethyleneglycol dimethacrylate, EGDMA: 30 eq) and free radical initiator (AIBN: 0.25 eq) were subsequently added and thermal polymerisation was performed at 60° C. for 24 hours. NIPs were prepared in an identical manner in the absence of template.

The preliminary rebinding studies were performed as follows:

| MIP | NIP |
|---|---|
| 50 mg MIP placed into 2 mL centrifuge | 50 mg MIP placed into 2 mL |
| tube and chloroform (1.5 mL) added. | centrifuge tube and |
| | chloroform (1.5 mL) added. |
| 0.5 mM template dissolved in 0.5 mL of | 0.5 mM template dissolved |
| CHCl₃ | in 0.5 mL of CHCl₃ |
| Shake for 30 minutes | Shake for 30 minutes |
| Centrifuge for 20 minutes | Centrifuge for 20 minutes |
| Collect aliquots of supernatant | Collect aliquots of supernatant |
| HPLC analysis | HPLC analysis |

MIP controls were treated the same, except the rebinding solution did not contain the template.

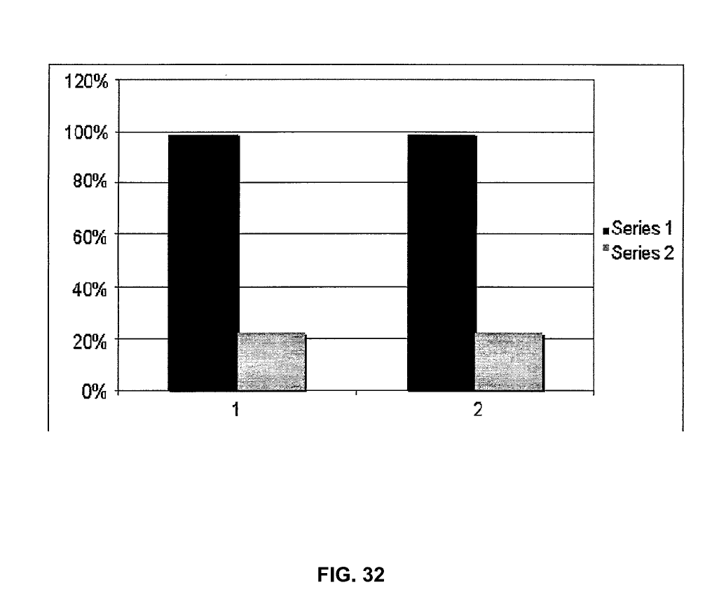

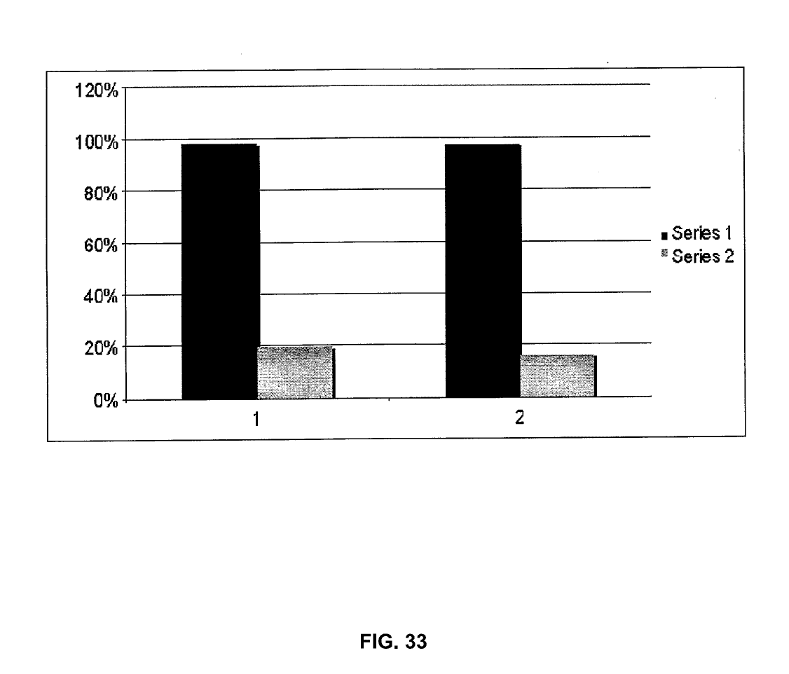

Binding data was analyzed as the percentage of the applied cholesteryl ferrulate, cholesterol or stigmasterol bound to the polymer. The concentration of the free cholesteryl ferulate, cholesterol or stigmasterol was determined from the 4-point calibration curve. The percentage of cholesteryl ferulate bound (FIGS. 32 and 33) could then be calculated as % bound=100−((C_{f}/Cᵢ)×100) where C_{f} is concentration of free cholesteryl ferulate and Cᵢ concentration of initial cholesteryl ferulate.

### “Cartridges” Rebinding Studies in General

| | |
|---|---|
| 100 mg of MIP place into a filtration tube | 100 mg of NIP place into |
| and methanol is added | a filtration tube and |
| | methanol is added |
| The template in chloroform (0.05 mM) is | The template in chloroform |
| added and allowed to absorb slowly | (0.05 mM) is added and |
| through the “MIP” column | allowed to absorb slowly |
| | through the “NIP” column |
| The performance of the MIP was assessed | The performance of the |
| at various stages: prior to elution (blank), | NIP was assessed at |
| 1 mL of methanol, 2 mL of MeOH, 3 mL | various stages: prior |
| of MeOH, 4 ml of MeOH. | to elution (blank), |
| | 1 mL of methanol, 2 mL of |
| | MeOH, 3 mL of MeOH, |
| | 4 mL of MeOH. |

Polymerisation Using “Covalent Approach” with Cholesteryl Ferrulate as a Template for Imprinting:

Molecularly imprinted polymers and their respective non-imprinted control polymers (NIPs) were prepared as follows: (4VP:crosslinker (1:10) where crosslinker is EGDMA) in CHCl₃ as a porogen using AIBN as radical initiator.

### Performance of the Covalently Imprinted Polymer and its Non-Imprinted Equivalent.

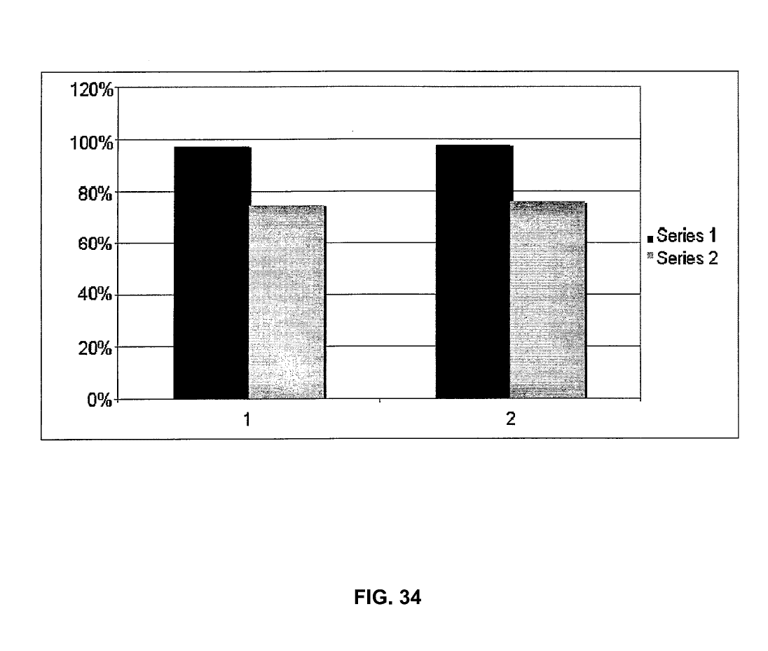

The performance is detailed in FIG. 34.

Future investigations will involve investigation in effects of porogen (such as ethanol, H₂O/TFA) in order to optimize the performance of the polymers and the selectivity.

### Polymerization Using “Hybrid Approach”:

The “hybrid polymer” was prepared with ratios of T:M:C as 1:3:30 for consistency. Where T is cholesterol, M is (E)-3-(4-(methacryloxy)-3-methoxyphenyl)acrylic acid and C is EDGMA.

### Performance of the Polymer:

The performance of the polymer indicates that the “hybrid approach” adds increased flexibility and selectivity in the substrates of interest.

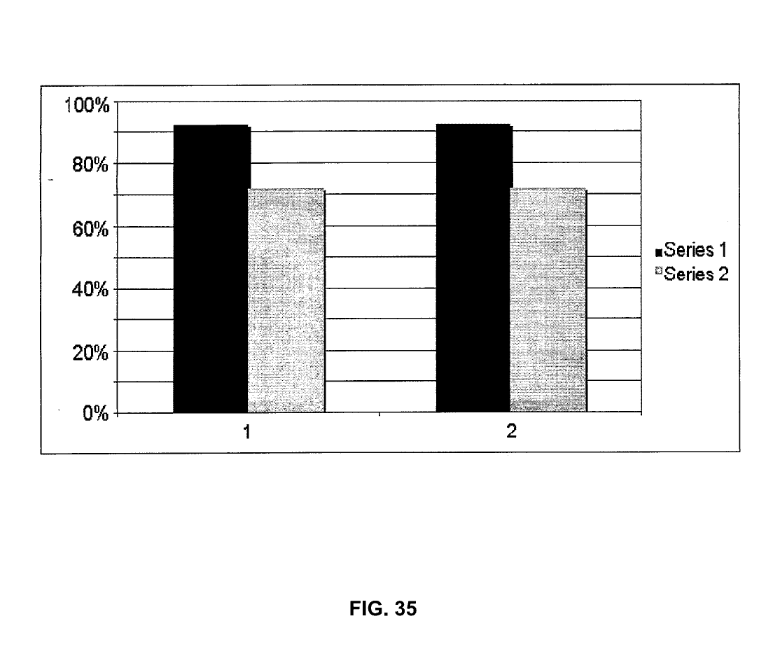

The hybrid polymer shows good selectivity with ester-like substrates and low-moderate levels of nonspecific binding (FIG. 35). The polymer composition is suitable for application to the crude mixtures of waste materials provided.

### D. Preparation of a Molecularly Imprinted Polymer for the Selective Recognisiton of the Bioactive Polyphenol (E)-Resveratrol

In this example, the design and preparation of a (E)-resveratrol imprinted polymer via non-covalent self-assembly and the assessment of its selectivity for (E)-resveratrol over structurally similar analogues is described.

### Materials and Methods

### Reagents.

Resveratrol-3-β-D-glucopyranoside (3,4′,5-trihydroxystilbene-3-β-D-glucopyranoside) 5, (E)-stilbene 8, 4-Vinylpyridine (4VP), ethyleneglycol dimethacrylate (EGDMA) and 2,2′-azobis(2-methylpropionitrile) (AIBN) were purchased from Sigma-Aldrich. All solvents used for MIP preparation and evaluation were HPLC grade.

### Equipment.

An Agilent Technologies 1100 LC system (Waldbronn, Germany) consisting of a binary pump with a vacuum degasser, auto-sampler with a 900 μL sample loop, thermostated column compartment and a diode-array detector was employed for the HPLC separation of the sample. Injected samples were analysed by RP-HPLC on a Zorbax Eclipse XDB-C18 column (4.6×150 mm, 5 μm particle size).

### Compounds.

The selected compounds were chosen to enable the comparative investigation of the number of potential binding sites and their relative positions, around a central E-stilbene core. The general synthetic process is summarized below in

### Scheme 4.

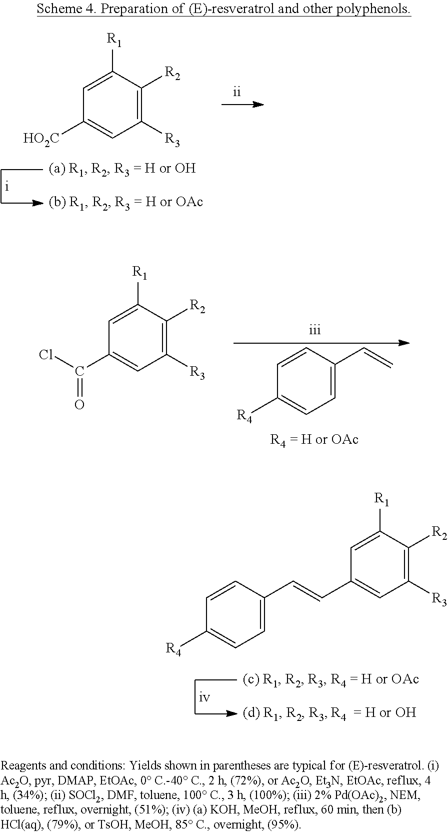

A detailed description for the preparation of (E)-resveratrol 1 and the hydroxylated stilbene analogues (E)-5-(4-hydroxystyryl)benzene-1,2,3-triol 2, (E)-5-styrylbenzene-1,3-diol 3, (E)-3-(4-hydroxystyryl)phenol 4, (E)-4-styrylphenol 6, (E)-3-styrylphenol 7, (and further polyphenols) is reported above. The process entailed conversion of a functionalized benzoic acid to its more activated acid chloride, which after distillation to remove solvents, was immediately on-reacted with an appropriate styrene. The coupling reaction was satisfactorily promoted with catalytic amounts of palladium acetate. This coupling reaction is reported to proceed via a chalcone intermediate. However, only the stilbene adduct was isolated under these conditions, indicating that complete decarbonylation has occurred simultaneously during the reaction. The E stereochemistry of the product was readily confirmed by the characteristic Jₜᵣₐₙₛ=16 Hz coupling constant in the ¹H NMR and no Z isomer was detected (expected J_{cis}=≦12 Hz).

### Molecular Modelling.

All modelling calculations were conducted using Spartan '08 for Windows V100 software package on a Pentium IV 2.0 GHz. Modelling procedures were based on previously described methods, whereby the semi-empirical equilibrium geometry level theory was applied using a PM3 force field to calculate the energy of formation values (ΔH_{f}), for template, monomer clusters and monomer-template clusters in the gas phase without consideration of solvent effects. Monomer cluster sizes ranging from 1 to 6 monomer units were modelled and the ΔH_{f} values determined for the interaction of the monomer with itself at these cluster sizes. The (E)-resveratrol structural file was then inserted into each cluster file with no pre-defined orientation imposed upon either template or monomer cluster. Equilibrium geometry was determined using an iterative approach. A minimum of three iterations yielded theoretical estimates of the average energy of formation (ΔEᵢ) for the complex, which was determined using the following equation:

ΔEᵢ=H_{f}_{_}_{Complex}−(ΔH_{f}_{_}_{Template}+ΔH_{f}_{_}_{Monomer})

### ¹H NMR Spectroscopy Titrations.

(E)-Resveratrol (23 mg, 0.1 min) dissolved in CD₃CN was titrated with increasing molar equivalents of 4-vinylpyridine (4VP). The ¹H NMR spectrum was recorded after each addition and the change in aromatic —OH shifts followed until the presence of H bonding interactions was evidenced by the consistent downfield shift of this aromatic —OH signal with increased additions. This process was continued until the aromatic —OH signal was no longer detectable due to peak broadening.

### MIP Preparation.

MIPs were prepared by dissolving the template (E)-resveratrol (228 mg, 1 mmol) in CH₃CN/EtOH (6 mL, 5:1 v/v) in a glass test tube to which the functional monomer 4VP (322 μL, 3 mmol) was added. The mixture was sonicated for 10 minutes and the cross-linker EGDMA (2.314 mL, 15 mmol) and the free radical initiator AIBN (51 mg, 0.31 mmol) were added. This pre-polymerisation mixture was sparged with N₂ (g) for 5 minutes and placed in a thermostatic water bath at 50° C. for 24 hours. A number of polymer products were annealed by heating at 60° C. for a further 24 hours. Polymers were then removed from reaction tubes, then crushed and ground using a Retsch 200 ball mill. The ground particles were subsequently sieved and the 63-100 μm size particles retained. Fines were removed by repeated cycles of suspension of the polymer particles in acetone and decanting the supernatant. The (E)-resveratrol template was removed from the MIP resin by repeated washings in MeOH containing 10% AcOH by volume (50 mL) with gently stirring. The washings were monitored by UV-Vis spectroscopy at 321 nm and repeated at least 3 times or until the template could no longer be detected. MIPs were then washed with MeOH to remove traces of AcOH, filtered and dried in vacuo. Non-imprinted control polymers (NIPs) were prepared in exactly the same manner but in the absence of the template molecule. A summary of MIP and NIP preparations is outlined in Table 12.

### MIP Evaluation.

(E)-Resveratrol saturation studies over the concentration range of 0-4 min in CH₃CN were conducted using both MIPs and NIPs at constant polymer weight. Comparison of binding events observed with the MIP and NIP reference materials revealed the extent to which imprinting has influenced adsorption of (E)-resveratrol. The polymer (30 mg) was weighed into a 1.7 mL Eppendorf tube and incubated with analyte solution (1.5 mL, 0-4 min) on a rotary mixer at 40 rpm for 18 hours. The mixture was then centrifuged at 13000 rpm for 15 minutes to pellet the ligand-bound polymer: an aliquot (200 μL) of the supernatant was removed and analysed by RP-HPLC with UV detection at 321 nm and the concentration of unbound (E)-resveratrol was determined from a linear 5 point calibration curve. This concentration was Subtraction of this value from the initial total analyte concentration yielded the amount of analyte bound (B), expressed as μmol/g polymer.

To further investigate non-specific surface binding resulting from interactions with the cross-linker and randomly dispersed functional monomer, static binding assays were conducted on both the MIP and NIP in parallel. The polymer (30 mg) was weighed into a 1.7 mL Eppendorf tube and (E)-resveratrol solution (1.5 mL, 0.5 min in CH₃CN) added. The resulting mixture was then treated and analysed as above.

TABLE 12
Summary of MIP preparations.

| | | | EDMA | |
|---|---|---|---|---|
| Polymer | (E)-Resveratrol | | (cross- | |
| Code | (Template) | 4VP (FM) | linker) | Porogen |
| P1 | 1 mmol | 3 mmol | 15 mmol | CH₃CN/EtOH |
| (equivalent | | | | 5:1 v/v (6 mL) |
| to MIP8)* | | | | |
| N1* | none | 3 mmol | 15 mmol | CH₃CN/EtOH |
| (equivalent | | | | 5:1 v/v (6 mL) |
| to NIP8) | | | | |
| P2 | 1 mmol | none | 15 mmol | CH₃CN/EtOH |
| | | | | 5:1 v/v (6 mL) |
| N2 | none | none | 15 mmol | CH₃CN/EtOH |
| | | | | 5:1 v/v (6 mL) |
| N3 | none | 3 mmol | none | CH₃CN/EtOH 5:1 |
| | | | | v/v (6 mL) |

All polymerizations were initiated with AIBN at 50° C. for 24 hours.
*Prepared with an additional 24 hour thermal annealing at 60° C.

### Results and Discussion

### Molecularly Imprinted Polymer Design.

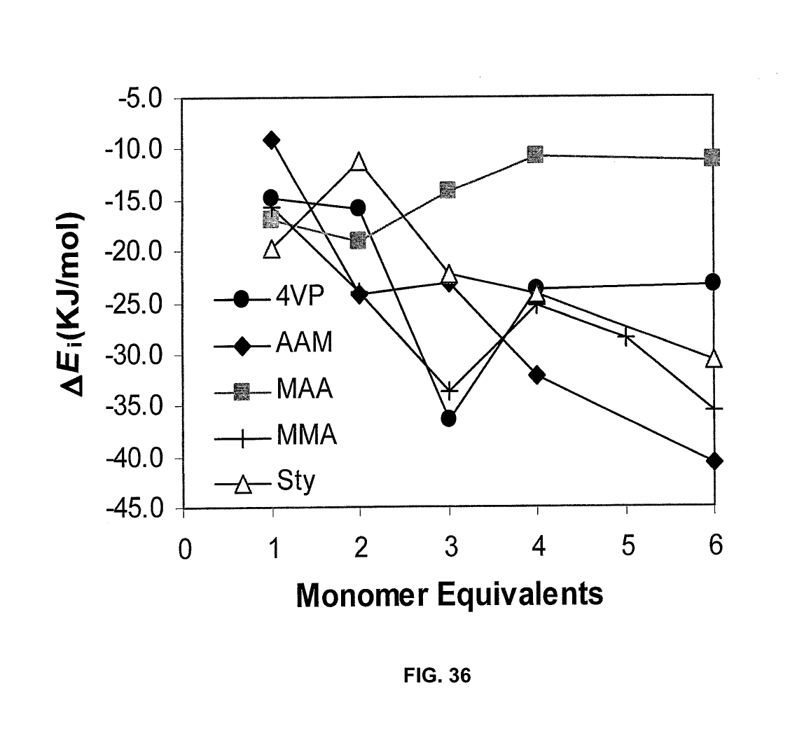

A number of techniques were used to assist in the rational design of an (E)-resveratrol imprinted polymer. Molecular modelling techniques were employed to estimate the strength of intermolecular interactions between (E)-resveratrol and a range of potential functional monomer clusters. This approach identified 4-vinylpyridine (4VP) as a suitable functional monomer (FM) and predicted that a 3:1 molar ratio of 4VP:(E)-resveratrol was optimal for the formation of the most stable pre-polymerisation complex (FIG. 36). These interactions were confirmed by ¹H NMR spectroscopy titration analysis, where titration with 4VP resulted in the chemical shift for the phenolic OH groups moving downfield by a total of approximately 0.8 ppm. Self-assembly interactions such as these involving pyridine and phenol molecules have been reported whereby multilayer clusters were formed via aromatic intermolecular O—H•••N hydrogen bonding interactions.

### Molecularly Imprinted Polymer Preparation.

Solid imprinted block copolymer monoliths were prepared by incorporating a small amount of EtOH into the porogen solution (CH₃CN:EtOH, 5:1 v/v) to increase the solubility of (E)-resveratrol without compromising H-bonding capabilities. This polar protic solvent may contribute to enhancement of aromatic π-π interactions between aromatic groups already clustered in close proximity, while also stabilising existing interactions within the phenolic-pyridinyl cluster systems.

### Molecularly Imprinted Polymer Evaluation.

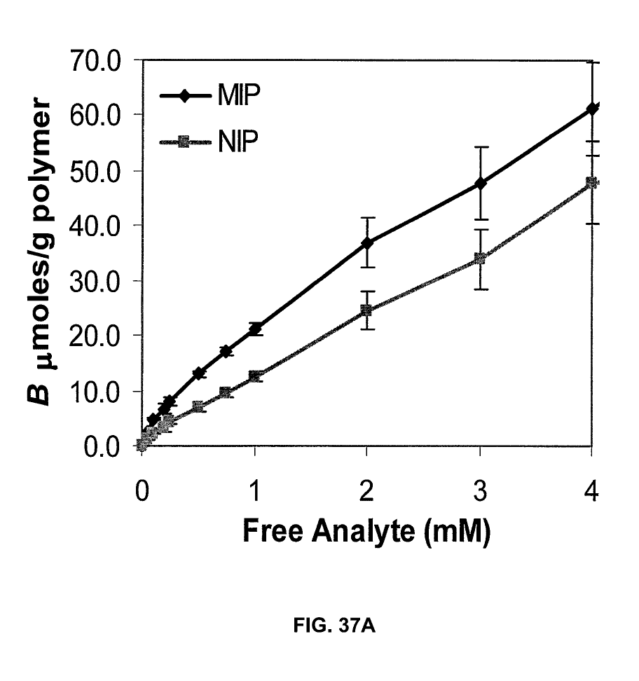

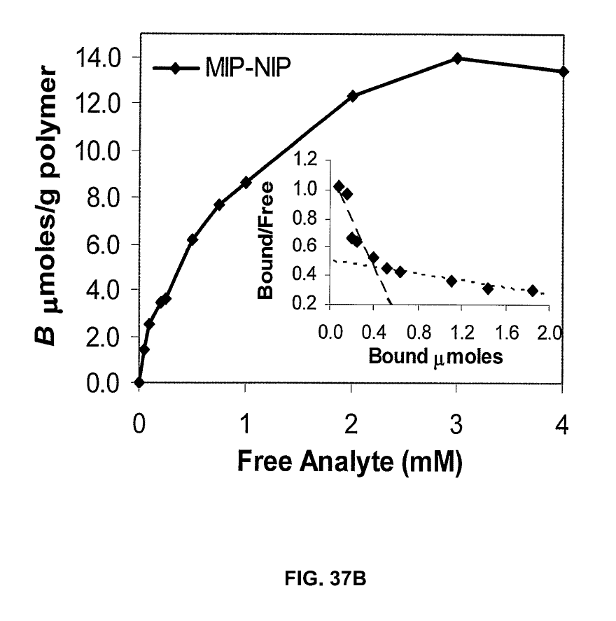

FIGS. 37A and 37B show the static binding isotherms derived for polymers P1 and N1 with (E)-resveratrol. The selective capacity (B_{MIP}−B_{NIP}=14 μmol/g) validates an imprinting effect resulting from the successful formation of (E)-resveratrol binding cavities or regions within the imprinted polymer. The lesser amounts of (E)-resveratrol bound by the non-imprinted control polymer N1 is most likely due to non-specific surface interactions with the randomly dispersed functional monomer. MIPs based on self-assembly may be variable from preparation to preparation hence, as these evaluations were conducted using multiple batches of P1, such variations most likely contributed to the large error bars. However, while the capacity from preparation to preparation was observed to be variable, the selective capacity remained essentially constant, suggesting that the imprinting effect is largely unaffected from batch to batch of similar MIPs.

Scatchard analysis revealed a nonlinear concave-upward curve with two distinct linear regions that is typically illustrative of (i) heterogeneity of binding sites, (ii) cooperativity of binding or (iii) multivalent ligand binding, of which (i) is typically considered to describe the binding of molecules to non-covalently prepared imprinted polymers.

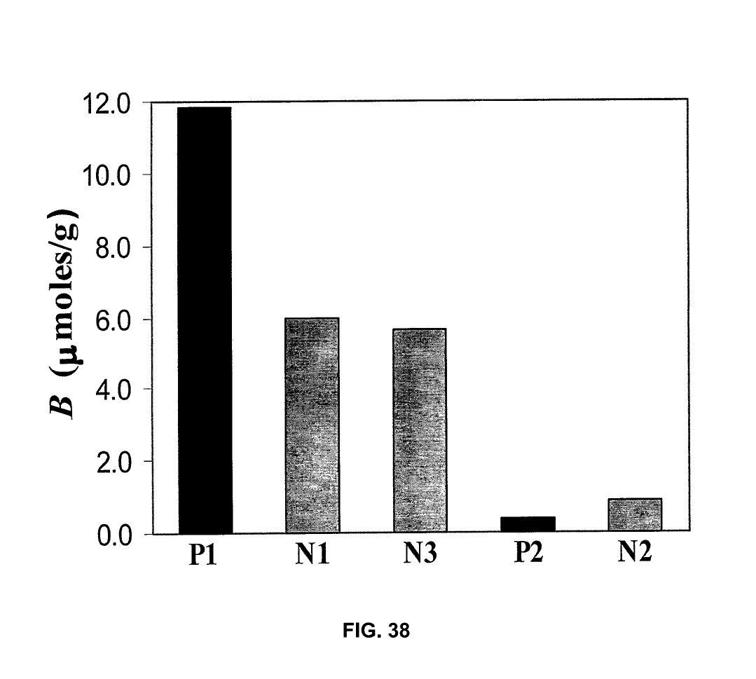

To confirm that non-specific surface interactions with randomly dispersed 4VP were responsible for the binding response of the non-imprinted polymer N1, the polymers P2 ((E)-resveratrol imprinted poly-EGDMA, no FM), N2 (non-imprinted poly-EGDMA, no FM) and N3 (non-imprinted poly-4VP, no cross-linker) were prepared. The respective affinity of these polymers for binding of (E)-resveratrol under the same conditions is shown in FIG. 38. Polymers P2 and N2 exhibited negligible (E)-resveratrol adsorption, indicating that the cross-linker EGDMA does not significantly contributed to the polymer binding responses. Additionally, the failure of P2 to recognise (E)-resveratrol suggests that the presence of binding cavities with the size and shape of (E)-resveratrol was insufficient to actively sequester this molecule from solution. This finding highlights the importance of appropriately topologically positioned complementary functional groups within the cavity site in addition to the appropriate size of the cavity. The moderate binding of (E)-resveratrol to N3 further verifies that the random dispersal of the functional monomer 4VP throughout the polymer matrix is responsible for non-specific molecular binding.

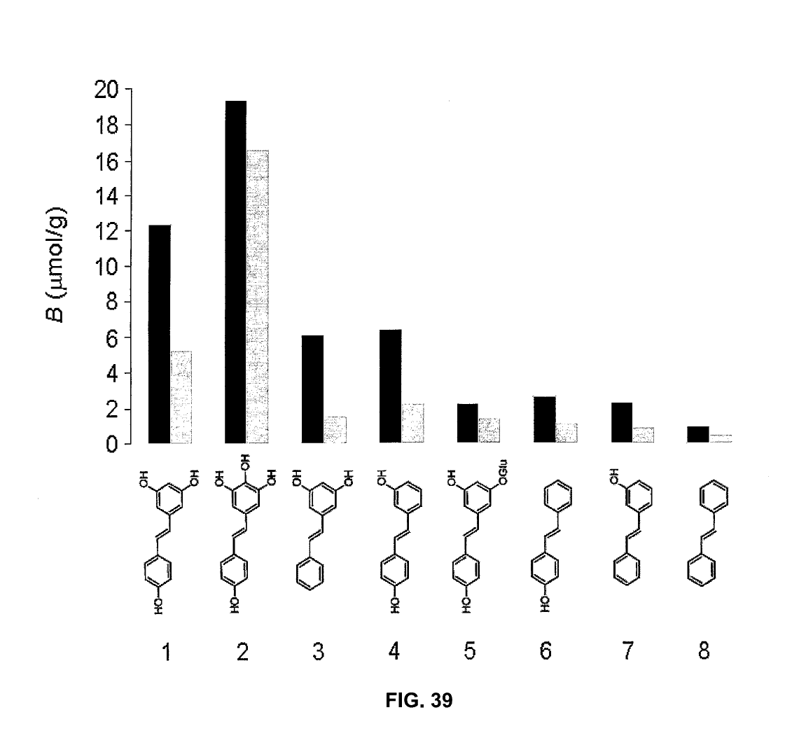

Single analyte (non-competitive) cross reactivity studies were employed to examine the influence of positions and numbers of hydrogen bonding OH groups present on the target molecule upon molecular recognition. The static binding affinities of (E)-resveratrol and a range of structural analogues towards the (E)-resveratrol imprinted polymer P1 were determined using equilibrium binding assays. The concentration of the bound analyte was determined by the difference between the initial analyte concentration and the concentration of remaining analyte in solution (FIG. 39, Table 13). This analysis showed that the number of phenolic OH groups clearly influenced the amount of specific binding to P1 and non-specific binding to N1 with the tetra-ol 2 displaying high levels of affinity to both P1 and N1. Analytes with fewer than three phenolic OH groups displayed minimal affinity to N1 (≦2.21 μmol/g). This observation is similar to that reported for an amino acid imprinted system (38), wherein non-template analytes possessing a greater number of functional groups displayed higher non-specific binding with randomly dispersed functional groups within the polymer matrix. The best binding was observed by the template (E)-resveratrol with good binding capacity of 12.36 μmol/g and imprinting factor (IF) (where IF=B_{MIP}/B_{NIP}) of 2.35. Analogues of (E)-resveratrol having one less phenolic OH group (analytes 3 and 4) demonstrated a reduction in the extent of analyte binding by approximately 50% of that observed for (E)-resveratrol, yet resulted in relatively higher recognition (IF=4.02 and 2.89, respectively), as non-specific binding of these molecules was significantly reduced. (E)-resveratrol analogues having two less phenolic OH groups (analytes 6 and 7) demonstrated further reduced specific binding to P1, with essentially no difference conferred by the relative position of the OH group. Binding of the (E)-stilbene 8 to PI was effectively abolished due to the absence of phenolic OH groups. Interestingly, the binding of the naturally occurring mono glycosylated derivative (E)-resveratrol-3-β-D-glucopyranoside 5 to P1 paralleled that observed for the monophenolic (E)-resveratrol analogues 6 and 7. The presence of the bulky glucose group presumably prevented the interaction of the mew positioned OH group within the binding cavity, thereby leaving the para positioned OH group as the only functionality capable of hydrogen bonding.

TABLE 13
Affinity of a range of structurally related polyphenols (0.5 mM in CH₃CN)
towards P1 and N1 under static equilibrium binding conditions
without competition.

| | MIP P1 | NIP N1 | | | |
|---|---|---|---|---|---|
| | Binding | Binding | Imprinting | | |
| Analyte | μmol/g | μmol/g | Factor | | Selectivity |
| per | polymer | polymer | IF | | α = (B_{MIP} − |
| FIG. 39 | B_{MIP} | B_{NIP} | (B_{MIP}/B_{NIP}) | B_{MIP} − B_{NIP} | B_{NIP})/B_{NIP} |
| 1 | 12.36 | 5.25 | 2.35 | 7.11 | 1.36 |
| 2 | 19.37 | 16.59 | 1.17 | 2.78 | 0.17 |
| 3 | 6.11 | 1.52 | 4.02 | 4.6 | 3.03 |
| 4 | 6.39 | 2.21 | 2.89 | 4.19 | 1.9 |
| 5 | 2.23 | 1.42 | 1.57 | 0.81 | 1.94 |
| 6 | 2.66 | 1.11 | 2.40 | 1.56 | 1.41 |
| 7 | 2.27 | 0.88 | 2.60 | 1.40 | 1.59 |
| 8 | 0.91 | 0.47 | 1.94 | 0.43 | 0.92 |

The binding of polyphenol analogues to polymer P1 was investigated under competitive conditions. Results from a competitive static cross-reactivity assay for an equimolar mixture of (E)-resveratrol 1 and analogues 2, 3, 4, 6 and 8 at 0.5 min each are shown in Table 14. To reduce the complexity of the mixture, analogues 5 and 7 were not included in this mixture due to the above demonstrated negligible affinity of these analogues for P1. Polymer P1 retained good recognition for (E)-resveratrol with imprinting factor (IF 2.26) and selectivity (α 1.26) (where α=B_{MIP}−B_{NIP})/B_{NIP}) parameters that were virtually unchanged from the single analyte experiment (IF 2.35, a 1.36). This result clearly demonstrates that P1 preferentially binds (E)-resveratrol over its structurally similar analogues. Although the binding capacity of P1 for (E)-resveratrol from the mixture was reduced (7.78 μmol/g), this may be a consequence of competition for available binding sites by the tetra-ol analogue. The tetra-ol analogue displayed effectively unchanged binding to P1 (19.84 μmol/g) with improved recognition (IF 1.41) and selectivity (a 0.41) compared to the non-competitive studies (19.37 μmol/g, IF 1.17, a 0.17). The increased IF may be a consequence of reduced nonspecific binding of (E)-resveratrol to randomly distributed 4VP throughout the polymer arising from non-specific binding of other analytes present in the mixture. (E)-resveratrol analogues 3 and 4 that displayed good recognition in single analyte assays (IF 4.02 and 2.89 respectively) were unable to compete with (E)-resveratrol 1 and compound 2 for available binding sites on P1, as manifested by reduced IF and α for both analogues. In accordance with the above static binding results for single analytes, compounds 6 and 8 demonstrated essentially negligible binding. Compound 8 continued to show the lowest value consistent with the least amount of recognition. These results, in accordance with those obtained for single analyte assays, emphasise the importance of the —OH groups with respect to their number and position in the core molecule. Analogues of (E)-resveratrol having at least two —OH groups in the mew and para positions on the aromatic rings (compounds 1, 2, 3, 4) clearly demonstrated moderate to good affinity, with good correlation between binding affinities and the number of aromatic-linked OH groups present.

TABLE 14
Competitive cross-reactivity towards a resveratrol MIP using a solution
containing several closely related polyphenols.

| | MIP | NIP | | | |
|---|---|---|---|---|---|
| | Binding | Binding | | | |
| | μmol/g | μmol/g | Imprinting | | Selectivity |
| | polymer | polymer | Factor | | α = (B_{MIP} − |
| Analyte | B_{MIP} | B_{NIP} | IF | B_{MIP} − B_{NIP} | B_{NIP})/B_{NIP} |
| 1 | 7.78 | 3.45 | 2.26 | 4.33 | 1.26 |
| 2 | 19.84 | 14.08 | 1.41 | 5.77 | 0.41 |
| 3 | 3.70 | 2.01 | 1.84 | 1.69 | 0.84 |
| 4 | 3.5 | 1.9 | 1.84 | 1.6 | 0.84 |
| 6 | 1.17 | 0.8 | 1.46 | 0.38 | 0.47 |
| 8 | 1.00 | 1.31 | 0.76 | −0.3 | −0.23 |

### Conclusion

An (E)-resveratrol-imprinted polymer has been prepared via non-covalent self-assembly and shown with both single and mixed analyte samples to have a highly specific molecular recognition for the template over similar polyphenolic analogues. Recognition of the compounds was influenced by the presence of aromatic OH groups and required at least two such groups in the mew and or para positions. Compounds with more than three aromatic OH groups exhibited strong affinity for the (E)-resveratrol imprinted polymer, but their molecular recognition was inhibited by a high level of non-specific binding. Superior recognition was observed for resveratrol which was able to interact with the binding cavity through three mew and para positioned aromatic OH groups having complementarity with the 3-dimensional binding cavity

### E. Polyphenolic Template Selectophores for Molecularly Imprinted Polymers

Reported here is the synthesis of the imine and amide selectophores of (E)-resveratrol. These selectophores are more easily and efficiently prepared than (E)-resveratrol, as both can be synthesized by a “single-pot” preparation. The performances of the new polyphenolic selectophore-imprinted polymers relative to (E)-resveratrol-imprinted polymer is also reported.

### Results and Discussion

### Selectophore Preparations

(E)-Resveratrol 1 can be visualized as a polyphenolic compound with an alkene constraint. Analogues with a variation at this constraint were then selected for synthesis, namely the imine, (E)-5-[(4-hydroxy-phenylimino)-methyl]-benzene-1,3-diol 2 and the amide, 3,5-dihydroxy-N-(4-hydroxyphenyl)benzamide 3. All three compounds have two resorcinol hydroxyls that are able to occupy the same 3-dimensional spaces, with the third phenolic hydroxyl then likely to adopt very similar but not identical positions.

Earlier studies in this laboratory used (E)-resveratrol as the original template for the preparation of MIPs. There are numerous reported methods for the synthesis of (E)-resveratrol, but most of these synthetic methods contravene both the Principles of Green Chemistry and modern industrial practicality. All of these methods invariably require multistep syntheses with resultant significant losses of materials, along with consequential purification processes, multiple handlings of noxious reagents, protection and deprotection processes and energy to drive the chemical reactions.

The process used for the synthesis of (E)-resveratrol is summarized in Scheme 5 and was used to transform 3,5-dihydroxybenzoic acid 4 to (E)-resveratrol 1.

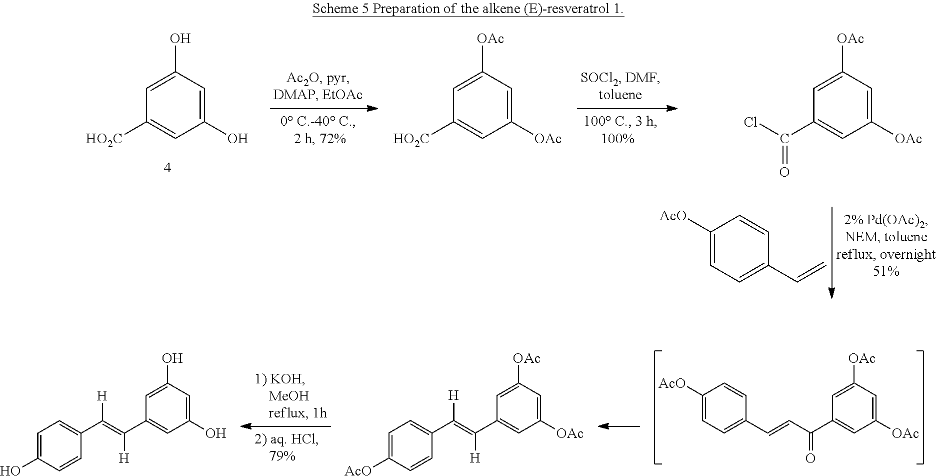

This multistep process returned the desired product in a 29% overall yield, but typically required >5 days to complete, and necessitated the generation and handling of corrosive and noxious materials, as well as chromatographic purifications.

The imine selectophore (E)-5-[(4-hydroxy-phenylimino)-methyl]-benzene-1,3-diol 2 was prepared from commercially available 4 aminophenol 5 and 3,5-dihydroxybenaldehyde 6 as shown in Scheme 6.

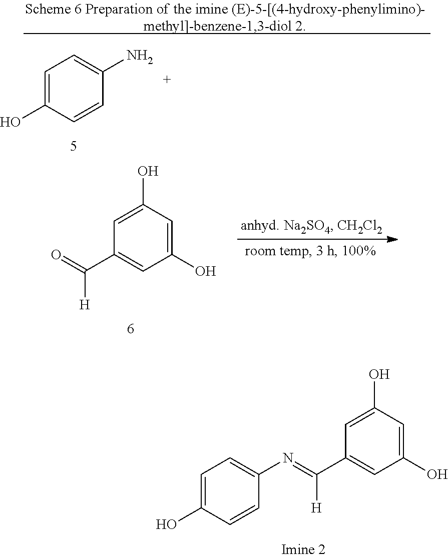 [SMILES: Nc1ccc(O)cc1 ; O=Cc1cc(O)cc(O)c1 ; Oc1ccc(/N=C/c2cc(O)cc(O)c2)cc1]

We have found this method to be highly advantageous in that it rapidly gave quantitative yields of clean product after a simple filtration workup. The stability of this imine product was confirmed by ¹H NMR studies in d₆-DMSO and CD₃CN, which showed that this compound remained unchanged in these solutions over a 6 day period. This imine selectophore also remained intact when used as the template molecule under the conditions employed for MIP preparation, and when used as an analyte to evaluate the binding properties of these MIPs.

The third selectophore example was the amide 3,5-dihydroxy-N-(4-hydroxyphenyl)benzamide 3. An authentic sample was initially prepared by the multistep procedure shown in Scheme 7.

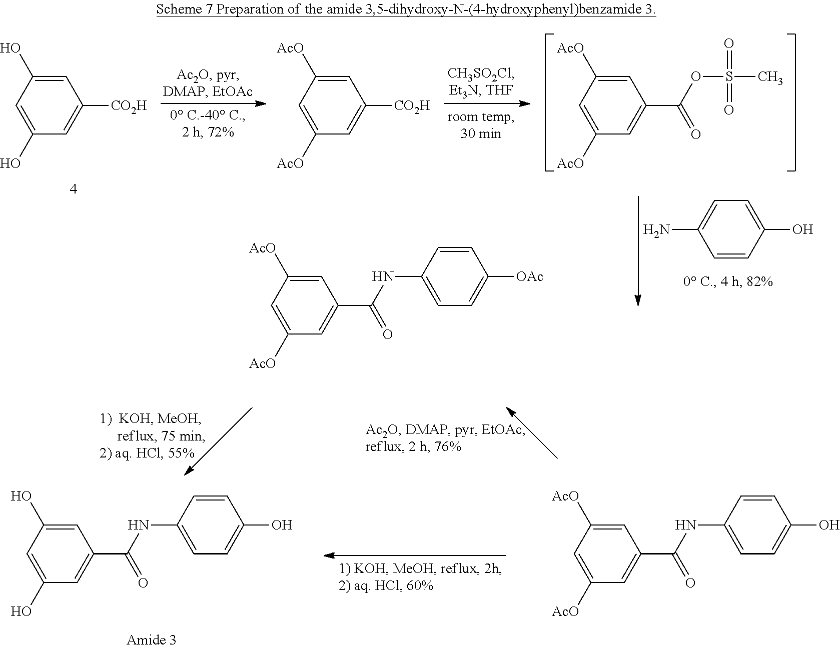

Full experimental details for this multistep preparation have been included in the attached electronic supplementary information. This multistep preparation of this amide 3 clearly suffered from the same “non-green” issues that were encountered in the synthesis of (E)-resveratrol 1. Our ongoing requirement for larger amounts of this amide 3 encouraged us to develop an alternative improved process, which is summarized in Scheme 8.

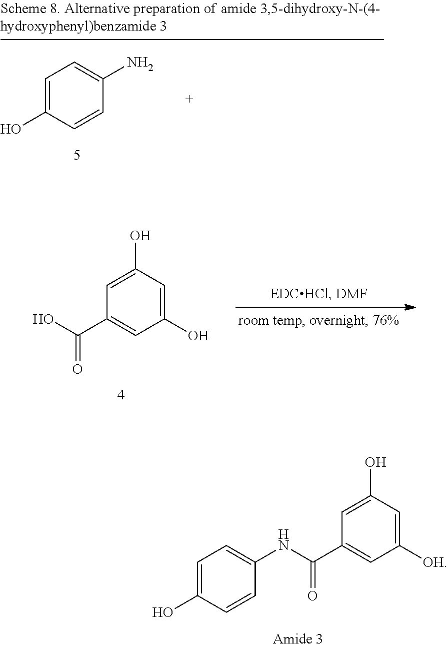 [SMILES: Nc1ccc(O)cc1 ; O=C(O)c1cc(O)cc(O)c1 ; O=C(Nc1ccc(O)cc1)c1cc(O)cc(O)c1]

Workup by removal of the solvent followed by trituration and washing with water rapidly returned the pure amide 3 in 76% yield. The ‘green’ advantage of this carbodiimide activated coupling is that the same starting reagents were used as for the reported multistep synthesis, but now the amide was produced in an improved overall yield, using a single-pot procedure, and did not require added energy, functional group protection and deprotection, or chromatographic purification.

### Molecularly Imprinted Polymers (MIPs)

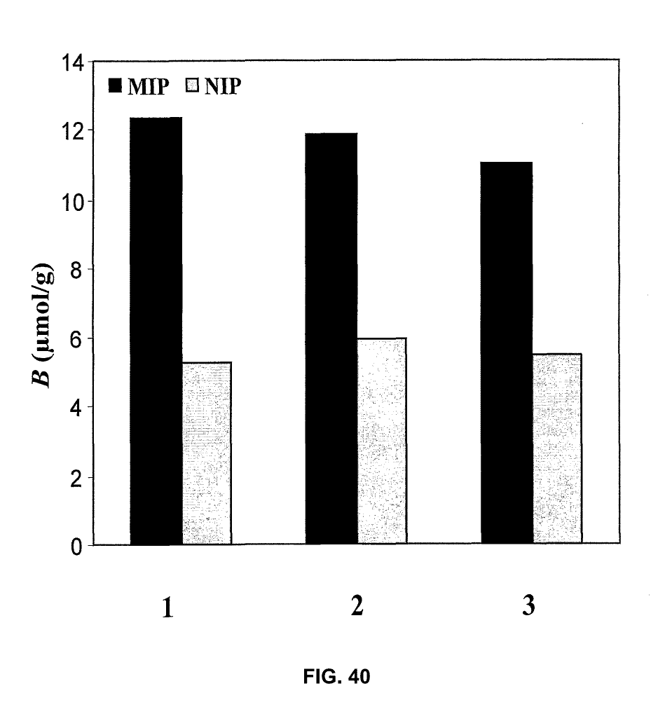

The three selectophores synthesized were used as templates for the preparation of MIPs, and as analytes to interrogate the binding characteristics of the new polymers. The preparation of the molecularly imprinted polymers (MIPs) briefly involved the thermally initiated free radical polymerization of a mixture of the selectophore template, the functional monomer and the crosslinker in a porogen. The resultant solid material was then ground and appropriate sized particles sieved off and washed with acid to desorb the template. Non-imprinted polymers (NIPs) were identically prepared in the absence of template molecule. Single analyte binding studies where performed using the (E)-resveratrol imprinted polymer (MIP_{RES}) and the corresponding NIP. in these studies, the polymers were incubated overnight with a freshly prepared solution of analyte, and the supernatant was then removed and its concentration determined by RP-HPLC. Subtraction from the original concentration gave the amount of analyte bound (B) by the polymer, and these results are summarized in FIG. 40.

Within this experimental design, MIP_{RES} displayed both similar binding capacity and comparable selective affinities (B_{MIP}−B_{NIP}) for the alkene, the amide and the imine molecules. The similarity of these binding parameters suggests that MIP_{RES} displays generic cross-reactivity towards each of these polyphenolic selectophores. This result is consistent with similar spatial conformations that all of these molecules adopt in solution. These selectophores are also likely to possess similar electronic characteristics as inductive variations associated with these three different linkages are likely to be moderated by both distance from the aromatic hydroxyl groups and the aromatic it clouds. Consequently, these selectophores are similarly bound at the recognition surfaces within these MIP cavities.

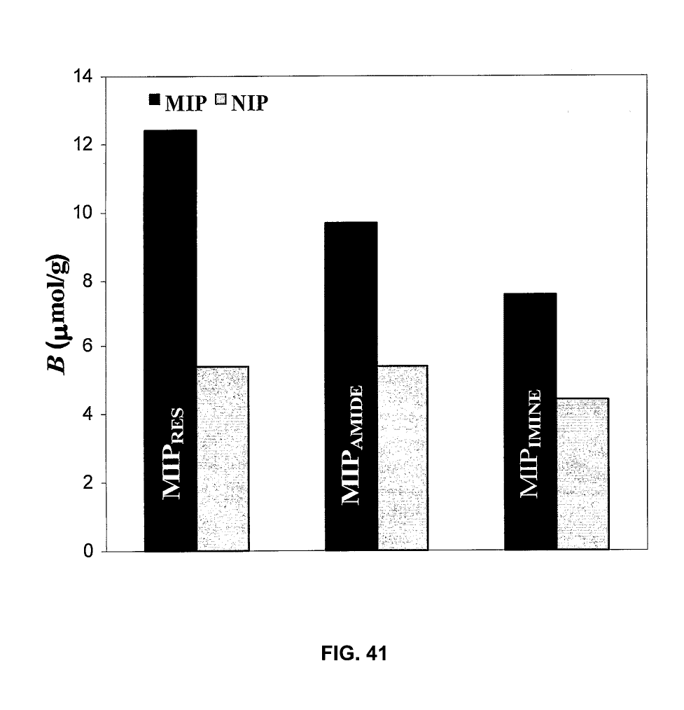

The suitability of the amide and imine selectophores as (E)-resveratrol template mimics was then assessed. Each of these compounds was used as templates for the preparation of new MIPs (i.e. MIP_{AMIDE} and MIP_{IMINE} respectively) using a similar protocol to that employed for the preparation of MIP_{RES}. Static binding assays were conducted as before, and the ability of these MIPs to recognise (E)-resveratrol are reported in FIG. 41.

The selectophore-imprinted MIP_{AMIDE} and MIP_{IMINE} both displayed selective affinity for (E)-resveratrol, but with reduced capacity compared to MIP_{RES}. Despite the fact that these molecules may have similar conformations in solution, the stereotopological spaces occupied by the pyridyl nitrogens of 4-vinylpyridine, which are the complementary functional binding groups during imprinting, must differ for all three MIPs. The decreased binding capacity and lower selective affinity observed for both MIP_{AMIDE} and MIP_{IMINE} may also reflect that these imprinted polymers have a diminished ability to bind and maintain a favoured conformation of the alkene (E)-resveratrol, a finding that may be a consequence of the increased relative structural rigidity of the alkene constraint.

### Conclusion

Methodologies to synthesize structural analogues with spatially defined functionalities, or selectophores, of the polyphenol (E)-resveratrol have been developed. These compounds were more easily and conveniently prepared using “greener” single-pot procedures. The selectophores were then used as template mimics for the preparation of MIPs, and these new polymers were found to display comparable, but not identical binding, towards this representative polyphenolic compound.

The approach is a technique that can be used to explore and characterize new MIPs, and our findings suggest that appropriate selectophores may be suitable templates to enable the more convenient preparation of generic MIPs which are capable of extracting structurally similar compounds.

### Experimental

AR solvents were used as purchased from the manufacturer except for dimethylformamide (DMF) which was dried over 4 Å molecular sieves, toluene over sodium wire, and pyridine and triethylamine over KOH pellets. Milli-Q distilled water was used for aqueous manipulations. Solvent mixtures are expressed as volume/volumes. Solvent extracts were dried over anhydrous sodium sulfate, filtered and then rotary evaporated to dryness at low pressures (>10 mbar) at 30-35° C. Analytical thin layer chromatography (TLC) was conducted on silica gel using Merck® 1.05554.001 plates. The components were visualised by (i) fluorescence at 254 nm and (ii) ethanolic phosphomolybdic acid solution dip and char. Silica gel column chromatography was conducted using Merck® 1.09385.1000. Melting points were determined by open glass capillary method and are uncorrected. NMR spectra were recorded on a Bruker DPX-300; ¹H at 300 MHz and ¹³C JMOD at 75 MHz. Deuterated solvents were used as indicated and the residual solvent peaks used for internal reference. J values are given in Hz. Low resolution electrospray ionisation mass spectra (ESI) were recorded using a Micromass Platform II API QMS Electrospray mass spectrometer in both positive (ESI⁺) and negative (ESI⁻) polarity. High-resolution electrospray mass spectra (HRMS) were recorded on a Bruker BioApex 47e Fourier Transform mass spectrometer. Mixtures were sonicated in a 37 kHz/150 W Elmasonic 5100 ultrasonic cleaning unit. Polymers were ground using a Retsch PM 200 Planetary Ball Mill and sized on a Retsch AS 200 sieve shaker.

### Synthesis of Polyphenolic Selectophores

### 3,5-Diacetoxybenzoic Acid

A suspension of 3,5-dihydroxybenzoic acid (15.40 g, 0.100 mol) in ethyl acetate (220 mL) was cooled in an ice-bath. Acetic anhydride (24.52 mL, 0.2421 mol), pyridine (16.16 mL, 0.1998 mol) and 4-(dimethylamino)pyridine (100 mg, 0.81855 mmol) were added and the reaction stirred at 0° C. for 60 minutes and then at room temperature overnight. Formic acid (5.12 mL, 0.1357 mmol) was added and the reaction poured onto ice (ca. 500 g). Further ethyl acetate (300 mL) was added and the organic phase separated and washed with water (2×200 mL), sat. aq. NaHCO₃ (100 mL), further water (2×200 mL), and then dried and evaporated to a white solid. Recrystallization from 5:1 EtOAc/hexane (120 mL) gave 2 crops of 3,5-diacetoxybenzoic acid (combined weight 17.07 g, 72%) as a white powder. R_{f} 0.20 (1:1 EtOAc/hexane), 0.39 (3:1 EtOAc/hexane); mp 161-162° C. (from EtOAc/hexane) (lit. mp: 157-159° C.); δ_{H}(CDCl₃) 2.29 (s, 6H, 2×OAc), 7.18 (pseudo t, 1H, J 2.1, 4-H) and 7.70 (pseudo d, 2H, J 2.1, 2-H, 6-H); δ_{C}(CD₃OD) 18.43, 118.94, 119.02, 131.70, 150.19, 165.46 and 168.17; m/z (ESI) 261 (MNa⁺, 100%).

### (E)-3,4′,5-Triacetoxystilbene

3,5-Diacetoxybenzoic acid (8.022 g, 33.706 mmol) suspended in a mixture of toluene (130 mL), DMF (500 μL) and thionyl chloride (16.00 mL, 220.6 mmol) was heated at 100° C. for three hours under an argon gas atmosphere. The solvents were removed by vacuum distillation and the residue re-suspended in toluene (85 mL) and sonicated under vacuum to remove dissolved gases. 4-Acetoxystyrene (5.74 mL, 37.5 mmol), N-ethylmorpholine (4.31 mL, 33.9 mmol) and palladium diacetate (35 mg, 0.16 mmol, 0.46 mole %) were added and the reaction heated to reflux for 2 hours. Further palladium diacetate (116 mg, 0.52 mmol, 1.54 mole %) was added and the reaction left to reflux overnight. On return to room temperature, ethyl acetate (500 mL) was added, the solution was washed with 0.1 M HCl (2×300 mL) and water (300 mL) and then dried and evaporated to return a brown solid. Purification with column chromatography (isocratically eluted with 2:1 Et₂O/hexane) gave 7.888 g of a white solid, shown by ¹H NMR to be predominantly the desired adduct. Further chromatography (gradient eluted starting with 4:1 hexane/EtOAc and finishing with 2:1 hexane/EtOAc) returned pure (E)-3,4′,5-triacetoxystilbene (6.071 g, 51%) as a white solid. R_{f} 0.29 (2:1 hexane/EtOAc); mp 112.5-113.0° C. (lit mp 116° C.); 5₁₁ (CDCl₃) 2.27 (s, 9H, 3×OAc), 6.80 (pseudo t, 1H, J 2.1, 4′-H), 6.93 (d, 1H, J 16.3, Hₜᵣₐₙₛ), 7.03 (d, 1H, J 16.3, Hₜᵣₐₙₛ), 7.04-7.09 (m, 4H, 3-H, 5-H, 2′-H, 6′-H) and 7.44-7.47 (m, 2H, 2-H, 6-H); δ_{C}(CDCl₃) 20.07, 113.39, 115.88, 120.88, 126.19, 126.64, 128.64, 133.45, 138.53, 149.46, 150.34, 167.91 and 168.30; m/z (ESI) 377 (MNa⁺, 100%), 378 (21).

### Alkene 1; (E)-Resveratrol

The reaction was conducted under an argon gas atmosphere. Potassium hydroxide (22 mg, 0.3922 mmol) dissolved in methanol (3.0 mL) was added to a suspension of (E)-3,4′,5-triacetoxystilbene (113 mg, 0.319 mmol) in methanol (10 mL). The solid immediately dissolved and the clear solution was gently heated to reflux for 60 minutes and a marked darkening in colouration noted. The volume was then reduced to half with rotary evaporation and the remaining solution acidified (pH 2) with 1M aq. HCl. Ethyl acetate (150 mL) was added and the reaction washed with sat. brine (3 20×20 mL), dried and evaporated to return a dark red solid. Purification with column chromatography (isocratically eluted with 100% EtOAc) gave (E)-resveratrol (58 mg, 79%) as a pale beige coloured solid. R_{f} 0.65 (EtOAc); mp 261.0-263.0° C. (lit mp 255-260° C.); δ_{H}(CD₃OD) 6.13 (pseudo t, 1H, J 2.2, 4-H), 6.41-6.42 (m, 2H, 2-H, 6-H), 6.71-6.79 (m, 3H, Hₜᵣₐₙₛ, 5′-H), 6.93 (d, 1H, J 16.3, Hₜᵣₐₙₛ) and 7.29-7.36 (m, 2H, Jₒᵣₜₕₒ 8.6, 2′-H, 6′-H); δ_{C} (CD₃OD) 100.30, 103.47, 114.12, 124.64, 126.43, 127.06, 128.07, 138.97, 155.89 and 157.20; m/z (ESI) 229 (MH⁺, 100%), 230 (23).

### Imine 2; (E)-5-[(4-Hydroxy-phenylimino)-methyl]-benzene-1,3-diol

A mixture of 3,5-dihydroxybenzaldehyde (300 mg, 2.174 mmol), 4-aminophenol (237 mg, 2.174 mmol) and anhyd. sodium sulphate (309 mg, 2.176 mmol) in dichloromethane (15 mL) was vigorously stirred at room temperature for 3 hours. Further anhyd. sodium sulphate (309 mg, 2.176 mmol) was added and the stirring continued for an additional one hour. Dichloromethane was removed by rotary evaporation and the white residue re-suspended in boiling ethanol (10 mL) and filtered whilst hot. The solid was washed in the funnel with further hot ethanol (10 mL). The clear filtrates were combined and rotary evaporated to dryness to give (E)-5-[(4-hydroxy-phenylimino)-methyl]benzene-1,3-diol (501 mg, 100%) as a pale pink solid. R_{f} 0.48 (2:1 EtOAc/hexane); darkens with heating with mp>340° C.; δ_{H} (d₆-DMSO) 6.36 (pseudo t, 1H, J 2.2, 4-H), 6.80-6.85 (m, 4H, 2-H, 6-H, 3′-H, 5′-H), 7.16-7.22 (m, 2H, Jₒᵣₜₕₒ 8.8, 2′-H, 6′-H), 8.43 (s, 1H, imine-H), 9.47 (bs, 3H, 3×phenolic-OH); δ_{C} (d₆-DMSO) 106.27, 107.41, 116.70, 123.42, 139.35, 143.61, 157.16, 158.38 and 159.62; m/z (ESI) 230 (MH⁺, 100%), 231 (13); m/z (HRESI) 252.0633 ([M+Na]⁺ C₁₃H₁₁NO₃Na⁺ requires 252.0637).

### Amide 3; 3,5-Dihydroxy-N-(4-hydroxyphenyl)benzamide

The reaction was conducted under a positive pressure of argon gas. 3,5-Dihydroxybenzoic acid (2.310 g, 15.000 mmol) and 4-aminophenol (1.962 g, 18.000 mmol) were dissolved in DMF (90.0 mL). N-Ethyl-N′-(3-dimethylaminopropyl)carbodiimide hydrochloride (3.450 g, 17.997 mmol) was added and the reaction stirred at room temperature overnight. The solvent was removed by rotary evaporation under high vacuum and the oily residue co-distilled with water until a solid formed. This solid was triturated with cold 0.01 M HCl (50 mL), filtered, and the pale purple coloured product washed in the funnel with cold water (4×20 mL). The solid was dried with desiccant under vacuum to give pure 3,5-dihydroxy-N-(4-hydroxyphenyl)benzamide (2.791 g, 76%); R_{f} 0.39 (4:1 EtOAc/hexane); mp 266.0-266.5° C.; δ_{H}(CD₃OD) 6.47 (pseudo t, 1H, Jₘₑₜₐ 2.2, 4-H), 6.77-6.82 (m, 4H, 3′-H, 5′-H, 2-H, 6-H), 7.43-7.46 (m, 2H, Jₒᵣₜₕₒ 8.9, 2′-H, 6′-H); δ_{H} (d₆-DMSO) 6.42 (pseudo t, 1H, Jₘₑₜₐ 2.2, 4-H), 6.72-6.78 (m, 4H, 3′-H, 5′-H, 2-H, 6-H), 7.51-7.56 (m, 2H, Jₒᵣₜₕₒ 8.9, 2′-H, 6′-H), 9.20 (s, 1H), 9.51 (s, 2H) and 9.83 (s, 1H); δ_{C}(CD₃OD) 104.41, 104.67, 113.92, 122.13, 129.10, 136.08, 153.26, 157.41 and 166.70; m/z (ESI) 244 ([M−H]⁻, 100%), 489 ([2M−H]⁻, 29); m/z (HRESI) 246.0764 ([M+H]⁺C₁₃H₁₂N₁O₄⁺ requires 246.0766).

### Preparation of Molecularly Imprinted Polymers

Molecularly imprinted polymers (MIPs) were prepared by dissolving the selectophore template (1 mmol) in porogen (5:1 acetonitrile/EtOH, 6 ml) in a disposable glass test tube. Acetone (6 mL) was the preferred porogen with the amide 3 due to its incomplete solubilization in the original mixture volume.¹⁰ The functional monomer 4 vinylpyridine (322 μL, 3 mmol) was added and the mixture sonicated for 10 minutes. Ethyleneglycol dimethacrylate (2.314 mL, 15 mmol) was then added as cross-linker and 2,2-azobis(isobutyro)nitrile (51 mg, 0.31 mmol) added as radical initiator. This pre-polymerisation mixture was sparged with nitrogen gas for 5 minutes before being heated in a thermostatic water bath at 50° C. for 24 hours. The solid polymers were removed from the reaction tubes, crushed with a mortar and pestle and finely ground using a Retsch 200 ball mill. Grounds were sieved and the 63-100 μm size particles retained. This size range was repeatedly suspended in acetone and the supernatant poured off to remove fines. Bound template was then removed from the polymer by repeated washings with methanolic 10% acetic acid until the washings were free of template (monitored by UV-Vis spectroscopy at 321 nm). Non-imprinted control polymers (NIPs) were prepared using the same procedure in the absence of a template molecule.

### Evaluation of Molecularly Imprinted Polymers

Experiments were conducted on both MIP and NIP in parallel under identical conditions.

Analyte solution (1.5 mL, 0.5 min in acetonitrile) was added to the polymer (30 mg) in an Eppendorf tube. This was mixed at 40 rpm for 18 hours and the polymer settled by centrifugation for 15 minutes at 13000 rpm. A 200 μL aliquot of the supernatant was removed and analysed by RP-HPLC with UV detection at 321 nm. Concentration was determined by comparison with a linear 5 point calibration curve. The amount of analyte bound (B) was determined as the difference between this and the initial value and reported in units of μmol/g polymer.

F. Enrichment of the Bioactive Polyphenol (E)-Resveratrol from Peanut by-Products Via Molecular Imprinting

A rapid technique for the isolation and enrichment of (E)-resveratrol and the determination of related polyphenols from peanut press waste using molecular imprinting technology is reported.

### Materials and Methods

### Compounds

4-Vinylpyridine (4VP), ethyleneglycol dimethacrylate (EGDMA) and 2,2′-azobis(2-methylpropionitrile) (AIBN), (+)-catechin hydrate (lyophilized prior to use), caffeic acid and (E)-piceid were obtained from Sigma-Aldrich (Sydney, NSW, Australia). 4-VP and EGDMA were purified immediately prior to use via vacuum distillation and alumina column chromatography, respectively. All solvents used were HPLC grade.

(E)-Resveratrol 1 was synthesized using the methodology reported above.

### MIP Preparation

An (E)-resveratrol imprinted polymer (MIP_{RES}) was prepared by dissolving (E)-resveratrol and 4-VP (in ratio 1:3 mole equivalents) in acetonitrile/EtOH (5:1 v/v). The resulting mixture was purged with N₂ (g) for 2 minutes and sonicated for 20 minutes prior to the addition of EGDMA (15 mole equivalents) and the free radical initiator AIBN. The mixture was then sealed and purged with N_{2(g)} for 2 minutes and then placed in a 50° C. water bath (18 hrs) followed by a thermal annealing treatment at 60° C. (24 hrs). A non-imprinted polymer (NIP) was prepared as a control in the same manner without the inclusion of the (E)-resveratrol template. The resulting hard bulk monoliths were ground using a Retsch 200 ball mill to produce a particle size distribution of 60-100 μm which were isolated by sieving. The template molecule was extracted by repeated washings with MeOH/AcOH (9:1 v/v) until the template was no longer visible in the extraction media by absorbance at 321 nm. The MIP particles were then washed with MeOH to remove traces of AcOH and the fines removed by repeated sedimentation in acetone. The remaining MIP particles were subsequently dried in vacuo at 40° C. overnight.

### Static Selectivity Studies

Selectivity studies were conducted for both MIP and NIP using a constant polymer amount of 30 mg. The NIP was used to determine the extent of non-specific binding resulting from interactions with the cross-linker and randomly dispersed functional monomer. The polymer was incubated in 1.5 mL of analyte solution (0.5 min) in acetonitrile. The resulting mixture was mixed at 40 rpm for 18 hours and then centrifuged at 13000 rpm for 15 minutes. An aliquot (200 μL) of the supernatant was removed and analysed by RP-HPLC by UV spectroscopy at 321 nm and the concentration of free analyte was determined from a linear 5 point calibration curve. The amount of bound analyte (B), expressed as μmol/g polymer, was calculated by subtracting the free analyte concentration from the initial total analyte solution concentration.

### MISPE Validation Studies

Small scale MISPE studies were conducted on SPE columns containing 100 mg of either MIP or NIP stationary phases. Polymeric stationary phase (100 mg) was slurry packed in MeOH into 3 mL syringe barrels fitted with polypropylene frits (20 μm pore size). The resulting SPE columns were subsequently conditioned with 1.5 mL (3 column volumes) of either acetonitrile or EtOH/H₂O (1:1, v/v) for organic and aqueous studies respectively, after which 1 mL of an (E)-resveratrol (0.5 min) solution in acetonitrile or EtOH/H₂O (1:1, v/v) was loaded on-column. Multiple selective clean up steps (1 mL each) were then applied to each column using either acetonitrile or 1% AcOH in EtOH/H₂O (1:1, v/v) after which each column was eluted using 10% AcOH in MeOH (2 mL). The fractions collected from the clean up and elution steps were evaporated to dryness, reconstituted to 1 mL in EtOH/H₂O (1:1, v/v) and analysed by RP-HPLC. The (E)-resveratrol concentration was determined using a 5 point calibration curve.

### Reversed-Phase Chromatography (RP-HPLC)

The RP-HPLC separation was performed on an Agilent Technologies 1100 LC system (Waldbronn, Germany) consisting of a binary pump with a vacuum degasser, auto-sampler with a 900 μL, sample loop, thermostated column compartment and a diode-array detector. Injected samples were analysed on a Zorbax Eclipse XDB-C₁₈ column (4.6×150 mm, 5 μm particle size) at 40° C. The mobile phase consisted of 0.1% AcOH in H₂O (solvent A) and 0.1% AcOH in EtOH/H₂O applying the following gradient: 0-2 min: 25% B isocratic, 2-6 min: 25-37.5% B, 6-9 min: 37.5% B isocratic, 9-12 min: 37.5-62.5% B, 12-15 min: 62.5% B isocratic, 15-18 min: 62.5-100% B, 18-22 min: 100% B isocratic, 22-25 min: 100-25% B, 25-29 min: 25% B isocratic. The flow rate was 0.5 mL min⁻¹. Injection volume was 5 μL with the UV-Vis diode array detector (80 Hz) set to the absorbance wavelengths of λ=280, 321 and 370 nm.

### LC-ESI-MS

All chromatographic separations were performed using an Agilent 1100 Capillary LC system (Agilent Technologies, Palo Alto, Calif., USA) coupled to an ion-trap MS system (Agilent 1100 Series LC/MSD-SL). Separation of the MIP eluate was performed using a Zorbax 300SB-C₁₈ (150 mm×0.3 mm I.D.) capillary column packed with 3.5 μm particles. The outlet of the column was directly connected to the electrospray source of the ion-trap mass spectrometer, with the UV detector bypassed. A 0.3 mg/mL MIP eluate sample in acetonitrile/H₂O (60:40, v/v) containing 0.1% FA was separated using a mobile phase consisting of 0.1% FA in H₂O (solvent A) and 0.1% FA in acetonitirle applying the following gradient: 0-15 min: 10% B, 15-65 min: 10-65% B, 65-70 min; 60-95% B. The flow rate was 4 μL min^{d} Injection volume was 0.2 μL. ESI-MS/MS analysis was carried out in the positive ion mode. The mass spectrometer was operated in a data-dependent mode where the two most intense ions in the precursor ion scan were subjected to subsequent automated MS/MS. All system control and data acquisition were conducted with Agilent ChemStation and MSD Trap Control software.

### Preparation of Peanut Press Waste Extract

Peanut press waste (200 g) in EtOH:H₂O (1000 mL, 4:1 v/v) was sonicated for 60 min, after which the mixture was filtered and the solvent evaporated to return 17.9 g of extract. A 10.0 g amount of this peanut press waste extract was reconstituted to 500 mL in EtOH/H₂O (1:1 v/v) in a volumetric flask and stored at 4° C. until required. Prior to use the peanut press waste extract was equilibrated at room temperature and sonicated to clarify the solution.

### MISPE of Peanut Press Waste Extract

MISPE studies were conducted using 1.0 g of either MIP_{RES} or the corresponding NIP as control stationary phase, to examine its ability to selectively retain and enrich (E)-resveratrol from the peanut press waste extract described above. Columns were conditioned using 3 column volumes each of MeOH, then EtOH and finally EtOH/H₂O (1:1, v/v) containing 0.1% AcOH. Peanut press waste extract (2×50 mL) was applied to each column. The breakthrough fractions were collected under vacuum and recycled back through the columns to maximize the interaction between the stationary phase and the polyphenolic components within the peanut press waste extract. Each column was then washed with EtOH/H₂O (1:1 v/v) (20 mL), followed by successive selective clean up steps comprising EtOH/H₂O (1:1 v/v) containing 1% AcOH (20 mL) and then acetone/H₂O (1:1 v/v) (30 mL) to disrupt non-specific binding. An acidic elution step with 10% AcOH in MeOH (25 mL) was then applied to desorb and remove the compounds of interest from each column. Elution fractions were combined, evaporated to dryness and made up to 1.0 mL in EtOH/H₂O (1:1 v/v) and analysed by RP-HPLC.

### Results & Discussion

### Molecularly Imprinted Polymers

(E)-resveratrol-imprinted polymer cavities were generated via an initial self-association between the acidic phenolic hydroxyl groups of (E)-resveratrol with the electron rich pyridinyl nitrogen of the 4VP functional monomer. The resultant pre-polymerization complex formed primarily from self-assembling hydrogen bonding interactions, may be stabilized by the participation of additional aromatic π-π interactions due to the close proximity of phenolic and pyridinyl aromatic groups. The self-assembled complex was then ‘frozen’ by polymerization of the styryl functionalities in the presence of the cross-linker EGDMA. Removal of the template molecule resulted in the generation of MIP cavities that are complementary to (E)-resveratrol.

The cross-reactivity of MIP_{RES} was evaluated using several structurally related, naturally occurring polyphenols: E-resveratrol, caffeic acid, (=)-catechin and (E)-piceid. For MIP_{RES}, the order of recognition (MIP-NIP) was (E)-resveratrol 1>caffeic acid 2>catechin 3>(E)-piceid 4 (Table 15). Although the binding capacity of MIP_{RES} for both caffeic acid and (+)-catechin is comparable to that observed for the template molecule, this is offset by increased non-specific binding of these compounds by the NIP. It can be concluded that the greater number of groups on caffeic acid and (+)-catechin, compared to (E)-resveratrol, that can form hydrogen bonds with the NIP is responsible for stronger non-selective binding and thus greater affinity to the NIP control polymer, thereby leading to reduced recognition. Negligible recognition of the glucosylated resveratrol molecule (E)-piceid, was observed, which is presumably due to the presence of the bulky glucose substituent preventing access to the MIP_{RES} binding cavities. Further, the reduced binding capacity of the NIP for (+)-catechin compared to (E)-resveratrol provides additional support for the involvement of H-bond interactions in non-specific binding to MIPs and NIPs. Based upon the selectivity displayed by MIP_{RES} towards (E)-resveratrol over other structurally related molecules, typical of those found in polyphenol-rich sources, it was anticipated that a MIP_{RES} based SPE extraction would selectively isolate the target (E)-resveratrol from a complex agricultural byproduct matrix.

TABLE 15
Cross-reactivity studies on MIP_{RES} and the respective NIP control polymer
with (E)-resveratrol 1, caffeic acid 2, (+)-catechin 3 and (E)-piceid 4.

| | | |
|---|---|---|
| | | Selectivity |
| Binding μmol/g | Recognition | (B_{MIP} − |

| Analyte | B_{MIP} | B_{NIP} | B_{MIP} − B_{NIP} | B_{NIP})/B_{NIP} |
|---|---|---|---|---|
| 1 | 10.28 | 4.19 | 6.09 | 1.45 |
| 2 | 9.27 | 6.07 | 3.20 | 0.53 |
| 3 | 9.63 | 7.44 | 2.19 | 0.29 |
| 4 | 2.23 | 1.42 | 1.66 | 0.57 |

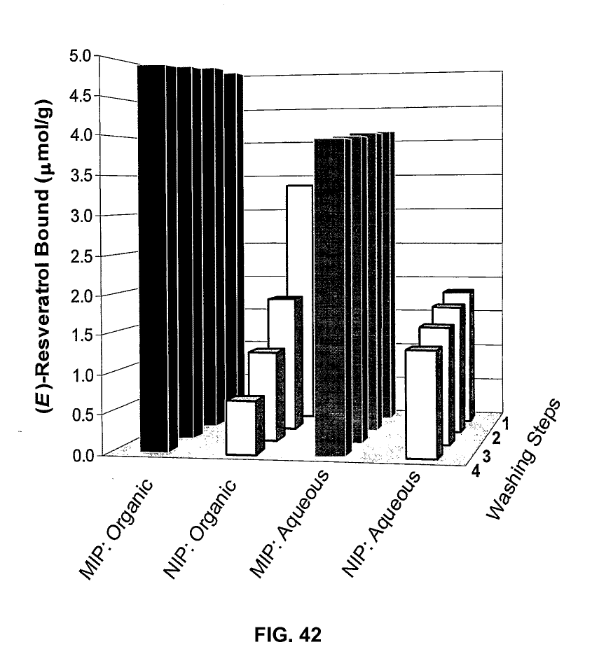

To establish that the use of MIP_{RES} as a MISPE sorbent for the refinement of (E)-resveratrol was viable, preliminary small scale experiments were conducted with MIP_{RES} stationary phase in both organic and aqueous environments. The binding capacity of MIP_{RES} for (E)-resveratrol was determined in this format, as was the effect of an aqueous clean up step to disrupt weak or non-specific binding compared to an organic clean up step (FIG. 42). Typically, organic solvents such as acetonitrile or dichloromethane are employed for the clean up step to minimise non-specific hydrophobic interactions and to disrupt non-specific or weak binding interactions. MIP_{RES} displayed superior binding capacity for (E)-resveratrol under organic conditions. However, the ability of MIP_{RES} to extract (E)-resveratrol from an aqueous solution, albeit with reduced efficacy, clearly demonstrates the applicability of using MIPs for the extraction and elution of this compound under conditions typically encountered in a processing or manufacturing environment (FIG. 42). Accordingly, larger gram scale columns containing either MIP_{RES} or the corresponding NIP control were employed as MISPE stationary phases for the extraction of (E)-resveratrol from an aqueous peanut press waste extract containing a complex mixture of bioactive polyphenols including (E)-resveratrol and catechin based oligomers.

### MISPE of Peanut Press Extract

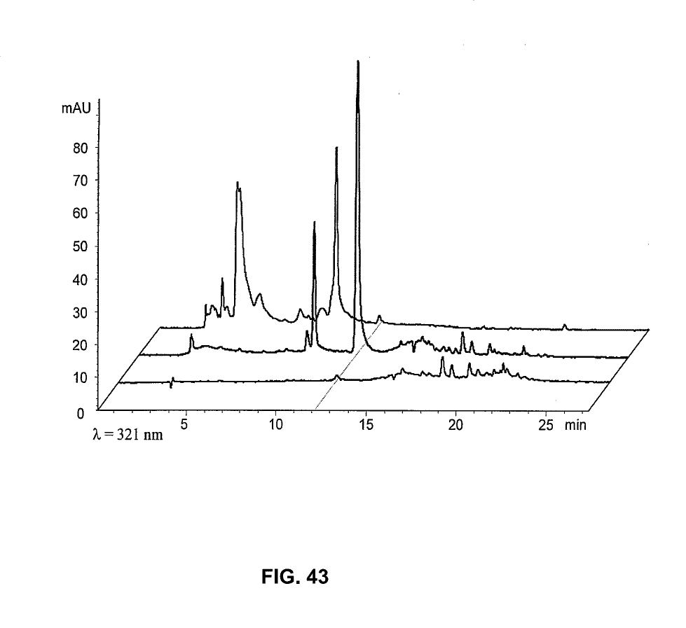

Peanut press waste is a by-product of peanut oil preparation, constituting the remains of the peanut and husk after pressing that contains a range of bioactive constituents including phytosterols, flavanols and other polyphenols. However, as this by-product is regularly disposed of as landfill or stock feed, these bioactives often remain underutilized. As peanuts and peanut derivatives are consumed in large quantities globally, the resulting peanut meal by-product presents a significant reservoir of bioactive components such as the bioactive polyphenol (E)-resveratrol, which is present in amounts ranging from 0.02-1.79 μg/g in various peanut market types. Therefore, we have employed MIP_{RES} in a MISPE format to selectively isolate and enrich (E)-resveratrol from a peanut meal liquid extract. RP-HPLC chromatograms of the peanut meal extract and subsequent MIP eluates are shown in FIG. 43. MIP_{RES} clearly resulted in significant sample clean up of the peanut meal extract and enrichment of (E)-resveratrol (Rₜ=12.2 min) and several unknown compounds. The amount of (E)-resveratrol present in the eluate from MIP_{RES} (39.5 μg/mL) signifies an approximate 60-fold enrichment of this important bioactive from a crude feedstock. This enrichment can be solely attributed to the (E)-resveratrol imprinting effect as the chromatogram of the NIP eluate shows no significant enrichment of (E)-resveratrol, which is present in similar quantity to that measured in the untreated peanut meal extract.

The MIP_{RES} eluate was analysed by tandem liquid chromatography-ESI-mass spectrometry (LC-ESI-MS) in positive ion mode, which confirmed the presence of (E)-resveratrol (Rₜ=12.2 minutes, 229 m/z [M+H]⁺) by comparison to an (E)-resveratrol standard.

In addition to (E)-resveratrol, the MIP_{RES} cavities also exhibited affinity for a secondary molecule as evidenced by the peak at Rₜ=9.9 min. This peak is associated with a more polar compound that was not retained by the NIP control polymer. LC-ESI-MS revealed the presence of mass peaks corresponding to [M+H]⁺ ions at 577 m/z and 865 m/z, both of which correlate with the expected mass of A-type procyanidin dimers and trimers, respectively.

### Conclusion

The application of a molecularly imprinted solid phase extraction technique has been demonstrated for the isolation and enrichment of bioactive polyphenols from peanut by-products. The use of an (E)-resveratrol imprinted polymer as the stationary phase column afforded both significant sample clean up and a 60-fold enrichment of (E)-resveratrol from an aqueous peanut press waste extract with minimal sample preparation.

### G. Tandem Molecularly Imprinted Solid Phase Extraction of Resveratrol and Related Polyphenols

In this example, a rapid tandem MISPE approach for the isolation and concentration of (E)-resveratrol and related polyphenols in high purity from peanut meal is reported.

### Materials and Methods

### Compounds

4-Vinylpyridine (4VP), ethyleneglycol dimethacrylate (EGDMA) and 2,2′-azobis(2-methylpropionitrile) (AIBN), catechin and piceid were obtained from Sigma-Aldrich (Sydney, NSW, Australia). 4-VP and EGDMA were purified immediately prior to use via vacuum distillation and alumina column chromatography, respectively. All solvents used were HPLC grade.

(E)-Resveratrol 1 and the structurally related polyphenolic analogue 3,5-dihydroxy-N-(4-hydroxyphenyl)benzamide 2 were synthesized as described above.

### MIP Preparation

Several MIPs and their non-templated counterparts (NIPs) were prepared as summarized in Table 16. MIP_{RES} was prepared at 50° C. (18 hours) followed by a thermal annealing treatment at 60° C. (24 hours). MIP_{AMIDE} was produced with a different porogen at 55° C. (40 hours). Both these preparations produced MIPs having comparable binding performance.

TABLE 16
Synthesis conditions of the MIPs used.

| | | Functional | | |
|---|---|---|---|---|
| | | monomer | Cross-linker | |
| Polymer | Template | (4VP) | (EGDMA) | Porogen |
| ⁺MIP_{RES} | 1 mmol (1) | 3 mmol | 15 mmol | CH₃CN/EtOH 5:1 |
| | | | | v/v |
| ⁺NIP_{RES} | — | 3 mmol | 15 mmol | CH₃CN/EtOH 5:1 |
| | | | | v/v |
| *MIP_{AMIDE} | 1 mmol (2) | 3 mmol | 15 mmol | CH₃COCH₃ |
| *NIP_{AMIDE} | — | 3 mmol | 15 mmol | CH₃COCH₃ |

⁺Prepared at 50° C. with thermal annealing treatment at 60° C. in 6 mL of porogen.
*Prepared at 55° C. in 5 mL porogen.

### Reversed-Phase Chromatography (RP-HPLC)

The RP-HPLC separation was performed on an Agilent Technologies 1100 LC system (Waldbronn, Germany) consisting of a binary pump with a vacuum degasser, auto-sampler with a 900 μL sample loop, thermostated column compartment and a diode-array detector. Injected samples were analysed on a Zorbax Eclipse XDB-C₁₃ column (4.6×150 mm, 5 μm particle size) at 40° C. The mobile phase consisted of 0.1% AcOH in H₂O (solvent A) and 0.1% AcOH in EtOH/H₂O or MeOH/H₂O (8:2 v/v) (solvent B), applying the following gradient: 0-2 min: 25% B isocratic, 2-6 min: 25-37.5% B, 6-9 min: 37.5% B isocratic, 9-12 min: 37.5-62.5% B, 12-15 min: 62.5% B isocratic, 15-18 min: 62.5-100% B, 18-22 min: 100% B isocratic, 22-25 min: 100-25% B, 25-29 min: 25% B isocratic. The flow rate was 0.5 mL min^{d} The injection volume was 5 μL with the UV-Vis diode array detector (80 Hz) set to the absorbance wavelengths of λ=280, 321 and 370 nm.

### Preparation of Peanut Meal Extract

Peanut meal (200 g) in EtOH:H₂O (1000.0 mL, 4:1 v/v) was sonicated for 60 min, after which the mixture was filtered and the solvent evaporated to return 17.9 g of extract. A peanut meal extract for use in subsequent experiments was prepared by adding 10.0 g of this material to a solution of EtOH/H₂O (1:1 v/v) and diluting to 500 mL in a volumetric flask and stored at 4° C. until required. Prior to use, the peanut meal extract was brought to room temperature and sonicated to clarify the solution.

### Resveratrol Saturation Studies

(E)-Resveratrol saturation studies over the concentration range of 0-2 min were conducted for both MIPs and their NIP counterparts using a constant amount of polymer (30 mg). The NIP response was used to determine the extent of non-specific binding resulting from interactions with the cross-linker and randomly dispersed functional monomer. The polymers were incubated with mixing at 40 rpm for 18 hours in 1.5 mL of acetonitrile containing resveratrol and then centrifuged at 13000 rpm for 15 minutes to pellet the polymers. An aliquot (200 μL) of the supernatant was removed and analysed by RP-HPLC with UV detection at 321 nm, and the concentration of unbound (E)-resveratrol was determined from a linear 5 point calibration curve. This concentration was subtracted from the total analyte solution concentration to derive the amount of analyte bound (B) expressed as μmol/g polymer.

### Static Selectivity Studies

Selectivity studies were conducted for both MIPs and NIPs using a constant amount of polymer (30 mg). The polymers were separately incubated with either resveratrol or the structurally similar analogues 3,5-dihydroxy-N-(4-hydroxyphenyl)benzamide 2, catechin 3 and (E)-piceid 4. Binding of each of these compounds was evaluated as described above.

### MISPE of Peanut Meal Extract

Preliminary MISPE studies were conducted using MIP_{RES} or MIP_{AMIDE} (250 mg) to investigate their ability to selectively retain and enrich resveratrol and other polyphenols from the peanut meal extract. A solution of the peanut meal extract (25 mL) was applied to each column and the breakthrough fractions were collected under vacuum and recycled back through the columns to maximize the interaction between the stationary phase and the polyphenolic components within the peanut meal extract. Each column was then washed with EtOH/H₂O (1:1 v/v, 3 column volumes, 6 mL), then with H₂O (10 mL) to remove unbound and/or weakly bound water-soluble components of the extract. A selective clean up step with aqueous acetone (acetone/H₂O, 1:1 v/v, 2 mL) was used to disrupt non-specific binding. An acidic elution step with 10% AcOH in MeOH (10×1 mL) was then used to desorb and remove the specifically bound compounds of interest from each polymer. The elution fractions were combined, then evaporated to dryness and reconstituted in 1.0 mL in EtOH/H₂O (1:1 v/v). These concentrated eluates were clarified by centrifugation and the supernatant removed for RP-HPLC analysis.

### Tandem MISPE of Peanut Meal Extract

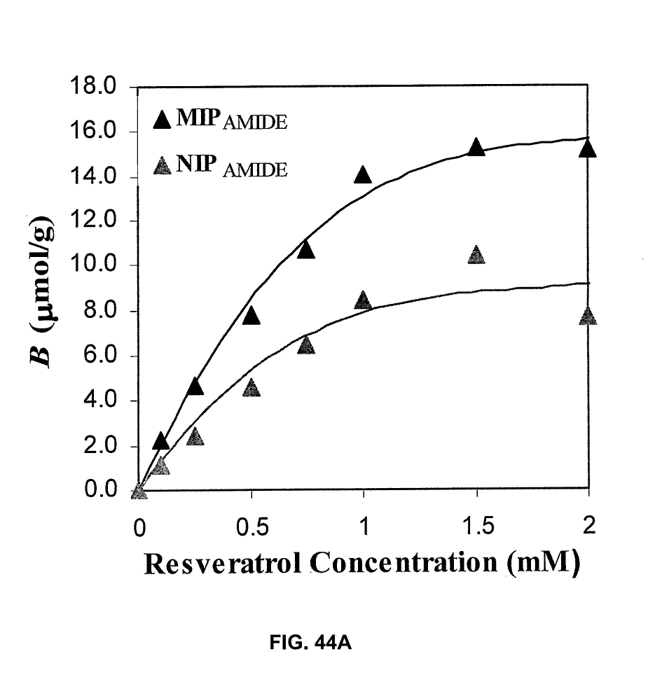

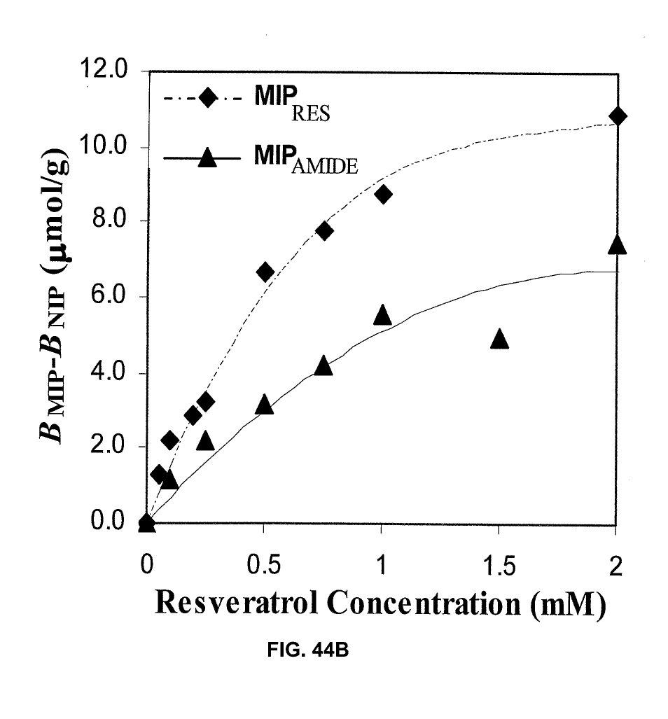

Separate MIP_{RES} and MIP_{AMIDE} columns (1000 mg each) were pre-conditioned with EtOH/H₂O (1:1 v/v) containing 1% AcOH (15 mL). Peanut meal extract (100 mL) was loaded onto the first of two MIP columns in series (MIP_{RES}) as illustrated in FIG. 44A. The flowthrough from the MIP_{RES} column was then loaded onto the MIP_{AMIDE} column and the flowthrough from MIP_{AMIDE} was collected for further investigations. The MIP_{RES} column was then washed with 100 mL of EtOH/H₂O (1:1 v/v) containing 1% AcOH and the wash fraction loaded onto MIP_{AMIDE}. Finally, both MIP columns were separately eluted using 3×10 mL of 10% AcOH in MeOH and the eluates were collected in 2 mL fractions (FIG. 44B). Samples of each of the flowthroughs and wash fractions were retained for analysis.

Prior to the second cycle (FIG. 44C), the MIP_{RES} and MIP_{AMIDE} columns were reconditioned with EtOH/H₂O (1:1 v/v) containing 0.1% AcOH and then the combined resveratrol-depleted extract and wash fractions were reloaded onto these columns in series as described above in order to isolate any remaining polyphenolic compounds. MIP_{RES} column was then washed with 25 mL of EtOH/H₂O (1:1 v/v) containing 1% AcOH and the wash fraction was loaded onto the MIP_{AMIDE} column and eluted using EtOH/H₂O (1:1 v/v) containing 1% AcOH.

The processed extract from two cycles of tandem MISPE was subsequently reloaded onto a larger column of MIP_{RES} (5 g) to extract any remaining polyphenolic compounds in the largest quantity possible. This column was pre-conditioned with MeOH containing 10% AcOH (3 column volumes), MeOH (3 column volumes) and 1% AcOH in EtOH/H₂O (1:1, v/v) (3 column volumes). Remaining processed peanut meal extract (comprising the flowthrough and washes from first MISPE application described above) was applied (100 mL) and the subsequent flowthrough collected. The column was then eluted with 1% AcOH in EtOH/H₂O (1:1, v/v) (50 mL).

### Results & Discussion

### Molecularly Imprinted Polymers

Molecularly imprinted polymers templated with either resveratrol 1 or 3,5-dihydroxy-N-(4-hydroxyphenyl)benzamide 2 were prepared using the functional monomer 4VP. The use of 4VP provided the opportunity for hydrogen bonding or ionic interactions between the electron rich pyridine nitrogen and the acidic phenolic groups in both of these template compounds. Aromatic π-π interactions may also participate in stabilising the pre-polymerisation complex which is subsequently ‘frozen’ during polymerization in the presence of the cross-linker EGDMA. The binding characteristics and performance of MIP_{RES} is reported above describing the preparation of an (E)-resveratrol selective MIP that is capable of the specific recognition of (E)-resveratrol present in a complex mixture comprising multiple structurally related analogues and other polyphenolics.

3,5-Dihydroxy-N-(4-hydroxyphenyl)benzamide 2 was synthesised as a template for preparing MIP_{AMIDE} that should generate “pseudo” (E)-resveratrol cavities within an imprinted polymer that are capable of binding not only the template molecule, but also (E)-resveratrol and potentially other structurally related compounds that are present in complex food waste matrices.

(E)-resveratrol saturation studies were conducted to determine the (E)-resveratrol binding affinity of MIP_{AMIDE}, (FIGS. 44A and 44B). Binding results for solutions containing (E)-resveratrol above 2 min, which are beyond the naturally occurring range of this compound, were not reproducible and are not shown. MIP_{AMIDE} displayed (E)-resveratrol binding capacity of approximately 15 μmol/g as determined from the asymptote of the static binding isotherm (FIG. 44A), which is about 20% lower than that obtained with the corresponding (E)-resveratrol-imprinted polymer. The extent of non-specific binding of (E)-resveratrol to NIP_{AMIDE} clearly shows that that the 3,5-Dihydroxy-N-(4-hydroxyphenyl)benzamide-imprinted cavities are responsible for the increased binding capacity of MIP_{AMIDE}. The selective binding capacity (expressed as MIP-NIP) for MIP_{AMIDE} (5-7 μmol/g, FIG. 44B) was however, considerably less than that observed for MIP_{RES} (9-11 μmol/g). This observation may be attributed to the 3-dimensional structural differences between the respective MIP cavities. These MIPs were generated using different template molecules, thus determining the complementary spatial orientation of the immobilized functional groups of the monomers within the respective MIP cavities and thereby maximizing the opportunity for interaction with their binding partners upon template rebinding. Hence, preparing MIP_{AMIDE} with the structurally similar resveratrol analogue 3,5-Dihydroxy-N-(4-hydroxyphenyl)benzamide 2 as the templating molecule produced molecular cavities having subtle differences in conformation with respect to the relative orientation of the functional monomers that reduced their capability to form strong interactions with the target molecule, (E)-resveratrol.

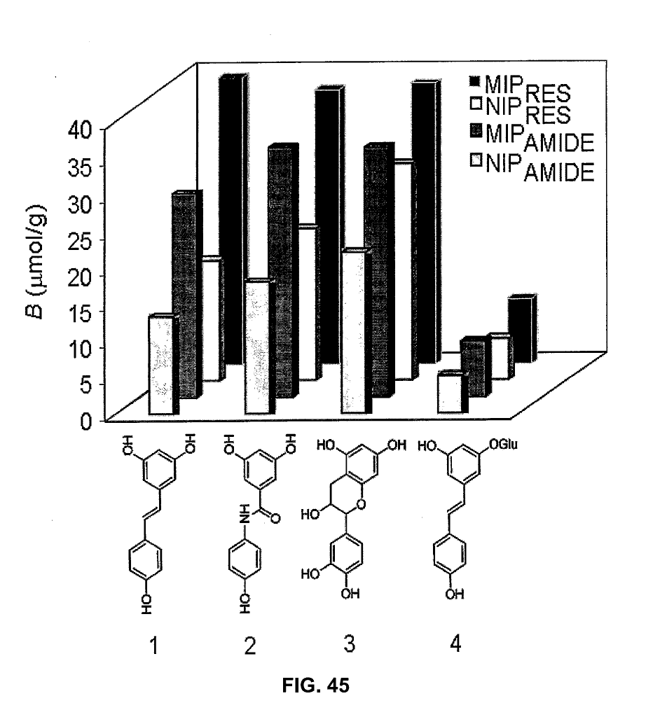

The cross-reactivity of both MIP_{RES} and MIP_{AMIDE} was evaluated via binding site interrogation using several structurally related polyphenols (FIG. 45). The order of selective recognition (MIP-NIP) of MIP_{RES} was (E)-resveratrol 1>amide 2>catechin 3>(E)-piceid 4, while the selective recognition of MIP_{AMIDE} was amide 2≧(E)-resveratrol 1>catechin 3>(E)-piceid 4. MIP_{AMIDE} displayed a high cross-reactivity towards (E)-resveratrol, displaying comparable selective binding for both this compound and the templating molecule 3,5-Dihydroxy-N-(4-hydroxyphenyl)benzamide 2. Both MIP_{RES} and MIP_{AMIDE} demonstrated similar binding capacities for catechin, albeit with reduced selectivity, that are comparable to binding of the respective template molecules. The presence of an increased number of possible H-bonding groups on the molecule resulted in a larger number of non-selective associations, thus leading to greater affinity by the NIPs, thereby decreasing specific recognition of catechin compared to that for the respective template molecules. MIP_{RES} and MIP_{AMIDE} displayed essentially no recognition for the glucosylated (E)-resveratrol molecule (E)-piceid, a result that is presumably due to the bulky glucose substituent preventing access to the MIP binding cavities.

### MISPE of Peanut Meal Extract

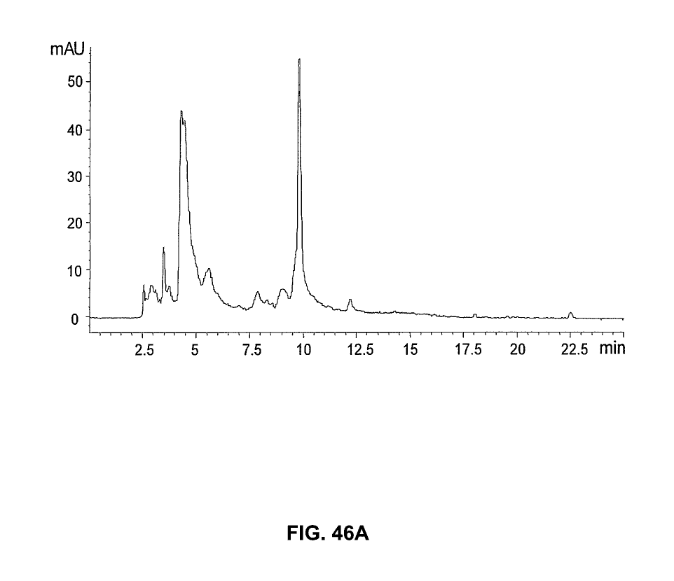

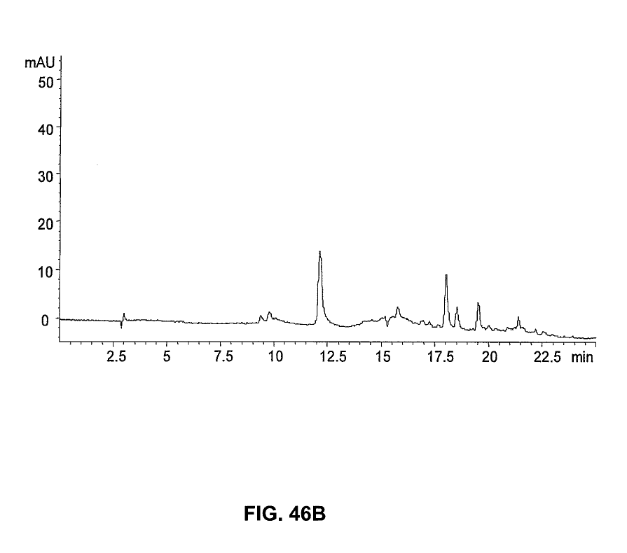

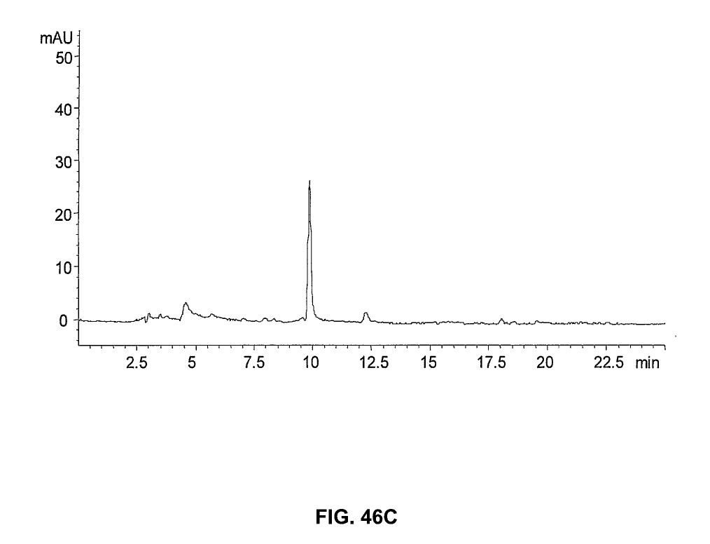

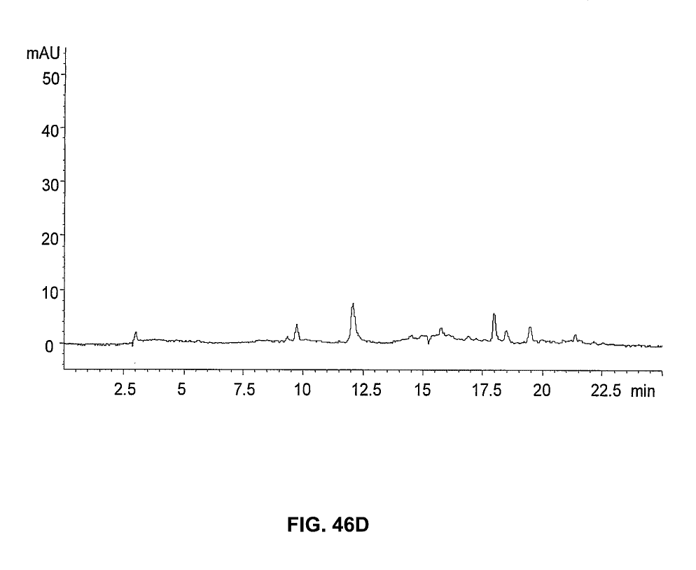

Peanut meal or peanut press is generated during peanut oil preparation and constitutes the remains of the peanut and husk after pressing. Peanuts contain variable amounts of (E)-resveratrol (0.02-1.79 μg/g) (11), amounts which exceed what we have observed in grape marc. Accordingly, binding of components present in a peanut meal extract, a complex mixture of bioactive polyphenols including (E)-resveratrol and catechin-based oligomers, was evaluated using MIP_{RES} and MIP_{AMIDE}. A liquid peanut meal extract was prepared and separately applied to either MIP_{RES} or MIP_{AMIDE} columns or the respective NIP controls. RP-HPLC chromatograms of the peanut meal extract and subsequent MIP eluates are shown in FIGS. 46A-46D. MIP_{RES} clearly significantly cleaned up the peanut meal extract and enriched (E)-resveratrol, which elutes at Rₜ=12.2 min, and several unidentified compounds (FIG. 46B). In contrast, MIP_{AMIDE} displayed preferential selectively for the unidentified compound(s) that eluted at Rₜ=9.7 min, whilst displaying negligible retention of (E)-resveratrol, which is present in the eluate at approximately similar levels as the peanut meal extract (FIG. 46C). Although MIP_{AMIDE} displays affinity for (E)-resveratrol as described above, the apparent lack of affinity for this molecule from complex mixtures may be a consequence of either higher affinity for other molecules that are present in the mixture or from Law of Mass Action effects arising from high abundance molecules having variable affinities for the MIP cavity. The level of non-specific binding of (E)-resveratrol by the NIP control column (FIG. 46D) was only marginally elevated above that present in the extract and also that bound by MIP_{AMIDE} although in the later case it is possible that the presence of the unknown effectively saturated the binding surface.

The eluate from MIP_{RES} (FIG. 46B) was analysed using tandem liquid chromatography-ESI-mass spectrometry (LC-ESI-MS) in positive ion mode, which confirmed that the peak at Rₜ=12 2 minutes consisted solely of (E)-resveratrol by comparison to an (E)-resveratrol standard which has a molecular mass of 229 m/z as the singly charged ion ([M+H]⁺). LC-ESI-MS was also used to examine the unknown compound(s) present in the MIP_{AMIDE} eluate at Rₜ=9.7 minutes, which revealed that this peak comprises a compound having a molecular mass of 577 m/z ([M+H]⁺) that is consistent with that of A-type procyanidins. Low molecular weight catechin oligomers, of which the A-type procyanidins predominate, are known antioxidants that are present in peanut skin in quantities approaching 9.5% by mass (12).

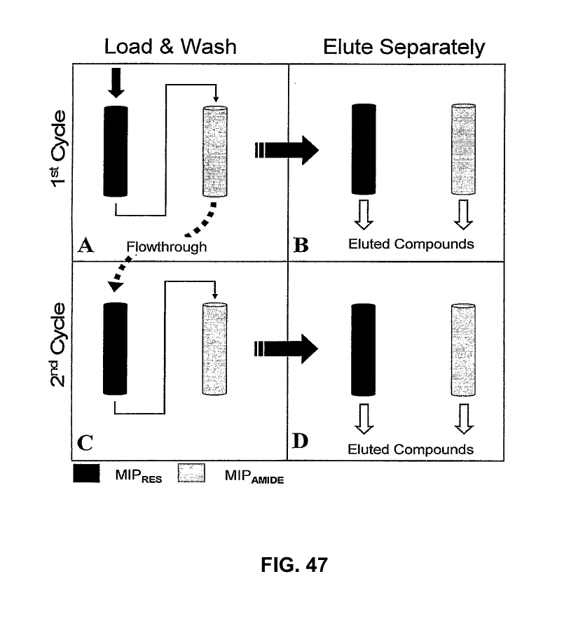

The concept of employing multiple MIPs in a tandem separation and isolation strategy, whereby multiple bioactive components may be captured and isolated from a single source, was explored as outlined in FIG. 47. The peanut meal extract was loaded onto two MIPs in sequence, MIP_{RES} followed by MIP_{AMIDE}, to isolate the primary molecule for which the MIPs exhibit the greatest selectivity. Re-loading of the depleted peanut meal extract onto these same eluted and washed tandem MIPs, served to isolate other residual, but equally valuable, polyphenol compounds.

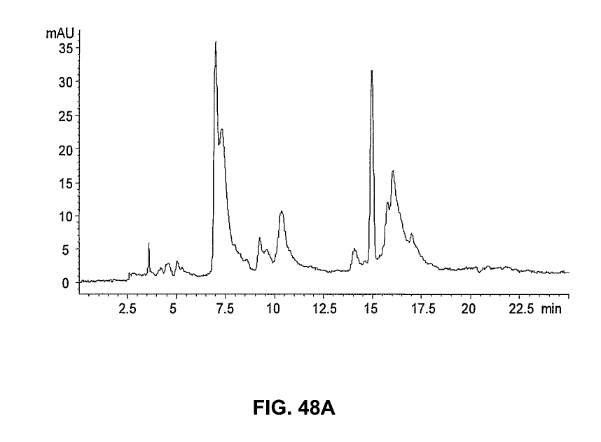

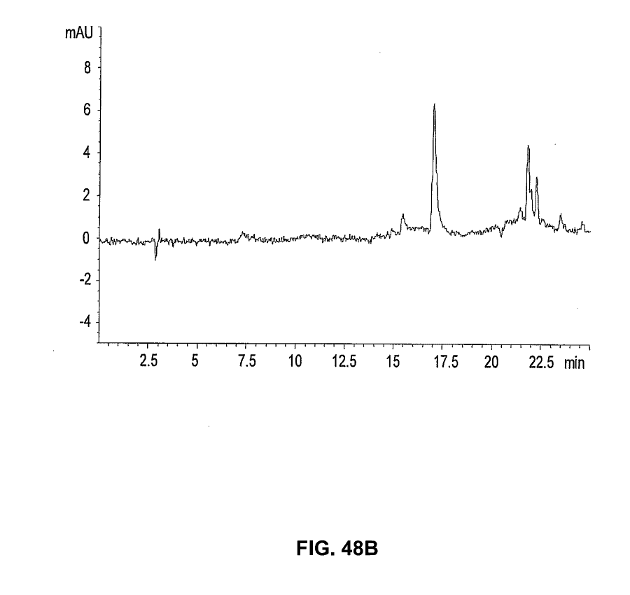

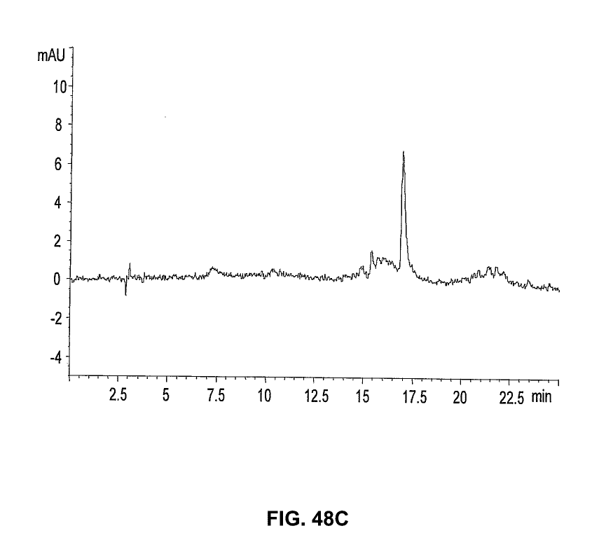

Typical RP-HPLC chromatograms from the tandem MISPE treatment of peanut meal extract are shown in FIGS. 48A-48C. The chromatogram of the peanut meal extract (FIG. 48A) shows a small peak for (E)-resveratrol at Rₜ=17 minutes, corresponding to 64 μg total (E)-resveratrol. A peak corresponding to (E)-resveratrol was similarly observed in the elutates from MIP_{RES} (FIG. 48B) and then from MIP_{AMIDE} (FIG. 48C). These results clearly illustrate the refining potential of MIP_{AMIDE} to also specifically capture (E)-resveratrol while excluding many of the non-bound compounds that remain in the flowthrough from MIP_{RES}.

The tandem combination of MIP_{RES} and MIP_{AMIDE} enabled the near quantitative (96%) recovery of resveratrol as summarised in Table 17. This result clearly signifies that both MIP columns are capable of selective retention and enrichment of (E)-resveratrol with almost complete recovery from the crude feed stock.

TABLE 17
Recovery of (E)-resveratrol from peanut meal extract by tandem MISPE in
different fractions of the eluant.

| | | |
|---|---|---|
| | Resveratrol | Total |
| Eluant Fractions | Recovery (μg)¹ | Recovery |

| (mL) | MIP_{RES} | MIP_{AMIDE} | μg | % |
|---|---|---|---|---|
| 1 | 1.61 | 0.78 | 2.39 | 3.72 |
| 2 | 5.16 | 4.35 | 9.51 | 14.83 |
| 3 | 5.47 | 5.14 | 10.61 | 16.54 |
| 4 | 4.40 | 5.78 | 10.18 | 15.88 |
| 5 | 3.01 | 3.68 | 6.69 | 10.43 |
| 6 | 2.34 | 3.01 | 5.35 | 8.34 |
| 7 | 2.43 | 2.38 | 4.81 | 7.50 |
| 8 | 2.26 | 2.09 | 4.35 | 6.78 |
| 9 | 1.91 | 1.57 | 3.48 | 5.43 |
| 10 | 1.80 | 0.69 | 2.49 | 3.88 |
| 11 | 1.39 | 0.46 | 1.85 | 2.88 |
| 12 | ND² | ND | ND | ND |
| Total | 31.81 | 29.93 | 61.71 | 96.21 |

¹Recovery determined for 1 mL reconstituted samples
²ND = Not detectable.

Surprisingly, the MIP_{AMIDE} column also displayed preferential selectivity towards (E)-resveratrol over the likely type A procyanidins (Rₜ=15 minutes, 577 m/z [M+H]⁺), a result in contrast to observations that showed that MIP_{AMIDE} preferentially bound the unknown compound compared to (E)-resveratrol (FIG. 46C). However, the tandem MISPE experiment was performed with a substantially larger peanut meal extract volume than that used in small-scale experiments. Therefore, it is apparent that MIP_{AMIDE} has selectively retained the larger quantity of (E)-resveratrol present that is in this peanut meal extract to the exclusion of procyanidin and that the MIP_{AMIDE} binding sites are effectively saturated with (E)-resveratrol, to the exclusion of other compounds which may be competing for these MIP_{AMIDE} binding sites. Our observation that MIP_{AMIDE} is capable of binding both (E)-resveratrol and procyanidin in small scale experiments clearly reflects the fact that saturation of available MIP_{AMIDE} binding sites was not achieved by the small quantities of available (E)-resveratrol thereby allowing procyanidin to bind to the remaining unoccupied MIP_{AMIDE} binding sites. Further, that procyanidin did not bind at all in the large scale experiment signifies that MIP_{AMIDE} exhibits lower affinity, specificity and selectivity for procyanidin than for (E)-resveratrol.

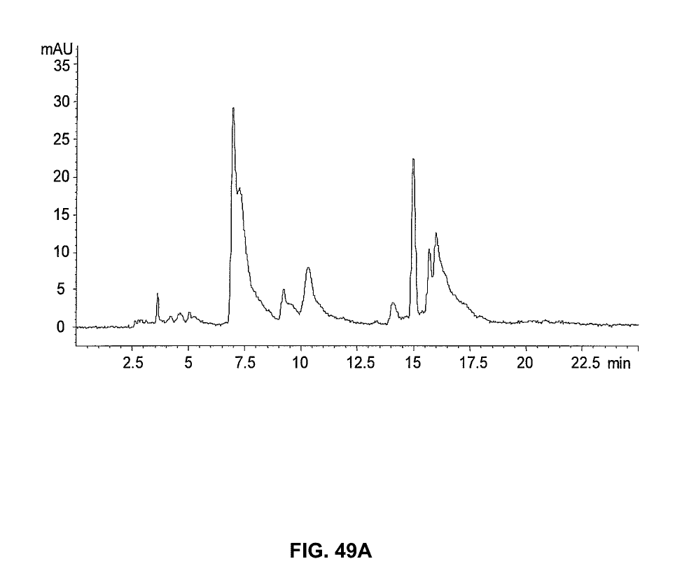

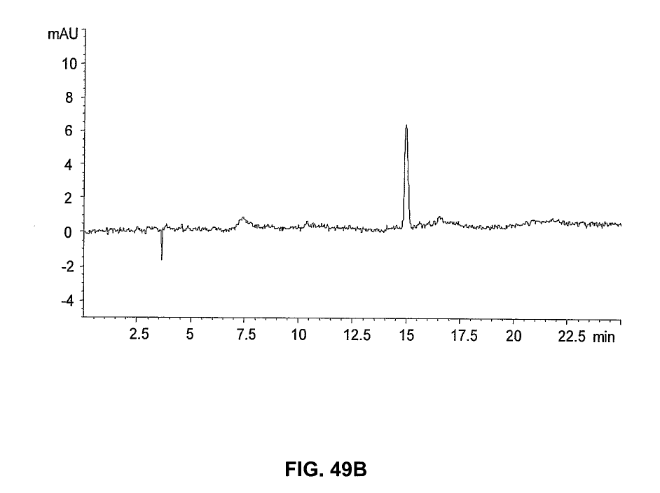

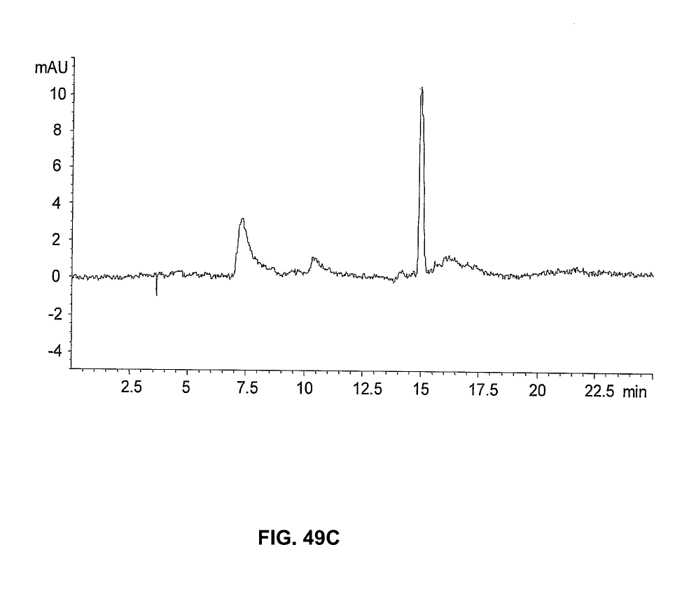

Since the tandem MISPE columns had selectively removed most of the (E)-resveratrol (96%) from the initial feed stock, it was anticipated that with diminished competition for available MIP binding sites both MIP_{RES} and MIP_{AMIDE} would selectively bind the procyanidin remaining in the resveratrol-depleted peanut meal extract. Consequently, the flowthrough and wash fractions from the processed peanut meal extract were combined and re-applied to the reconditioned tandem MIP_{RES} and MIP_{AMIDE} columns. The RP-HPLC chromatograms of the extracts recovered from the MIP_{RES} and MIP_{AMIDE} columns, obtained using the same conditions as above, showed that (E)-resveratrol did not bind or elute from either column (FIGS. 49A-49C). However, as expected, the peak corresponding to procyanidin (R_{f}=15 min) was clearly present in the chromatograms from both MIP columns Both MIP_{RES} and MIP_{AMIDE} retained similar amounts of procyanidin (FIGS. 49B and 49C). These results confirm that (i) all of the measurable (E)-resveratrol had been successfully removed from the extract by the first round of tandem MISPE treatment of the peanut meal extract and (ii) that both MIP_{RES} and MIP_{AMIDE} could successfully concentrate the A-type procyanidins upon the depletion of (E)-resveratrol from this extract.

### CONCLUSIONS

The findings reported here demonstrate the ability of molecularly imprinted polymers (MIPs) to selectively fractionate a mixture comprising polyphenols such as resveratrol and A-type procyanidins from peanut meal extract without the requirement for extensive sample pre-treatment. These MIPs may also be utilized in such a manner that more than one polyphenol may be separately isolated or enriched from the same feed stock with a rapid tandem MISPE (i.e., MIPs in series) approach.

### H. The Use of “Teabag” MIPs in Separating Components of a Complex Feedstock

This technology represents a new approach for the practical application of MIPs. Extraction of bioactives from processing waste streams (e.g., peanut by-products, winery grape seeds and skins, apple juice production wastes) may be a considerable logistic exercise, due to the large quantities of material, from geographically diverse locations, to isolate and return much smaller amounts of target bioactives. Therefore, it may be more advantageous to consider initial separation or pre-concentration of bulk-scale materials at the site of waste generation, which would result in material that is more easily handled and combined for final separations and purifications at a single dedicated processing facility. Alternatively, extraction may be performed at, or close to, the site of waste generation using low technology applications for the initial process of extraction of valuable materials from waste streams. An exemplar methodology could be based on the use of “tea bags” filled with MIPs for isolating target molecules from the feed stock.

“Teabags” for use in these investigations have been made from cotton-based materials, Gilson® 63 μm sieve mesh and Sigma Aldrich dialysis tubing cellulose membrane (12 kDa MWCO). The bags were held in an electroplated metal tea infuser to protect them from physical damage during stirring. They are robust for periods of several days and tolerate soaking in mixtures of ethanol, water and acetic acid.

Binding experiments using MIPs in a teabag were conducted. The MIPs used in the resveratrol binding experiments were MIP₈ (as numbered in the patent application), MIP_{E}, MIP_{AMIDE}, and MIP_{IMINE} as set out in the Examples above. The MIPs used in the phytosterol biding experiments were prepared using a 1:3:30 ratio of template:4-VP:EDGMA.

### Use of “Teabag” MIPs in Static Binding Systems for Phytosterols:

MIPs were templated with ergosterol, campesterol, cholesterol, stigmasterol, cholesteryl ferrulate, ferrulic acid and coumarin as described above. MIPs were packed in teabag polymer bags, and then placed in the crude plant extract for 18 hours. The teabag was subsequently removed from the mixture and transferred to a solution of 10% acetic acid in MeOH for 4 hours. The teabag was removed and the solution was evaporated to dryness; the resulting solid was reconstituted to approx 0.5 min in 20% ACN/MeOH, and analyzed by HPLC and ESI/MS. The performance MIPs were evaluated using extracts from avocado oil, sesame seed oil, wheat oil, grape seed oil, β-sitosterol vitamin supplement, and a green tea extract, derived from Lipton Green Tea™. The individual components extracted were identified. Several different polymers could be place singularly or severally in individual bags to simultaneously and concurrent extract the maximum amount and number of compounds of interest from the extract solutions. Recycling of individual MIPs was also established and the results demonstrated the reusability of these MIPs without significant deterioration in performance. The peaks in the ¹³C NMR spectrum allow for an analysis of the various captured and isolated phytosterols. For example, with the bound fraction eluted from a loaded campesterol-templated MIP, the peaks in the ¹³C NMR spectrum reveal only pure campesterol confirming that campesterol only has been specifically and selectively absorbed by this MIP. On the other hand with the cholesterol-templated MIP, the stigmasterol present in the crude extract was significantly bound suggesting that cholesterol can be used as a biomimetic template (in a manner analogous to the green resveratrol—the imine analogue of resveratrol), for molecular imprinting of more expensive and difficult to produce polymers.

### Use of “Teabag” MIPs in Static Binding Systems for Resveratrol

A corresponding experiment was carried out for binding of resveratrol to “teabag” MIPs using MIP₈ (as numbered in the patent application), MIP_{E}, MIP_{AMIDE}, and MIP_{IMINE} using the following feedstock examples: peanut skin and mash by-products, winery grape seeds and skins, apple juice production wastes and a green tea extract, derived from Lipton Green Tea™.

### I Sequential Application of MIPs

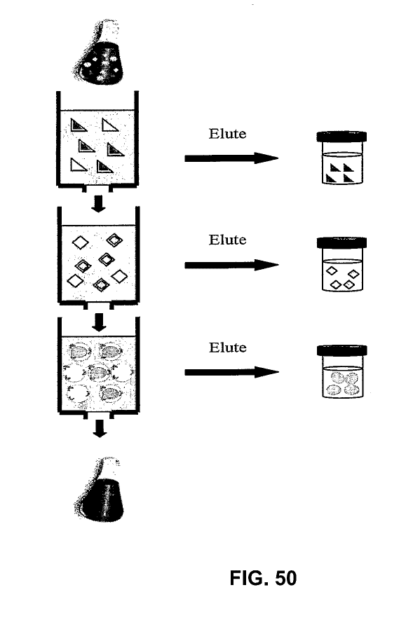

FIG. 50 provides an example of a sequential application of MIPs for the isolation of multiple targets from a feed extract.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### NON-PATENT REFERENCES

1. Xiang, H.-Y.; Zhou, C.-S.; Zhong, S.-A.; Lei, Q.-F., “Synthesis of resveratrol imprinted polymer and its application in separation of active ingredient in Polygonum cuspidatum extracts.” Yingyong Huaxue 2005, 22, (7), 739-743.
2. Ma, S.; Zhuang, X.; Wang, H.; Liu, H.; Li, J.; Dong, X., “Preparation and characterisation of trans-Resveratrol imprinted polymers.” Analytical Letters 2007, 40, 321-333.
3. Cao, H.; Xiao, J. B.; Xu, M., Evaluation of new selective molecularly imprinted polymers for the extraction of resveratrol from Polygonum cuspidatum. Macromolecular Research 2006, 14, (3), 324-330.
4. Ki, C. D.; Oh, C.; Oh, S.-G.; Chang, J. Y., “The use of a thermally reversible bond for molecular imprinting of silica spheres.” Journal of the American Chemical Society 2002, 124, (50), 14838-14839.
5. Spencer, A., “Selective Preparation of Non-Symmetrically Substituted Divinylbenzenes by Palladium Catalysed Arylations of Alkenes with Bromobenzoic Acid Derivatives”, J. Organomet. Chem., 1984, 265, 323-331
6. Romero-Peréz et al⁶. J. Agric. Food Chem., Vol. 49, No. 1, 2001, 210-215.
7. D. A. Learmonth, Bioconjugate Chem., 2003, 14, 262-267
8. Roberts et al, Eur. J. Med. Chem., (1994), 29, 841-854
9. Dolle, R. E.; Kruse, L. I., J. Org. Chem. 1985, 51, 4047-4053
10. Condo Jr, A. M.; Baker, D. C.; Moreau, R. A.; and Hicks, K. B., J. Agric. Food Chem., (2001), 49, 4961-4964, “Improved Method for the Synthesis of trans-Feruloyl-β-sitostanol.”

1-22. (canceled)

23. A method of at least partially separating a compound from a sample, by exposing the sample to a molecularly imprinted polymer (MIP);
wherein the compound comprises a trans-ethylene linker;
wherein the MIP has been prepared by a method comprising the steps of:
polymerizing a monomer comprising one or more non-covalent bonding sites and a cross-linking agent in the presence of a template and porogen; and
subsequently removing the template; and
wherein the template is an analogue of the compound, the template comprising an imine, amide or secondary amine linker in place of the trans-ethylene linker.

24. A method as claimed in claim 23, wherein the method comprises the steps of:
(i) preparing a chromatographic column comprising the MIP; (ii) passing the sample through the column; and (iii) collecting fractions of the sample from the column.

25. A method as claimed in claim 23, wherein the compound is a polyphenol.

26. A method as claimed in claim 25, wherein the compound is resveratrol.

27. A method as claimed in claim 23, wherein the template is (E)-5-[(4-hydroxy-phenylimino)-methyl]-benzene-1,3-diol or 3,5-dihydroxy-N-(4-hydroxyphenyl)benzamide.

28. A method as claimed in claim 23, wherein the MIP is encased in a permeable mesh.

29. A method as claimed in claim 23, wherein the sample is selected from the group consisting of agricultural waste, agricultural products, a chemical reaction mixture and a prepared mixture of compounds.

30. A method as claimed in claim 23, wherein the sample is a foodstuff.

31. A method as claimed in claim 23, wherein the MIP has been prepared using a pre-polymerisation complex, the pre-polymerisation complex comprising:
one or more monomers each comprising one or more non-covalent bonding sites; and
a template;
wherein the template comprises one or more non-covalent bonding sites complementary to the one or more non-covalent bonding sites of the monomer.

32. A method as claimed in claim 23, wherein the MIP comprises polymerized 4-vinylpyridine together with a polymerized cross-linking agent.
